(19) United States
(12) Patent Application Publication
(10) Pub. No.: US 2023/0128001 A1
(43) Pub. Date: Apr. 27, 2023

(54) HIV ANTIGENS AND MHC COMPLEXES

(71) Applicant: Gritstone bio, Inc., Emeryville, CA (US)

(72) Inventors: Roman Yelensky, Newton, MA (US); James Xin Sun, Newton, MA (US); Joshua Klein, Brookline, MA (US); Karin Jooss, Emeryville, CA (US); Ciaran Daniel Scallan, San Francisco, CA (US); Leonid Gitlin, Foster City, CA (US)

(21) Appl. No.: 17/817,312

(22) Filed: Aug. 3, 2022

Related U.S. Application Data
(63) Continuation of application No. 17/564,920, filed on Dec. 29, 2021, now abandoned.
Application No. 17/564,920 is a continuation of application No. PCT/US2020/040630, filed on Jul. 2, 2020.
(60) Provisional application No. 63/029,981, filed on May 26, 2020.
(60) Provisional application No. 62/869,877, filed on Jul. 2, 2019.

(51) Int. Cl. A61K 39/21; A61P 31/18; C12N 15/86

(52) U.S. Cl. CPC A61K 39/21; A61P 31/18; C12N 15/86; A61K 2039/545

(57) ABSTRACT

Disclosed herein are compositions that include antigen-encoding nucleic acid sequences and/or antigen peptides. Also disclosed are nucleotides, cells, and methods associated with the compositions including their use as vaccines against infectious diseases such as HIV.

### CROSS REFERENCE TO RELATED APPLICATIONS

This application is a continuation of U.S. application Ser. No. 17/564,920 filed Dec. 29, 2021, which is a continuation of International Patent Application No. PCT/US2020/040630 filed Jul. 2, 2020, which claims the benefit of and priority to U.S. Provisional Patent Application No. 62/869,877 filed Jul. 2, 2019 and U.S. Provisional Patent Application No. 63/029,981 filed on May 26, 2020, the entire disclosure of each of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Jun. 29, 2020, is named GSO-034WO_SL.txt and is 5,642,000 bytes in size.

### BACKGROUND

Infectious diseases, such as human immunodeficiency virus (HIV), remain difficult to prevent, treat, and/or cure. Therapeutic vaccines are promising, but for diseases such as HIV, they have not achieved therapeutic efficacy such that they can be deployed as a therapy or preventative vaccines for the population.

One question for vaccine design is how to identify and include the “best” therapeutic antigens for eliciting an anti-HIV response. Existing methods for identifying and predicting presentation of antigens have only achieved low positive predictive value (PPV) and serves as a significant impediment to vaccine design. If vaccines are designed using predictions with a low PPV, most patients are unlikely to receive a therapeutic antigen and fewer still are likely to receive more than one (even assuming all presented peptides are immunogenic). Thus, antigen vaccination with current methods is unlikely to succeed in preventing infections of infectious disease.

In addition to the challenges of current antigen prediction methods certain challenges also exist with the available vector systems that can be used for antigen delivery in humans, many of which are derived from humans. For example, many humans have pre-existing immunity to human viruses as a result of previous natural exposure, and this immunity can be a major obstacle to the use of recombinant human viruses for antigen delivery for treating infectious diseases.

### SUMMARY

Disclosed herein is a composition for delivery of an antigen expression system, the antigen expression system comprising: a vector backbone comprising a chimpanzee adenovirus vector, optionally wherein the chimpanzee adenovirus vector is a ChAdV68 vector, or an alphavirus vector, optionally wherein the alphavirus vector is a Venezuelan equine encephalitis virus vector, the vector backbone comprising at least one HIV MHC class I antigen-encoding nucleic acid sequence comprising a MHC class I epitope encoding nucleic acid sequence, optionally wherein the MHC class I epitope encoding nucleic acid sequence encodes a MHC class I epitope comprising at least one HIV epitope selected from the group consisting of the sequences shown in SEQ ID NOs: 325-22349. In various embodiments, the at least one HIV epitope is selected from the group consisting of the sequences shown in SEQ ID NOs: 4113, 4114, 4115, 4427, 4439, 4494, 4495, 4545, 4561, 4956, 4968, 4975, 4982, 5259, 5261, 5459, 5460, 5610, 5643, and 5661. In various embodiments, the antigen expression system comprises 2, 3, 4, 5, 6, 7, 8, 9, 10, 11, 12, 13, 14, 15, 16, 17, 18, 19, or 20 HIV MHC class I antigen-encoding nucleic acid sequences, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID Nos: 325-22349. In various embodiments, each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 4113, 4114, 4115, 4427, 4439, 4494, 4495, 4545, 4561, 4956, 4968, 4975, 4982, 5259, 5261, 5459, 5460, 5610, 5643, and 5661.

Additionally disclosed herein is a composition for delivery of one or more antigens, the composition comprising one or more HIV MHC class I antigens or one or more nucleic acid sequences encoding one or more HIV MHC class I antigens, each HIV MHC class I antigen comprising a MHC class I epitope comprising at least one HIV epitope selected from the group consisting of the sequences shown in SEQ ID Nos: 325-22349. In various embodiments, the at least one HIV epitope is selected from the group consisting of the sequences shown in SEQ ID NOs: 4113, 4114, 4115, 4427, 4439, 4494, 4495, 4545, 4561, 4956, 4968, 4975, 4982, 5259, 5261, 5459, 5460, 5610, 5643, and 5661. In various embodiments, the composition comprises 2, 3, 4, 5, 6, 7, 8, 9, 10, 11, 12, 13, 14, 15, 16, 17, 18, 19, or 20 HIV MHC class I antigens, wherein each HIV MHC class I antigen comprises a MHC class I epitope comprising at least one HIV epitope selected from the group consisting of the sequences shown in SEQ ID Nos: 325-22349. In various embodiments, each HIV MHC class I antigen comprises a MHC class I epitope comprising at least one HIV epitope selected from the group consisting of the sequences shown in SEQ ID NOs: 4113, 4114, 4115, 4427, 4439, 4494, 4495, 4545, 4561, 4956, 4968, 4975, 4982, 5259, 5261, 5459, 5460, 5610, 5643, and 5661.

In various embodiments, the MHC class I epitopes are selected by performing the steps of: (a) obtaining at least one of exome, transcriptome, or whole genome nucleotide sequencing, wherein the nucleotide sequencing data is used to obtain data representing peptide sequences of each of a set of antigens; (b) inputting the peptide sequence of each antigen into a presentation model to generate a set of numerical likelihoods that each of the antigens is presented by one or more of the MHC proteins, the set of numerical likelihoods having been identified at least based on received mass spectrometry data; and (c) selecting a subset of the set of antigens based on the set of numerical likelihoods to generate a set of selected antigens which are used to generate the MHC class I epitopes.

Additionally disclosed herein is a composition for delivery of an antigen expression system comprising one or more vectors, the one or more vectors comprising: (a) a vector backbone, wherein the backbone comprises: (i) at least one promoter nucleotide sequence, and (ii) at least one polyadenylation (poly(A)) sequence; and (b) an antigen cassette, wherein the antigen cassette comprises: (i) at least one antigen-encoding nucleic acid sequence, comprising: (I) at least one HIV MHC class I antigen-encoding nucleic acid sequence, comprising: (A) a MHC class I epitope encoding nucleic acid sequence, wherein the MHC class I epitope encoding nucleic acid sequence encodes a MHC class I epitope comprising at least one HIV epitope selected from the group consisting of the sequences shown in SEQ ID Nos: 325-22349, (B) optionally, a 5′ linker sequence, and (C) optionally, a 3′ linker sequence; (ii) optionally, a second promoter nucleotide sequence operably linked to the antigen-encoding nucleic acid sequence; and (iii) optionally, at least one MHC class II antigen-encoding nucleic acid sequence; (iv) optionally, at least one nucleic acid sequence encoding a GPGPG (SEQ ID NO: 57) amino acid linker sequence; and (v) optionally, at least one second poly(A) sequence, wherein the second poly(A) sequence is a native poly(A) sequence or an exogenous poly(A) sequence to the vector backbone.

Additionally disclosed herein is a composition for delivery of an antigen expression system comprising one or more vectors, the one or more vectors comprising: (a) a vector backbone, wherein the backbone comprises: (i) at least one promoter nucleotide sequence, and (ii) at least one polyadenylation (poly(A)) sequence; and (b) an antigen cassette, wherein the antigen cassette comprises: (i) at least one antigen-encoding nucleic acid sequence, comprising: (I) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 325-2165, wherein each of the HIV MHC class I antigen-encoding nucleic acid sequences further comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II antigen-encoding nucleic acid sequence; (iv) optionally, at least one nucleic acid sequence encoding a GPGPG (SEQ ID NO: 58) amino acid linker sequence; and (v) optionally, at least one second poly(A) sequence, wherein the second poly(A) sequence is a native poly(A) sequence or an exogenous poly(A) sequence to the vector backbone.

Additionally disclosed herein is a composition for delivery of an antigen expression system comprising one or more vectors, the one or more vectors comprising: (a) a vector backbone, wherein the backbone comprises: (i) at least one promoter nucleotide sequence, and (ii) at least one polyadenylation (poly(A)) sequence; and (b) an antigen cassette, wherein the antigen cassette comprises: (i) at least one antigen-encoding nucleic acid sequence, comprising: (I) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 2166-4106, wherein each of the HIV MHC class I antigen-encoding nucleic acid sequences further comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II antigen-encoding nucleic acid sequence; (iv) optionally, at least one nucleic acid sequence encoding a GPGPG (SEQ ID NO: 59) amino acid linker sequence; and (v) optionally, at least one second poly(A) sequence, wherein the second poly(A) sequence is a native poly(A) sequence or an exogenous poly(A) sequence to the vector backbone.

Additionally disclosed herein is a composition for delivery of an antigen expression system comprising one or more vectors, the one or more vectors comprising: (a) a vector backbone, wherein the backbone comprises: (i) at least one promoter nucleotide sequence, and (ii) at least one polyadenylation (poly(A)) sequence; and (b) an antigen cassette, wherein the antigen cassette comprises: (i) at least one antigen-encoding nucleic acid sequence, comprising: (I) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 4107-6241, wherein each of the HIV MHC class I antigen-encoding nucleic acid sequences further comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II antigen-encoding nucleic acid sequence; (iv) optionally, at least one nucleic acid sequence encoding a GPGPG (SEQ ID NO: 60) amino acid linker sequence; and (v) optionally, at least one second poly(A) sequence, wherein the second poly(A) sequence is a native poly(A) sequence or an exogenous poly(A) sequence to the vector backbone.

Additionally disclosed herein is a composition for delivery of an antigen expression system comprising one or more vectors, the one or more vectors comprising: (a) a vector backbone, wherein the backbone comprises: (i) at least one promoter nucleotide sequence, and (ii) at least one polyadenylation (poly(A)) sequence; and (b) an antigen cassette, wherein the antigen cassette comprises: (i) at least one antigen-encoding nucleic acid sequence, comprising: (I) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 6242-8389, wherein each of the HIV MHC class I antigen-encoding nucleic acid sequences further comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II antigen-encoding nucleic acid sequence; (iv) optionally, at least one nucleic acid sequence encoding a GPGPG (SEQ ID NO: 61) amino acid linker sequence; and (v) optionally, at least one second poly(A) sequence, wherein the second poly(A) sequence is a native poly(A) sequence or an exogenous poly(A) sequence to the vector backbone.

Additionally disclosed herein is a composition for delivery of an antigen expression system comprising one or more vectors, the one or more vectors comprising: (a) a vector backbone, wherein the backbone comprises: (i) at least one promoter nucleotide sequence, and (ii) at least one polyadenylation (poly(A)) sequence; and (b) an antigen cassette, wherein the antigen cassette comprises: (i) at least one antigen-encoding nucleic acid sequence, comprising: (I) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 8930-10626, wherein each of the HIV MHC class I antigen-encoding nucleic acid sequences further comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II antigen-encoding nucleic acid sequence; (iv) optionally, at least one nucleic acid sequence encoding a GPGPG (SEQ ID NO: 62) amino acid linker sequence; and (v) optionally, at least one second poly(A) sequence, wherein the second poly(A) sequence is a native poly(A) sequence or an exogenous poly(A) sequence to the vector backbone.

A composition for delivery of an antigen expression system comprising one or more vectors, the one or more vectors comprising: (a) a vector backbone, wherein the backbone comprises: (i) at least one promoter nucleotide sequence, and (ii) at least one polyadenylation (poly(A)) sequence; and (b) an antigen cassette, wherein the antigen cassette comprises: (i) at least one antigen-encoding nucleic acid sequence, comprising: (I) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 10627-12810, wherein each of the HIV MHC class I antigen-encoding nucleic acid sequences further comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II antigen-encoding nucleic acid sequence; (iv) optionally, at least one nucleic acid sequence encoding a GPGPG (SEQ ID NO: 63) amino acid linker sequence; and (v) optionally, at least one second poly(A) sequence, wherein the second poly(A) sequence is a native poly(A) sequence or an exogenous poly(A) sequence to the vector backbone.

Additionally disclosed herein is a composition for delivery of an antigen expression system comprising one or more vectors, the one or more vectors comprising: (a) a vector backbone, wherein the backbone comprises: (i) at least one promoter nucleotide sequence, and (ii) at least one polyadenylation (poly(A)) sequence; and (b) an antigen cassette, wherein the antigen cassette comprises: (i) at least one antigen-encoding nucleic acid sequence, comprising: (I) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 12811-15079, wherein each of the HIV MHC class I antigen-encoding nucleic acid sequences further comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II antigen-encoding nucleic acid sequence; (iv) optionally, at least one nucleic acid sequence encoding a GPGPG (SEQ ID NO: 64) amino acid linker sequence; and (v) optionally, at least one second poly(A) sequence, wherein the second poly(A) sequence is a native poly(A) sequence or an exogenous poly(A) sequence to the vector backbone.

Additionally disclosed herein is a composition for delivery of an antigen expression system comprising one or more vectors, the one or more vectors comprising: (a) a vector backbone, wherein the backbone comprises: (i) at least one promoter nucleotide sequence, and (ii) at least one polyadenylation (poly(A)) sequence; and (b) an antigen cassette, wherein the antigen cassette comprises: (i) at least one antigen-encoding nucleic acid sequence, comprising: (I) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 15080-17174, wherein each of the HIV MHC class I antigen-encoding nucleic acid sequences further comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II antigen-encoding nucleic acid sequence; (iv) optionally, at least one nucleic acid sequence encoding a GPGPG (SEQ ID NO: 65) amino acid linker sequence; and (v) optionally, at least one second poly(A) sequence, wherein the second poly(A) sequence is a native poly(A) sequence or an exogenous poly(A) sequence to the vector backbone.

Additionally disclosed herein is a composition for delivery of an antigen expression system comprising one or more vectors, the one or more vectors comprising: (a) a vector backbone, wherein the backbone comprises: (i) at least one promoter nucleotide sequence, and (ii) at least one polyadenylation (poly(A)) sequence; and (b) an antigen cassette, wherein the antigen cassette comprises: (i) at least one antigen-encoding nucleic acid sequence, comprising: (I) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 17175-19388, wherein each of the HIV MHC class I antigen-encoding nucleic acid sequences further comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II antigen-encoding nucleic acid sequence; (iv) optionally, at least one nucleic acid sequence encoding a GPGPG (SEQ ID NO: 66) amino acid linker sequence; and (v) optionally, at least one second poly(A) sequence, wherein the second poly(A) sequence is a native poly(A) sequence or an exogenous poly(A) sequence to the vector backbone.

Additionally disclosed herein is a composition for delivery of an antigen expression system comprising one or more vectors, the one or more vectors comprising: (a) a vector backbone, wherein the backbone comprises: (i) at least one promoter nucleotide sequence, and (ii) at least one polyadenylation (poly(A)) sequence; and (b) an antigen cassette, wherein the antigen cassette comprises: (i) at least one antigen-encoding nucleic acid sequence, comprising: (I) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 19389-21003, wherein each of the HIV MHC class I antigen-encoding nucleic acid sequences further comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II antigen-encoding nucleic acid sequence; (iv) optionally, at least one nucleic acid sequence encoding a GPGPG (SEQ ID NO: 67) amino acid linker sequence; and (v) optionally, at least one second poly(A) sequence, wherein the second poly(A) sequence is a native poly(A) sequence or an exogenous poly(A) sequence to the vector backbone.

Additionally disclosed herein is a composition for delivery of an antigen expression system comprising one or more vectors, the one or more vectors comprising: (a) a vector backbone, wherein the backbone comprises: (i) at least one promoter nucleotide sequence, and (ii) at least one polyadenylation (poly(A)) sequence; and (b) an antigen cassette, wherein the antigen cassette comprises: (i) at least one antigen-encoding nucleic acid sequence, comprising: (I) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 21004-22349, wherein each of the HIV MHC class I antigen-encoding nucleic acid sequences further comprises; (A) optionally, a 5′ linker sequence, and (B) optionally, a 3′ linker sequence; (ii) optionally, a second promoter nucleotide sequence operably linked to the antigen-encoding nucleic acid sequence; and (iii) optionally, at least one MHC class II antigen-encoding nucleic acid sequence; (iv) optionally, at least one nucleic acid sequence encoding a GPGPG (SEQ ID NO: 68) amino acid linker sequence; and (v) optionally, at least one second poly(A) sequence, wherein the second poly(A) sequence is a native poly(A) sequence or an exogenous poly(A) sequence to the vector backbone.

Additionally disclosed herein is a composition for delivery of an antigen expression system comprising one or more vectors, the one or more vectors comprising: (a) a vector backbone, wherein the vector backbone comprises (i) a chimpanzee adenovirus vector, optionally wherein the chimpanzee adenovirus vector is a ChAdV68 vector, or an alphavirus vector, optionally wherein the alphavirus vector is a Venezuelan equine encephalitis virus vector, and (ii) a 26S promoter nucleotide sequence, and (iii) a polyadenylation (poly(A)) sequence; and (b) an antigen cassette integrated between the 26S promoter nucleotide sequence and the poly(A) sequence, wherein the antigen cassette comprises: (i) at least one antigen-encoding nucleic acid sequence, comprising: (I) at least 10, 11, 12, 13, 14, 15, 16, 17, 18, 19, or 20 HIV MHC class I antigen-encoding nucleic acid sequences linearly linked to each other and each comprising: (A) a MHC class I epitope encoding nucleic acid sequence, wherein the MHC class I epitope encoding nucleic acid sequence encodes a MHC class I epitope 7-15 amino acids in length, and wherein at least one of the MHC class I epitopes is selected from the group consisting of epitope sequences of any one of SEQ ID Nos: 325-22349, (B) a 5′ linker sequence, wherein the 5′ linker sequence encodes a native N-terminal amino acid sequence of the MHC class I epitope, and wherein the 5′ linker sequence encodes a peptide that is at least 3 amino acids in length, (C) a 3′ linker sequence, wherein the 3′ linker sequence encodes a native C-terminal acid sequence of the MHC class I epitope, and wherein the 3′ linker sequence encodes a peptide that is at least 3 amino acids in length, and wherein the antigen cassette is operably linked to the 26S promoter nucleotide sequence, wherein each of the MHC class I antigen-encoding nucleic acid sequences encodes a polypeptide that is between 13 and 25 amino acids in length, and wherein each 3′ end of each MHC class I antigen-encoding nucleic acid sequence is linked to the 5′ end of the following MHC class I antigen-encoding nucleic acid sequence with the exception of the final MHC class I antigen-encoding nucleic acid sequence in the antigen cassette; and (ii) at least two MHC class II antigen-encoding nucleic acid sequences comprising: (I) a PADRE MHC class II sequence, (II) a Tetanus toxoid MHC class II sequence, (III) a first nucleic acid sequence encoding a GPGPG (SEQ ID NO: 69) amino acid linker sequence linking the PADRE MHC class II sequence and the Tetanus toxoid MHC class II sequence, (IV) a second nucleic acid sequence encoding a GPGPG (SEQ ID NO: 70) amino acid linker sequence linking the 5′ end of the at least two MHC class II antigen-encoding nucleic acid sequences to the HIV MHC class I antigen-encoding nucleic acid sequences, (V) optionally, a third nucleic acid sequence encoding a GPGPG (SEQ ID NO: 71) amino acid linker sequence at the 3′ end of the at least two MHC class II antigen-encoding nucleic acid sequences.

In various embodiments, an ordered sequence of each element of the antigen cassette is described in the formula, from 5′ to 3′, comprising Pₐ-(L5_{b}-N_{c}-L3_{d})_{X}-(G5ₑ-U_{f})_{Y}-G3_{g} wherein P comprises the second promoter nucleotide sequence, where a=0 or 1, N comprises one of the MHC class I epitope encoding nucleic acid sequences, where c=1, L5 comprises the 5′ linker sequence, where b=0 or 1, L3 comprises the 3′ linker sequence, where d=0 or 1, G5 comprises one of the at least one nucleic acid sequences encoding a GPGPG (SEQ ID NO: 72) amino acid linker, where e=0 or 1, G3 comprises one of the at least one nucleic acid sequences encoding a GPGPG (SEQ ID NO: 73) amino acid linker, where g=0 or 1, U comprises one of the at least one MHC class II antigen-encoding nucleic acid sequence, where f=1, X=1 to 400, where for each X the corresponding N_{c} is a epitope encoding nucleic acid sequence, and Y=0, 1, or 2, where for each Y the corresponding U_{f} is an antigen-encoding nucleic acid sequence.

In various embodiments, for each X the corresponding N_{c} is a distinct MHC class I epitope encoding nucleic acid sequence. In various embodiments, for each Y the corresponding U_{f} is a distinct MHC class II antigen-encoding nucleic acid sequence.

In various embodiments, a=0, b=1, d=1, e=1, g=1, h=1, X=20, Y=2, the at least one promoter nucleotide sequence is a single 26S promoter nucleotide sequence provided by the backbone, the at least one polyadenylation poly(A) sequence is a poly(A) sequence of at least 100 consecutive A nucleotides (SEQ ID NO: 74) provided by the backbone, each N encodes a MHC class I epitope 7-15 amino acids in length, L5 is a native 5′ linker sequence that encodes a native N-terminal amino acid sequence of the MHC I epitope, and wherein the 5′ linker sequence encodes a peptide that is at least 3 amino acids in length, L3 is a native 3′ linker sequence that encodes a native nucleic-terminal acid sequence of the MHC I epitope, and wherein the 3′ linker sequence encodes a peptide that is at least 3 amino acids in length, U is each of a PADRE class II sequence and a Tetanus toxoid MHC class II sequence, the vector backbone comprises a chimpanzee adenovirus vector, optionally wherein the chimpanzee adenovirus vector is a ChAdV68 vector, or an alphavirus vector, optionally wherein the alphavirus vector is a Venezuelan equine encephalitis virus vector, and each of the MHC class I antigen-encoding nucleic acid sequences encodes a polypeptide that is between 13 and 25 amino acids in length.

In various embodiments, the composition further comprising a nanoparticulate delivery vehicle. In various embodiments, the nanoparticulate delivery vehicle is a lipid nanoparticle (LNP). In various embodiments, the LNP comprises ionizable amino lipids. In various embodiments, the ionizable amino lipids comprise MC3-like (dilinoleylmethyl-4-dimethylaminobutyrate) molecules. In various embodiments, the nanoparticulate delivery vehicle encapsulates the antigen expression system. In various embodiments, the antigen cassette is integrated between the at least one promoter nucleotide sequence and the at least one poly(A) sequence. In various embodiments, wherein the at least one promoter nucleotide sequence is operably linked to the antigen-encoding nucleic acid sequence. In various embodiments, the one or more vectors comprise one or more +-stranded RNA vectors. In various embodiments, the one or more +-stranded RNA vectors comprise a 5′ 7-methylguanosine (m7g) cap. In various embodiments, the one or more +-stranded RNA vectors are produced by in vitro transcription. In various embodiments, the one or more vectors are self-replicating within a mammalian cell. In various embodiments, the backbone comprises at least one nucleotide sequence of an Aura virus, a Fort Morgan virus, a Venezuelan equine encephalitis virus, a Ross River virus, a Semliki Forest virus, a Sindbis virus, or a Mayaro virus. In various embodiments, the backbone comprises at least one nucleotide sequence of a Venezuelan equine encephalitis virus. In various embodiments, the backbone comprises at least sequences for nonstructural protein-mediated amplification, a 26S promoter sequence, a poly(A) sequence, a nonstructural protein 1 (nsP1) gene, a nsP2 gene, a nsP3 gene, and a nsP4 gene encoded by the nucleotide sequence of the Aura virus, the Fort Morgan virus, the Venezuelan equine encephalitis virus, the Ross River virus, the Semliki Forest virus, the Sindbis virus, or the Mayaro virus. In various embodiments, the backbone comprises at least sequences for nonstructural protein-mediated amplification, a 26S promoter sequence, and a poly(A) sequence encoded by the nucleotide sequence of the Aura virus, the Fort Morgan virus, the Venezuelan equine encephalitis virus, the Ross River virus, the Semliki Forest virus, the Sindbis virus, or the Mayaro virus.

In various embodiments, sequences for nonstructural protein-mediated amplification are selected from the group consisting of: an alphavirus 5′ UTR, a 51-nt CSE, a 24-nt CSE, a 26S subgenomic promoter sequence, a 19-nt CSE, an alphavirus 3′ UTR, or combinations thereof. In various embodiments, the backbone does not encode structural virion proteins capsid, E2 and E1. In various embodiments, the antigen cassette is inserted in place of structural virion proteins within the nucleotide sequence of the Aura virus, the Fort Morgan virus, the Venezuelan equine encephalitis virus, the Ross River virus, the Semliki Forest virus, the Sindbis virus, or the Mayaro virus. In various embodiments, the Venezuelan equine encephalitis virus comprises the sequence of SEQ ID NO:3 or SEQ ID NO:5 In various embodiments, the Venezuelan equine encephalitis virus comprises the sequence of SEQ ID NO:3 or SEQ ID NO:5 further comprising a deletion between base pair 7544 and 11175. In various embodiments, the backbone comprises the sequence set forth in SEQ ID NO:6 or SEQ ID NO:7

In various embodiments, the antigen cassette is inserted at position 7544 to replace the deletion between base pairs 7544 and 11175 as set forth in the sequence of SEQ ID NO:3 or SEQ ID NO:5 In various embodiments, the insertion of the antigen cassette provides for transcription of a polycistronic RNA comprising the nsP1-4 genes and the at least one antigen-encoding nucleic acid sequence, wherein the nsP1-4 genes and the at least one antigen-encoding nucleic acid sequence are in separate open reading frames. In various embodiments, the backbone comprises at least one nucleotide sequence of a chimpanzee adenovirus vector. In various embodiments, the chimpanzee adenovirus vector is a ChAdV68 vector. In various embodiments, the at least one promoter nucleotide sequence is the native 26S promoter nucleotide sequence encoded by the backbone. In various embodiments, the at least one promoter nucleotide sequence is an exogenous RNA promoter. In various embodiments, the second promoter nucleotide sequence is a 26S promoter nucleotide sequence. In various embodiments, the second promoter nucleotide sequence comprises multiple 26S promoter nucleotide sequences, wherein each 26S promoter nucleotide sequence provides for transcription of one or more of the separate open reading frames. In various embodiments, the one or more vectors are each at least 300nt in size.

In various embodiments, the one or more vectors are each at least 1 kb in size. In various embodiments, the one or more vectors are each 2 kb in size. In various embodiments, the one or more vectors are each less than 5 kb in size. In various embodiments, at least one of the at least one antigen-encoding nucleic acid sequences encodes a polypeptide sequence or portion thereof that is presented by MHC class I protein. In various embodiments, each antigen-encoding nucleic acid sequence is linked directly to one another. In various embodiments, at least one of the at least one antigen-encoding nucleic acid sequences is linked to a distinct antigen-encoding nucleic acid sequence with a nucleic acid sequence encoding a linker. In various embodiments, the linker links two MHC class I epitope sequences or an MHC class I epitope sequence to an MHC class II sequence. In various embodiments, the linker is selected from the group consisting of: (1) consecutive glycine residues, at least 2, 3, 4, 5, 6, 7, 8, 9, or 10 residues in length (SEQ ID NO: 75); (2) consecutive alanine residues, at least 2, 3, 4, 5, 6, 7, 8, 9, or 10 residues in length (SEQ ID NO: 76); (3) two arginine residues (RR); (4) alanine, alanine, tyrosine (AAY); (5) a consensus sequence at least 2, 3, 4, 5, 6, 7, 8, 9, or 10 amino acid residues in length that is processed efficiently by a mammalian proteasome; and (6) one or more native sequences flanking the antigen derived from the cognate protein of origin and that is at least 2, 3, 4, 5, 6, 7, 8, 9, 10, 11, 12, 13, 14, 15, 16, 17, 18, 19, 20, or 2-20 amino acid residues in length. In various embodiments, the linker links two MHC class II sequences or an MHC class II sequence to an MHC class I epitope sequence. In various embodiments, the linker comprises the sequence GPGPG (SEQ ID NO: 77). In various embodiments, at least one sequence of the at least one antigen-encoding nucleic acid sequences is linked, operably or directly, to a separate or contiguous sequence that enhances the expression, stability, cell trafficking, processing and presentation, and/or immunogenicity of the at least one antigen-encoding nucleic acid sequences.

In various embodiments, the separate or contiguous sequence comprises at least one of: a ubiquitin sequence, a ubiquitin sequence modified to increase proteasome targeting (e.g., the ubiquitin sequence contains a Gly to Ala substitution at position 76), an immunoglobulin signal sequence (e.g., IgK), a major histocompatibility class I sequence, lysosomal-associated membrane protein (LAMP)-1, human dendritic cell lysosomal-associated membrane protein, and a major histocompatibility class II sequence; optionally wherein the ubiquitin sequence modified to increase proteasome targeting is A76. In various embodiments, the at least one antigen-encoding nucleic acid sequence comprises at least 2-10, 2, 3, 4, 5, 6, 7, 8, 9, 10, 11, 12, 13, 14, 15, 16, 17, 18, 19, or 20 nucleic acid sequences. In various embodiments, the at least one HIV MHC class I antigen-encoding nucleic acid sequence or the at least one antigen-encoding nucleic acid sequence comprises at least 15-20, 11-100, 11-200, 11-300, 11-400, or up to 400 nucleic acid sequences. In various embodiments, wherein the at least one HIV MHC class I antigen-encoding nucleic acid sequence or the at least one antigen-encoding nucleic acid sequence comprises at least 2-400 nucleic acid sequences and wherein at least two of the antigen-encoding nucleic acid sequences encode polypeptide sequences or portions thereof that are presented by MHC class I protein. In various embodiments, at least two of the antigen-encoding nucleic acid sequences encode polypeptide sequences or portions thereof that are presented by MHC class I protein.

In various embodiments, when administered to the subject and translated, at least one of the antigens encoded by the at least one HIV MHC class I antigen-encoding nucleic acid or the at least one of the MHC class I epitopes are presented on antigen presenting cells resulting in an immune response. In various embodiments, the at least one HIV MHC class I antigen-encoding nucleic acid sequence, when administered to the subject and translated, at least one of the antigens are presented on antigen presenting cells resulting in an immune response, and optionally wherein the expression of each of the at least one antigen-encoding nucleic acid sequences is driven by the at least one promoter nucleotide sequence. In various embodiments, each MHC class I antigen-encoding nucleic acid sequence encodes a polypeptide sequence between 8 and 35 amino acids in length, optionally 9-17, 9-25, 8, 9, 10, 11, 12, 13, 14, 15, 16, 17, 18, 19, 20, 21, 22, 23, 24, 25, 26, 27, 28, 29, 30, 31, 32, 33, 34 or 35 amino acids in length. In various embodiments, the at least one MHC class II antigen-encoding nucleic acid sequence is present. In various embodiments, the at least one MHC class II antigen-encoding nucleic acid sequence is 12-20, 12, 13, 14, 15, 16, 17, 18, 19, 20, or 20-40 amino acids in length. In various embodiments, the at least one MHC class II antigen-encoding nucleic acid sequence is present and comprises at least one universal MHC class II antigen-encoding nucleic acid sequence, optionally wherein the at least one universal sequence comprises at least one of Tetanus toxoid and PADRE. In various embodiments, the at least one promoter nucleotide sequence or the second promoter nucleotide sequence is inducible. In various embodiments, the at least one promoter nucleotide sequence or the second promoter nucleotide sequence is non-inducible. In various embodiments, the at least one poly(A) sequence comprises a poly(A) sequence native to the backbone. In various embodiments, the at least one poly(A) sequence comprises a poly(A) sequence exogenous to the backbone. In various embodiments, the at least one poly(A) sequence is operably linked to at least one of the at least one antigen-encoding nucleic acid sequences. In various embodiments, the at least one poly(A) sequence is at least 20, at least 30, at least 40, at least 50, at least 60, at least 70, at least 80, or at least 90 consecutive A nucleotides (SEQ ID NO: 78). In various embodiments, the at least one poly(A) sequence is at least 100 consecutive A nucleotides (SEQ ID NO: 79).

In various embodiments, the antigen expression system further comprises at least one of: an intron sequence, a woodchuck hepatitis virus posttranscriptional regulatory element (WPRE) sequence, an internal ribosome entry sequence (IRES) sequence, a nucleotide sequence encoding a 2A self cleaving peptide sequence, a nucleotide sequence encoding a Furin cleavage site, or a sequence in the 5′ or 3′ non-coding region known to enhance the nuclear export, stability, or translation efficiency of mRNA that is operably linked to at least one of the at least one antigen-encoding nucleic acid sequences. In various embodiments, the antigen expression system further comprises a reporter gene, including but not limited to, green fluorescent protein (GFP), a GFP variant, secreted alkaline phosphatase, luciferase, a luciferase variant, or a detectable peptide or epitope. In various embodiments, the detectable peptide or epitope is selected from the group consisting of an HA tag, a Flag tag, a His-tag, or a V5 tag. In various embodiments, the at least one MHC class I antigen-encoding nucleic acid sequence is selected by performing the steps of: (a) obtaining at least one of exome, transcriptome, or whole genome nucleotide sequencing, wherein the nucleotide sequencing data is used to obtain data representing peptide sequences of each of a set of antigens; (b) inputting the peptide sequence of each antigen into a presentation model to generate a set of numerical likelihoods that each of the antigens is presented by one or more of the MHC proteins, the set of numerical likelihoods having been identified at least based on received mass spectrometry data; and (c) selecting a subset of the set of antigens based on the set of numerical likelihoods to generate a set of selected antigens which are used to generate the at least one MHC class I antigen-encoding nucleic acid sequence.

In various embodiments, each of the MHC class I epitope encoding nucleic acid sequences is selected by performing the steps of: (a) obtaining at least one of exome, transcriptome, or whole genome nucleotide sequencing data, wherein the nucleotide sequencing data is used to obtain data representing peptide sequences of each of a set of antigens; (b) inputting the peptide sequence of each antigen into a presentation model to generate a set of numerical likelihoods that each of the antigens is presented by one or more MHC proteins, the set of numerical likelihoods having been identified at least based on received mass spectrometry data; and (c) selecting a subset of the set of antigens based on the set of numerical likelihoods to generate a set of selected antigens which are used to generate the at least 20 MHC class I antigen-encoding nucleic acid sequences. In various embodiments, a number of the set of selected antigens is 2-20. In various embodiments, the presentation model represents dependence between: (a) presence of a pair of a particular one of the MHC alleles and a particular amino acid at a particular position of a peptide sequence; and (b) likelihood of presentation, by the particular one of the MHC alleles of the pair, of such a peptide sequence comprising the particular amino acid at the particular position.

In various embodiments, selecting the set of selected antigens comprises selecting antigens that have an increased likelihood of being presented relative to unselected antigens based on the presentation model, optionally wherein the selected antigens have been validated as being presented by one or more specific HLA alleles. In various embodiments, selecting the set of selected antigens comprises selecting antigens that have an increased likelihood of being capable of inducing an immune response in response to presence of HIV in the subject relative to unselected antigens based on the presentation model. In various embodiments, selecting the set of selected antigens comprises selecting antigens that have an increased likelihood of being capable of being presented to naïve T cells by professional antigen presenting cells (APCs) relative to unselected antigens based on the presentation model, optionally wherein the APC is a dendritic cell (DC). In various embodiments, selecting the set of selected antigens comprises selecting antigens that have a decreased likelihood of being subject to inhibition via central or peripheral tolerance relative to unselected antigens based on the presentation model. In various embodiments, selecting the set of selected antigens comprises selecting antigens that have a decreased likelihood of being capable of inducing an autoimmune response to normal tissue in the subject relative to unselected antigens based on the presentation model.

In various embodiments, exome or transcriptome nucleotide sequencing data is obtained by performing next generation sequencing (NGS) or any massively parallel sequencing approach. In various embodiments, the antigen cassette comprises junctional epitope sequences formed by adjacent sequences in the antigen cassette. In various embodiments, at least one or each junctional epitope sequence has an affinity of greater than 500 nM for MHC. In various embodiments, each junctional epitope sequence is non-self. In various embodiments, each of the MHC class I epitopes is predicted or validated to be capable of presentation by at least one HLA allele present in at least 5% of a population. In various embodiments, each of the MHC class I epitopes is predicted or validated to be capable of presentation by at least one HLA allele, wherein each antigen/HLA pair has an antigen/HLA prevalence of at least 0.01% in a population. In various embodiments, each of the MHC class I epitopes is predicted or validated to be capable of presentation by at least one HLA allele, wherein each antigen/HLA pair has an antigen/HLA prevalence of at least 0.1% in a population.

Additionally disclosed herein is a pharmaceutical composition comprising the composition describe above and a pharmaceutically acceptable carrier. In various embodiments, the composition further comprises an adjuvant.

Additionally disclosed herein is an isolated nucleotide sequence or set of isolated nucleotide sequences comprising the antigen cassette of any of the above compositions and one or more elements obtained from the sequence of SEQ ID NO:3 or SEQ ID NO:5, optionally wherein the one or more elements are selected from the group consisting of the sequences necessary for nonstructural protein-mediated amplification, the 26S promoter nucleotide sequence, the poly(A) sequence, and the nsP1-4 genes of the sequence set forth in SEQ ID NO:3 or SEQ ID NO:5, and optionally wherein the nucleotide sequence is cDNA. In various embodiments, the sequence or set of isolated nucleotide sequences comprises the antigen cassette of any of the above compositions inserted at position 7544 of the sequence set forth in SEQ ID NO:6 or SEQ ID NO:7. In various embodiments, the isolated nucleotide sequence further comprises: a T7 or SP6 RNA polymerase promoter nucleotide sequence 5′ of the one or more elements obtained from the sequence of SEQ ID NO:3 or SEQ ID NO:5; and optionally, one or more restriction sites 3′ of the poly(A) sequence. In various embodiments, the antigen cassette of any of the above compositions is inserted at position 7563 of SEQ ID NO:8 or SEQ ID NO:9.

Additionally disclosed herein is a vector or set of vectors comprising the nucleotide sequence described above. Additionally disclosed herein is an isolated cell comprising the nucleotide sequence or set of isolated nucleotide sequences described above, optionally wherein the cell is a BHK-21, CHO, HEK293 or variants thereof, 911, HeLa, A549, LP-293, PER.C6, or AE1-2a cell.

Additionally disclosed herein is a method for treating a subject with HIV, the method comprising administering to the subject the composition of any of the above compositions or the pharmaceutical composition described above. Additionally disclosed herein is a method for inducing an immune response in a subject, the method comprising administering to the subject the composition of any of the above compositions or the pharmaceutical composition described above. In various embodiments, the subject expresses at least one HLA allele predicted or known to present at least one of the MHC class I epitopes encoded by the one or more vectors of the antigen expression system. In various embodiments, the composition is administered intramuscularly (IM), intradermally (ID), subcutaneously (SC), or intravenously (IV).

In various embodiments, the composition is administered intramuscularly. In various embodiments, the method further comprises administering to the subject a second vaccine composition. In various embodiments, the second vaccine composition is administered prior to the administration of the composition or the pharmaceutical composition. In various embodiments, the second vaccine composition is administered subsequent to the administration of the composition or the pharmaceutical composition. In various embodiments, the second vaccine composition is the same as the composition or the pharmaceutical composition. In various embodiments, the second vaccine composition is different from the composition or the pharmaceutical composition. In various embodiments, the second vaccine composition comprises a chimpanzee adenovirus vector encoding at least one antigen-encoding nucleic acid sequence. In various embodiments, the at least one antigen-encoding nucleic acid sequence encoded by the chimpanzee adenovirus vector is the same as the at least one antigen-encoding nucleic acid sequence of any of the above compositions.

Additionally disclosed herein is a method of manufacturing the antigen expression system described above, the method comprising: (a) obtaining a linearized DNA sequence comprising the backbone and the antigen cassette; (b) in vitro transcribing the linearized DNA sequence by addition of the linearized DNA sequence to an in vitro transcription reaction containing all the necessary components to transcribe the linearized DNA sequence into RNA, optionally further comprising in vitro addition of the m7g cap to the resulting RNA; and (c) isolating the one or more vectors from the in vitro transcription reaction. In various embodiments, the linearized DNA sequence is generated by linearizing a DNA plasmid sequence or by amplification using PCR. In various embodiments, the DNA plasmid sequence is generated using one of bacterial recombination or full genome DNA synthesis or full genome DNA synthesis with amplification of synthesized DNA in bacterial cells. In various embodiments, isolating the one or more vectors from the in vitro transcription reaction involves one or more of phenol chloroform extraction, silica column based purification, or similar RNA purification methods.

Additionally disclosed herein is a method of manufacturing the composition for delivery of the antigen expression system, the method comprising: (a) providing components for the nanoparticulate delivery vehicle; (b) providing the antigen expression system; and (c) providing conditions sufficient for the nanoparticulate delivery vehicle and the antigen expression system to produce the composition for delivery of the antigen expression system. In various embodiments, the conditions are provided by microfluidic mixing.

Additionally disclosed herein is a method of assessing a subject having HIV, comprising the steps of: a) determining or having determined a HIV subtype of the HIV of the subject; b) determining or having determined whether the subject expresses a HLA allele predicted or known to present a MHC class I epitope encoded by an antigen-encoding nucleic acid sequence in an antigen-based vaccine, and c) determining or having determined that the subject is a candidate for therapy with the antigen-based vaccine when the subject expresses the HLA allele, and the HIV subtype expresses the MHC class I epitope encoded by the antigen-encoding nucleic acid sequence in the antigen-based vaccine, wherein the MHC class I epitope comprises at least one MHC class I epitope sequence selected from the group consisting of epitope sequences of any one of SEQ ID Nos: 325-22349, and d) optionally, administering or having administered the antigen-based vaccine to the subject. In various embodiments, the HLA allele expressed by the subject is selected from the group consisting of HLA alleles in Tables 35-45.

Additionally disclosed herein is a method of assessing a subject having HIV, comprising the steps of: a) determining or having determined the HIV of the subject is HIV subtype A1; b) determining or having determined whether the subject expresses a HLA allele predicted or known to present a MHC class I epitope encoded by an antigen-encoding nucleic acid sequence in an antigen-based vaccine, and c) determining or having determined that the subject is a candidate for therapy with the antigen-based vaccine when the subject expresses the HLA allele, and the HIV subtype expresses the MHC class I epitope encoded by the antigen-encoding nucleic acid sequence in the antigen-based vaccine, wherein the MHC class I epitope comprises at least one MHC class I epitope sequence comprising at least one HIV epitope selected from the group consisting of the sequences shown in SEQ ID NOs: 325-2165, and d) optionally, administering or having administered the antigen-based vaccine to the subject. In various embodiments, the HLA allele expressed by the subject is selected from the group consisting of HLA alleles in Table 35.

Additionally disclosed herein is a method of assessing a subject having HIV, comprising the steps of: a) determining or having determined the HIV of the subject is HIV subtype A2; b) determining or having determined whether the subject expresses a HLA allele predicted or known to present a MHC class I epitope encoded by an antigen-encoding nucleic acid sequence in an antigen-based vaccine, and c) determining or having determined that the subject is a candidate for therapy with the antigen-based vaccine when the subject expresses the HLA allele, and the HIV subtype expresses the MHC class I epitope encoded by the antigen-encoding nucleic acid sequence in the antigen-based vaccine, wherein the MHC class I epitope comprises at least one MHC class I epitope sequence comprising at least one HIV epitope selected from the group consisting of the sequences shown in SEQ ID NOs: 2166-4106, and d) optionally, administering or having administered the antigen-based vaccine to the subject. In various embodiments, the HLA allele expressed by the subject is selected from the group consisting of HLA alleles in Table 36.

Additionally disclosed herein is a method of assessing a subject having HIV, comprising the steps of: a) determining or having determined the HIV of the subject is HIV subtype B; b) determining or having determined whether the subject expresses a HLA allele predicted or known to present a MHC class I epitope encoded by an antigen-encoding nucleic acid sequence in an antigen-based vaccine, and c) determining or having determined that the subject is a candidate for therapy with the antigen-based vaccine when the subject expresses the HLA allele, and the HIV subtype expresses the MHC class I epitope encoded by the antigen-encoding nucleic acid sequence in the antigen-based vaccine, wherein the MHC class I epitope comprises at least one MHC class I epitope sequence comprising at least one HIV epitope selected from the group consisting of the sequences shown in SEQ ID NOs: 4107-6241, and d) optionally, administering or having administered the antigen-based vaccine to the subject. In various embodiments, the HLA allele expressed by the subject is selected from the group consisting of HLA alleles in Table 37.

Additionally disclosed herein is a method of assessing a subject having HIV, comprising the steps of: a) determining or having determined the HIV of the subject is HIV subtype C; b) determining or having determined whether the subject expresses a HLA allele predicted or known to present a MHC class I epitope encoded by an antigen-encoding nucleic acid sequence in an antigen-based vaccine, and c) determining or having determined that the subject is a candidate for therapy with the antigen-based vaccine when the subject expresses the HLA allele, and the HIV subtype expresses the MHC class I epitope encoded by the antigen-encoding nucleic acid sequence in the antigen-based vaccine, wherein the MHC class I epitope comprises at least one MHC class I epitope sequence comprising at least one HIV epitope selected from the group consisting of the sequences shown in SEQ ID NOs: 6242-8389, and d) optionally, administering or having administered the antigen-based vaccine to the subject. In various embodiments, the HLA allele expressed by the subject is selected from the group consisting of HLA alleles in Table 38.

Additionally disclosed herein is a method of assessing a subject having HIV, comprising the steps of: a) determining or having determined the HIV of the subject is HIV subtype D; b) determining or having determined whether the subject expresses a HLA allele predicted or known to present a MHC class I epitope encoded by an antigen-encoding nucleic acid sequence in an antigen-based vaccine, and c) determining or having determined that the subject is a candidate for therapy with the antigen-based vaccine when the subject expresses the HLA allele, and the HIV subtype expresses the MHC class I epitope encoded by the antigen-encoding nucleic acid sequence in the antigen-based vaccine, wherein the MHC class I epitope comprises at least one MHC class I epitope sequence comprising at least one HIV epitope selected from the group consisting of the sequences shown in SEQ ID NOs: 8930-10626, and d) optionally, administering or having administered the antigen-based vaccine to the subject. In various embodiments, the HLA allele expressed by the subject is selected from the group consisting of HLA alleles in Table 39.

Additionally disclosed herein is a method of assessing a subject having HIV, comprising the steps of: a) determining or having determined the HIV of the subject is HIV subtype F1; b) determining or having determined whether the subject expresses a HLA allele predicted or known to present a MHC class I epitope encoded by an antigen-encoding nucleic acid sequence in an antigen-based vaccine, and c) determining or having determined that the subject is a candidate for therapy with the antigen-based vaccine when the subject expresses the HLA allele, and the HIV subtype expresses the MHC class I epitope encoded by the antigen-encoding nucleic acid sequence in the antigen-based vaccine, wherein the MHC class I epitope comprises at least one MHC class I epitope sequence selected from the group consisting of epitope sequences from any one of SEQ ID NOs: 10627-12810, and d) optionally, administering or having administered the antigen-based vaccine to the subject. In various embodiments, the HLA allele expressed by the subject is selected from the group consisting of HLA alleles in Table 40.

Additionally disclosed herein is a method of assessing a subject having HIV, comprising the steps of: a) determining or having determined the HIV of the subject is HIV subtype F2; b) determining or having determined whether the subject expresses a HLA allele predicted or known to present a MHC class I epitope encoded by an antigen-encoding nucleic acid sequence in an antigen-based vaccine, and c) determining or having determined that the subject is a candidate for therapy with the antigen-based vaccine when the subject expresses the HLA allele, and the HIV subtype expresses the MHC class I epitope encoded by the antigen-encoding nucleic acid sequence in the antigen-based vaccine, wherein the MHC class I epitope comprises at least one MHC class I epitope sequence selected from the group consisting of epitope sequences from any one of SEQ ID NOs: 12811-15079, and d) optionally, administering or having administered the antigen-based vaccine to the subject. In various embodiments, the HLA allele expressed by the subject is selected from the group consisting of HLA alleles in Table 41.

Additionally disclosed herein is a method of assessing a subject having HIV, comprising the steps of: a) determining or having determined the HIV of the subject is HIV subtype G; b) determining or having determined whether the subject expresses a HLA allele predicted or known to present a MHC class I epitope encoded by an antigen-encoding nucleic acid sequence in an antigen-based vaccine, and c) determining or having determined that the subject is a candidate for therapy with the antigen-based vaccine when the subject expresses the HLA allele, and the HIV subtype expresses the MHC class I epitope encoded by the antigen-encoding nucleic acid sequence in the antigen-based vaccine, wherein the MHC class I epitope comprises at least one MHC class I epitope sequence selected from the group consisting of epitope sequences from any one of SEQ ID NOs: 15080-17174, and d) optionally, administering or having administered the antigen-based vaccine to the subject. In various embodiments, the HLA allele expressed by the subject is selected from the group consisting of HLA alleles in Table 42.

Additionally disclosed herein is a method of assessing a subject having HIV, comprising the steps of: a) determining or having determined the HIV of the subject is HIV subtype H; b) determining or having determined whether the subject expresses a HLA allele predicted or known to present a MHC class I epitope encoded by an antigen-encoding nucleic acid sequence in an antigen-based vaccine, and c) determining or having determined that the subject is a candidate for therapy with the antigen-based vaccine when the subject expresses the HLA allele, and the HIV subtype expresses the MHC class I epitope encoded by the antigen-encoding nucleic acid sequence in the antigen-based vaccine, wherein the MHC class I epitope comprises at least one MHC class I epitope sequence comprising at least one HIV epitope selected from the group consisting of the sequences shown in SEQ ID NOs: 17175-19388, and d) optionally, administering or having administered the antigen-based vaccine to the subject. In various embodiments, the HLA allele expressed by the subject is selected from the group consisting of HLA alleles in Table 43.

Additionally disclosed herein is a method of assessing a subject having HIV, comprising the steps of: a) determining or having determined the HIV of the subject is HIV subtype J; b) determining or having determined whether the subject expresses a HLA allele predicted or known to present a MHC class I epitope encoded by an antigen-encoding nucleic acid sequence in an antigen-based vaccine, and c) determining or having determined that the subject is a candidate for therapy with the antigen-based vaccine when the subject expresses the HLA allele, and the HIV subtype expresses the MHC class I epitope encoded by the antigen-encoding nucleic acid sequence in the antigen-based vaccine, wherein the MHC class I epitope comprises at least one MHC class I epitope sequence comprising at least one HIV epitope selected from the group consisting of the sequences shown in SEQ ID NOs: 19389-21003, and d) optionally, administering or having administered the antigen-based vaccine to the subject. In various embodiments, the HLA allele expressed by the subject is selected from the group consisting of HLA alleles in Table 44.

Additionally disclosed herein is a method of assessing a subject having HIV, comprising the steps of: a) determining or having determined the HIV of the subject is HIV subtype K; b) determining or having determined whether the subject expresses a HLA allele predicted or known to present a MHC class I epitope encoded by an antigen-encoding nucleic acid sequence in an antigen-based vaccine, and c) determining or having determined that the subject is a candidate for therapy with the antigen-based vaccine when the subject expresses the HLA allele, and the HIV subtype expresses the MHC class I epitope encoded by the antigen-encoding nucleic acid sequence in the antigen-based vaccine, wherein the MHC class I epitope comprises at least one MHC class I epitope sequence comprising at least one HIV epitope selected from the group consisting of the sequences shown in SEQ ID NOs: 21004-22349, and d) optionally, administering or having administered the antigen-based vaccine to the subject. In various embodiments, the HLA allele expressed by the subject is selected from the group consisting of HLA alleles in Table 45.

In various embodiments, determining or having determined a HIV subtype of the HIV of the subject comprises obtaining a dataset indicating the HIV subtype from a third party that has processed a sample from the subject. In various embodiments, determining or having determined whether the subject expresses a HLA allele comprises obtaining a dataset from a third party that has processed a sample from the subject. In various embodiments, determining or having determined whether the subject expresses a HLA allele comprises obtaining a sample from the subject and assaying the sample using a method selected from the group consisting of: exome sequencing, targeted exome sequencing, transcriptome sequencing, Sanger sequencing, PCR-based genotyping assays, mass-spectrometry based methods, microarray, Nanostring, ISH, and IHC. In various embodiments, the sample is selected from tissue, bodily fluid, blood, spinal fluid, or needle aspirate. In various embodiments, the HLA allele has an HLA frequency of at least 1%.

Additionally disclosed herein is a method for treating a subject, the method comprising administering to the subject an antigen-based vaccine, wherein the antigen-based vaccine comprises: 1) at least one MHC class I epitope expressed by a HIV subtype, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID Nos: 325-22349. Additionally disclosed herein is a method for treating a subject with HIV, the method comprising administering to the subject an antigen-based vaccine, wherein the antigen-based vaccine comprises) at least one MHC class I epitope expressed by a HIV subtype, wherein the HIV subtype is HIV subtype A1, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID NOs: 325-2165.

Additionally disclosed herein is a method for treating a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype A2, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID NOs: 2166-4106.

Additionally disclosed herein is a method for treating a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype B, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID NOs: 4107-6241.

Additionally disclosed herein is a method for treating a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype C, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID NOs: 6242-8389.

Additionally disclosed herein is a method comprising administering to the subject an antigen-based vaccine, wherein the antigen-based vaccine comprises: 1) at least one MHC class I epitope expressed by a HIV subtype, wherein the HIV subtype is HIV subtype D, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID NOs: 8930-10626.

Additionally disclosed herein is a method for treating a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype F1, or 2) a MHC class I epitope encoding nucleic acid sequence encoding the at least one MHC class I epitope, wherein the at least one MHC class I epitope comprises at least one HIV epitope sequence selected from the group consisting of the sequences shown in SEQ ID NOs: 10627-12810.

Additionally disclosed herein is a method for treating a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype F2, or 2) a MHC class I epitope encoding nucleic acid sequence encoding the at least one MHC class I epitope, wherein the at least one MHC class I epitope comprises at least one HIV epitope sequence selected from the group consisting of the sequences shown in SEQ ID NOs: 12811-15079.

Additionally disclosed herein is a method for treating a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype G, or 2) a MHC class I epitope encoding nucleic acid sequence encoding the at least one MHC class I epitope, wherein the at least one MHC class I epitope comprises at least one HIV epitope sequence selected from the group consisting of the sequences shown in SEQ ID NOs: 15080-17174.

Additionally disclosed herein is a method for treating a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype H, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID NOs: 17175-19388.

Additionally disclosed herein is a method for treating a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype J, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID NOs: 19389-21003.

Additionally disclosed herein is a method for treating a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype K, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID NOs: 21004-22349.

Additionally disclosed herein is a method for inducing an immune response in a subject with HIV, the method comprising administering to the subject an antigen-based vaccine, wherein the antigen-based vaccine comprises: 1) at least one MHC class I epitope expressed by a HIV subtype, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID Nos: 325-22349.

Additionally disclosed herein is a method for inducing an immune response in a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype A1, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID NOs: 325-2165.

Additionally disclosed herein is a method for inducing an immune response in a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype A2, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID NOs: 2166-4106.

Additionally disclosed herein is a method for inducing an immune response in a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype B, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID NOs: 4107-6241.

A method for inducing an immune response in a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype C, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID NOs: 6242-8389.

Additionally disclosed herein is a method for inducing an immune response in a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype D, or 2) a MHC class I epitope encoding nucleic acid sequence encoding the at least one MHC class I epitope, wherein the at least one MHC class I epitope comprises a MHC class I epitope sequence selected from a group consisting of epitope sequences from any one of SEQ ID NOs: 8930-10626.

Additionally disclosed herein is a method for inducing an immune response in a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype F1, or 2) a MHC class I epitope encoding nucleic acid sequence encoding the at least one MHC class I epitope, wherein the at least one MHC class I epitope comprises at least one HIV epitope sequence selected from the group consisting of the sequences shown in SEQ ID NOs: 10627-12810.

Additionally disclosed herein is a method for inducing an immune response in a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype F2, or 2) a MHC class I epitope encoding nucleic acid sequence encoding the at least one MHC class I epitope, wherein the at least one MHC class I epitope comprises at least one HIV epitope sequence selected from the group consisting of the sequences shown in SEQ ID NOs: 12811-15079.

Additionally disclosed herein is a method for inducing an immune response in a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype G, or 2) a MHC class I epitope encoding nucleic acid sequence encoding the at least one MHC class I epitope, wherein the at least one MHC class I epitope comprises at least one HIV epitope sequence selected from the group consisting of the sequences shown in SEQ ID NOs: 15080-17174.

Additionally disclosed herein is a method for inducing an immune response in a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype H, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID NOs: 17175-19388.

Additionally disclosed herein is a method for inducing an immune response in a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype J, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID NOs: 19389-21003.

Additionally disclosed herein is a method for inducing an immune response in a subject with HIV, the method comprising administering to the subject an antigen-based vaccine, wherein the antigen-based vaccine comprises: 1) at least one MHC class I epitope expressed by a HIV subtype, wherein the HIV subtype is HIV subtype K, or 2) a MHC class I epitope encoding nucleic acid sequence encoding the at least one MHC class I epitope, wherein the at least one MHC class I epitope comprises a MHC class I epitope sequence comprising at least one HIV epitope selected from the group consisting of the sequences shown in SEQ ID NOs: 21004-22349.

In various embodiments, the subject expresses at least one HLA allele predicted or known to present the at least one MHC class I epitope sequence. In various embodiments, the method further comprises: prior to administering to the subject the antigen-based vaccine, determining that the subject is a candidate for receiving the antigen-based vaccine, wherein the determination comprises identifying that 1) the subject expresses an HLA allele known to or predicted to present the at least one MHC class I epitope and 2) the subject has been exposed to or is susceptible to exposure to the HIV subtype. In various embodiments, the at least one HLA allele is selected from the group consisting of HLA alleles in Tables 35-45.

In various embodiments, wherein the antigen-based vaccine comprises an antigen expression system. In various embodiments, the antigen expression system comprises any one of the antigen expression systems described above. In various embodiments, the antigen-based vaccine comprises any one of the pharmaceutical compositions.

In various embodiments, each MHC class I epitope comprises a sequence selected from the group consisting of epitope sequences of any one of SEQ ID NOs: 325-2165. In various embodiments, each MHC class I epitope comprises a sequence selected from the group consisting of epitope sequences of any one of SEQ ID NOs: 2166-4106. In various embodiments, each MHC class I epitope comprises a sequence selected from the group consisting of epitope sequences of any one of SEQ ID NOs: 4107-6241. In various embodiments, each MHC class I epitope comprises a sequence selected from the group consisting of epitope sequences of any one of SEQ ID NOs: 6242-8389. In various embodiments, each MHC class I epitope comprises a sequence selected from the group consisting of epitope sequences of any one of SEQ ID NOs: 8930-10626. In various embodiments, each MHC class I epitope comprises a sequence selected from the group consisting of epitope sequences of any one of SEQ ID NOs: 10627-12810. In various embodiments, each MHC class I epitope comprises a sequence selected from the group consisting of epitope sequences of any one of SEQ ID NOs: 12811-15079. In various embodiments, each MHC class I epitope comprises a sequence selected from the group consisting of epitope sequences of any one of SEQ ID NOs: 15080-17174. In various embodiments, each MHC class I epitope comprises a sequence selected from the group consisting of epitope sequences of any one of SEQ ID NOs: 17175-19388. In various embodiments, each MHC class I epitope comprises a sequence selected from the group consisting of epitope sequences of any one of SEQ ID NOs: 19389-21003. In various embodiments, each MHC class I epitope comprises a sequence selected from the group consisting of epitope sequences of any one of SEQ ID NOs: 21004-22349.

Additionally disclosed herein is a method of assessing a subject having HIV, comprising the steps of: a) determining or having determined that the subject expresses a HLA allele; b) obtaining or having obtained sequencing data of HIV present in that subject; c) selecting candidate epitope sequences for inclusion in an antigen-based vaccine, wherein a first candidate epitope sequence is selected from the group consisting of epitope sequences from any one of SEQ ID Nos: 325-22349, and wherein a second candidate epitope sequence is a mutated epitope sequence, each of the first and second candidate epitope sequences predicted to be presented by the HLA allele expressed by the subject; d) generating the antigen-based vaccine including the selected candidate epitope sequences; and e) optionally, administering or having administered the antigen-based vaccine to the subject.

Additionally disclosed herein is a method for treating a subject having HIV, comprising the steps of: a) determining or having determined that the subject expresses a HLA allele; b) obtaining or having obtained sequencing data of HIV present in that subject; c) selecting candidate epitope sequences for inclusion in an antigen-based vaccine, wherein a first candidate epitope sequence is selected from the group consisting of epitope sequences from any one of SEQ ID Nos: 325-22349, and wherein a second candidate epitope sequence is a mutated epitope sequence, each of the first and second candidate epitope sequences predicted to be presented by the HLA allele expressed by the subject; d) generating the antigen-based vaccine including the selected candidate epitope sequences; and e) optionally, administering or having administered the antigen-based vaccine to the subject.

In various embodiments, epitope sequences of any one of SEQ ID Nos: 325-22349 are identified by applying a presentation model trained on HLA presented peptides sequenced by mass spectrometry. In various embodiments, the presentation model exhibits a precision value of 0.28 at a 40% recall rate. In various embodiments the presentation model exhibits an AUC of 0.24.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 illustrates development of an in vitro T cell activation assay. Schematic of the assay in which the delivery of a vaccine cassette to antigen presenting cells, leads to expression, processing and MHC-restricted presentation of distinct peptide antigens. Reporter T cells engineered with T cell receptors that match the specific peptide-MHC combination become activated resulting in luciferase expression.

FIG. 2A illustrates evaluation of linker sequences in short cassettes and shows five class I MHC restricted epitopes (epitopes 1 through 5) concatenated in the same position relative to each other followed by two universal class II MHC epitopes (MHC-II). Various iterations were generated using different linkers. In some cases the T cell epitopes are directly linked to each other. In others, the T cell epitopes are flanked on one or both sides by its natural sequence. In other iterations, the T cell epitopes are linked by the non-natural sequences AAY, RR, and DPP.

FIG. 2B illustrates evaluation of linker sequences in short cassettes and shows sequence information on the T cell epitopes embedded in the short cassettes. Figure discloses SEQ ID NOS 274-280, respectively, in order of appearance.

FIG. 3 illustrates evaluation of cellular targeting sequences added to model vaccine cassettes. The targeting cassettes extend the short cassette designs with ubiquitin (Ub), signal peptides (SP) and/or transmembrane (TM) domains, feature next to the five marker human T cell epitopes (epitopes 1 through 5) also two mouse T cell epitopes SIINFEKL (SEQ ID NO: 80) (SII) and SPSYAYHQF (SEQ ID NO: 81) (A5), and use either the non-natural linker AAY- or natural linkers flanking the T cell epitopes on both sides (25mer).

FIG. 4A illustrates in vivo evaluation of the impact of epitope position in long 21-mer cassettes and shows the design of long cassettes entails five marker class I epitopes (epitopes 1 through 5) contained in their 25-mer natural sequence (linker=natural flanking sequences), spaced with additional well-known T cell class I epitopes (epitopes 6 through 21) contained in their 25-mer natural sequence, and two universal class II epitopes (MHC-II0, with only the relative position of the class I epitopes varied.

FIG. 4B illustrates in vivo evaluation of the impact of epitope position in long 21-mer cassettes and shows the sequence information on the T cell epitopes used. Figure discloses SEQ ID NOS 281-301, respectively, in order of appearance.

FIG. 5A illustrates final cassette design for preclinical IND-enabling studies and shows the design of the final cassettes comprises 20 MHC I epitopes contained in their 25-mer natural sequence (linker=natural flanking sequences), composed of 6 non-human primate (NHP) epitopes, 5 human epitopes, 9 murine epitopes, as well as 2 universal MHC class II epitopes.

FIG. 5B illustrates final cassette design for preclinical IND-enabling studies and shows the sequence information for the T cell epitopes used that are presented on class I MHC of non-human primate, mouse and human origin, as well as sequences of 2 universal MHC class II epitopes PADRE and Tetanus toxoid. Figure discloses SEQ ID NOS 302-323, respectively, in order of appearance.

FIG. 6A illustrates ChAdV68.4WTnt.GFP virus production after transfection. HEK293A cells were transfected with ChAdV68.4WTnt.GFP DNA using the calcium phosphate protocol. Viral replication was observed 10 days after transfection and ChAdV68.4WTnt.GFP viral plaques were visualized using light microscopy (40× magnification).

FIG. 6B illustrates ChAdV68.4WTnt.GFP virus production after transfection. HEK293A cells were transfected with ChAdV68.4WTnt.GFP DNA using the calcium phosphate protocol. Viral replication was observed 10 days after transfection and ChAdV68.4WTnt.GFP viral plaques were visualized using fluorescent microscopy at 40× magnification.

FIG. 6C illustrates ChAdV68.4WTnt.GFP virus production after transfection. HEK293A cells were transfected with ChAdV68.4WTnt.GFP DNA using the calcium phosphate protocol. Viral replication was observed 10 days after transfection and ChAdV68.4WTnt.GFP viral plaques were visualized using fluorescent microscopy at 100× magnification.

FIG. 7A illustrates ChAdV68.5WTnt.GFP virus production after transfection. HEK293A cells were transfected with ChAdV68.5WTnt.GFP DNA using the lipofectamine protocol. Viral replication (plaques) was observed 10 days after transfection. A lysate was made and used to reinfect a T25 flask of 293A cells. ChAdV68.5WTnt.GFP viral plaques were visualized and photographed 3 days later using light microscopy (40× magnification)

FIG. 7B illustrates ChAdV68.5WTnt.GFP virus production after transfection. HEK293A cells were transfected with ChAdV68.5WTnt.GFP DNA using the lipofectamine protocol. Viral replication (plaques) was observed 10 days after transfection. A lysate was made and used to reinfect a T25 flask of 293A cells. ChAdV68.5WTnt.GFP viral plaques were visualized and photographed 3 days later using fluorescent microscopy at 40× magnification.

FIG. 7C illustrates ChAdV68.5WTnt.GFP virus production after transfection. HEK293A cells were transfected with ChAdV68.5WTnt.GFP DNA using the lipofectamine protocol. Viral replication (plaques) was observed 10 days after transfection. A lysate was made and used to reinfect a T25 flask of 293A cells. ChAdV68.5WTnt.GFP viral plaques were visualized and photographed 3 days later using fluorescent microscopy at 100× magnification.

FIG. 8 illustrates the viral particle production scheme.

FIG. 9 illustrates the alphavirus derived VEE self-replicating RNA (srRNA) vector.

FIG. 10 illustrates in vivo reporter expression after inoculation of C57BL/6J mice with VEE-Luciferase srRNA. Shown are representative images of luciferase signal following immunization of C57BL/6J mice with VEE-Luciferase srRNA (10 ug per mouse, bilateral intramuscular injection, MC3 encapsulated) at various timepoints.

FIG. 11A illustrates T-cell responses measured 14 days after immunization with VEE srRNA formulated with MC3 LNP in B16-OVA tumor bearing mice. B16-OVA tumor bearing C57BL/6J mice were injected with 10 ug of VEE-Luciferase srRNA (control), VEE-UbAAY srRNA (Vax), VEE-Luciferase srRNA and anti-CTLA-4 (aCTLA-4) or VEE-UbAAY srRNA and anti-CTLA-4 (Vax+aCTLA-4). In addition, all mice were treated with anti-PD1 mAb starting at day 7. Each group consisted of 8 mice. Mice were sacrificed and spleens and lymph nodes were collected 14 days after immunization. SIINFEKL (SEQ ID NO: 82)-specific T-cell responses were assessed by IFN-gamma ELISPOT and are reported as spot-forming cells (SFC) per 106 splenocytes. Lines represent medians.

FIG. 11B illustrates T-cell responses measured 14 days after immunization with VEE srRNA formulated with MC3 LNP in B16-OVA tumor bearing mice. B16-OVA tumor bearing C57BL/6J mice were injected with 10 ug of VEE-Luciferase srRNA (control), VEE-UbAAY srRNA (Vax), VEE-Luciferase srRNA and anti-CTLA-4 (aCTLA-4) or VEE-UbAAY srRNA and anti-CTLA-4 (Vax+aCTLA-4). In addition, all mice were treated with anti-PD1 mAb starting at day 7. Each group consisted of 8 mice. Mice were sacrificed and spleens and lymph nodes were collected 14 days after immunization. SIINFEKL (SEQ ID NO: 83)-specific T-cell responses were assessed by MHCI-pentamer staining, reported as pentamer positive cells as a percent of CD8 positive cells. Lines represent medians.

FIG. 12A illustrates antigen-specific T-cell responses following heterologous prime/boost in B16-OVA tumor bearing mice. B16-OVA tumor bearing C57BL/6J mice were injected with adenovirus expressing GFP (Ad5-GFP) and boosted with VEE-Luciferase srRNA formulated with MC3 LNP (Control) or Ad5-UbAAY and boosted with VEE-UbAAY srRNA (Vax). Both the Control and Vax groups were also treated with an IgG control mAb. A third group was treated with the Ad5-GFP prime/VEE-Luciferase srRNA boost in combination with anti-CTLA-4 (aCTLA-4), while the fourth group was treated with the Ad5-UbAAY prime/VEE-UbAAY boost in combination with anti-CTLA-4 (Vax+aCTLA-4). In addition, all mice were treated with anti-PD-1 mAb starting at day 21. T-cell responses were measured by IFN-gamma ELISPOT. Mice were sacrificed and spleens and lymph nodes collected at 14 days post immunization with adenovirus.

FIG. 12B illustrates antigen-specific T-cell responses following heterologous prime/boost in B16-OVA tumor bearing mice. B16-OVA tumor bearing C57BL/6J mice were injected with adenovirus expressing GFP (Ad5-GFP) and boosted with VEE-Luciferase srRNA formulated with MC3 LNP (Control) or Ad5-UbAAY and boosted with VEE-UbAAY srRNA (Vax). Both the Control and Vax groups were also treated with an IgG control mAb. A third group was treated with the Ad5-GFP prime/VEE-Luciferase srRNA boost in combination with anti-CTLA-4 (aCTLA-4), while the fourth group was treated with the Ad5-UbAAY prime/VEE-UbAAY boost in combination with anti-CTLA-4 (Vax+aCTLA-4). In addition, all mice were treated with anti-PD-1 mAb starting at day 21. T-cell responses were measured by IFN-gamma ELISPOT. Mice were sacrificed and spleens and lymph nodes collected at 14 days post immunization with adenovirus and 14 days post boost with srRNA (day 28 after prime).

FIG. 12C illustrates antigen-specific T-cell responses following heterologous prime/boost in B16-OVA tumor bearing mice. B16-OVA tumor bearing C57BL/6J mice were injected with adenovirus expressing GFP (Ad5-GFP) and boosted with VEE-Luciferase srRNA formulated with MC3 LNP (Control) or Ad5-UbAAY and boosted with VEE-UbAAY srRNA (Vax). Both the Control and Vax groups were also treated with an IgG control mAb. A third group was treated with the Ad5-GFP prime/VEE-Luciferase srRNA boost in combination with anti-CTLA-4 (aCTLA-4), while the fourth group was treated with the Ad5-UbAAY prime/VEE-UbAAY boost in combination with anti-CTLA-4 (Vax+aCTLA-4). In addition, all mice were treated with anti-PD-1 mAb starting at day 21. T-cell responses were measured by MHC class I pentamer staining. Mice were sacrificed and spleens and lymph nodes collected at 14 days post immunization with adenovirus.

FIG. 12D illustrates antigen-specific T-cell responses following heterologous prime/boost in B16-OVA tumor bearing mice. B16-OVA tumor bearing C57BL/6J mice were injected with adenovirus expressing GFP (Ad5-GFP) and boosted with VEE-Luciferase srRNA formulated with MC3 LNP (Control) or Ad5-UbAAY and boosted with VEE-UbAAY srRNA (Vax). Both the Control and Vax groups were also treated with an IgG control mAb. A third group was treated with the Ad5-GFP prime/VEE-Luciferase srRNA boost in combination with anti-CTLA-4 (aCTLA-4), while the fourth group was treated with the Ad5-UbAAY prime/VEE-UbAAY boost in combination with anti-CTLA-4 (Vax+aCTLA-4). In addition, all mice were treated with anti-PD-1 mAb starting at day 21. T-cell responses were measured by MHC class I pentamer staining. Mice were sacrificed and spleens and lymph nodes collected at 14 days post immunization with adenovirus and 14 days post boost with srRNA (day 28 after prime).

FIG. 13A illustrates antigen-specific T-cell responses following heterologous prime/boost in CT26 (Balb/c) tumor bearing mice. Mice were immunized with Ad5-GFP and boosted 15 days after the adenovirus prime with VEE-Luciferase srRNA formulated with MC3 LNP (Control) or primed with Ad5-UbAAY and boosted with VEE-UbAAY srRNA (Vax). Both the Control and Vax groups were also treated with an IgG control mAb. A separate group was administered the Ad5-GFP/VEE-Luciferase srRNA prime/boost in combination with anti-PD-1 (aPD1), while a fourth group received the Ad5-UbAAY/VEE-UbAAY srRNA prime/boost in combination with an anti-PD-1 mAb (Vax+aPD1). T-cell responses to the AH1 peptide were measured using IFN-gamma ELISPOT. Mice were sacrificed and spleens and lymph nodes collected at 12 days post immunization with adenovirus.

FIG. 13B illustrates antigen-specific T-cell responses following heterologous prime/boost in CT26 (Balb/c) tumor bearing mice. Mice were immunized with Ad5-GFP and boosted 15 days after the adenovirus prime with VEE-Luciferase srRNA formulated with MC3 LNP (Control) or primed with Ad5-UbAAY and boosted with VEE-UbAAY srRNA (Vax). Both the Control and Vax groups were also treated with an IgG control mAb. A separate group was administered the Ad5-GFP/VEE-Luciferase srRNA prime/boost in combination with anti-PD-1 (aPD1), while a fourth group received the Ad5-UbAAY/VEE-UbAAY srRNA prime/boost in combination with an anti-PD-1 mAb (Vax+aPD1). T-cell responses to the AH1 peptide were measured using IFN-gamma ELISPOT. Mice were sacrificed and spleens and lymph nodes collected at 12 days post immunization with adenovirus and 6 days post boost with srRNA (day 21 after prime).

FIG. 14 illustrates ChAdV68 eliciting T-Cell responses to mouse tumor antigens in mice. Mice were immunized with ChAdV68.5WTnt.MAG25mer, and T-cell responses to the MHC class I epitope SIINFEKL (SEQ ID NO: 84) (OVA) were measured in C57BL/6J female mice and the MHC class I epitope AH1-A5 measured in Balb/c mice. Mean spot forming cells (SFCs) per 10⁶ splenocytes measured in ELISpot assays presented. Error bars represent standard deviation.

FIGS. 15A, 15B, 15C and 15D illustrate antigen-specific cellular immune responses measured using ELISpot. Antigen-specific IFN-gamma production to six different mamu A01 restricted epitopes was measured in PBMCs for the VEE-MAG25mer srRNA-LNP1 (30 μg) (FIG. 15A), VEE-MAG25mer srRNA-LNP1 (100 μg) (FIG. 15B), or VEE-MAG25mer srRNA-LNP2 (100 μg) (FIG. 15C) homologous prime/boost or the ChAdV68.5WTnt.MAG25mer/VEE-MAG25mer srRNA heterologous prime/boost group (FIG. 15D) using ELISpot 1, 2, 3, 4, 5, 6, 8, 9, or 10 weeks after the first boost immunization (6 rhesus macaques per group). Results are presented as mean spot forming cells (SFC) per 10⁶ PBMCs for each epitope in a stacked bar graph format. Values for each animal were normalized to the levels at pre-bleed (week 0).

FIG. 16 shows antigen-specific cellular immune response measured using ELISpot. Antigen-specific IFN-gamma production to six different mamu A01 restricted epitopes was measured in PBMCs after immunization with the ChAdV68.5WTnt.MAG25mer/VEE-MAG25mer srRNA heterologous prime/boost regimen using ELISpot prior to immunization and 4, 5, 6, 7, 8, 10, 11, 12, 13, 14, 15, 16, 17, 18, 19, 20, 21, 22, 23 or 24 weeks after the initial immunization. Results are presented as mean spot forming cells (SFC) per 10⁶ PBMCs for each epitope (6 rhesus macaques per group) in a stacked bar graph format.

FIG. 17 shows antigen-specific cellular immune response measured using ELISpot. Antigen-specific IFN-gamma production to six different mamu A01 restricted epitopes was measured in PBMCs after immunization with the VEE-MAG25mer srRNA LNP2 homologous prime/boost regimen using ELISpot prior to immunization and 4, 5, 6, 7, 8, 10, 11, 12, 13, 14, or 15 weeks after the initial immunization. Results are presented as mean spot forming cells (SFC) per 10⁶ PBMCs for each epitope (6 rhesus macaques per group) in a stacked bar graph format.

FIG. 18 shows antigen-specific cellular immune response measured using ELISpot. Antigen-specific IFN-gamma production to six different mamu A01 restricted epitopes was measured in PBMCs after immunization with the VEE-MAG25mer srRNA LNP1 homologous prime/boost regimen using ELISpot prior to immunization and 4, 5, 6, 7, 8, 10, 11, 12, 13, 14, or 15 weeks after the initial immunization. Results are presented as mean spot forming cells (SFC) per 10⁶ PBMCs for each epitope (6 rhesus macaques per group) in a stacked bar graph format.

FIG. 19A and FIG. 19B show example peptide spectrums generated from Promega's dynamic range standard. Figure discloses SEQ ID NO: 324.

FIG. 20 illustrates the general TCR sequencing strategy and workflow.

FIG. 21 illustrates the general organization of the model epitopes from the various species for large antigen cassettes that had either 30 (L), 40 (XL) or 50 (XXL) epitopes.

FIG. 22 shows ChAd vectors express long cassettes as indicated by the above Western blot using an anti-class II (PADRE) antibody that recognizes a sequence common to all cassettes. HEK293 cells were infected with chAd68 vectors expressing large cassettes (chAd68-50XXL, chAd68-40XL & chAd68-30L) of variable size. Infections were set up at a MOI of 0.2. Twenty-four hours post infection MG132 a proteasome inhibitor was added to a set of the infected wells (indicated by the plus sign). Another set of virus treated wells were not treated with MG132 (indicated by minus sign). Uninfected HEK293 cells (293F) were used as a negative control. Forty-eight hours post infection cell pellets were harvested and analyzed by SDS/PAGE electrophoresis, and immunoblotting using a rabbit anti-Class II PADRE antibody. A HRP anti-rabbit antibody and ECL chemiluminescent substrate was used for detection.

FIG. 23 shows CD8+ immune responses in chAd68 large cassette immunized mice, detected against AH1 (top) and SIINFEKL (SEQ ID NO: 85) (bottom) by ICS. Data is presented as IFNg+ cells against the model epitope as % of total CD8 cells

FIG. 24 shows CD8+ responses to LD-AH1+(top) and Kb-SIINFEKL+ (SEQ ID NO: 86) (bottom) Tetramers post chAd68 large cassette vaccination. Data is presented as % of total CD8 cells reactive against the model Tetramer peptide complex. *p<0.05, **p<0.01 by ANOVA with Tukey's test. All p-values compared to MAG 20-antigen cassette.

FIG. 25 shows CD8+ immune responses in alphavirus large cassette treated mice, detected against AH1 (top) and SIINFEKL (SEQ ID NO: 87) ((bottom) by ICS. Data is presented as IFNg+ cells against the model epitope as % of total CD8 cells. *p<0.05, **p<0.01, ***p<0.001 by ANOVA with Tukey's test. All p-values compared to MAG 20-antigen cassette.

FIG. 26 illustrates the vaccination strategy used to evaluate immunogenicity of the antigen-cassette containing vectors in rhesus macaques. Triangles indicate chAd68 vaccination (1e12 vp/animal) at weeks 0 & 32. Circles represent alphavirus vaccination at weeks 0, 4, 12, 20, 28 & 32. Squares represent administration of an anti-CTLA4 antibody.

FIG. 27 shows a time course of CD8+ anti-epitope responses in Rhesus Macaques dosed with chAd-MAG alone (Group 4). Mean SFC/1e6 splenocytes is shown.

FIG. 28 shows a time course of CD8+ anti-epitope responses in Rhesus Macaques dosed with chAd-MAG plus anti-CTLA4 antibody (Ipilimumab) delivered IV. (Group 5). Mean SFC/1e6 splenocytes is shown.

FIG. 29 shows a time course of CD8+ anti-epitope responses in Rhesus Macaques dosed with chAd-MAG plus anti-CTLA4 antibody (Ipilimumab) delivered SC (Group 6). Mean SFC/1e6 splenocytes is shown.

FIG. 30 shows antigen-specific memory responses generated by ChAdV68/samRNA vaccine protocol measured by ELISpot. Results are presented as individual dot plots, with each dot representing a single animal. Pre-immunization baseline (left panel) and memory response at 18 months post-prime (right panel) are shown.

FIG. 31 shows memory cell phenotyping of antigen-specific CD8+ T-cells by flow cytometry using combinatorial tetramer staining and CD45RA/CCR7 co-staining.

FIG. 32 shows the distribution of memory cell types within the sum of the four Mamu-A*01 tetramer+CD8+ T-cell populations at study month 18. Memory cells were characterized as follows: CD45RA+CCR7+=naïve, CD45RA+CCR7−=effector (Teff), CD45RA-CCR7+=central memory (Tcm), CD45RA-CCR7-=effector memory (Tem).

FIG. 33 shows frequency of CD8+ T cells recognizing the CT26 tumor antigen AH1 in CT26 tumor-bearing mice. P values determined using the one-way ANOVA with Tukey's multiple comparisons test; **P<0.001, *P<0.05. ChAdV=ChAdV68.5WTnt.MAG25mer; aCTLA4=anti-CTLA4 antibody, clone 9D9.

FIG. 34 depicts a flow process for providing an antigen-based vaccine to the subject, in accordance with one embodiment.

FIG. 35 depicts a flow process for providing an antigen-based vaccine to the subject, in accordance with a second embodiment.

FIG. 36 depicts the predictive capacity of the EDGE model in comparison to a public prediction tool for predicting HIV epitopes that are presented by class I HLA alleles.

### DETAILED DESCRIPTION

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

As used herein the term “antigen” is a substance that induces an immune response.

As used herein the term “antigen-based vaccine” is a vaccine composition based on one or more antigens, e.g., a plurality of antigens. The vaccines can be nucleotide-based (e.g., virally based, RNA based, or DNA based), protein-based (e.g., peptide based), or a combination thereof.

As used herein the term “candidate antigen” refers to an antigen selected for inclusion in an antigen-based vaccine.

As used herein the term “candidate epitope sequence” refers to an epitope sequence on a candidate antigen selected for inclusion in an antigen-based vaccine.

As used herein the term “coding region” is the portion(s) of a gene that encode protein.

As used herein, the term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Alternatively, sequence similarity or dissimilarity can be established by the combined presence or absence of particular nucleotides, or, for translated sequences, amino acids at selected sequence positions (e.g., sequence motifs).

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

As used herein the term “epitope” is the specific portion of an antigen typically bound by an antibody or T cell receptor.

As used herein the term “immunogenic” is the ability to elicit an immune response, e.g., via T cells, B cells, or both.

As used herein the term “HLA binding affinity” “MHC binding affinity” means affinity of binding between a specific antigen and a specific HLA or MHC allele.

As used herein the term “variant” is a difference between a subject's nucleic acids and the reference human genome used as a control.

As used herein the term “variant call” is an algorithmic determination of the presence of a variant, typically from sequencing.

As used herein the term “polymorphism” is a germline variant, i.e., a variant found in all DNA-bearing cells of an individual.

As used herein the term “somatic variant” is a variant arising in non-germline cells of an individual.

As used herein the term “allele” is a version of a gene or a version of a genetic sequence or a version of a protein.

As used herein the term “HLA type” is the complement of HLA gene alleles.

As used herein the term “exome” is a subset of the genome that codes for proteins. An exome can be the collective exons of a genome.

As used herein the term “logistic regression” is a regression model for binary data from statistics where the logit of the probability that the dependent variable is equal to one is modeled as a linear function of the dependent variables.

As used herein the term “neural network” is a machine learning model for classification or regression consisting of multiple layers of linear transformations followed by element-wise nonlinearities typically trained via stochastic gradient descent and back-propagation.

As used herein the term “proteome” is the set of all proteins expressed and/or translated by a cell, group of cells, or individual.

As used herein the term “peptidome” is the set of all peptides presented by MHC-I or MHC-II on the cell surface. The peptidome may refer to a property of a cell or a collection of cells.

As used herein the term “ELISPOT” means Enzyme-linked immunosorbent spot assay—which is a common method for monitoring immune responses in humans and animals.

As used herein the term “dextramers” is a dextran-based peptide-MHC multimers used for antigen-specific T-cell staining in flow cytometry.

As used herein the term “tolerance or immune tolerance” is a state of immune non-responsiveness to one or more antigens, e.g. self-antigens.

As used herein the term “central tolerance” is a tolerance affected in the thymus, either by deleting self-reactive T-cell clones or by promoting self-reactive T-cell clones to differentiate into immunosuppressive regulatory T-cells (Tregs).

As used herein the term “peripheral tolerance” is a tolerance affected in the periphery by downregulating or anergizing self-reactive T-cells that survive central tolerance or promoting these T cells to differentiate into Tregs.

The term “sample” can include a single cell or multiple cells or fragments of cells or an aliquot of body fluid, taken from a subject, by means including venipuncture, excretion, ejaculation, massage, biopsy, needle aspirate, lavage sample, scraping, surgical incision, or intervention or other means known in the art.

The term “subject” encompasses a cell, tissue, or organism, human or non-human, whether in vivo, ex vivo, or in vitro, male or female. The term subject is inclusive of mammals including humans.

The term “mammal” encompasses both humans and non-humans and includes but is not limited to humans, non-human primates, canines, felines, murines, bovines, equines, and porcines.

The term “clinical factor” refers to a measure of a condition of a subject, e.g., disease activity or severity. “Clinical factor” encompasses all markers of a subject's health status, including non-sample markers, and/or other characteristics of a subject, such as, without limitation, age and gender. A clinical factor can be a score, a value, or a set of values that can be obtained from evaluation of a sample (or population of samples) from a subject or a subject under a determined condition. A clinical factor can also be predicted by markers and/or other parameters such as gene expression surrogates. Clinical factors can include past indications (e.g., patient history) and smoking history.

The term “alphavirus” refers to members of the family Togaviridae, and are positive-sense single-stranded RNA viruses. Alphaviruses are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis and its derivative strain TC-83. Alphaviruses are typically self-replicating RNA viruses.

The term “alphavirus backbone” refers to minimal sequence(s) of an alphavirus that allow for self-replication of the viral genome. Minimal sequences can include conserved sequences for nonstructural protein-mediated amplification, a nonstructural protein 1 (nsP1) gene, a nsP2 gene, a nsP3 gene, a nsP4 gene, and a polyA sequence, as well as sequences for expression of subgenomic viral RNA including a 26S promoter element.

The term “sequences for nonstructural protein-mediated amplification” includes alphavirus conserved sequence elements (CSE) well known to those in the art. CSEs include, but are not limited to, an alphavirus 5′ UTR, a 51-nt CSE, a 24-nt CSE, or other 26S subgenomic promoter sequence, a 19-nt CSE, and an alphavirus 3′ UTR.

The term “RNA polymerase” includes polymerases that catalyze the production of RNA polynucleotides from a DNA template. RNA polymerases include, but are not limited to, bacteriophage derived polymerases including T3, T7, and SP6.

The term “lipid” includes hydrophobic and/or amphiphilic molecules. Lipids can be cationic, anionic, or neutral. Lipids can be synthetic or naturally derived, and in some instances biodegradable. Lipids can include cholesterol, phospholipids, lipid conjugates including, but not limited to, polyethyleneglycol (PEG) conjugates (PEGylated lipids), waxes, oils, glycerides, fats, and fat-soluble vitamins. Lipids can also include dilinoleylmethyl-4-dimethylaminobutyrate (MC3) and MC3-like molecules.

The term “lipid nanoparticle” or “LNP” includes vesicle like structures formed using a lipid containing membrane surrounding an aqueous interior, also referred to as liposomes. Lipid nanoparticles includes lipid-based compositions with a solid lipid core stabilized by a surfactant. The core lipids can be fatty acids, acylglycerols, waxes, and mixtures of these surfactants. Biological membrane lipids such as phospholipids, sphingomyelins, bile salts (sodium taurocholate), and sterols (cholesterol) can be utilized as stabilizers. Lipid nanoparticles can be formed using defined ratios of different lipid molecules, including, but not limited to, defined ratios of one or more cationic, anionic, or neutral lipids. Lipid nanoparticles can encapsulate molecules within an outer-membrane shell and subsequently can be contacted with target cells to deliver the encapsulated molecules to the host cell cytosol. Lipid nanoparticles can be modified or functionalized with non-lipid molecules, including on their surface. Lipid nanoparticles can be single-layered (unilamellar) or multi-layered (multilamellar). Lipid nanoparticles can be complexed with nucleic acid. Unilamellar lipid nanoparticles can be complexed with nucleic acid, wherein the nucleic acid is in the aqueous interior. Multilamellar lipid nanoparticles can be complexed with nucleic acid, wherein the nucleic acid is in the aqueous interior, or to form or sandwiched between

Abbreviations: MHC: major histocompatibility complex; HLA: human leukocyte antigen, or the human MHC gene locus; NGS: next-generation sequencing; PPV: positive predictive value; FFPE: formalin-fixed, paraffin-embedded; NMD: nonsense-mediated decay; DC: dendritic cell.

It should be noted that, as used in the specification and the appended claims, the singular forms “a,” “an,” and “the” include plural referents unless the context clearly dictates otherwise.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

All references, issued patents and patent applications cited within the body of the specification are hereby incorporated by reference in their entirety, for all purposes.

### II. Methods of Identifying Antigens

Methods disclosed herein describe identifying candidate antigens for inclusion in a personalized antigen-based vaccine. Candidate antigens represent antigens of an infectious disease, such as HIV, that are likely to be presented on the cell surface of immune cells, including professional antigen presenting cells such as dendritic cells, and/or are likely to be immunogenic, for a particular subject.

As an example, one such method may comprise the steps of: obtaining HIV sequencing data, wherein the HIV sequencing data is used to obtain data representing peptide sequences of each of a set of antigens; inputting the peptide sequence of each antigen into one or more presentation models to generate a set of numerical likelihoods that each of the antigens is presented by one or more MHC proteins of the subject, the set of numerical likelihoods having been identified at least based on received mass spectrometry data; and selecting a subset of the set of antigens based on the set of numerical likelihoods to generate a set of selected antigens. In one aspect, each antigen in the set of antigens is encoded by coding regions in genes in the HIV genome (e.g., env, gag, Negative factor (nef), pol, rev, trans-activator of transcription (Tat), viral infectivity factor (vif), viral protein r (vir), or viral protein u (viu)).

The presentation model can comprise a statistical regression or a machine learning (e.g., deep learning) model trained on a set of reference data (also referred to as a training data set) comprising a set of corresponding labels, wherein the set of reference data is obtained from each of a plurality of distinct subjects where optionally some subjects are infected with HIV. The reference data can further comprise mass spectrometry data, sequencing data, RNA sequencing data, expression profiling data, and proteomics data for single-allele cell lines engineered to express a predetermined MHC allele that are subsequently exposed to synthetic protein, normal human cell lines, and fresh and frozen primary samples, and T cell assays (e.g., ELISPOT). In certain aspects, the set of reference data includes each form of reference data.

The presentation model can comprise a set of features derived at least in part from the set of reference data, and wherein the set of features comprises at least one of allele dependent-features and allele-independent features. In certain aspects each feature is included.

Methods for identifying candidate antigens also include generating an output for constructing a personalized antigen-based vaccine by identifying one or more antigens of HIV that are likely to be presented. As an example, one such method may comprise the steps of: obtaining HIV sequencing data, wherein the HIV sequencing data is used to obtain data representing peptide sequences of each of a set of antigens; encoding the peptide sequences of each of the antigens into a corresponding numerical vector, each numerical vector including information regarding a plurality of amino acids that make up the peptide sequence and a set of positions of the amino acids in the peptide sequence; inputting the numerical vectors, using a computer processor, into a deep learning presentation model to generate a set of presentation likelihoods for the set of antigens, each presentation likelihood in the set representing the likelihood that a corresponding antigen is presented by MHC proteins of class I MHC alleles; selecting a subset of the set of antigens based on the set of presentation likelihoods to generate a set of selected antigens; and generating the output for constructing the personalized antigen-based vaccine based on the set of selected antigens. In one aspect, each antigen in the set of antigens is encoded by genes in the HIV genome (e.g., env, gag, nef, pol, rev, tat, vif, vir, or viu).

Specific methods for identifying antigens are known to those skilled in the art, for example the methods described in more detail in international patent application publications WO/2017/106638, WO/2018/195357, and WO/2018/208856, each herein incorporated by reference, in their entirety, for all purposes.

A method of treating a subject is disclosed herein, comprising performing the steps of any of the antigen identification methods described herein, and further comprising obtaining an antigen-based vaccine comprising the set of selected antigens, and administering the antigen-based vaccine to the subject, wherein, optionally, the subject has HIV.

A method disclosed herein can also include identifying one or more T cells that are antigen-specific for at least one of the antigens in the subset. In some embodiments, the identification comprises co-culturing the one or more T cells with one or more of the antigens in the subset under conditions that expand the one or more antigen-specific T cells. In further embodiments, the identification comprises contacting the one or more T cells with a tetramer comprising one or more of the antigens in the subset under conditions that allow binding between the T cell and the tetramer. In even further embodiments, the method disclosed herein can also include identifying one or more T cell receptors (TCR) of the one or more identified T cells. In certain embodiments, identifying the one or more T cell receptors comprises sequencing the T cell receptor sequences of the one or more identified T cells. The method disclosed herein can further comprise genetically engineering a plurality of T cells to express at least one of the one or more identified T cell receptors; culturing the plurality of T cells under conditions that expand the plurality of T cells; and infusing the expanded T cells into the subject. In some embodiments, genetically engineering the plurality of T cells to express at least one of the one or more identified T cell receptors comprises cloning the T cell receptor sequences of the one or more identified T cells into an expression vector; and transfecting each of the plurality of T cells with the expression vector. In some embodiments, the method disclosed herein further comprises culturing the one or more identified T cells under conditions that expand the one or more identified T cells; and infusing the expanded T cells into the subject.

Also disclosed herein is an isolated T cell that is antigen-specific for at least one selected antigen in the subset.

Also disclosed herein is a method for manufacturing a HIV vaccine, comprising the steps of: obtaining HIV sequencing data, wherein the HIV sequencing data is used to obtain data representing peptide sequences of each of a set of antigens; inputting the peptide sequence of each antigen into one or more presentation models to generate a set of numerical likelihoods that each of the antigens is presented by one or more MHC alleles, the set of numerical likelihoods having been identified at least based on received mass spectrometry data; and selecting a subset of the set of antigens based on the set of numerical likelihoods to generate a set of selected antigens; and producing or having produced a HIV vaccine comprising the set of selected antigens. In one aspect, each antigen in the set of antigens is encoded by genes in the HIV genome (e.g., env, gag, nef, pol, rev, tat, vif, vir, or viu).

Also disclosed herein is an antigen-based vaccine including a set of selected antigens selected by performing the method comprising the steps of: obtaining HIV sequencing data, wherein the HIV sequencing data is used to obtain data representing peptide sequences of each of a set of antigens; inputting the peptide sequence of each antigen into one or more presentation models to generate a set of numerical likelihoods that each of the antigens is presented by one or more MHC alleles, the set of numerical likelihoods having been identified at least based on received mass spectrometry data; and selecting a subset of the set of antigens based on the set of numerical likelihoods to generate a set of selected antigens; and producing or having produced a HIV vaccine comprising the set of selected antigens. In one aspect, each antigen in the set of antigens is encoded by genes in the HIV genome (e.g., env, gag, nef, pol, rev, tat, vif, vir, or viu).

The antigen-based vaccine may include one or more of a nucleotide sequence, a polypeptide sequence, RNA, DNA, a cell, a plasmid, or a vector.

The antigen-based vaccine may include one or more antigens that is immunogenic in the subject.

The antigen-based vaccine may not include one or more antigens that induce an autoimmune response against normal tissue in the subject.

The antigen-based vaccine may include an adjuvant.

The antigen-based vaccine may include an excipient.

A method disclosed herein may also include selecting antigens that have an increased likelihood of being presented by immune cells of the subject relative to unselected antigens based on the presentation model.

A method disclosed herein may also include selecting antigens that have an increased likelihood of being capable of being presented to naïve T cells by professional antigen presenting cells (APCs) relative to unselected antigens based on the presentation model, optionally wherein the APC is a dendritic cell (DC).

A method disclosed herein may also include selecting antigens that have an increased likelihood of being capable of inducing a HIV-specific immune response in the subject relative to unselected antigens based on the presentation model.

A method disclosed herein may also include selecting antigens that have a decreased likelihood of being subject to inhibition via central or peripheral tolerance relative to unselected antigens based on the presentation model.

A method disclosed herein may also include selecting antigens that have a decreased likelihood of being capable of inducing an autoimmune response to normal tissue in the subject relative to unselected antigens based on the presentation model.

The exome or transcriptome nucleotide sequencing and/or expression data may be obtained by performing sequencing on the tissue.

The sequencing may be next generation sequencing (NGS) or any massively parallel sequencing approach.

The set of numerical likelihoods may be further identified by at least MHC-allele interacting features comprising at least one of: the predicted affinity with which the MHC allele and the antigen encoded peptide bind; the predicted stability of the antigen encoded peptide-MHC complex; the sequence and length of the antigen encoded peptide; the probability of presentation of antigen encoded peptides with similar sequence in cells from other individuals expressing the particular MHC allele as assessed by mass-spectrometry proteomics or other means; the expression levels of the particular MHC allele in the subject in question (e.g. as measured by RNA-seq or mass spectrometry); the overall antigen encoded peptide-sequence-independent probability of presentation by the particular MHC allele in other distinct subjects who express the particular MHC allele; the overall antigen encoded peptide-sequence-independent probability of presentation by MHC alleles in the same family of molecules (e.g., HLA-A, HLA-B, HLA-C, HLA-DQ, HLA-DR, HLA-DP) in other distinct subjects.

The set of numerical likelihoods are further identified by at least MHC-allele noninteracting features comprising at least one of: the C- and N-terminal sequences flanking the antigen encoded peptide within its source protein sequence; the presence of protease cleavage motifs in the antigen encoded peptide, optionally weighted according to the expression of corresponding proteases in tissue (as measured by RNA-seq or mass spectrometry); the turnover rate of the source protein as measured in the appropriate cell type; the length of the source protein, the level of expression of proteasome, immunoproteasome, thymoproteasome, or other proteases (which may be measured by RNA-seq, proteome mass spectrometry, or immunohistochemistry); the expression of the source gene (e.g., env, gag, nef, pol, rev, tat, vif, vir, or viu) of the antigen encoded peptide (e.g., as measured by RNA-seq or mass spectrometry); features describing the properties of the domain of the source protein containing the peptide, for example: secondary or tertiary structure (e.g., alpha helix vs beta sheet); alternative splicing; the probability of presentation of peptides from the source protein of the antigen encoded peptide in question in other distinct subjects; the probability that the peptide will not be detected or over-represented by mass spectrometry due to technical biases; the expression of various gene modules/pathways as measured by RNASeq (which need not contain the source protein of the peptide) that are informative about the state of the immune cells; the probability that the peptide binds to the TAP or the measured or predicted binding affinity of the peptide to the TAP; the expression level of TAP (which may be measured by RNA-seq, proteome mass spectrometry, immunohistochemistry); presence or absence of functional germline polymorphisms, including, but not limited to: in genes encoding the proteins involved in the antigen presentation machinery (e.g., B2M, HLA-A, HLA-B, HLA-C, TAP-1, TAP-2, TAPBP, CALR, CNX, ERP57, HLA-DM, HLA-DMA, HLA-DMB, HLA-DO, HLA-DOA, HLA-DOB, HLA-DP, HLA-DPA1, HLA-DPB1, HLA-DQ, HLA-DQA1, HLA-DQA2, HLA-DQB1, HLA-DQB2, HLA-DR, HLA-DRA, HLA-DRB1, HLA-DRB3, HLA-DRB4, HLA-DRB5 or any of the genes coding for components of the proteasome or immunoproteasome); and HIV subtype (e.g., A1, A2, B, C, D, F1, F2, G, H, J, and K); smoking history.

A method disclosed herein may also include obtaining an antigen-based vaccine comprising the set of selected antigens or a subset thereof, optionally further comprising administering the antigen-based vaccine to the subject.

At least one of the antigens in the set of candidate antigens, when in polypeptide form, may include at least one of: a binding affinity with MHC with an IC50 value of less than 1000 nM, for MHC Class I polypeptides a length of 8-15, 8, 9, 10, 11, 12, 13, 14, or 15 amino acids, for MHC Class II polypeptides a length of 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the polypeptide in the parent protein sequence promoting proteasome cleavage, and presence of sequence motifs promoting TAP transport. For MHC Class II, presence of sequence motifs within or near the peptide promoting cleavage by extracellular or lysosomal proteases (e.g., cathepsins) or HLA-DM catalyzed HLA binding.

A method disclosed herein can also include selecting a subset of antigens, wherein the subset of antigens is selected because each has an increased likelihood that it is presented on the surface of HIV relative to one or more other antigens.

A method disclosed herein can also include selecting a subset of candidate antigens, In one aspect, the subset of candidate antigens is selected because each has an increased likelihood that it is capable of inducing a HIV-specific immune response in the subject relative to one or more other antigens. In one aspect, the subset of candidate antigens is selected because each has an increased likelihood that it is capable of being presented to naïve T cells by professional antigen presenting cells (APCs) relative to one or more distinct antigens, optionally wherein the APC is a dendritic cell (DC). In one aspect, the subset of candidate antigens is selected because each has a decreased likelihood that it is subject to inhibition via central or peripheral tolerance relative to one or more other antigens. In one aspect, the subset of antigens is selected because each has a decreased likelihood that it is capable of inducing an autoimmune response to normal tissue in the subject relative to one or more other antigens.

The practice of the methods herein will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### III. Identifying HIV Epitope Sequences

Also disclosed herein are methods for the identification of HIV epitope sequences. In one aspect, HIV epitope sequences are identified from HIV nucleotide sequences that are sequenced from the HIV genome.

The HIV nucleotide sequences can be encoded by one of nine HIV genes including env, gag, nef, pol, rev, tat, vif, vpr, and vpu. Sequencing of the HIV genome can be done on a nucleic acid sample obtained from any cell type or tissue. For example, a HIV sample can be obtained from a bodily fluid, e.g., blood, obtained by known techniques (e.g. venipuncture) or saliva.

HIV nucleotide sequence information can be generated directly from millions of individual molecules of nucleic acids obtained from HIV. Real-time single molecule sequencing-by-synthesis technologies rely on the detection of fluorescent nucleotides as they are incorporated into a nascent strand of DNA that is complementary to the template being sequenced. In one method, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers function as a fixed position site for sequence determination using multiple cycles of synthesis, detection, and chemical cleavage of the dye-linker to remove the dye. Each cycle consists of adding the polymerase/labeled nucleotide mixture, rinsing, imaging and cleavage of dye. In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain. Other sequencing-by-synthesis technologies also exist.

Any suitable sequencing-by-synthesis platform can be used to generate HIV nucleic acid sequences. As described above, four major sequencing-by-synthesis platforms are currently available: the Genome Sequencers from Roche/454 Life Sciences, the IG Analyzer from Illumina/Solexa, the SOLiD system from Applied BioSystems, and the Heliscope system from Helicos Biosciences. Sequencing-by-synthesis platforms have also been described by Pacific BioSciences and VisiGen Biotechnologies. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids can be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair as described in, e.g., US Patent Application No. 2006/0252077) can be linked to each fragment to be captured on a surface coated with a respective second member of that coupling pair.

Subsequent to the capture, the sequence can be analyzed, for example, by single molecule detection/sequencing, e.g., as described in the Examples and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide can be incorporated and multiple lasers can be utilized for stimulation of incorporated nucleotides.

Sequencing can also include other massively parallel sequencing or next generation sequencing (NGS) techniques and platforms. Additional examples of massively parallel sequencing techniques and platforms are the Illumina HiSeq or MiSeq, Thermo PGM or Proton, the Pac Bio RS II or Sequel, Qiagen's Gene Reader, and the Oxford Nanopore MinION. Additional similar current massively parallel sequencing technologies can be used, as well as future generations of these technologies.

In some aspects, HIV nucleotide sequences of different HIV categories, types, and subtypes are obtained from available open-source databases (e.g., the Los Alamos National Lab's HIV database).

Having obtained the HIV nucleotide sequences, HIV epitope sequences are extracted from the HIV nucleotide sequences. As one example, extraction of the HIV epitope sequences can be conducted by employing a sliding window, where length of the sliding window corresponds to the length of a HIV epitope sequence. To illustrate the extraction process, the sliding window is applied to a first HIV nucleotide sequence. The set of nucleotide base sequences in the sliding window is extracted as a first HIV epitope sequence. The sliding window is shifted by one nucleotide base and the next set of nucleotide base sequences in the shifted sliding window is a second HIV epitope sequence. This process repeats until the sliding window has been applied across all HIV nucleotide sequences.

In one aspect, each HIV epitope sequence is 18, 19, 20, 21, 22, 23, 24, 25, 26, 27, 28, 29, 30, 31, 32, 33, 34, 35, 36, 37, 38, or 39 nucleotide bases in length (e.g., 6-13 amino acid sequences in length). In one aspect, each HIV epitope sequence is 24, 25, 26, 27, 28, 29, 30, 31, 32, or 33 nucleotide bases in length (e.g., 8-11 amino acid sequences in length).

Additionally, a variety of methods are available for detecting the presence of a particular mutation in an HIV sequence. Advancements in this field have provided accurate, easy, and inexpensive large-scale SNP genotyping. For example, several techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods utilize amplification of a target genetic region, typically by PCR. Still other methods, based on the generation of small signal molecules by invasive cleavage followed by mass spectrometry or immobilized padlock probes and rolling-circle amplification. Several of the methods known in the art for detecting specific mutations are summarized below.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

Several methods have been developed to facilitate analysis of single nucleotide polymorphisms in genomic DNA or cellular RNA. For example, a single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide(s) present in the polymorphic site of the target molecule is complementary to that of the nucleotide derivative used in the reaction. This method has the advantage that it does not require the determination of large amounts of extraneous sequence data.

A solution-based method can be used for determining the identity of a nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. can be a heterogeneous phase assay, in which the primer or the target molecule is immobilized to a solid phase.

### IV. Antigens

Antigens can include nucleotides or polypeptides. For example, an antigen can be an RNA sequence that encodes for a polypeptide sequence. Antigens useful in vaccines can therefore include nucleotide sequences or polypeptide sequences. In one aspect, antigen peptides can be described in the context of their coding sequence where an antigen includes the nucleotide sequence (e.g., DNA or RNA) that codes for the related polypeptide sequence. In one aspect, antigens bind to MHC proteins, and therefore, can be presented by antigen presenting cells such that epitope sequences on the antigens can bind to T cell receptors. In some scenarios, antigens bind to MHC class I proteins. In some scenarios, antigens bind to MHC class II proteins. In some scenarios, antigens bind to both MHC class I and class II proteins.

Antigens may be derived from either of the two major categories of HIV (HIV-1 or HIV-2). Additionally, antigens may be derived from the different types of HIV-1 including Group N, Group O, or Group P. Additionally, antigens derived from Group N may be from one of subtypes A1, A2, B, C, D, F1, F2, G, H, J, or K.

Antigens (and corresponding epitope sequences) derived from HIV may differ depending on the category, type, or subtype of HIV. For example, epitope sequences of HIV antigens derived from different HIV subtypes are shown in the second column of Tables 35-45. Additionally, there are a number of epitope sequences that are invariant across the HIV subtypes. Therefore, certain epitope sequences are included in more than one of Tables 35-45.

One or more polypeptides encoded by an antigen nucleotide sequence can comprise at least one of: a binding affinity with MHC with an IC50 value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport. For MHC Class II peptides a length 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the peptide promoting cleavage by extracellular or lysosomal proteases (e.g., cathepsins) or HLA-DM catalyzed HLA binding.

One or more antigens can be presented on HIV.

One or more antigens can be immunogenic in a subject, e.g., capable of eliciting a T cell response or a B cell response in the subject. Optionally, the subject may have HIV.

One or more antigens that induce an autoimmune response in a subject can be excluded from consideration in the context of vaccine generation for a subject that optionally has HIV.

The size of at least one antigenic peptide molecule can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the antigenic peptide molecules are equal to or less than 50 amino acids.

Antigenic peptides and polypeptides can be: for MHC Class I, 15 residues or less in length and usually consist of between about 8 and about 11 residues, particularly 9 or 10 residues; for MHC Class II, 6-30 residues, inclusive.

If desirable, a longer peptide can be designed in several ways. In one case, when presentation likelihoods of peptides on HLA alleles are predicted or known, a longer peptide could consist of either: (1) individual presented peptides with an extensions of 2-5 amino acids toward the N- and C-terminus of each corresponding gene product; (2) a concatenation of some or all of the presented peptides with extended sequences for each. Use of a longer peptide allows endogenous processing by patient cells and may lead to more effective antigen presentation and induction of T cell responses.

Antigenic peptides and polypeptides can be presented on a HLA protein. In some aspects, an antigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In some aspects, antigenic peptides and polypeptides do not induce an autoimmune response and/or invoke immunological tolerance when administered to a subject.

Antigenic peptides and polypeptides having a desired activity or property can be modified to provide certain desired attributes, e.g., improved pharmacological characteristics, while increasing or at least retaining substantially all of the biological activity of the unmodified peptide to bind the desired MHC molecule and activate the appropriate T cell. For instance, antigenic peptide and polypeptides can be subject to various changes, such as substitutions, either conservative or non-conservative, where such changes might provide for certain advantages in their use, such as improved MHC binding, stability or presentation. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications can be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

Modifications of peptides and polypeptides with various amino acid mimetics or unnatural amino acids can be particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half-life of the peptides can be conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4 degrees C.) for 15 minutes and then spun to pellet the precipitated serum proteins. The presence of the peptides is then determined by reversed-phase HPLC using stability-specific chromatography conditions.

The peptides and polypeptides can be modified to provide desired attributes other than improved serum half-life. For instance, the ability of the peptides to induce CTL activity can be enhanced by linkage to a sequence which contains at least one epitope that is capable of inducing a T helper cell response. Immunogenic peptides/T helper conjugates can be linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. Alternatively, the peptide can be linked to the T helper peptide without a spacer.

An antigenic peptide can be linked to the T helper peptide either directly or via a spacer either at the amino or carboxy terminus of the peptide. The amino terminus of either the antigenic peptide or the T helper peptide can be acylated. Exemplary T helper peptides include tetanus toxoid 830-843, influenza 307-319, malaria circumsporozoite 382-398 and 378-389.

Proteins or peptides can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

In a further aspect an antigen includes a nucleic acid (e.g. polynucleotide) that encodes an antigenic peptide or portion thereof. The polynucleotide can be, e.g., DNA, cDNA, PNA, CNA, RNA (e.g., mRNA), either single- and/or double-stranded, or native or stabilized forms of polynucleotides, such as, e.g., polynucleotides with a phosphorothiate backbone, or combinations thereof and it may or may not contain introns. A still further aspect provides an expression vector capable of expressing a polypeptide or portion thereof. Expression vectors for different cell types are well known in the art and can be selected without undue experimentation. Generally, DNA is inserted into an expression vector, such as a plasmid, in proper orientation and correct reading frame for expression. If necessary, DNA can be linked to the appropriate transcriptional and translational regulatory control nucleotide sequences recognized by the desired host, although such controls are generally available in the expression vector. The vector is then introduced into the host through standard techniques. Guidance can be found e.g. in Sambrook et al. (1989) Molecular Cloning, A Laboratory Manual, Cold Spring Harbor Laboratory, Cold Spring Harbor, N.Y.

### V. Vaccine Compositions

Also disclosed herein is an immunogenic composition, e.g., a vaccine composition, capable of raising a specific immune response, e.g., a HIV-specific immune response. Vaccine compositions typically comprise one or more antigens selected using a method described herein.

In one aspect, the vaccine composition includes one antigen with an epitope sequence selected from any one of SEQ ID Nos: 325-22349. In other aspects, the vaccine composition includes a plurality of antigens with epitope sequences selected from any one of SEQ ID Nos: 325-22349. In such scenarios, at least two of the plurality of antigens can be distinct peptides. By distinct polypeptides is meant that the peptides vary by length, amino acid sequence, or both.

In some aspects, the vaccine composition includes one or more epitope encoding nucleic acid sequences. In one aspect, the epitope encoding nucleic acid sequences are MHC class I epitope encoding nucleic acid sequences. Each epitope encoding nucleic acid sequence can encode for an antigen with epitope sequences selected from any one of SEQ ID Nos: 325-22349.

In some aspects, a vaccine composition can contain between 1 and 30 peptides, 2, 3, 4, 5, 6, 7, 8, 9, 10, 11, 12, 13, 14, 15, 16, 17, 18, 19, 20, 21, 22, 23, 24, 25, 26, 27, 28, 29, or 30 different peptides, 6, 7, 8, 9, 10 11, 12, 13, or 14 different peptides, or 12, 13 or 14 different peptides. In various embodiments, the peptides included in the vaccine composition include an epitope sequence selected from any one of SEQ ID Nos: 325-22349 shown in Tables 35-45. Peptides can include post-translational modifications. A vaccine can contain between 1 and 100 or more nucleotide sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different nucleotide sequences, 6, 7, 8, 9, 10 11, 12, 13, or 14 different nucleotide sequences, or 12, 13 or 14 different nucleotide sequences. A vaccine can contain between 1 and 30 antigen sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen sequences, 6, 7, 8, 9, 10 11, 12, 13, or 14 different antigen sequences, or 12, 13 or 14 different antigen sequences. A vaccine can contain between 1 and 30 antigen-encoding sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen-encoding sequences, 6, 7, 8, 9, 10 11, 12, 13, or 14 different antigen-encoding sequences, or 12, 13 or 14 different antigen-encoding sequences. In various embodiments, the antigen-encoding sequences encode for antigens that comprise epitope sequences selected from any one of SEQ ID Nos: 325-22349 shown in Tables 35-45.

Further details of the selection of candidate epitope sequences or antigen-encoding nucleic acid sequences that are to be included in the vaccine composition are described below.

In one embodiment, different peptides and/or polypeptides or nucleotide sequences encoding them are selected so that the peptides and/or polypeptides capable of associating with different MHC molecules, such as different MHC class I molecules and/or different MHC class II molecules. In some aspects, one vaccine composition comprises coding sequence for peptides and/or polypeptides capable of associating with the most frequently occurring MHC class I molecules and/or different MHC class II molecules. Hence, vaccine compositions can comprise different fragments capable of associating with at least 2 preferred, at least 3 preferred, or at least 4 preferred MHC class I molecules and/or different MHC class II molecules.

The vaccine composition can be capable of raising a specific cytotoxic T-cells response and/or a specific helper T-cell response.

Antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more antigen peptides. The sequences may be flanked by non-mutated sequences, may be separated by linkers or may be preceded with one or more sequences targeting a subcellular compartment (See, e.g., Gros et al., Prospective identification of antigen-specific lymphocytes in the peripheral blood of melanoma patients, Nat Med. (2016) 22 (4):433-8, Stronen et al., Targeting of cancer antigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20(13):3401-10). Upon introduction into a host, infected cells express the antigens, and thereby elicit a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of antigens, e.g., Salmonella typhi vectors, and the like will be apparent to those skilled in the art from the description herein.

V.A. Antigen Vaccine Sequence Selection

Selected candidate antigens with epitope sequences are included in antigen-based vaccines. In various embodiments, epitope sequences for candidate antigens are selected using a presentation model, as is described in further detail below in reference to the presentation model. In various embodiments, epitope sequences for candidate antigens are selected from a Los Alamos National Lab's HIV database, such as the Los Alamos Best-defined (“A-list”) CTL epitopes,¹⁰⁸ which is incorporated by reference in its entirety. In various embodiments, epitope sequences for candidate antigens are selected using a presentation model that is deployed to evaluate epitope sequences from the Los Alamos Best-defined (“A-list”) CTL epitopes.¹⁰⁸ Although the subsequent description refers to inclusion of antigenic peptides in the antigen-based vaccine, one skilled in the art may understand that the subsequent description can be applied for the inclusion of antigen-encoding nucleic acid sequences in an antigen cassette, where the antigen-encoding nucleic acid sequences encode for these antigenic peptides. Further details of the antigen cassette are discussed below.

In one aspect, each antigen-based vaccine may be developed for patients with a haplotype that includes one or more particular HLA alleles. Therefore, a patient with a particular HLA can be treated or vaccinated with an antigen-based vaccine that is developed specifically for the particular HLA allele. In some aspects, each antigen-based vaccine is developed for patients with a haplotype that includes particular combinations of HLA alleles. In one embodiment, the particular combination of HLA alleles is known to be expressed by a population of individuals of a particular ancestral descent. Thus, a patient who is of that ancestral descent is also likely to express the combination of HLA alleles and therefore, can be a candidate for a vaccine that includes antigens that are likely to be presented by the expressed combination of HLA alleles. In some aspects, an antigen-based vaccine can be developed with a sufficient number of antigens such that a patient of any ancestral descent is likely to present a subset of the antigens included in the antigen-based vaccine. In other words, with a sufficient number of antigens in the antigen-based vaccine, such an antigen-based vaccine can be efficacious for any patient.

As an example, antigen-based vaccines can be developed for any one or more of the following HLA alleles: A0101, A0201, A0203, A0204, A0205, A0206, A0207, A0208, A0301, A0302, A1101, A2301, A2402, A2501, A2601, A2602, A2603, A2901, A2902, A3001, A3002, A3004, A3101, A3201, A3301, A3303, A6801, A6802, B0702, B0801, B1301, B1302, B1401, B1402, B1501, B1502, B1503, B1510, B1513, B1801, B2702, B2705, B3501, B3502, B3503, B3508, B3512, B3701, B3801, B3901, B3906, B4001, B4002, B4006, B4102, B4402, B4403, B4405, B4601, B4801, B4901, B5001, B5101, B5401, B5501, B5502, B5601, B5701, B5801, C0102, C0202, C0302, C0303, C0304, C0401, C0501, C0602, C0701, C0702, C0704, C0801, C0802, C0803, C1203, C1402, C1403, C1502, C1601, C1602, C1604, C1701. Antigens for inclusion in the antigen-based vaccine can be selected by reference to Tables 35-45 (e.g., any one of SEQ ID Nos: 325-22349), where each relevant epitope sequence of an antigen for inclusion is selected by identifying rows that list the particular HLA allele that the antigen-based vaccine is developed for. Notably, certain epitope sequences are invariant across multiple HIV subtypes and therefore, appear across multiple tables in Tables 35-45. In some aspects, antigens for inclusion in the antigen-based vaccine can each include epitope sequences that appear in more than one of Tables 35-45. Additionally, antigens for inclusion in the antigen-based vaccine can be selected from a list of validated HIV epitopes. Examples of validated HIV epitopes can be found in the journal article “Best-Characterized HIV-1 CTL Epitopes: The 2013 Update” (which refers to validated HIV epitopes as “best defined HIV CTL epitopes in Table I-A-1), which is hereby incorporated by reference in its entirety.¹⁰⁵ Additional examples of validated HIV epitopes can be found in the journal article “The 2019 Optimal HIV CTL epitopes update: Growing diversity in epitope length and HLA restriction” which is hereby incorporated by reference in its entirety.¹⁰⁹

For example, referring to the first row of Table 35, if an antigen-based vaccine is developed for the A2501 HLA allele, then the epitope sequence “DTIAIAVAGW (SEQ ID NO: 756)” can be selected for inclusion. Such an antigen-based vaccine can include additional epitope sequences from Tables 35-45 that share a row with the A2501 HLA allele. For example, referring to Table 36, the epitope sequence “DTIAVAVAEW (SEQ ID NO: 2606)” can additionally be selected for inclusion in the antigen-based vaccine.

In some aspects, antigen-based vaccines can be developed for combinations of the aforementioned HLA alleles. For example, if certain combinations of HLA alleles are known to be expressed together by subjects, then an antigen-based vaccine can be developed for the combination of expressed HLA alleles. In some aspects, 1, 2, 3, 4, 5, 6, 7, 8, 9, or 10 HLA alleles are included in the combination of HLA alleles. Antigens for inclusion in the antigen-based vaccine can be selected by reference to Tables 35-45 (e.g., any one of SEQ ID Nos: 325-22349), where each relevant epitope sequence of an antigen for inclusion is selected by identifying rows that list any one HLA allele in the combination of HLA alleles.

In one aspect, each antigen-based vaccine may be developed for patients that are infected, exposed, or susceptible to infection by a particular category, type, or subtype of HIV. Therefore, a patient can be treated or vaccinated with an antigen-based vaccine that is developed specifically for the particular category, type, or subtype of HIV that the patient is infected, exposed, or susceptible to infection to.

For example, antigen-based vaccines can be developed for any one of the categories (e.g., HIV-1 or HIV-2), types (Group N, Group O, or Group P), or subtypes (A1, A2, B, C, D, F1, F2, G, H, J, or K) of HIV. Antigens for inclusion in the antigen-based vaccine can be selected by reference to Tables 35-45 (e.g., any one of SEQ ID Nos: 325-22349).

In various embodiments, an antigen-based vaccine developed for HIV subtype A1 can include one or more antigens with HIV epitope sequences shown in Table 35 (e.g., any one of SEQ ID NOs: 325-2165).

In various embodiments, an antigen-based vaccine developed for HIV subtype A2 can include one or more antigens with HIV epitope sequences shown in Table 36 (e.g., any one of SEQ ID NOs: 2166-4106).

In various embodiments, an antigen-based vaccine developed for HIV subtype B can include one or more antigens with HIV epitope sequences shown in Table 37 (e.g., any one of SEQ ID NOs: 2166-4106).

In various embodiments, an antigen-based vaccine developed for HIV subtype C can include one or more antigens with HIV epitope sequences shown in Table 38 (e.g., any one of SEQ ID NOs: 6242-8389).

In various embodiments, an antigen-based vaccine developed for HIV subtype D can include one or more antigens with HIV epitope sequences shown in Table 39 (e.g., any one of SEQ ID NOs: 8930-10626).

In various embodiments, an antigen-based vaccine developed for HIV subtype F1 can include one or more antigens with HIV epitope sequences shown in Table 40 (e.g., any one of SEQ ID NOs: 10627-12810).

In various embodiments, an antigen-based vaccine developed for HIV subtype F2 can include one or more antigens with HIV epitope sequences shown in Table 41 (e.g., any one of SEQ ID NOs: 12811-15079).

In various embodiments, an antigen-based vaccine developed for HIV subtype G can include one or more antigens with HIV epitope sequences shown in Table 42 (e.g., any one of SEQ ID NOs: 15080-17174).

In various embodiments, an antigen-based vaccine developed for HIV subtype H can include one or more antigens with HIV epitope sequences shown in Table 43 (e.g., any one of SEQ ID NOs: 17175-19388).

In various embodiments, an antigen-based vaccine developed for HIV subtype J can include one or more antigens with HIV epitope sequences shown in Table 44 (e.g., any one of SEQ ID NOs: 19389-21003).

In various embodiments, an antigen-based vaccine developed for HIV subtype K can include one or more antigens with HIV epitope sequences shown in Table 45 (e.g., any one of SEQ ID NOs: 21004-22349).

In one aspect, each antigen-based vaccine may be developed for patients taking into consideration both 1) the patient's HLA type that includes the expression of one or more particular HLA alleles and 2) the particular category, type, or subtype of HIV that the patient is infected, exposed to, or susceptible to exposure to. As an example, a patient that expresses a particular HLA allele and who is exposed to or susceptible to exposure to a subtype of HIV can be treated or vaccinated with an antigen-based vaccine that is developed specifically for the subtype of HIV and the patient's expressed HLA allele. Antigens for inclusion in the antigen-based vaccine can be selected by reference to one of Tables 35-45 (e.g., any one of SEQ ID Nos: 325-22349), where each relevant epitope sequence of an antigen for inclusion is selected by identifying rows in that Table that list a particular HLA allele.

For example, an antigen-based vaccine can be developed for HIV subtype A1 and for patients with the B4102 HLA allele. Referring to Table 35, a first antigen with epitope sequence “AEVVQKVTM (SEQ ID NO: 1594)” and a second antigen with epitope sequence “AEVVQKVVM (SEQ ID NO: 1595)” can be selected for inclusion in the antigen-based vaccine. Such an antigen-based vaccine can include additional HIV epitope sequences from Table 35 (e.g., any of SEQ ID NOs: 1594-1642) that share a row with the B4102 HLA allele. As another example, an antigen-based vaccine can be developed for HIV subtype A2 and for patients with the B4001 HLA allele. Referring to Table 36, a first antigen with epitope sequence “TESNDTITL (SEQ ID NO: 3424)” and a second antigen with epitope sequence “AEDPEREVL (SEQ ID NO: 3425)” can be selected for inclusion in the antigen-based vaccine. Such an antigen-based vaccine can include additional HIV epitope sequences from Table 36 that share a row with the B4001 HLA allele (e.g., any of SEQ ID NOs: 3424-3458).

In particular embodiments, one or more HIV epitope sequences or one or more HIV epitope sequences encoded by a HIV epitope encoding sequence that are selected for inclusion in an antigen-based vaccine can include any of SEQ ID NOs: 325-328, 2166-2178, 4107-4113, 6242-6248, 8390-8397, 10627-10633, 12811-12820, 15080-15086, 17175-17184, 19389-19396, or 21004-21009.

In particular embodiments, one or more HIV epitope sequences or one or more HIV epitope sequences encoded by HIV epitope encoding sequences that are selected for inclusion in an antigen-based vaccine can include any of SEQ ID NOs: 329-353, 2179-2200, 4114-4134, 6249-6270, 8398-8415, 10634-10654, 12821-12850, 15087-15107, 17185-17213, 19397-19420, or 21010-21031.

In particular embodiments, one or more HIV epitope sequences or one or more HIV epitope sequences encoded by HIV epitope encoding sequences that are selected for inclusion in an antigen-based vaccine can include any of SEQ ID NOs: 354-403, 2201-2248, 4135-4177, 6271-6315, 8416-8474, 10655-10700, 12851-12912, 15108-15155, 17214-17264, 19421-19463, or 21032-21064.

In particular embodiments, one or more HIV epitope sequences or one or more HIV epitope sequences encoded by HIV epitope encoding sequences that are selected for inclusion in an antigen-based vaccine can include any of SEQ ID NOs: 404-469, 2249-2326, 4178-4261, 6316-6400, 8475-8558, 10701-10768, 12913-12994, 15156-15214, 17265-17349, 19464-19518, 21065-21117.

In particular embodiments, one or more HIV epitope sequences or one or more HIV epitope sequences encoded by HIV epitope encoding sequences that are selected for inclusion in an antigen-based vaccine can include any of 470-526, 2327-2379, 6401-6450, 8559-8626, 10769-10822, 12995-13056, 15215-15263, 17350-17405, 19519-19570, and 21118-21161.

In particular embodiments, one or more HIV epitope sequences or one or more HIV epitope sequences encoded by HIV epitope encoding sequences that are selected for inclusion in an antigen-based vaccine can include any of SEQ ID NOs: 527-565, 2380-2421, 6451-6492, 8627-8671, 10823-10867, 10357-13098, 15264-15292, 17406-17448, 19571-19604, and 21162-21192.

In particular embodiments, one or more HIV epitope sequences or one or more HIV epitope sequences encoded by HIV epitope encoding sequences that are selected for inclusion in an antigen-based vaccine can include any of 566-587, 2422-2438, 6493-6509, 8672-8689, 10868-10887, 13099-13125, 15293-15307, 17449-17473, 19605-19618, and 21193-21205.

In particular embodiments, one or more HIV epitope sequences or one or more HIV epitope sequences encoded by HIV epitope encoding sequences that are selected for inclusion in an antigen-based vaccine can include any of SEQ ID NOs: 588-630, 2439-2477, 6510-6548, 8690-8733, 10888-10931, 13126-13179, 15308-15336, 17474-17512, 19619-19649, and 21206-21233.

In particular embodiments, one or more HIV epitope sequences or one or more HIV epitope sequences encoded by HIV epitope encoding sequences that are selected for inclusion in an antigen-based vaccine can include any of 631-650, 2478-2501, 6549-6573, 8734-8761, 10932-10969, 13180-13224, 15337-15354, 17513-17543, 19650-19665, and 21234-21247.

In particular embodiments, one or more HIV epitope sequences or one or more HIV epitope sequences encoded by HIV epitope encoding sequences that are selected for inclusion in an antigen-based vaccine can include any of 651-682, 2502-2541, 6574-6618, 8762-8809, 10970-11026, 13225-13290, 15355-15396, 17544-17603, 19666-19697, and 21248-21274.

In particular embodiments, one or more HIV epitope sequences or one or more HIV epitope sequences encoded by HIV epitope encoding sequences that are selected for inclusion in an antigen-based vaccine can include any of SEQ ID NOs: 683-726, 2542-2583, 6619-6668, 8810-8862, 11027-11087, 13291-13370, 15397-15451, 17604-17652, 19698-19726, and 21275-21309.

In particular embodiments, one or more HIV epitope sequences or one or more HIV epitope sequences encoded by HIV epitope encoding sequences that are selected for inclusion in an antigen-based vaccine can include any of SEQ ID NOs: 727-741, 2584-2593, 6669-6685, 8863-8871, 11088-11103, 13371-13385, 15452-15465, 17653-17667, 19727-19738, and 21310-21317. Such an antigen-based vaccine can be useful for treating a patient who expresses the HLA allele A2301.

In particular embodiments, one or more HIV epitope sequences or one or more HIV epitope sequences encoded by HIV epitope encoding sequences that are selected for inclusion in an antigen-based vaccine can include any of SEQ ID NOs: 742-755, 2594-2605, 6686-6698, 8872-8885, 11104-11116, 13386-13397, 15466-15479, 17668-17679, 19739-19750, and 21318-21323. Such an antigen-based vaccine can be useful for treating a patient who expresses the HLA allele A2402.

In particular embodiments, one or more HIV epitope sequences or one or more HIV epitope sequences encoded by HIV epitope encoding sequences that are selected for inclusion in an antigen-based vaccine can include any of SEQ ID NOs: SEQ ID NOs: 756-769, 2606-2622, 6699-6711, 8886-8903, 11117-11132, 13398-13414, 15480-15505, 17680-17693, 19751-19760, and 21324-21333. Such an antigen-based vaccine can be useful for treating a patient who expresses the HLA allele 2501.

In particular embodiments, one or more HIV epitope sequences or one or more HIV epitope sequences encoded by HIV epitope encoding sequences that are selected for inclusion in an antigen-based vaccine can include any of SEQ ID NOs: 770-783, 2623-2640, 6712-6728, 8904-8927, 11133-11155, 13415-13433, 15506-15533, 17694-17714, 19761-19773, and 21334-21346. Such an antigen-based vaccine can be useful for treating a patient who expresses the HLA allele 2601.

In particular embodiments, one or more HIV epitope sequences or one or more HIV epitope sequences encoded by HIV epitope encoding sequences that are selected for inclusion in an antigen-based vaccine can include any of SEQ ID NOs: SEQ ID NOs: 784-790, 2641-2652, 6729-6739, 8928-8937, 11156-11168, 13434-13446, 1553-15550, 17715-17723, 19774-19782, and 21347-21353. Such an antigen-based vaccine can be useful for treating a patient who expresses the HLA allele 2602.

In particular embodiments, one or more HIV epitope sequences or one or more HIV epitope sequences encoded by HIV epitope encoding sequences that are selected for inclusion in an antigen-based vaccine can include any of SEQ ID NOs: 791-802, 2653-2671, 6740-6759, 8938-8959, 11169-11189, 13447-13464, 15551-15569, 17724-17739, 19783-19797, and 21354-21360. Such an antigen-based vaccine can be useful for treating a patient who expresses the HLA allele 2603.

In particular embodiments, one or more HIV epitope sequences or one or more HIV epitope sequences encoded by HIV epitope encoding sequences that are selected for inclusion in an antigen-based vaccine can include any of SEQ ID NOs: 803-814, 2672-2679, 6760-6768, 8960-8976, 11190-11195, 13465-13474, 15570-15588, 17740-17751, 19798-19808, and 21361-21366. Such an antigen-based vaccine can be useful for treating a patient who expresses the HLA allele A2901.

In particular embodiments, one or more HIV epitope sequences or one or more HIV epitope sequences encoded by HIV epitope encoding sequences that are selected for inclusion in an antigen-based vaccine can include any of SEQ ID NOs: 815-828, 2680-2698, 6769-6784, 8977-9000,11196-11210, 13475-13493, 15589-15612, 17752-17773, 19809-19821, and 21367-21376. Such an antigen-based vaccine can be useful for treating a patient who expresses the HLA allele A2902.

In particular embodiments, one or more HIV epitope sequences or one or more HIV epitope sequences encoded by HIV epitope encoding sequences that are selected for inclusion in an antigen-based vaccine can include any of SEQ ID NOs: SEQ ID NOs: 829-842, 2699-2707, 6785-6793, 9001-9012, 11211-11216, 13494-13501, 15613-15617, 17774-17781, 19822-19828, and 21377-21383). Such an antigen-based vaccine can be useful for treating a patient who expresses the HLA allele A3001.

In particular embodiments, one or more HIV epitope sequences or one or more HIV epitope sequences encoded by HIV epitope encoding sequences that are selected for inclusion in an antigen-based vaccine can include any of SEQ ID NOs: SEQ ID NOs: 843-857, 2708-2722, 6794-6807, 9013-9040, 11217-11235, 13502-13519, 15618-15636, 17782-17809, 19829-19843, and 21384-21390). Such an antigen-based vaccine can be useful for treating a patient who expresses the HLA allele A3002.

In particular embodiments, one or more HIV epitope sequences or one or more HIV epitope sequences encoded by HIV epitope encoding sequences that are selected for inclusion in an antigen-based vaccine can include any of SEQ ID NOs: 858-864, 2723-2728, 6808-6817, 9041-9060, 11236-11246, 13520-13530, 15637-15649, 17810-17828, 19844-19850, and 21391-21393). Such an antigen-based vaccine can be useful for treating a patient who expresses the HLA allele A3004.

In particular embodiments, one or more HIV epitope sequences or one or more HIV epitope sequences encoded by HIV epitope encoding sequences that are selected for inclusion in an antigen-based vaccine can include any of SEQ ID NOs: 865-895, 2729-2757, 6818-6846, 9061-9082, 11247-11272, 13531-13558, 15650-15683, 17829-17862, 19851-19869, and 21394-21407). Such an antigen-based vaccine can be useful for treating a patient who expresses the HLA allele A3101.

In particular embodiments, one or more HIV epitope sequences or one or more HIV epitope sequences encoded by HIV epitope encoding sequences that are selected for inclusion in an antigen-based vaccine can include any of SEQ ID NOs: 896-899, 2758-2761, 6847-6850, 9083-9091, 11273-11275, 13559-13567, 15684-15688, 17863-17870, 19870-19874, and 21408-21409). Such an antigen-based vaccine can be useful for treating a patient who expresses the HLA allele A3201.

In particular embodiments, one or more HIV epitope sequences or one or more HIV epitope sequences encoded by HIV epitope encoding sequences that are selected for inclusion in an antigen-based vaccine can include any of SEQ ID NOs: 900-920, 2762-2793, 6851-6880, 9092-9112, 11276-11300, 13568-13585, 15689-15707, 17871-17900, 19875-19898, and 21410-21425. Such an antigen-based vaccine can be useful for treating a patient who expresses the HLA allele A3301.

In particular embodiments, one or more HIV epitope sequences or one or more HIV epitope sequences encoded by HIV epitope encoding sequences that are selected for inclusion in an antigen-based vaccine can include any of SEQ ID NOs: 921-955, 2794-2851, 6881-6935, 9113-9164, 11301-11346, 13586-13619, 15708-15742, 17901-17964, 19899-19933, and 21426-21459. Such an antigen-based vaccine can be useful for treating a patient who expresses the HLA allele A3303.

In particular embodiments, one or more HIV epitope sequences or one or more HIV epitope sequences encoded by HIV epitope encoding sequences that are selected for inclusion in an antigen-based vaccine can include any of SEQ ID NOs: 956-997, 2852-2908, 6936-6986, 9165-9228, 11347-11410, 13620-13667, 15743-15785, 17965-18029, 19934-19986, and 21460-24192. Such an antigen-based vaccine can be useful for treating a patient who expresses the HLA allele A6801.

In particular embodiments, one or more HIV epitope sequences or one or more HIV epitope sequences encoded by HIV epitope encoding sequences that are selected for inclusion in an antigen-based vaccine can include any of SEQ ID NOs: 998-1032, 2909-2946, 6897-7037, 9229-9292, 11411-11461, 13668-13715, 15786-15828, 18030-18068, 19987-20027, and 24193-21523). Such an antigen-based vaccine can be useful for treating a patient who expresses the HLA allele A6802.

In particular embodiments, one or more HIV epitope sequences or one or more HIV epitope sequences encoded by HIV epitope encoding sequences that are selected for inclusion in an antigen-based vaccine can include any of SEQ ID NOs: 1033-1050, 2947-2969, 7038-7065, 9293-9312, 11462-11485, 13716-13738, 15829-15849, 18069-18091, 20028-20038, and 21524-21540. Such an antigen-based vaccine can be useful for treating a patient who expresses the HLA allele B0702.

In particular embodiments, one or more HIV epitope sequences or one or more HIV epitope sequences encoded by HIV epitope encoding sequences that are selected for inclusion in an antigen-based vaccine can include any of SEQ ID NOs: 1051-1066, 2970-2984, 7066-7078, 9313-9325, 11486-11497, 13739-13752, 15850-15862, 18092-18112, 20039-20051, and 21541-21549). Such an antigen-based vaccine can be useful for treating a patient who expresses the HLA allele B0801.

In particular embodiments, one or more HIV epitope sequences or one or more HIV epitope sequences encoded by HIV epitope encoding sequences that are selected for inclusion in an antigen-based vaccine can include any of SEQ ID NOs: 1067-1080, 2985-2999, 7079-7095, 9326-9347, 11498-11516, 13753-13767, 15863-15875, 18113-18128, 20052-20062, and 21550-21557). Such an antigen-based vaccine can be useful for treating a patient who expresses the HLA allele B1301.

In particular embodiments, one or more HIV epitope sequences or one or more HIV epitope sequences encoded by HIV epitope encoding sequences that are selected for inclusion in an antigen-based vaccine can include any of SEQ ID NOs: 1081-1117, 3000-3052, 7096-7140, 9348-9406, 11517-11557, 13768-13821, 15876-15923, 18129-18178, 20063-20093, and 21558-21593. Such an antigen-based vaccine can be useful for treating a patient who expresses the HLA allele B1302.

In particular embodiments, one or more HIV epitope sequences or one or more HIV epitope sequences encoded by HIV epitope encoding sequences that are selected for inclusion in an antigen-based vaccine can include any of SEQ ID NOs: 1118-1125, 3053-3058, 7141-7145, 9407-9411, 11558-11562, 13822-13827, 15924-15931, 18179-18185, 20094-20098, and 21594-21599. Such an antigen-based vaccine can be useful for treating a patient who expresses the HLA allele B1401.

In particular embodiments, one or more HIV epitope sequences or one or more HIV epitope sequences encoded by HIV epitope encoding sequences that are selected for inclusion in an antigen-based vaccine can include any of SEQ ID NOs: 1126-1139, 3059-3070, 7146-7159, 9412-9418, 11563-11574, 13828-13837, 15932-15943, 18186-18197, 20099-20109, and 21600-21606. Such an antigen-based vaccine can be useful for treating a patient who expresses the HLA allele B1402.

In particular embodiments, one or more HIV epitope sequences or one or more HIV epitope sequences encoded by HIV epitope encoding sequences that are selected for inclusion in an antigen-based vaccine can include any of SEQ ID NOs: 1140-1192, 3071-3111, 7160-7211, 9419-9481, 11575-11633, 13838-13895, 15944-16001, 18198-18259, 20110-20141, and 21607-21635. Such an antigen-based vaccine can be useful for treating a patient who expresses the HLA allele B1501.

In particular embodiments, one or more HIV epitope sequences or one or more HIV epitope sequences encoded by HIV epitope encoding sequences that are selected for inclusion in an antigen-based vaccine can include any of SEQ ID NOs: 1193-1220, 3112-3135, 7212-7247, 9482-9501, 11634-11670, 13896-13937, 16002-16036, 18260-18300, 20142-20165, and 21636-21656. Such an antigen-based vaccine can be useful for treating a patient who expresses the HLA allele B1502.

In particular embodiments, one or more HIV epitope sequences or one or more HIV epitope sequences encoded by HIV epitope encoding sequences that are selected for inclusion in an antigen-based vaccine can include any of SEQ ID NOs: 1221-1245, 3136-3152, 7248-7273, 9502-9526, 11671-11693, 13938-13968, 16037-16065, 18301-18324, 20166-20179, and 21657-21669. Such an antigen-based vaccine can be useful for treating a patient who expresses the HLA allele B1503.

In particular embodiments, one or more HIV epitope sequences or one or more HIV epitope sequences encoded by HIV epitope encoding sequences that are selected for inclusion in an antigen-based vaccine can include any of SEQ ID NOs: 1246-1266, 3153-3178, 7274-7296, 9527-9548, 11694-11722, 13969-13995, 16066-16083, 18325-18352, 20180-20200, and 21670-21689. Such an antigen-based vaccine can be useful for treating a patient who expresses the HLA allele B1510.

In particular embodiments, one or more HIV epitope sequences or one or more HIV epitope sequences encoded by HIV epitope encoding sequences that are selected for inclusion in an antigen-based vaccine can include any of SEQ ID NOs: 1267-1270, 3179-3183, 7297-7300, 9549-9551, 11723-11725, 13996-14005, 16084-16091, 18353-18358, 20201-20205, and 21690-21692. Such an antigen-based vaccine can be useful for treating a patient who expresses the HLA allele B1513.

In particular embodiments, one or more HIV epitope sequences or one or more HIV epitope sequences encoded by HIV epitope encoding sequences that are selected for inclusion in an antigen-based vaccine can include any of SEQ ID NOs: 1271-1286, 3184-3203, 7301-7328, 9552-9565, 11726-11742, 14006-14024, 16092-16107, 18359-18375, 20206-20224, and 21693-21705. Such an antigen-based vaccine can be useful for treating a patient who expresses the HLA allele B1801.

In particular embodiments, one or more HIV epitope sequences or one or more HIV epitope sequences encoded by HIV epitope encoding sequences that are selected for inclusion in an antigen-based vaccine can include any of SEQ ID NOs: 1287-1304, 3204-3225, 7329-7355, 9566-9594, 11743-11756, 14025-14048, 16108-16135, 18376-18408, 20225-20241, and 21706-21716. Such an antigen-based vaccine can be useful for treating a patient who expresses the HLA allele B2702.

In particular embodiments, one or more HIV epitope sequences or one or more HIV epitope sequences encoded by HIV epitope encoding sequences that are selected for inclusion in an antigen-based vaccine can include any of SEQ ID NOs: 1305-1319, 3226-3234, 7356-7370, 9595-9610, 11757-11771, 14049-14063, 16136-16145, 18409-18422, 20242-20254, and 21717-21723. Such an antigen-based vaccine can be useful for treating a patient who expresses the HLA allele B2705.

In particular embodiments, one or more HIV epitope sequences or one or more HIV epitope sequences encoded by HIV epitope encoding sequences that are selected for inclusion in an antigen-based vaccine can include any of SEQ ID NOs: 1320-1338, 3235-3260, 7371-7405, 9611-9641, 11772-11812, 14064-14095, 16146-16186, 18423-18463, 20255-20279, and 21724-21745. Such an antigen-based vaccine can be useful for treating a patient who expresses the HLA allele B3501.

In particular embodiments, one or more HIV epitope sequences or one or more HIV epitope sequences encoded by HIV epitope encoding sequences that are selected for inclusion in an antigen-based vaccine can include any of SEQ ID NOs: 1339-1349, 3261-3272, 7406-7424, 9642-9661, 11813-11833, 14096-14112, 16187-16205, 18464-18482, 20280-20291, and 21746-21754. Such an antigen-based vaccine can be useful for treating a patient who expresses the HLA allele B3502.

In particular embodiments, one or more HIV epitope sequences or one or more HIV epitope sequences encoded by HIV epitope encoding sequences that are selected for inclusion in an antigen-based vaccine can include any of SEQ ID NOs: 1350-1373, 3273-3298, 7425-7457, 9662-9697, 11834-11877, 14113-14148, 16206-16238, 18483-18513, 20292-20316, and 21755-21772. Such an antigen-based vaccine can be useful for treating a patient who expresses the HLA allele B3503.

In particular embodiments, one or more HIV epitope sequences or one or more HIV epitope sequences encoded by HIV epitope encoding sequences that are selected for inclusion in an antigen-based vaccine can include any of SEQ ID NOs: 1374-1386, 3299-3309, 7458-7477, 9698-9719, 11878-11899, 14149-14166, 16239-16256, 18514-18538, 20317-20331, and 21773-21786. Such an antigen-based vaccine can be useful for treating a patient who expresses the HLA allele B3508.

In particular embodiments, one or more HIV epitope sequences or one or more HIV epitope sequences encoded by HIV epitope encoding sequences that are selected for inclusion in an antigen-based vaccine can include any of SEQ ID NOs: 1387-1405, 3310-3326, 7478-7498, 9720-9744, 11900-11930, 14167-14185, 16257-16280, 18539-18560, 20332-20344, and 21787-21799. Such an antigen-based vaccine can be useful for treating a patient who expresses the HLA allele B3512.

In particular embodiments, one or more HIV epitope sequences or one or more HIV epitope sequences encoded by HIV epitope encoding sequences that are selected for inclusion in an antigen-based vaccine can include any of SEQ ID NOs: 1406-1425, 3327-3338, 7499-7512, 9745-9757, 11931-11944, 14186-14196, 16281-16291, 18561-18572, 20345-20359, and 21800-21808. Such an antigen-based vaccine can be useful for treating a patient who expresses the HLA allele B3701.

In particular embodiments, one or more HIV epitope sequences or one or more HIV epitope sequences encoded by HIV epitope encoding sequences that are selected for inclusion in an antigen-based vaccine can include any of SEQ ID NOs: 1426-1451, 3339-3367, 7513-7533, 9758-9782, 11945-11970, 14197-14219, 16292-16310, 18573-18599, 20360-20381, and 21809-21828. Such an antigen-based vaccine can be useful for treating a patient who expresses the HLA allele B3801.

In particular embodiments, one or more HIV epitope sequences or one or more HIV epitope sequences encoded by HIV epitope encoding sequences that are selected for inclusion in an antigen-based vaccine can include any of SEQ ID NOs: 1452-1476, 3368-3391, 7534-7551, 9783-9802, 11971-11992, 14220-14242, 16311-16323, 18600-18619, 20382-20395, and 21829-21844. Such an antigen-based vaccine can be useful for treating a patient who expresses the HLA allele B3901.

In particular embodiments, one or more HIV epitope sequences or one or more HIV epitope sequences encoded by HIV epitope encoding sequences that are selected for inclusion in an antigen-based vaccine can include any of SEQ ID NOs: 1477-1499, 3392-3423, 7552-7571, 9803-9831, 11993-12020, 14243-14277, 16324-16349, 18620-18653, 20396-20411, and 21845-21861. Such an antigen-based vaccine can be useful for treating a patient who expresses the HLA allele B3906.

In particular embodiments, one or more HIV epitope sequences or one or more HIV epitope sequences encoded by HIV epitope encoding sequences that are selected for inclusion in an antigen-based vaccine can include any of SEQ ID NOs: 1500-1527, 3424-3458, 7572-7614, 9832-9867, 12021-12057, 14278-14309, 16350-16384, 18654-18686, 20412-20431, and 21862-21888. Such an antigen-based vaccine can be useful for treating a patient who expresses the HLA allele B4001.

In particular embodiments, one or more HIV epitope sequences or one or more HIV epitope sequences encoded by HIV epitope encoding sequences that are selected for inclusion in an antigen-based vaccine can include any of SEQ ID NOs: 1528-1576, 3459-3497, 7615-7665, 9868-9913, 12058-12110, 14310-14359, 16385-16431, 18687-18736, 20432-20460, and 21889-21924). Such an antigen-based vaccine can be useful for treating a patient who expresses the HLA allele B4002.

In particular embodiments, one or more HIV epitope sequences or one or more HIV epitope sequences encoded by HIV epitope encoding sequences that are selected for inclusion in an antigen-based vaccine can include any of SEQ ID NOs: 1577-1593, 3498-3517, 7666-7689, 9914-9942, 12111-12136, 14360-14380, 16432-16463, 18737-18759, 20461-20479, and 21925-21940. Such an antigen-based vaccine can be useful for treating a patient who expresses the HLA allele B4006.

In particular embodiments, one or more HIV epitope sequences or one or more HIV epitope sequences encoded by HIV epitope encoding sequences that are selected for inclusion in an antigen-based vaccine can include any of SEQ ID NOs: 1594-1642, 3518-3554, 7690-7742, 9943-9988, 12137-12175, 14381-14429, 16437-16510, 18760-18811, 20480-20512, and 21941-21975. Such an antigen-based vaccine can be useful for treating a patient who expresses the HLA allele B4102.

In particular embodiments, one or more HIV epitope sequences or one or more HIV epitope sequences encoded by HIV epitope encoding sequences that are selected for inclusion in an antigen-based vaccine can include any of SEQ ID NOs: 1643-1663, 3555-3575, 7743-7772, 9989-10011, 12176-12202, 14430-14448, 16510-16527, 18812-18834, 20513-20530, and 21976-21992. Such an antigen-based vaccine can be useful for treating a patient who expresses the HLA allele B4402.

In particular embodiments, one or more HIV epitope sequences or one or more HIV epitope sequences encoded by HIV epitope encoding sequences that are selected for inclusion in an antigen-based vaccine can include any of SEQ ID NOs: 1664-1697, 3576-3611, 7773-7826, 10012-10058, 12203-12254, 14449-14493, 16528-16562, 18835-18883, 20531-20564, and 21993-22024. Such an antigen-based vaccine can be useful for treating a patient who expresses the HLA allele B4403.

In particular embodiments, one or more HIV epitope sequences or one or more HIV epitope sequences encoded by HIV epitope encoding sequences that are selected for inclusion in an antigen-based vaccine can include any of SEQ ID NOs: 1698-1745, 3612-3674, 7827-7903, 10059-10134, 12255-12327, 14494-14560, 16563-16633, 18884-18953, 20565-20613, and 22025-22067. Such an antigen-based vaccine can be useful for treating a patient who expresses the HLA allele B4405.

In particular embodiments, one or more HIV epitope sequences or one or more HIV epitope sequences encoded by HIV epitope encoding sequences that are selected for inclusion in an antigen-based vaccine can include any of SEQ ID NOs: 1746-1752, 3675-3679, 7904-7910, 10135-10146, 12328-12339, 14561-14574, 16634-16645, 18954-18957, 20614-20619, and 22068-22069. Such an antigen-based vaccine can be useful for treating a patient who expresses the HLA allele B4601.

In particular embodiments, one or more HIV epitope sequences or one or more HIV epitope sequences encoded by HIV epitope encoding sequences that are selected for inclusion in an antigen-based vaccine can include any of SEQ ID NOs: 1753-1785, 3680-3695, 7911-7926, 10147-10161, 12340-12359, 14575-14596, 16646-16664, 18958-18974, 20620-20636, and 22070-22081. Such an antigen-based vaccine can be useful for treating a patient who expresses the HLA allele B4801.

In particular embodiments, one or more HIV epitope sequences or one or more HIV epitope sequences encoded by HIV epitope encoding sequences that are selected for inclusion in an antigen-based vaccine can include any of SEQ ID NOs: 1786-1824, 3696-3719, 7927-7967, 10162-10207, 12360-12395, 14597-14634, 16665-16709, 18975-19013, 20637-20656, and 22082-22109. Such an antigen-based vaccine can be useful for treating a patient who expresses the HLA allele B4901.

In particular embodiments, one or more HIV epitope sequences or one or more HIV epitope sequences encoded by HIV epitope encoding sequences that are selected for inclusion in an antigen-based vaccine can include any of SEQ ID NOs: 1825-1855, 3720-3755, 7968-8008, 10208-10251, 12396-12438, 14635-14675, 16710-16748, 19014-19051, 20657-20682, and 22110-22129. Such an antigen-based vaccine can be useful for treating a patient who expresses the HLA allele B5001.

In particular embodiments, one or more HIV epitope sequences or one or more HIV epitope sequences encoded by HIV epitope encoding sequences that are selected for inclusion in an antigen-based vaccine can include any of SEQ ID NOs: 1856-1872, 3756-3789, 8009-8037, 10252-10287, 12439-12467, 14676-14708, 16749-16783, 19052-19076, 20683-20711, and 22130-22158. Such an antigen-based vaccine can be useful for treating a patient who expresses the HLA allele B5101.

In particular embodiments, one or more HIV epitope sequences or one or more HIV epitope sequences encoded by HIV epitope encoding sequences that are selected for inclusion in an antigen-based vaccine can include any of SEQ ID NOs: 1873-1900, 3790-3823, 8038-8075, 10288-10327, 12468-12507, 14709-14745, 16784-16826, 19077-19108, 207120-20748, and 22159-22178. Such an antigen-based vaccine can be useful for treating a patient who expresses the HLA allele B5401.

In particular embodiments, one or more HIV epitope sequences or one or more HIV epitope sequences encoded by HIV epitope encoding sequences that are selected for inclusion in an antigen-based vaccine can include any of SEQ ID NOs: 1901-1907, 3824-3827, 8076-8088, 10328-10341, 12508-12520, 14746-14756, 16827-16841, 19109-19113, 20749-20759, and 22179-22184. Such an antigen-based vaccine can be useful for treating a patient who expresses the HLA allele B5501.

In particular embodiments, one or more HIV epitope sequences or one or more HIV epitope sequences encoded by HIV epitope encoding sequences that are selected for inclusion in an antigen-based vaccine can include any of SEQ ID NOs: 1908-1924, 3828-3843, 8089-8109, 10342-10364, 12521-12543, 14757-14777, 16842-16867, 19114-19135, 20760-20785, and 22185-22194. Such an antigen-based vaccine can be useful for treating a patient who expresses the HLA allele B5502.

In particular embodiments, one or more HIV epitope sequences or one or more HIV epitope sequences encoded by HIV epitope encoding sequences that are selected for inclusion in an antigen-based vaccine can include any of SEQ ID NOs: 1925-1945, 3844-3865, 8110-8136, 10365-10392, 12544-12565, 14778-14802, 16868-16897, 19136-19156, 20786-20810, and 22195-22209. Such an antigen-based vaccine can be useful for treating a patient who expresses the HLA allele B5601.

In particular embodiments, one or more HIV epitope sequences or one or more HIV epitope sequences encoded by HIV epitope encoding sequences that are selected for inclusion in an antigen-based vaccine can include any of SEQ ID NOs: 1946-1985, 3866-3908, 8137-8188, 10393-10441, 12566-12606, 14803-14849, 16898-16956, 19157-19202, 20811-20848, and 22210-22234. Such an antigen-based vaccine can be useful for treating a patient who expresses the HLA allele B5701.

In particular embodiments, one or more HIV epitope sequences or one or more HIV epitope sequences encoded by HIV epitope encoding sequences that are selected for inclusion in an antigen-based vaccine can include any of SEQ ID NOs: 1986-2019, 3909-3942, 8189-8218, 10442-10467, 12607-12632, 14850-14873, 16957-16992, 19203-19232, 20849-20875, and 22235-22252. Such an antigen-based vaccine can be useful for treating a patient who expresses the HLA allele B5801.

In particular embodiments, one or more HIV epitope sequences or one or more HIV epitope sequences encoded by HIV epitope encoding sequences that are selected for inclusion in an antigen-based vaccine can include any of SEQ ID NOs: 2020-2026, 3943-3945, 8219-8224, 10468-10472, 12633-12644, 14874-14881, 16993-16996, 19233-19242, 20876-20880, and 22253-22255. Such an antigen-based vaccine can be useful for treating a patient who expresses the HLA allele C0102.

In particular embodiments, one or more HIV epitope sequences or one or more HIV epitope sequences encoded by HIV epitope encoding sequences that are selected for inclusion in an antigen-based vaccine can include any of SEQ ID NOs: 2027-2028, 3946-3947, 8225-8227, 10473-10476, 12645-12647, 14882-14887, 16997-16999, 19243-19245, 20881-20883, and 22256-22262. Such an antigen-based vaccine can be useful for treating a patient who expresses the HLA allele C0202.

In particular embodiments, one or more HIV epitope sequences or one or more HIV epitope sequences encoded by HIV epitope encoding sequences that are selected for inclusion in an antigen-based vaccine can include any of SEQ ID NOs: 2029-2034, 3948-3956, 8228-8233, 10477-10484, 12648-12657, 14888-14900, 17000-17007, 19246-19253, 20884-20888, and 22263-22266. Such an antigen-based vaccine can be useful for treating a patient who expresses the HLA allele C0302.

In particular embodiments, one or more HIV epitope sequences or one or more HIV epitope sequences encoded by HIV epitope encoding sequences that are selected for inclusion in an antigen-based vaccine can include any of SEQ ID NOs: 2035-2039, 3957-3962, 8234-8239, 10485-10491, 12658-12663, 14901-14911, 17008-17016, 19254-19257, 20889-20893, and 22267-22272. Such an antigen-based vaccine can be useful for treating a patient who expresses the HLA allele C0303.

In particular embodiments, one or more HIV epitope sequences or one or more HIV epitope sequences encoded by HIV epitope encoding sequences that are selected for inclusion in an antigen-based vaccine can include any of SEQ ID NOs: 2040-2047, 3963-3974, 8240-8250, 10492-10502, 12664-12676, 14912-14927, 17017-17029, 19258-19270, 20894-20901, and 22273-22274. Such an antigen-based vaccine can be useful for treating a patient who expresses the HLA allele C0304.

In particular embodiments, one or more HIV epitope sequences or one or more HIV epitope sequences encoded by HIV epitope encoding sequences that are selected for inclusion in an antigen-based vaccine can include any of SEQ ID NOs: 2048-2052, 3975-3979, 8251-8257, 10503-10505, 12677-12680, 14928-14932, 17030-17033, 19271-19277, 20902-20903, and 22275-22281. Such an antigen-based vaccine can be useful for treating a patient who expresses the HLA allele C0401.

In particular embodiments, one or more HIV epitope sequences or one or more HIV epitope sequences encoded by HIV epitope encoding sequences that are selected for inclusion in an antigen-based vaccine can include any of SEQ ID NOs: 2053-2057, 3980-3992, 8258-8262, 10506-10514, 12681-12692, 14933-14944, 17034-17041, 19278-19288, 20904-20911, and 22282-22283. Such an antigen-based vaccine can be useful for treating a patient who expresses the HLA allele C0501.

In particular embodiments, one or more HIV epitope sequences or one or more HIV epitope sequences encoded by HIV epitope encoding sequences that are selected for inclusion in an antigen-based vaccine can include any of SEQ ID NOs: 2058-2059, 3993-3995, 8263, 10515-10518, 12693-12697, 14945-14948, 17042-17045, 19289-19290, 20912-20913, 22284-22295). Such an antigen-based vaccine can be useful for treating a patient who expresses the HLA allele C0602.

In particular embodiments, one or more HIV epitope sequences or one or more HIV epitope sequences encoded by HIV epitope encoding sequences that are selected for inclusion in an antigen-based vaccine can include any of SEQ ID NOs: 8264 and 17046. Such an antigen-based vaccine can be useful for treating a patient who expresses the HLA allele C0701.

In particular embodiments, one or more HIV epitope sequences or one or more HIV epitope sequences encoded by HIV epitope encoding sequences that are selected for inclusion in an antigen-based vaccine can include any of SEQ ID NOs: 2060, 3996-3997, 12698, and 14949). Such an antigen-based vaccine can be useful for treating a patient who expresses the HLA allele C0702.

In particular embodiments, one or more HIV epitope sequences or one or more HIV epitope sequences encoded by HIV epitope encoding sequences that are selected for inclusion in an antigen-based vaccine can include any of SEQ ID NOs: 2061, 3998, 10519, and 17047. Such an antigen-based vaccine can be useful for treating a patient who expresses the HLA allele C0704.

In particular embodiments, one or more HIV epitope sequences or one or more HIV epitope sequences encoded by HIV epitope encoding sequences that are selected for inclusion in an antigen-based vaccine can include any of SEQ ID NOs: 2062-2079, 3999-4013, 8265-8274, 10520-10533, 12699-12721, 14950-14974, 17048-17069, 19291-19304, 20914-20923, and 22284-22295. Such an antigen-based vaccine can be useful for treating a patient who expresses the HLA allele C0801.

In particular embodiments, one or more HIV epitope sequences or one or more HIV epitope sequences encoded by HIV epitope encoding sequences that are selected for inclusion in an antigen-based vaccine can include any of SEQ ID NOs: 2080-2088, 4014-4031, 8275-8288, 10534-10545, 12722-12739, 14975-14987, 17070-1076, 19305-19321, 20924-20929, and 22296-22300). Such an antigen-based vaccine can be useful for treating a patient who expresses the HLA allele C0802.

In particular embodiments, one or more HIV epitope sequences or one or more HIV epitope sequences encoded by HIV epitope encoding sequences that are selected for inclusion in an antigen-based vaccine can include any of SEQ ID NOs: 2089-2100, 4032-4035, 8289-8295, 10546-10548, 12740-12742, 14988-14997, 17077-17079, 19322-19324, 20930-20938, and 22301-22304. Such an antigen-based vaccine can be useful for treating a patient who expresses the HLA allele C0803.

In particular embodiments, one or more HIV epitope sequences or one or more HIV epitope sequences encoded by HIV epitope encoding sequences that are selected for inclusion in an antigen-based vaccine can include any of SEQ ID NOs: 2101-2105, 4036-4043, 8296-8302, 10549-10555, 102743-12748, 14998-15007, 17080-17089, 19325-19332, 20939-20947, and 22305-22310. Such an antigen-based vaccine can be useful for treating a patient who expresses the HLA allele C1203.

In particular embodiments, one or more HIV epitope sequences or one or more HIV epitope sequences encoded by HIV epitope encoding sequences that are selected for inclusion in an antigen-based vaccine can include any of SEQ ID NOs: 2106-2122, 4044-4058, 8303-8329, 10556-10574, 12749-12763, 15008-15025, 17090-17108, 19333-19348, 20948-20962, and 22311-22320. Such an antigen-based vaccine can be useful for treating a patient who expresses the HLA allele C1402.

In particular embodiments, one or more HIV epitope sequences or one or more HIV epitope sequences encoded by HIV epitope encoding sequences that are selected for inclusion in an antigen-based vaccine can include any of SEQ ID NOs: 2123-2133, 4059-4069, 8330-8342, 10575-10587, 12764-12772 15026-15035, 17109-17124, 19349-19361, 20963-20970, and 22321-22327. Such an antigen-based vaccine can be useful for treating a patient who expresses the HLA allele C1403.

In particular embodiments, one or more HIV epitope sequences or one or more HIV epitope sequences encoded by HIV epitope encoding sequences that are selected for inclusion in an antigen-based vaccine can include any of SEQ ID NOs: 2134-2138, 4070-4074, 8343-8354, 10588-10591, 12773-12778, 15036-15040, 17125-17135, 19362-19366, 20971-20978, and 22328-22332. Such an antigen-based vaccine can be useful for treating a patient who expresses the HLA allele C1502.

In particular embodiments, one or more HIV epitope sequences or one or more HIV epitope sequences encoded by HIV epitope encoding sequences that are selected for inclusion in an antigen-based vaccine can include any of SEQ ID NOs: 2139-2143, 4075-4079, 8355-8358, 10592-10595, 12779-12782, 15041-15048, 17136-17144, 19367-19370, 20979-20983, and 22333-22334. Such an antigen-based vaccine can be useful for treating a patient who expresses the HLA allele C1601.

In particular embodiments, one or more HIV epitope sequences or one or more HIV epitope sequences encoded by HIV epitope encoding sequences that are selected for inclusion in an antigen-based vaccine can include any of SEQ ID NOs: 2144-2151, 4080-4089, 8359-8367, 10596-10602, 12783-12792, 15049-15058, 17145-17157, 19371-19376, 20984-20992, and 22335-22340. Such an antigen-based vaccine can be useful for treating a patient who expresses the HLA allele C1602.

In particular embodiments, one or more HIV epitope sequences or one or more HIV epitope sequences encoded by HIV epitope encoding sequences that are selected for inclusion in an antigen-based vaccine can include any of SEQ ID NOs: 2152-2160, 4090-4098, 8368-8381, 10603-10615, 12793-12803, 15059-15069, 17158-17165, 19377-19382, 20994-20998, and 22341-22345. Such an antigen-based vaccine can be useful for treating a patient who expresses the HLA allele C1604.

In particular embodiments, one or more HIV epitope sequences or one or more HIV epitope sequences encoded by HIV epitope encoding sequences that are selected for inclusion in an antigen-based vaccine can include any of SEQ ID NOs: 2161-2165, 4099-4106, 8382-8389, 10616-10626, 12804-12810, 15070-15079, 17166-17174, 19383-19388, 20999-21003, and 22346-22349. Such an antigen-based vaccine can be useful for treating a patient who expresses the HLA allele C1701.

In various embodiments, an antigen-based vaccine can be generated to include at least one HIV epitope sequence, or at least one HIV-epitope encoding sequence that encodes for the at least one HIV epitope sequence, that is predicted (e.g., as predicted by a presentation model) to most likely be presented by a HLA allele. In various embodiments, an antigen-based vaccine can include one or more HIV-epitope encoding sequences that encode for one or more HIV epitope sequences selected from any of SEQ ID NOs: 4178, 4178 and 5329, 5239, 756, 1594, 3184, 6851, 6936, 7773, 10970, 11027, 11028, 12508, 13291, 13768, 13838, 14597, 14874, 16634, 20396, 20480, and 21755. In various embodiments, an antigen-based vaccine can include one or more HIV epitope sequences selected from any of SEQ ID NOs: 4178, 4178 and 5329, 5239, 756, 1594, 3184, 6851, 6936, 7773, 10970, 11027, 11028, 12508, 13291, 13768, 13838, 14597, 14874, 16634, 20396, 20480, and 21755.

In various embodiments, an antigen-based vaccine can be generated for a particular HIV subtype. For example, all selected HIV epitopes are derived from a HIV subtype and are predicted to be presented by one or more HLA alleles. In various embodiments, the selected epitopes are from HIV subtype B and are predicted to be presented by frequently expressed HLA alleles (e.g., any of A0101, A0201, A0301, A1101, A2301, A2402, B0702, B0801, B3501, B4001, B4402, and B4403). Example epitope sequences and corresponding SEQ ID NOs that can be selected for HIV subtype B are shown below in Table 1:

TABLE 1
Candidate epitope sequences

| SEQ ID | HIV | Epitope | | HIV |
|---|---|---|---|---|
| NO: | Subtype | sequence | HLA | Protein |
| 4113 | B | GSEELRSLY | A0101 | gag |
| 4114 | B | VLAEAMSQV | A0201 | gag |
| 4115 | B | SLYNTVATL | A0201 | gag |
| 4427 | B | VTNSGAIMMQK | A0301 | gag |
| 4439 | B | NSATIMMQK | A0301 | gag |
| 4494 | B | VTNSATIMMQK | A1101 | gag |
| 4495 | B | NSGAIMMQK | A1101 | gag |
| 4545 | B | NYTNLIYTL | A2301 | env |
| 4561 | B | NYTSLIYTL | A2402 | env |
| 4956 | B | YPLTSLRSL | B0702 | gag |
| 4968 | B | TPQDLNTML | B0702 | gag |
| 4975 | B | YPLTALKSL | B0801 | gag |
| 4982 | B | ELKSLFNTV | B0801 | gag |
| 5259 | B | PPIPVGEIY | B3501 | gag |
| 5261 | B | NPPIPVGEIY | B3501 | gag |
| 5459 | B | IEIKDTKEAL | B4001 | gag |
| 5460 | B | IEVKDTKEAL | B4001 | gag |
| 5610 | B | AEQASQEVKNW | B4402 | gag |
| 5643 | B | TENSSQVSQNY | B4403 | gag |
| 5661 | B | QETIDKELY | B4403 | gag |

In various embodiments, an antigen-based vaccine can include one or more HIV epitope sequences selected from SEQ ID NOs: 4113, 4114, 4115, 4427, 4439, 4494, 4495, 4545, 4561, 4956, 4968, 4975, 4982, 5259, 5261, 5459, 5460, 5610, 5643, and 5661. In various embodiments, an antigen-based vaccine can include one or more HIV-epitope encoding sequences that encode for one or more HIV epitope sequences selected from SEQ ID NOs: 4113, 4114, 4115, 4427, 4439, 4494, 4495, 4545, 4561, 4956, 4968, 4975, 4982, 5259, 5261, 5459, 5460, 5610, 5643, and 5661. In various embodiments, an antigen-based vaccine can include two, three, four, five, six, seven, eight, nine, ten, eleven, twelve, thirteen, fourteen, fifteen, sixteen, seventeen, eighteen, nineteen, or twenty HIV epitope sequences selected from SEQ ID NOs: 4113, 4114, 4115, 4427, 4439, 4494, 4495, 4545, 4561, 4956, 4968, 4975, 4982, 5259, 5261, 5459, 5460, 5610, 5643, and 5661. In various embodiments, an antigen-based vaccine can include two, three, four, five, six, seven, eight, nine, ten, eleven, twelve, thirteen, fourteen, fifteen, sixteen, seventeen, eighteen, nineteen, or twenty HIV-epitope encoding sequences that encode for two, three, four, five, six, seven, eight, nine, ten, eleven, twelve, thirteen, fourteen, fifteen, sixteen, seventeen, eighteen, nineteen, or twenty HIV epitope sequences selected from SEQ ID NOs: 4113, 4114, 4115, 4427, 4439, 4494, 4495, 4545, 4561, 4956, 4968, 4975, 4982, 5259, 5261, 5459, 5460, 5610, 5643, and 5661.

V.B. Antigen Cassette

“Antigen cassette” refers to the combination of a selected antigen or plurality of antigens and the other regulatory elements necessary to transcribe the antigen(s) and express the transcribed product. An antigen or plurality of antigens can be operatively linked to regulatory components in a manner which permits transcription. Such components include conventional regulatory elements that can drive expression of the antigen(s) in a cell transfected with the viral vector. Thus the antigen cassette can also contain a selected promoter which is linked to the antigen(s) and located, with other, optional regulatory elements, within the selected viral sequences of the recombinant vector. Cassettes can include one or more antigens with epitope sequences selected from any one of SEQ ID Nos: 325-22349.

Useful promoters can be constitutive promoters or regulated (inducible) promoters, which will enable control of the amount of antigen(s) to be expressed. For example, a desirable promoter is that of the cytomegalovirus immediate early promoter/enhancer [see, e.g., Boshart et al, Cell, 41:521-530 (1985)]. Another desirable promoter includes the Rous sarcoma virus LTR promoter/enhancer. Still another promoter/enhancer sequence is the chicken cytoplasmic beta-actin promoter [T. A. Kost et al, Nucl. Acids Res., 11(23):8287 (1983)]. Other suitable or desirable promoters can be selected by one of skill in the art.

The antigen cassette can also include nucleic acid sequences heterologous to the viral vector sequences including sequences providing signals for efficient polyadenylation of the transcript (poly(A), poly-A or pA) and introns with functional splice donor and acceptor sites. A common poly-A sequence which is employed in the exemplary vectors of this invention is that derived from the papovavirus SV-40. The poly-A sequence generally can be inserted in the cassette following the antigen-based sequences and before the viral vector sequences. A common intron sequence can also be derived from SV-40, and is referred to as the SV-40 T intron sequence. An antigen cassette can also contain such an intron, located between the promoter/enhancer sequence and the antigen(s). Selection of these and other common vector elements are conventional [see, e.g., Sambrook et al, “Molecular Cloning. A Laboratory Manual.”, 2d edit., Cold Spring Harbor Laboratory, New York (1989) and references cited therein] and many such sequences are available from commercial and industrial sources as well as from Genbank.

An antigen cassette can have one or more antigens. For example, a given cassette can include 1-10, 1-20, 1-30, 10-20, 15-25, 15-20, 1, 2, 3, 4, 5, 6, 7, 8, 9, 10, 11, 12, 13, 14, 15, 16, 17, 18, 19, 20, or more antigens. Antigens can be linked directly to one another. Antigens can also be linked to one another with linkers. Antigens can be in any orientation relative to one another including N to C or C to N.

As above stated, the antigen cassette can be located in the site of any selected deletion in the viral vector, such as the site of the E1 gene region deletion or E3 gene region deletion, among others which may be selected.

The antigen cassette can be described using the following formula to describe the ordered sequence of each element, from 5′ to 3′:

(Pₐ-(L5_{b}-N_{c}-L3_{d})_{X})_{Z}-(P2ₕ-(G5ₑ-U_{f})_{Y})_{W}-G3_{g}

wherein P and P2 comprise promoter nucleotide sequences, N comprises an MHC class I epitope encoding nucleic acid sequence, L5 comprises a 5′ linker sequence, L3 comprises a 3′ linker sequence, G5 comprises a nucleic acid sequences encoding an amino acid linker, G3 comprises one of the at least one nucleic acid sequences encoding an amino acid linker, U comprises an MHC class II antigen-encoding nucleic acid sequence, where for each X the corresponding Nc is a epitope encoding nucleic acid sequence, where for each Y the corresponding Uf is an antigen-encoding nucleic acid sequence. The composition and ordered sequence can be further defined by selecting the number of elements present, for example where a=0 or 1, where b=0 or 1, where c=1, where d=0 or 1, where e=0 or 1, where f=1, where g=0 or 1, where h=0 or 1, X=1 to 400, Y=0, 1, 2, 3, 4 or 5, Z=1 to 400, and W=0, 1, 2, 3, 4 or 5.

In one example, elements present include where a=0, b=1, d=1, e=1, g=1, h=0, X=10, Y=2, Z=1, and W=1, describing where no additional promoter is present (i.e. only the promoter nucleotide sequence provided by the RNA alphavirus backbone is present), 20 MHC class I epitope are present, a 5′ linker is present for each N, a 3′ linker is present for each N, 2 MHC class II epitopes are present, a linker is present linking the two MHC class II epitopes, a linker is present linking the 5′ end of the two MHC class II epitopes to the 3′ linker of the final MHC class I epitope, and a linker is present linking the 3′ end of the two MHC class II epitopes to the to the RNA alphavirus backbone. Examples of linking the 3′ end of the antigen cassette to the RNA alphavirus backbone include linking directly to the 3′ UTR elements provided by the RNA alphavirus backbone, such as a 3′ 19-nt CSE. Examples of linking the 5′ end of the antigen cassette to the RNA alphavirus backbone include linking directly to a 26S promoter sequence, an alphavirus 5′ UTR, a 51-nt CSE, or a 24-nt CSE.

Other examples include: where a=1 describing where a promoter other than the promoter nucleotide sequence provided by the RNA alphavirus backbone is present; where a=1 and Z is greater than 1 where multiple promoters other than the promoter nucleotide sequence provided by the RNA alphavirus backbone are present each driving expression of 1 or more distinct MHC class I epitope encoding nucleic acid sequences; where h=1 describing where a separate promoter is present to drive expression of the MHC class II antigen-encoding nucleic acid sequences; and where g=0 describing the MHC class II antigen-encoding nucleic acid sequence, if present, is directly linked to the RNA alphavirus backbone.

Other examples include where each MHC class I epitope that is present can have a 5′ linker, a 3′ linker, neither, or both. In examples where more than one MHC class I epitope is present in the same antigen cassette, some MHC class I epitopes may have both a 5′ linker and a 3′ linker, while other MHC class I epitopes may have either a 5′ linker, a 3′ linker, or neither. In other examples where more than one MHC class I epitope is present in the same antigen cassette, some MHC class I epitopes may have either a 5′ linker or a 3′ linker, while other MHC class I epitopes may have either a 5′ linker, a 3′ linker, or neither.

In examples where more than one MHC class II epitope is present in the same antigen cassette, some MHC class II epitopes may have both a 5′ linker and a 3′ linker, while other MHC class II epitopes may have either a 5′ linker, a 3′ linker, or neither. In other examples where more than one MHC class II epitope is present in the same antigen cassette, some MHC class II epitopes may have either a 5′ linker or a 3′ linker, while other MHC class II epitopes may have either a 5′ linker, a 3′ linker, or neither.

The promoter nucleotide sequences P and/or P2 can be the same as a promoter nucleotide sequence provided by the RNA alphavirus backbone. For example, the promoter sequence provided by the RNA alphavirus backbone, Pn and P2, can each comprise a 26S subgenomic promoter. The promoter nucleotide sequences P and/or P2 can be different from the promoter nucleotide sequence provided by the RNA alphavirus backbone, as well as can be different from each other.

The 5′ linker L5 can be a native sequence or a non-natural sequence. Non-natural sequence include, but are not limited to, AAY, RR, and DPP. The 3′ linker L3 can also be a native sequence or a non-natural sequence. Additionally, L5 and L3 can both be native sequences, both be non-natural sequences, or one can be native and the other non-natural. For each X, the amino acid linkers can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each X, the amino acid linkers can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

The amino acid linker G5, for each Y, can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each Y, the amino acid linkers can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

The amino acid linker G3 can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. G3 can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

For each X, each N can encode a MHC class I epitope 7-15 amino acids in length. For each X, each N can also encode a MHC class I epitope 5, 6, 7, 8, 9, 10, 11, 12, 13, 14, 15, 16, 17, 18, 19, 20, 21, 22, 23, 24, 25, 26, 27, 28, 29, or 30 amino acids in length. For each X, each N can also encodes a MHC class I epitope at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

V.C. Antigen Prioritization

More candidate antigens may be available for vaccine inclusion than the vaccine technology can support. Additionally, uncertainty about various aspects of the antigen analysis may remain and tradeoffs may exist between different properties of candidate vaccine antigens. Thus, in place of predetermined filters at each step of the selection process, an integrated multi-dimensional model can be considered that places candidate antigens in a space with at least the following axes and optimizes selection using an integrative approach.
1. Risk of auto-immunity or tolerance (risk of germline) (lower risk of auto-immunity is typically preferred)
2. Probability of sequencing artifact (lower probability of artifact is typically preferred)
3. Probability of immunogenicity (higher probability of immunogenicity is typically preferred)
4. Probability of presentation (higher probability of presentation is typically preferred)
5. Gene expression (higher expression is typically preferred)
6. Coverage of HLA genes (larger number of HLA molecules involved in the presentation of a set of antigens may lower the probability that HIV will escape immune attack via downregulation or mutation of HLA molecules)
7. Coverage of HLA classes (covering both HLA-I and HLA-II may increase the probability of therapeutic response and decrease the probability of HIV escape)

Additionally, optionally, antigens can be deprioritized (e.g., excluded) from the vaccination if they are predicted to be presented by proteins corresponding to lost or inactivated HLA alleles. HLA allele loss can occur by either somatic mutation, loss of heterozygosity, or homozygous deletion of the locus. Methods for detection of HLA allele somatic mutation are well known in the art, e.g. (Shukla et al., 2015). Methods for detection of somatic LOH and homozygous deletion (including for HLA locus) are likewise well described. (Carter et al., 2012; McGranahan et al., 2017; Van Loo et al., 2010). Antigens can also be deprioritized if mass-spectrometry data indicates a predicted antigen is not presented by a predicted HLA allele.

V.D. Alphavirus

V.D.1. Alphavirus Biology

Alphaviruses are members of the family Togaviridae, and are positive-sense single stranded RNA viruses. Members are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis virus and its derivative strain TC-83 (Strauss Microbrial Review 1994). A natural alphavirus genome is typically around 12 kb in length, the first two-thirds of which contain genes encoding non-structural proteins (nsPs) that form RNA replication complexes for self-replication of the viral genome, and the last third of which contains a subgenomic expression cassette encoding structural proteins for virion production (Frolov RNA 2001).

A model lifecycle of an alphavirus involves several distinct steps (Strauss Microbrial Review 1994, Jose Future Microbiol 2009). Following virus attachment to a host cell, the virion fuses with membranes within endocytic compartments resulting in the eventual release of genomic RNA into the cytosol. The genomic RNA, which is in a plus-strand orientation and comprises a 5′ methylguanylate cap and 3′ polyA tail, is translated to produce non-structural proteins nsP1-4 that form the replication complex. Early in infection, the plus-strand is then replicated by the complex into a minus-stand template. In the current model, the replication complex is further processed as infection progresses, with the resulting processed complex switching to transcription of the minus-strand into both full-length positive-strand genomic RNA, as well as the 26S subgenomic positive-strand RNA containing the structural genes. Several conserved sequence elements (CSEs) of alphavirus have been identified to potentially play a role in the various RNA replication steps including; a complement of the 5′ UTR in the replication of plus-strand RNAs from a minus-strand template, a 51-nt CSE in the replication of minus-strand synthesis from the genomic template, a 24-nt CSE in the junction region between the nsPs and the 26S RNA in the transcription of the subgenomic RNA from the minus-strand, and a 3′ 19-nt CSE in minus-strand synthesis from the plus-strand template.

Following the replication of the various RNA species, virus particles are then typically assembled in the natural lifecycle of the virus. The 26S RNA is translated and the resulting proteins further processed to produce the structural proteins including capsid protein, glycoproteins E1 and E2, and two small polypeptides E3 and 6K (Strauss 1994). Encapsidation of viral RNA occurs, with capsid proteins normally specific for only genomic RNA being packaged, followed by virion assembly and budding at the membrane surface.

V.D.2. Alphavirus as a Delivery Vector

Alphaviruses (including alphavirus sequences, features, and other elements) can be used to generate alphavirus-based delivery vectors (also be referred to as alphavirus vectors, alphavirus viral vectors, alphavirus vaccine vectors, self-replicating RNA (srRNA) vectors, or self-amplifying RNA (samRNA) vectors). Alphaviruses have previously been engineered for use as expression vector systems (Pushko 1997, Rheme 2004). Alphaviruses offer several advantages, particularly in a vaccine setting where heterologous antigen expression can be desired. Due to its ability to self-replicate in the host cytosol, alphavirus vectors are generally able to produce high copy numbers of the expression cassette within a cell resulting in a high level of heterologous antigen production. Additionally, the vectors are generally transient, resulting in improved biosafety as well as reduced induction of immunological tolerance to the vector. The public, in general, also lacks pre-existing immunity to alphavirus vectors as compared to other standard viral vectors, such as human adenovirus. Alphavirus based vectors also generally result in cytotoxic responses to infected cells. Cytotoxicity, to a certain degree, can be important in a vaccine setting to properly illicit an immune response to the heterologous antigen expressed. However, the degree of desired cytotoxicity can be a balancing act, and thus several attenuated alphaviruses have been developed, including the TC-83 strain of VEE. Thus, an example of an antigen expression vector described herein can utilize an alphavirus backbone that allows for a high level of antigen expression, elicits a robust immune response to antigen, does not elicit an immune response to the vector itself, and can be used in a safe manner. Furthermore, the antigen expression cassette can be designed to elicit different levels of an immune response through optimization of which alphavirus sequences the vector uses, including, but not limited to, sequences derived from VEE or its attenuated derivative TC-83.

Several expression vector design strategies have been engineered using alphavirus sequences (Pushko 1997). In one strategy, a alphavirus vector design includes inserting a second copy of the 26S promoter sequence elements downstream of the structural protein genes, followed by a heterologous gene (Frolov 1993). Thus, in addition to the natural non-structural and structural proteins, an additional subgenomic RNA is produced that expresses the heterologous protein. In this system, all the elements for production of infectious virions are present and, therefore, repeated rounds of infection of the expression vector in non-infected cells can occur.

Another expression vector design makes use of helper virus systems (Pushko 1997). In this strategy, the structural proteins are replaced by a heterologous gene. Thus, following self-replication of viral RNA mediated by still intact non-structural genes, the 26S subgenomic RNA provides for expression of the heterologous protein. Traditionally, additional vectors that expresses the structural proteins are then supplied in trans, such as by co-transfection of a cell line, to produce infectious virus. A system is described in detail in U.S. Pat. No. 8,093,021, which is herein incorporated by reference in its entirety, for all purposes. The helper vector system provides the benefit of limiting the possibility of forming infectious particles and, therefore, improves biosafety. In addition, the helper vector system reduces the total vector length, potentially improving the replication and expression efficiency. Thus, an example of an antigen expression vector described herein can utilize an alphavirus backbone wherein the structural proteins are replaced by an antigen cassette, the resulting vector both reducing biosafety concerns, while at the same time promoting efficient expression due to the reduction in overall expression vector size.

V.D.3. Alphavirus Production In Vitro

Alphavirus delivery vectors are generally positive-sense RNA polynucleotides. A convenient technique well-known in the art for RNA production is in vitro transcription (IVT). In this technique, a DNA template of the desired vector is first produced by techniques well-known to those in the art, including standard molecular biology techniques such as cloning, restriction digestion, ligation, gene synthesis, and polymerase chain reaction (PCR). The DNA template contains a RNA polymerase promoter at the 5′ end of the sequence desired to be transcribed into RNA. Promoters include, but are not limited to, bacteriophage polymerase promoters such as T3, T7, or SP6. The DNA template is then incubated with the appropriate RNA polymerase enzyme, buffer agents, and nucleotides (NTPs). The resulting RNA polynucleotide can optionally be further modified including, but limited to, addition of a 5′ cap structure such as 7-methylguanosine or a related structure, and optionally modifying the 3′ end to include a polyadenylate (polyA) tail. The RNA can then be purified using techniques well-known in the field, such as phenol-chloroform extraction.

V.D.4. Delivery Via Lipid Nanoparticle

An aspect to consider in vaccine vector design is immunity against the vector itself (Riley 2017). This may be in the form of preexisting immunity to the vector itself, such as with certain human adenovirus systems, or in the form of developing immunity to the vector following administration of the vaccine. The latter is an important consideration if multiple administrations of the same vaccine are performed, such as separate priming and boosting doses, or if the same vaccine vector system is to be used to deliver different antigen cassettes. For example, efficacy of foreign vectors may be reduced if those vectors are targeted by neutralizing antibodies.

An alternative strategy is the use of nanomaterials to deliver expression vectors (Riley 2017). Nanomaterial vehicles, importantly, can be made of non-immunogenic materials and generally avoid eliciting immunity to the delivery vector itself. These materials can include, but are not limited to, lipids, inorganic nanomaterials, and other polymeric materials. Lipids can be cationic, anionic, or neutral. The materials can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soulable vitamins.

Lipid nanoparticles (LNPs) are an attractive delivery system due to the amphiphilic nature of lipids enabling formation of membranes and vesicle like structures (Riley 2017). In general, these vesicles deliver the expression vector by absorbing into the membrane of target cells and releasing nucleic acid into the cytosol. In addition, LNPs can be further modified or functionalized to facilitate targeting of specific cell types. Another consideration in LNP design is the balance between targeting efficiency and cytotoxicity. Lipid compositions generally include defined mixtures of cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids.

Nucleic-acid vectors, such as expression vectors, exposed directly to serum can have several undesirable consequences, including degradation of the nucleic acid by serum nucleases or off-target stimulation of the immune system by the free nucleic acids. Therefore, encapsulation of the alphavirus vector can be used to avoid degradation, while also avoiding potential off-target affects. In certain examples, an alphavirus vector is fully encapsulated within the delivery vehicle, such as within the aqueous interior of an LNP. Encapsulation of the alphavirus vector within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with the alphavirus delivery vector and other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the expression vectors can be further treated or modified to prepare them for administration.

V.E. Chimpanzee Adenovirus (ChAd)

V.E.1. Viral Delivery with Chimpanzee Adenovirus

Vaccine compositions for delivery of one or more antigens can be created by providing adenovirus nucleotide sequences of chimpanzee origin, a variety of novel vectors, and cell lines expressing chimpanzee adenovirus genes. A nucleotide sequence of a chimpanzee C68 adenovirus (also referred to herein as ChAdV68) can be used in a vaccine composition for antigen delivery (See SEQ ID NO: 1). Use of C68 adenovirus derived vectors is described in further detail in U.S. Pat. No. 6,083,716, which is herein incorporated by reference in its entirety, for all purposes.

In a further aspect, provided herein is a recombinant adenovirus comprising the DNA sequence of a chimpanzee adenovirus such as C68 and an antigen cassette operatively linked to regulatory sequences directing its expression. The recombinant virus is capable of infecting a mammalian, preferably a human, cell and capable of expressing the antigen cassette product in the cell. In this vector, the native chimpanzee E1 gene, and/or E3 gene, and/or E4 gene can be deleted. An antigen cassette can be inserted into any of these sites of gene deletion. The antigen cassette can include an antigen against which a primed immune response is desired.

In another aspect, provided herein is a mammalian cell infected with a chimpanzee adenovirus such as C68.

In still a further aspect, a novel mammalian cell line is provided which expresses a chimpanzee adenovirus gene (e.g., from C68) or functional fragment thereof.

In still a further aspect, provided herein is a method for delivering an antigen cassette into a mammalian cell comprising the step of introducing into the cell an effective amount of a chimpanzee adenovirus, such as C68, that has been engineered to express the antigen cassette.

Still another aspect provides a method for eliciting an immune response in a mammalian host to treat HIV. The method can comprise the step of administering to the host an effective amount of a recombinant chimpanzee adenovirus, such as C68, comprising an antigen cassette that encodes one or more antigens from HIV against which the immune response is targeted.

Also disclosed is a non-simian mammalian cell that expresses a chimpanzee adenovirus gene obtained from the sequence of SEQ ID NO: 1. The gene can be selected from the group consisting of the adenovirus E1A, E1B, E2A, E2B, E3, E4, L1, L2, L3, L4 and L5 of SEQ ID NO: 1.

Also disclosed is a nucleic acid molecule comprising a chimpanzee adenovirus DNA sequence comprising a gene obtained from the sequence of SEQ ID NO: 1. The gene can be selected from the group consisting of said chimpanzee adenovirus ETA, E1B, E2A, E2B, E3, E4, L1, L2, L3, L4 and L5 genes of SEQ ID NO: 1. In some aspects the nucleic acid molecule comprises SEQ ID NO: 1. In some aspects the nucleic acid molecule comprises the sequence of SEQ ID NO: 1, lacking at least one gene selected from the group consisting of ETA, E1B, E2A, E2B, E3, E4, L1, L2, L3, L4 and L5 genes of SEQ ID NO: 1.

Also disclosed is a vector comprising a chimpanzee adenovirus DNA sequence obtained from SEQ ID NO: 1 and an antigen cassette operatively linked to one or more regulatory sequences which direct expression of the cassette in a heterologous host cell, optionally wherein the chimpanzee adenovirus DNA sequence comprises at least the cis-elements necessary for replication and virion encapsidation, the cis-elements flanking the antigen cassette and regulatory sequences. In some aspects, the chimpanzee adenovirus DNA sequence comprises a gene selected from the group consisting of E1A, E1B, E2A, E2B, E3, E4, L1, L2, L3, L4 and L5 gene sequences of SEQ ID NO: 1. In some aspects the vector can lack the E1A and/or E1B gene.

Also disclosed herein is a host cell transfected with a vector disclosed herein such as a C68 vector engineered to expression an antigen cassette. Also disclosed herein is a human cell that expresses a selected gene introduced therein through introduction of a vector disclosed herein into the cell.

Also disclosed herein is a method for delivering an antigen cassette to a mammalian cell comprising introducing into said cell an effective amount of a vector disclosed herein such as a C68 vector engineered to expression the antigen cassette.

Also disclosed herein is a method for producing an antigen comprising introducing a vector disclosed herein into a mammalian cell, culturing the cell under suitable conditions and producing the antigen.

V.E.2. E1-Expressing Complementation Cell Lines

To generate recombinant chimpanzee adenoviruses (Ad) deleted in any of the genes described herein, the function of the deleted gene region, if essential to the replication and infectivity of the virus, can be supplied to the recombinant virus by a helper virus or cell line, i.e., a complementation or packaging cell line. For example, to generate a replication-defective chimpanzee adenovirus vector, a cell line can be used which expresses the E1 gene products of the human or chimpanzee adenovirus; such a cell line can include HEK293 or variants thereof. The protocol for the generation of the cell lines expressing the chimpanzee E1 gene products (Examples 3 and 4 of U.S. Pat. No. 6,083,716) can be followed to generate a cell line which expresses any selected chimpanzee adenovirus gene.

An AAV augmentation assay can be used to identify a chimpanzee adenovirus E1-expressing cell line. This assay is useful to identify E1 function in cell lines made by using the E1 genes of other uncharacterized adenoviruses, e.g., from other species. That assay is described in Example 4B of U.S. Pat. No. 6,083,716.

A selected chimpanzee adenovirus gene, e.g., E1, can be under the transcriptional control of a promoter for expression in a selected parent cell line. Inducible or constitutive promoters can be employed for this purpose. Among inducible promoters are included the sheep metallothionine promoter, inducible by zinc, or the mouse mammary tumor virus (MMTV) promoter, inducible by a glucocorticoid, particularly, dexamethasone. Other inducible promoters, such as those identified in International patent application WO95/13392, incorporated by reference herein can also be used in the production of packaging cell lines. Constitutive promoters in control of the expression of the chimpanzee adenovirus gene can be employed also.

A parent cell can be selected for the generation of a novel cell line expressing any desired C68 gene. Without limitation, such a parent cell line can be HeLa [ATCC Accession No. CCL 2], A549 [ATCC Accession No. CCL 185], KB [CCL 17], Detroit [e.g., Detroit 510, CCL 72] and WI-38 [CCL 75] cells. Other suitable parent cell lines can be obtained from other sources. Parent cell lines can include CHO, HEK293 or variants thereof, 911, HeLa, A549, LP-293, PER.C6, or AE1-2a.

An E1-expressing cell line can be useful in the generation of recombinant chimpanzee adenovirus E1 deleted vectors. Cell lines constructed using essentially the same procedures that express one or more other chimpanzee adenoviral gene products are useful in the generation of recombinant chimpanzee adenovirus vectors deleted in the genes that encode those products. Further, cell lines which express other human Ad E1 gene products are also useful in generating chimpanzee recombinant Ads.

V.E.3. Recombinant Viral Particles as Vectors

The compositions disclosed herein can comprise viral vectors, that deliver at least one antigen to cells. Such vectors comprise a chimpanzee adenovirus DNA sequence such as C68 and an antigen cassette operatively linked to regulatory sequences which direct expression of the cassette. The C68 vector is capable of expressing the cassette in an infected mammalian cell. The C68 vector can be functionally deleted in one or more viral genes. An antigen cassette comprises at least one antigen under the control of one or more regulatory sequences such as a promoter. Optional helper viruses and/or packaging cell lines can supply to the chimpanzee viral vector any necessary products of deleted adenoviral genes.

The term “functionally deleted” means that a sufficient amount of the gene region is removed or otherwise altered, e.g., by mutation or modification, so that the gene region is no longer capable of producing one or more functional products of gene expression. Mutations or modifications that can result in functional deletions include, but are not limited to, nonsense mutations such as introduction of premature stop codons and removal of canonical and non-canonical start codons, mutations that alter mRNA splicing or other transcriptional processing, or combinations thereof. If desired, the entire gene region can be removed.

Modifications of the nucleic acid sequences forming the vectors disclosed herein, including sequence deletions, insertions, and other mutations may be generated using standard molecular biological techniques and are within the scope of this invention.

V.E.4. Construction of the Viral Plasmid Vector

The chimpanzee adenovirus C68 vectors include recombinant, defective adenoviruses, that is, chimpanzee adenovirus sequences functionally deleted in the Ela or E1b genes, and optionally bearing other mutations, e.g., temperature-sensitive mutations or deletions in other genes. It is anticipated that these chimpanzee sequences are also useful in forming hybrid vectors from other adenovirus and/or adeno-associated virus sequences. Homologous adenovirus vectors prepared from human adenoviruses are described in the published literature [see, for example, Kozarsky I and II, cited above, and references cited therein, U.S. Pat. No. 5,240,846].

In the construction of useful chimpanzee adenovirus C68 vectors for delivery of an antigen cassette to a human (or other mammalian) cell, a range of adenovirus nucleic acid sequences can be employed in the vectors. A vector comprising minimal chimpanzee C68 adenovirus sequences can be used in conjunction with a helper virus to produce an infectious recombinant virus particle. The helper virus provides essential gene products required for viral infectivity and propagation of the minimal chimpanzee adenoviral vector. When only one or more selected deletions of chimpanzee adenovirus genes are made in an otherwise functional viral vector, the deleted gene products can be supplied in the viral vector production process by propagating the virus in a selected packaging cell line that provides the deleted gene functions in trans.

V.E.5. Recombinant Minimal Adenovirus

A minimal chimpanzee Ad C68 virus is a viral particle containing just the adenovirus cis-elements necessary for replication and virion encapsidation. That is, the vector contains the cis-acting 5′ and 3′ inverted terminal repeat (ITR) sequences of the adenoviruses (which function as origins of replication) and the native 5′ packaging/enhancer domains (that contain sequences necessary for packaging linear Ad genomes and enhancer elements for the E1 promoter). See, for example, the techniques described for preparation of a “minimal” human Ad vector in International Patent Application WO96/13597 and incorporated herein by reference.

V.E.6. Other Defective Adenoviruses

Recombinant, replication-deficient adenoviruses can also contain more than the minimal chimpanzee adenovirus sequences. These other Ad vectors can be characterized by deletions of various portions of gene regions of the virus, and infectious virus particles formed by the optional use of helper viruses and/or packaging cell lines.

As one example, suitable vectors may be formed by deleting all or a sufficient portion of the C68 adenoviral immediate early gene Ela and delayed early gene E1b, so as to eliminate their normal biological functions. Replication-defective E1-deleted viruses are capable of replicating and producing infectious virus when grown on a chimpanzee adenovirus-transformed, complementation cell line containing functional adenovirus Ela and E1b genes which provide the corresponding gene products in trans. Based on the homologies to known adenovirus sequences, it is anticipated that, as is true for the human recombinant E1-deleted adenoviruses of the art, the resulting recombinant chimpanzee adenovirus is capable of infecting many cell types and can express antigen(s), but cannot replicate in most cells that do not carry the chimpanzee E1 region DNA unless the cell is infected at a very high multiplicity of infection.

As another example, all or a portion of the C68 adenovirus delayed early gene E3 can be eliminated from the chimpanzee adenovirus sequence which forms a part of the recombinant virus.

Chimpanzee adenovirus C68 vectors can also be constructed having a deletion of the E4 gene. Still another vector can contain a deletion in the delayed early gene E2a.

Deletions can also be made in any of the late genes L1 through L5 of the chimpanzee C68 adenovirus genome. Similarly, deletions in the intermediate genes IX and IVa2 can be useful for some purposes. Other deletions may be made in the other structural or non-structural adenovirus genes.

The above discussed deletions can be used individually, i.e., an adenovirus sequence can contain deletions of E1 only. Alternatively, deletions of entire genes or portions thereof effective to destroy or reduce their biological activity can be used in any combination. For example, in one exemplary vector, the adenovirus C68 sequence can have deletions of the E1 genes and the E4 gene, or of the E1, E2a and E3 genes, or of the E1 and E3 genes, or of E1, E2a and E4 genes, with or without deletion of E3, and so on. As discussed above, such deletions can be used in combination with other mutations, such as temperature-sensitive mutations, to achieve a desired result.

The cassette comprising antigen(s) can be inserted optionally into any deleted region of the chimpanzee C68 Ad virus. Alternatively, the cassette can be inserted into an existing gene region to disrupt the function of that region, if desired.

V.E.7. Helper Viruses

Depending upon the chimpanzee adenovirus gene content of the viral vectors employed to carry the antigen cassette, a helper adenovirus or non-replicating virus fragment can be used to provide sufficient chimpanzee adenovirus gene sequences to produce an infective recombinant viral particle containing the cassette.

Useful helper viruses contain selected adenovirus gene sequences not present in the adenovirus vector construct and/or not expressed by the packaging cell line in which the vector is transfected. A helper virus can be replication-defective and contain a variety of adenovirus genes in addition to the sequences described above. The helper virus can be used in combination with the E1-expressing cell lines described herein.

For C68, the “helper” virus can be a fragment formed by clipping the C terminal end of the C68 genome with SspI, which removes about 1300 bp from the left end of the virus. This clipped virus is then co-transfected into an E1-expressing cell line with the plasmid DNA, thereby forming the recombinant virus by homologous recombination with the C68 sequences in the plasmid.

Helper viruses can also be formed into poly-cation conjugates as described in Wu et al, J. Biol. Chem., 264:16985-16987 (1989); K. J. Fisher and J. M. Wilson, Biochem. J., 299:49 (Apr. 1, 1994). Helper virus can optionally contain a reporter gene. A number of such reporter genes are known to the art. The presence of a reporter gene on the helper virus which is different from the antigen cassette on the adenovirus vector allows both the Ad vector and the helper virus to be independently monitored. This second reporter is used to enable separation between the resulting recombinant virus and the helper virus upon purification.

V.E.8. Assembly of Viral Particle and Infection of a Cell Line

Assembly of the selected DNA sequences of the adenovirus, the antigen cassette, and other vector elements into various intermediate plasmids and shuttle vectors, and the use of the plasmids and vectors to produce a recombinant viral particle can all be achieved using conventional techniques. Such techniques include conventional cloning techniques of cDNA, in vitro recombination techniques (e.g., Gibson assembly), use of overlapping oligonucleotide sequences of the adenovirus genomes, polymerase chain reaction, and any suitable method which provides the desired nucleotide sequence. Standard transfection and co-transfection techniques are employed, e.g., CaPO4 precipitation techniques or liposome-mediated transfection methods such as lipofectamine. Other conventional methods employed include homologous recombination of the viral genomes, plaquing of viruses in agar overlay, methods of measuring signal generation, and the like.

For example, following the construction and assembly of the desired antigen cassette-containing viral vector, the vector can be transfected in vitro in the presence of a helper virus into the packaging cell line. Homologous recombination occurs between the helper and the vector sequences, which permits the adenovirus-antigen sequences in the vector to be replicated and packaged into virion capsids, resulting in the recombinant viral vector particles.

The resulting recombinant chimpanzee C68 adenoviruses are useful in transferring an antigen cassette to a selected cell. In in vivo experiments with the recombinant virus grown in the packaging cell lines, the E1-deleted recombinant chimpanzee adenovirus demonstrates utility in transferring a cassette to a non-chimpanzee, preferably a human, cell.

V.E.9. Use of the Recombinant Virus Vectors

The resulting recombinant chimpanzee C68 adenovirus containing the antigen cassette thus provides an efficient gene transfer vehicle which can deliver antigen(s) to a subject in vivo or ex vivo.

The above-described recombinant vectors are administered to humans according to published methods for gene therapy. A chimpanzee viral vector bearing an antigen cassette can be administered to a patient, preferably suspended in a biologically compatible solution or pharmaceutically acceptable delivery vehicle, as described herein. A suitable vehicle includes sterile saline. Other aqueous and non-aqueous isotonic sterile injection solutions and aqueous and non-aqueous sterile suspensions known to be pharmaceutically acceptable carriers and well known to those of skill in the art may be employed for this purpose.

The chimpanzee adenoviral vectors are administered in sufficient amounts to transduce the human cells and to provide sufficient levels of antigen transfer and expression to provide a therapeutic benefit without undue adverse or with medically acceptable physiological effects, which can be determined by those skilled in the medical arts. Conventional and pharmaceutically acceptable routes of administration include, but are not limited to, direct delivery to the liver, intranasal, intravenous, intramuscular, subcutaneous, intradermal, oral and other parental routes of administration. Routes of administration may be combined, if desired.

Dosages of the viral vector will depend primarily on factors such as the condition being treated, the age, weight and health of the patient, and may thus vary among patients. The dosage will be adjusted to balance the therapeutic benefit against any side effects and such dosages may vary depending upon the therapeutic application for which the recombinant vector is employed. The levels of expression of antigen(s) can be monitored to determine the frequency of dosage administration.

Recombinant, replication defective adenoviruses can be administered in a “pharmaceutically effective amount”, that is, an amount of recombinant adenovirus that is effective in a route of administration to transfect the desired cells and provide sufficient levels of expression of the selected gene to provide a vaccinal benefit, i.e., some measurable level of protective immunity. C68 vectors comprising an antigen cassette can be co-administered with adjuvant. Adjuvant can be separate from the vector (e.g., alum) or encoded within the vector, in particular if the adjuvant is a protein. Adjuvants are well known in the art.

Conventional and pharmaceutically acceptable routes of administration include, but are not limited to, intranasal, intramuscular, intratracheal, subcutaneous, intradermal, rectal, oral and other parental routes of administration. Routes of administration may be combined, if desired, or adjusted depending upon the immunogen or the disease. For example, in prophylaxis of rabies, the subcutaneous, intratracheal and intranasal routes are preferred. The route of administration primarily will depend on the nature of the disease being treated.

The levels of immunity to antigen(s) can be monitored to determine the need, if any, for boosters. Following an assessment of antibody titers in the serum, for example, optional booster immunizations may be desired

V.F. Pharmaceutical Compositions

A vaccine composition can be a pharmaceutical composition that further comprises an adjuvant and/or a carrier. Examples of useful adjuvants and carriers are given herein below. A composition can be associated with a carrier such as a protein or an antigen-presenting cell such as a dendritic cell (DC) capable of presenting the peptide to a T-cell.

Adjuvants are any substance whose admixture into a vaccine composition increases or otherwise modifies the immune response to an antigen. Carriers can be scaffold structures, for example a polypeptide or a polysaccharide, to which an antigen, is capable of being associated. Optionally, adjuvants are conjugated covalently or non-covalently.

The ability of an adjuvant to increase an immune response to an antigen is typically manifested by a significant or substantial increase in an immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response.

Suitable adjuvants include, but are not limited to 1018 ISS, alum, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives can readily be determined by the skilled artisan without undue experimentation. Additional adjuvants include colony-stimulating factors, such as Granulocyte Macrophage Colony Stimulating Factor (GM-CSF, sargramostim).

A vaccine composition can comprise more than one different adjuvant. Furthermore, a therapeutic composition can comprise any adjuvant substance including any of the above or combinations thereof. It is also contemplated that a vaccine and an adjuvant can be administered together or separately in any appropriate sequence.

A carrier (or excipient) can be present independently of an adjuvant. In some aspects, the carrier is present in conjunction with the adjuvant. The function of a carrier can for example be to increase the molecular weight to increase activity or immunogenicity, to confer stability, to increase the biological activity, or to increase serum half-life. Furthermore, a carrier can aid presenting peptides to T-cells. A carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. However, tetanus toxoid and/or diptheria toxoid are suitable carriers. Alternatively, the carrier can be dextrans for example sepharose.

Additional examples of carriers can be acqueous carriers such as water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments a vaccine composition additionally contains at least one antigen presenting cell.

In some aspects, any of the above compositions further comprise a nanoparticulate delivery vehicle. The nanoparticulate delivery vehicle, in some aspects, may be a lipid nanoparticle (LNP) or liposomes. In some aspects, the LNP comprises ionizable amino lipids. In some aspects, the ionizable amino lipids comprise MC3-like (dilinoleylmethyl-4-dimethylaminobutyrate) molecules. In some aspects, the nanoparticulate delivery vehicle encapsulates the antigen expression system.

In some aspects, any of the above compositions further comprise a plurality of LNPs, wherein the LNPs comprise: the antigen expression system; a cationic lipid; a non-cationic lipid; and a conjugated lipid that inhibits aggregation of the LNPs, wherein at least about 95% of the LNPs in the plurality of LNPs either: have a non-lamellar morphology; or are electron-dense.

In some aspects, the non-cationic lipid is a mixture of (1) a phospholipid and (2) cholesterol or a cholesterol derivative.

In some aspects, the conjugated lipid that inhibits aggregation of the LNPs is a polyethyleneglycol (PEG)-lipid conjugate. In some aspects, the PEG-lipid conjugate is selected from the group consisting of: a PEG-diacylglycerol (PEG-DAG) conjugate, a PEG dialkyloxypropyl (PEG-DAA) conjugate, a PEG-phospholipid conjugate, a PEG-ceramide (PEG-Cer) conjugate, and a mixture thereof. In some aspects the PEG-DAA conjugate is a member selected from the group consisting of a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, a PEG-distearyloxypropyl (C₁₈) conjugate, and a mixture thereof.

In some aspects, the antigen expression system is fully encapsulated in the LNPs.

In some aspects, the non-lamellar morphology of the LNPs comprises an inverse hexagonal (H_{II}) or cubic phase structure.

In some aspects, the cationic lipid comprises from about 10 mol % to about 50 mol % of the total lipid present in the LNPs. In some aspects, the cationic lipid comprises from about 20 mol % to about 50 mol % of the total lipid present in the LNPs. In some aspects, the cationic lipid comprises from about 20 mol % to about 40 mol % of the total lipid present in the LNPs.

In some aspects, the non-cationic lipid comprises from about 10 mol % to about 60 mol % of the total lipid present in the LNPs. In some aspects, the non-cationic lipid comprises from about 20 mol % to about 55 mol % of the total lipid present in the LNPs. In some aspects, the non-cationic lipid comprises from about 25 mol % to about 50 mol % of the total lipid present in the LNPs.

In some aspects, the conjugated lipid comprises from about 0.5 mol % to about 20 mol % of the total lipid present in the LNPs. In some aspects, the conjugated lipid comprises from about 2 mol % to about 20 mol % of the total lipid present in the LNPs. In some aspects, the conjugated lipid comprises from about 1.5 mol % to about 18 mol % of the total lipid present in the LNPs.

In some aspects, greater than 95% of the LNPs have a non-lamellar morphology. In some aspects, greater than 95% of the LNPs are electron dense.

In some aspects, any of the above compositions further comprise a plurality of LNPs, wherein the LNPs comprise: a cationic lipid comprising from 50 mol % to 65 mol % of the total lipid present in the LNPs; a conjugated lipid that inhibits aggregation of LNPs comprising from 0.5 mol % to 2 mol % of the total lipid present in the LNPs; and a non-cationic lipid comprising either: a mixture of a phospholipid and cholesterol or a derivative thereof, wherein the phospholipid comprises from 4 mol % to 10 mol % of the total lipid present in the LNPs and the cholesterol or derivative thereof comprises from 30 mol % to 40 mol % of the total lipid present in the LNPs; a mixture of a phospholipid and cholesterol or a derivative thereof, wherein the phospholipid comprises from 3 mol % to 15 mol % of the total lipid present in the LNPs and the cholesterol or derivative thereof comprises from 30 mol % to 40 mol % of the total lipid present in the LNPs; or up to 49.5 mol % of the total lipid present in the LNPs and comprising a mixture of a phospholipid and cholesterol or a derivative thereof, wherein the cholesterol or derivative thereof comprises from 30 mol % to 40 mol % of the total lipid present in the LNPs.

In some aspects, any of the above compositions further comprise a plurality of LNPs, wherein the LNPs comprise: a cationic lipid comprising from 50 mol % to 85 mol % of the total lipid present in the LNPs; a conjugated lipid that inhibits aggregation of LNPs comprising from 0.5 mol % to 2 mol % of the total lipid present in the LNPs; and a non-cationic lipid comprising from 13 mol % to 49.5 mol % of the total lipid present in the LNPs.

In some aspects, the phospholipid comprises dipalmitoylphosphatidylcholine (DPPC), distearoylphosphatidylcholine (DSPC), or a mixture thereof.

In some aspects, the conjugated lipid comprises a polyethyleneglycol (PEG)-lipid conjugate. In some aspects, the PEG-lipid conjugate comprises a PEG-diacylglycerol (PEG-DAG) conjugate, a PEG-dialkyloxypropyl (PEG-DAA) conjugate, or a mixture thereof. In some aspects, the PEG-DAA conjugate comprises a PEG-dimyristyloxypropyl (PEG-DMA) conjugate, a PEG-distearyloxypropyl (PEG-DSA) conjugate, or a mixture thereof. In some aspects, the PEG portion of the conjugate has an average molecular weight of about 2,000 daltons.

In some aspects, the conjugated lipid comprises from 1 mol % to 2 mol % of the total lipid present in the LNPs.

In some aspects, the LNP comprises a compound having a structure of Formula I:

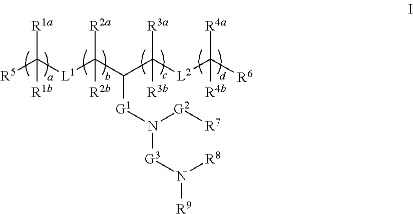

or a pharmaceutically acceptable salt, tautomer, prodrug or stereoisomer thereof, wherein: L¹ and L² are each independently -0(C=0)-, —(C=0)0-, —C(=0)-, —0-, —S(0)ₓ—, —S—S—, —C(=0)S—, —SC(=0)-, —R^{a}C(=0)-, —C(═0) R^{a}—, —R^{a}C(=0)R^{a}—, —OC(═0)R^{a}—, —R^{a}C(=0)0- or a direct bond; G¹ is Ci-C₂ alkylene, —(C=0)-, -0(C=0)-, —SC(=0)-, —R^{a}C(=0)- or a direct bond: —C(=0)-, —(C=0)0-, —C(=0)S—, —C(=0)R^{a}— or a direct bond; G is C₁-C₆ alkylene; R^{a} is H or C1-C12 alkyl; R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond; R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond; R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond; R^{4a} and R^{4b} are, at each occurrence, independently either: (a) H or C1-C12 alkyl; or (b) R^{4a} is H or C1-C12 alkyl, and R^{4b} together with the carbon atom to which it is bound is taken together with an adjacent R^{4b} and the carbon atom to which it is bound to form a carbon-carbon double bond; R⁵ and R⁶ are each independently H or methyl; R⁷ is C4-C20 alkyl; R⁸ and R⁹ are each independently C1-C12 alkyl; or R⁸ and R⁹, together with the nitrogen atom to which they are attached, form a 5, 6 or 7-membered heterocyclic ring; a, b, c and d are each independently an integer from 1 to 24; and x is 0, 1 or 2.

In some aspects, the LNP comprises a compound having a structure of Formula II:

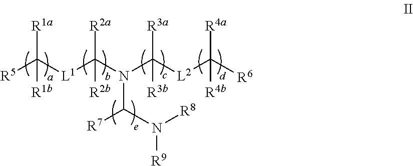

or a pharmaceutically acceptable salt, tautomer, prodrug or stereoisomer thereof, wherein: L¹ and L² are each independently -0(C=0)-, —(C=0)0- or a carbon-carbon double bond; R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond; R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond; R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond; R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond; R⁵ and R⁶ are each independently methyl or cycloalkyl; R⁷ is, at each occurrence, independently H or C₁-C₁₂ alkyl; R⁸ and R⁹ are each independently unsubstituted C1-C12 alkyl; or R⁸ and R⁹, together with the nitrogen atom to which they are attached, form a 5, 6 or 7-membered heterocyclic ring comprising one nitrogen atom; a and d are each independently an integer from 0 to 24; b and c are each independently an integer from 1 to 24; and e is 1 or 2, provided that: at least one of R^{1a}, R^{2a}, R^{3a} or R^{4a} is C1-C12 alkyl, or at least one of L¹ or L² is -0(C=0)- or —(C=0)0-; and R^{1a} and R^{1b} are not isopropyl when a is 6 or n-butyl when a is 8.

In some aspects, any of the above compositions further comprise one or more excipients comprising a neutral lipid, a steroid, and a polymer conjugated lipid. In some aspects, the neutral lipid comprises at least one of 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), and 1,2-Dioleoyl-sn-glycero-3-phosphoethanolamine (DOPE). In some aspects, the neutral lipid is DSPC.

In some aspects, the molar ratio of the compound to the neutral lipid ranges from about 2:1 to about 8:1.

In some aspects, the steroid is cholesterol. In some aspects, the molar ratio of the compound to cholesterol ranges from about 2:1 to 1:1.

In some aspects, the polymer conjugated lipid is a pegylated lipid. In some aspects, the molar ratio of the compound to the pegylated lipid ranges from about 100:1 to about 25:1. In some aspects, the pegylated lipid is PEG-DAG, a PEG polyethylene (PEG-PE), a PEG-succinoyl-diacylglycerol (PEG-S-DAG), PEG-cer or a PEG dialkyoxypropylcarbamate. In some aspects, the pegylated lipid has the following structure III:

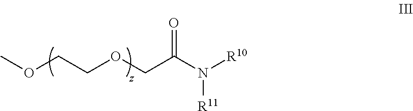

or a pharmaceutically acceptable salt, tautomer or stereoisomer thereof, wherein: R¹⁰ and R¹¹ are each independently a straight or branched, saturated or unsaturated alkyl chain containing from 10 to 30 carbon atoms, wherein the alkyl chain is optionally interrupted by one or more ester bonds; and z has a mean value ranging from 30 to 60. In some aspects, R¹⁰ and R¹¹ are each independently straight, saturated alkyl chains having 12 to 16 carbon atoms. In some aspects, the average z is about 45.

In some aspects, the LNP self-assembles into non-bilayer structures when mixed with polyanionic nucleic acid. In some aspects, the non-bilayer structures have a diameter between 60 nm and 120 nm. In some aspects, the non-bilayer structures have a diameter of about 70 nm, about 80 nm, about 90 nm, or about 100 nm. In some aspects, wherein the nanoparticulate delivery vehicle has a diameter of about 100 nm.

In some aspects, a targeting ligand can be included with the lipid nanoparticle. For example, the targeting ligand can be incorporated into the liposome and can include antibodies or fragments thereof specific for cell surface determinants of the desired immune system cells.

Also disclosed herein is a pharmaceutical composition comprising any of the compositions disclosed herein (such as an alphavirus-based or ChAd-based vector disclosed herein) and a pharmaceutically acceptable adjuvant and/or carrier.

### VI. Therapeutic Methods

Also provided is a method of inducing a HIV specific immune response in a subject, vaccinating against HIV (e.g., a prophylactic treatment), treating and or alleviating a symptom of HIV in a subject by administering to the subject one or more antigens such as a plurality of antigens identified using methods disclosed herein.

In some aspects, a subject has been diagnosed with HIV, at risk of contracting HIV, or at risk of exposure to HIV. A subject can be a human, dog, cat, horse or any animal in which a HIV specific immune response is desired.

A vaccine composition can be administered such that the amount of one or more antigens in the vaccine composition is sufficient to induce a CTL response.

A vaccine composition can be administered alone or in combination with other therapeutic agents. A therapeutic agent is for example, anti-retrovirals such as nucleoside reverse transcriptase inhibitors (NRTIs), nonnucleoside reverse transcriptase inhibitors (NNRTIs), protease inhibitors (PIs), fusion inhibitors, Entry inhibitors—CCr5 co-receptor antagonist, or HIV integrase strand transfer inhibitors.

The optimum amount of each antigen to be included in a vaccine composition and the optimum dosing regimen can be determined. For example, an antigen or its variant can be prepared for intravenous (i.v.) injection, sub-cutaneous (s.c.) injection, intradermal (i.d.) injection, intraperitoneal (i.p.) injection, intramuscular (i.m.) injection, parenteral, topical, nasal, oral, or local administration. Methods of injection include s.c., i.d., i.p., i.m., and i.v. Methods of DNA or RNA injection include i.d., i.m., s.c., i.p. and i.v. Other methods of administration of the vaccine composition are known to those skilled in the art.

Compositions comprising an antigen can be administered to an individual already suffering from HIV. In therapeutic applications, compositions are administered to a patient in an amount sufficient to elicit an effective CTL response to the HIV antigen and to cure or at least partially arrest symptoms, complications, and/or progression of HIV. An amount adequate to accomplish this is defined as “therapeutically effective dose.” Amounts effective for this use will depend on, e.g., the composition, the manner of administration, the stage and severity of HIV being treated, the weight and general state of health of the patient, and the judgment of the prescribing physician. In some instances, the vaccine composition can be administered sequentially where subsequent administrations represent boosting doses. Such boosting doses can be further administered until at least symptoms are substantially abated and for a period

A vaccine can be compiled so that the selection, number and/or amount of antigens present in the composition is specific for a particular category, type, or subtype of HIV and specific for a patient. Additionally, the selection can be dependent on the status (e.g., early stage or late stage) of the disease, earlier treatment regimens, the immune status of the patient, and the HLA-haplotype of the patient. Furthermore, a vaccine can contain individualized components, according to personal needs of the particular patient. Examples include varying the selection of antigens according to the expression of the antigen in the particular patient or adjustments for secondary treatments following a first round or scheme of treatment.

### VII. Selecting a Subject for Administering an Antigen-Based Vaccine

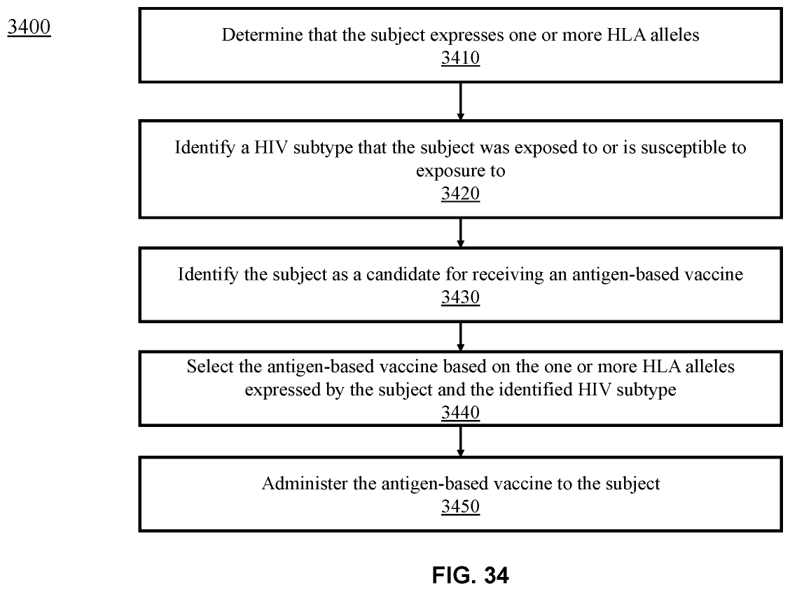

A subject can be identified as a candidate for receiving an antigen-based vaccine through the use of various diagnostic methods. Reference is made to FIG. 34 depicts a flow process 3400 for providing an antigen-based vaccine to the subject, in accordance with one embodiment.

In one aspect, patient selection for antigen vaccination is performed by considering the subject's HLA type. In one aspect, patient selection for antigen vaccination is performed by considering a HIV subtype that the subject was exposed to or will likely become exposed to. In some aspects, patient selection for antigen vaccination is performed by considering both 1) the subject's HLA type and 2) a HIV subtype that the subject was exposed to or will likely become exposed to.

As an example, a subject is considered eligible for the vaccine therapy if 1) the subject carries an HLA allele predicted or known to present an antigen with an epitope sequence included in a vaccine, and 2) the subject was exposed to a HIV subtype that expresses the antigen with the epitope sequence. As another example, the subject is considered eligible for the vaccine therapy if 1) the subject carries an HLA allele predicted or known to present an antigen with an epitope sequence included in a vaccine, and 2) the patient is susceptible to exposure to a particular HIV subtype that expresses the antigen with the epitope sequence.

VII.A. Isolation and Detection of HLA Peptides

At step 3410, whether the subject expresses one or more HLA alleles is determined. In one aspect, the one or more HLA alleles are class I HLA alleles, class II HLA alleles, or both class I and class II HLA alleles.

In one aspect, determining whether the subject expresses one or more HLA alleles involves a population-based analysis. More specifically, determining whether the subject expresses one or more HLA alleles includes determining the origin of the subject and further identifying one or more HLA alleles that are known to be commonly expressed by the population of individuals of that origin. Examples of an origin can be ethnicity, geographic location, birth location, or ancestry. In one embodiment, an HLA allele is considered commonly expressed by the population of individuals of an origin if there is a greater than 95% chance that an individual of that origin expresses that HLA allele. In some embodiments, an HLA allele is considered commonly expressed by the population of individuals of an origin if there is a greater than a 50, 55, 60, 65, 70, 75, 80, 85, or 90% chance that an individual of that origin expresses that HLA allele. For example, a subject is determined to be of European origin and individuals of European origin are known to express one or more HLA alleles. Thus, the subject of European origin is determined to express the known one or more HLA alleles expressed by individuals of European origin. Common expression of HLA alleles based on an origin can be found in available databases such as http://www.ebi.ac.uk/imgt/hla/ambig.html.

In one aspect, determining whether the subject expresses one or more HLA alleles involves identifying the haplotype of the patient though high-throughput sequencing or Sanger sequencing diagnostic methods. Example patient haplotypes are documented in the column entitled “HLA alleles” in Tables 35-45. First, isolation of HLA-peptide molecules is performed using classic immunoprecipitation (IP) methods on a sample. In some aspects, the sample is a tissue sample and prior to IP, the tissue sample is lysed and solubilized. A clarified lysate is used for HLA specific IP.

Immunoprecipitation is performed using antibodies coupled to beads where the antibody is specific for HLA molecules. For a pan-Class I HLA immunoprecipitation, a pan-Class I CR antibody is used, for Class II HLA-DR, an HLA-DR antibody is used. Antibody is covalently attached to NHS-sepharose beads during overnight incubation. After covalent attachment, the beads were washed and aliquoted for IP. Immunoprecipitations can also be performed with antibodies that are not covalently attached to beads. Typically this is done using sepharose or magnetic beads coated with Protein A and/or Protein G to hold the antibody to the column. Some antibodies that can be used to selectively enrich MHC/peptide complex are listed below.

| Antibody Name | Specificity |
|---|---|
| W6/32 | Class I HLA-A, B, C |
| L243 | Class II-HLA-DR |
| Tu36 | Class II-HLA-DR |
| LN3 | Class II-HLA-DR |
| Tu39 | Class II-HLA-DR, DP, DQ |

The clarified tissue lysate is added to the antibody beads for the immunoprecipitation. After immunoprecipitation, the beads are removed from the lysate and the lysate stored for additional experiments, including additional IPs. The IP beads are washed to remove non-specific binding and the HLA/peptide complex is eluted from the beads using standard techniques. The protein components are removed from the peptides using a molecular weight spin column or C18 fractionation. The resultant peptides are taken to dryness by SpeedVac evaporation and in some instances are stored at −20 C prior to MS analysis.

Dried peptides are reconstituted in an HPLC buffer suitable for reverse phase chromatography and loaded onto a C-18 microcapillary HPLC column for gradient elution in a Fusion Lumos mass spectrometer (Thermo). MS1 spectra of peptide mass/charge (m/z) were collected in the Orbitrap detector at high resolution followed by MS2 low resolution scans collected in the ion trap detector after HCD fragmentation of the selected ion. Additionally, MS2 spectra can be obtained using either CID or ETD fragmentation methods or any combination of the three techniques to attain greater amino acid coverage of the peptide. MS2 spectra can also be measured with high resolution mass accuracy in the Orbitrap detector.

MS2 spectra from each analysis are searched against a protein database using Comet and the peptide identification are scored using Percolator. Additional sequencing is performed using PEAKS studio (Bioinformatics Solutions Inc.) and other search engines or sequencing methods can be used including spectral matching and de novo sequencing.

In one aspect, the subject is deemed to be expressing an HLA allele if the HLA allele has an HLA frequency of at least 0.5%. In some aspects, the subject deemed to be expressing an HLA allele if the HLA allele has a HLA frequency of at least 1%, 2%, 3%, 4%, or 5%.

VII.B.1. MS Limit of Detection Studies in Support of Comprehensive HLA Peptide Sequencing.

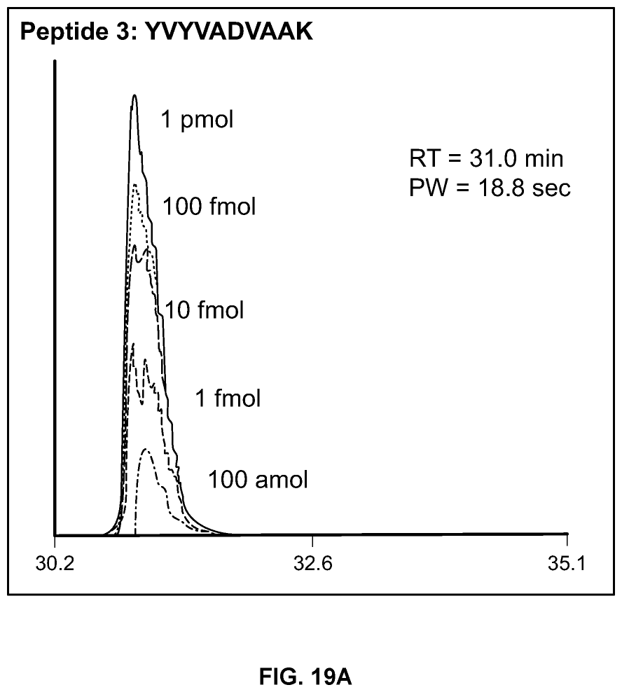

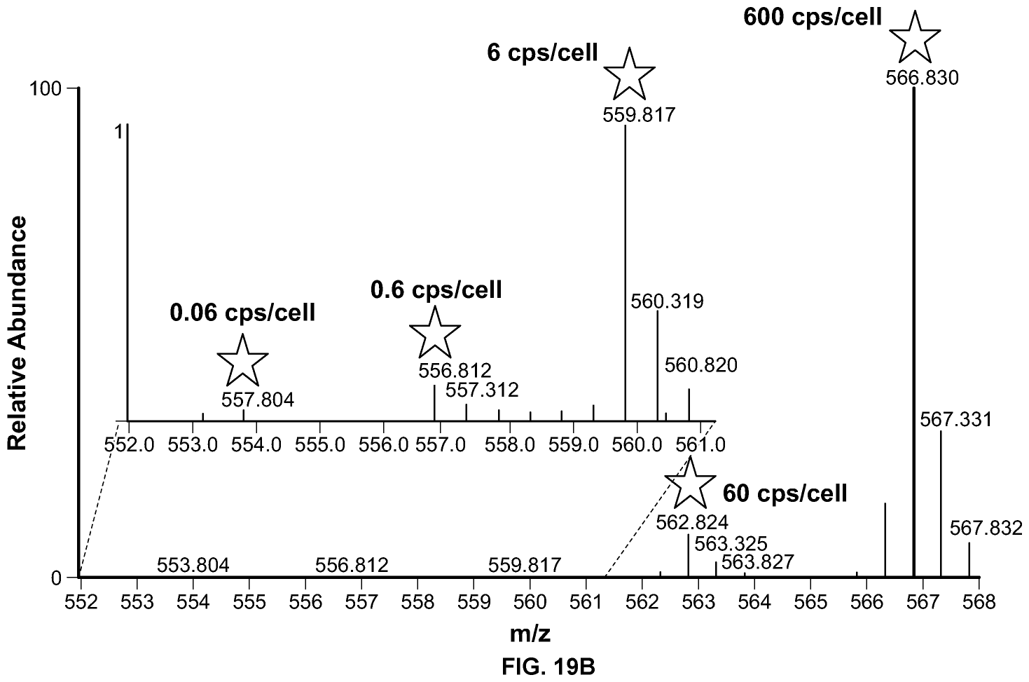

Using the peptide YVYVADVAAK (SEQ ID NO: 94), it was determined what the limits of detection are using different amounts of peptide loaded onto the LC column. The amounts of peptide tested were 1 pmol, 100 fmol, 10 fmol, 1 fmol, and 100 amol. (Table 2) The results are shown in FIGS. 19A and 19B. These results indicate that the lowest limit of detection (LoD) is in the attomol range (10⁻¹⁸), that the dynamic range spans five orders of magnitude, and that the signal to noise appears sufficient for sequencing at low femtomol ranges (10⁻¹⁵).

TABLE 2

| Peptide m/z | Loaded on Column | | Copies/Cell in 1e9cells |
|---|---|---|---|
| 566.830 | 1 | pmol | 600 |
| 562.823 | 100 | fmol | 60 |
| 559.816 | 10 | fmol | 6 |
| 556.810 | 1 | fmol | 0.6 |
| 553.802 | 100 | amol | 0.06 |

VII.B. Identifying HIV Subtype

Returning to FIG. 34, at step 3420, a HIV subtype that the subject has been exposed to or a HIV subtype that the subject is susceptible to is identified.

To identify a HIV subtype that a subject has been exposed to, a test sample is obtained from the subject. The test sample can be any of blood, seminal fluid, ocular lens fluid, cerebral spinal fluid, saliva, synovial fluid, peritoneal fluid, amniotic fluid, tissue, or needle aspirate. A HIV isolate is extracted from the test sample. In one aspect, extraction includes separating cellular components in the test sample from HIV isolate through centrifugation and the HIV isolate can be retained in the supernatant. In one aspect, extraction includes lysing and solubilizing the test sample. The lysate can be further clarified (e.g., centrifuged/filtered) to obtain a HIV isolate.

Detection of the HIV subtype in the HIV isolates can be conducted using enzyme-linked immunosorbent assay (ELISA), dot blot assays, HIV spot and comb tests, immunofluorescence tests, or Western blot. In some aspects, detection of the HIV subtype in the HIV isolate is conducted by amplifying the viral nucleic acid in the HIV isolates (e.g., polymerase chain reaction). The HIV isolates are mixed with amplification reagents and a set of primers to amplify target sequences of the particular HIV subtype. The amplified target sequences can then be detected using a variety of detection technologies. For example, exposure of the target sequences to probes would form a probe/sequence product, which can be further detected as an indication of the presence of a particular HIV subtype. Example primers and probes for detecting particular HIV subtypes are described in WO 2003020878, which is hereby incorporated by reference in its entirety.

A patient can be susceptible to exposure to a particular HIV subtype based on the prevalence of HIV subtypes at the patient's current geographic location or the patient's future, planned geographic destination. For example, a patient can be susceptible to HIV subtype A1 and A2 if the patient is located at or planning to travel to Central and East African countries. A patient can be susceptible to HIV subtype B if the patient is located at or planning to travel to West and Central Europe, North or South America, Australia, or Southeast Asia. A patient can be susceptible to HIV subtype C if the patient is located at or planning to travel to Sub-Saharan Africa, India, or Brazil. A patient can be susceptible to HIV subtype D if the patient is located at or planning to travel to North Africa or the Middle East. A patient can be susceptible to HIV subtype F1 or F2 if the patient is located at or planning to travel to South or Southeast Asia. A patient can be susceptible to HIV subtype G if the patient is located at or planning to travel to West or Central Africa. A patient can be susceptible to HIV subtypes H if the patient is located at or planning to travel to Central Africa. A patient can be susceptible to HIV subtype J if the patient is located at or planning to travel to North, Central, or West Africa, or the Carribean. A patient can be susceptible to HIV subtype K if the patient is located at or planning to travel to the Democratic Republic of Congo or Cameroon.

In some embodiments, an understanding of the HIV subtype is not needed and therefore, step 3420 need not be performed. For example, if a vaccine includes sufficient antigens such that the vaccine can be predicted to be efficacious against multiple HIV subtypes, then identification if the particular HIV subtype for this subject is not needed.

VII.C. Candidate Patient

Returning to FIG. 34, at step 3430, the subject is identified as a candidate for receiving an antigen-based vaccine. Generally, the subject is identified as a candidate if the subject expresses a HLA allele (determined at step 3410) and the HLA allele is known or predicted to likely present a HIV antigen with an epitope sequence that is expressed by the identified HIV subtype (identified at step 3420). Tables 35-45 show pairings of HLA alleles and epitope sequences, where each HLA allele in a pair is predicted to present a corresponding epitope sequence.

At step 3440, an antigen-based vaccine is selected based on the HLA alleles expressed by the subject and the identified HIV subtype. In one aspect, the antigen-based vaccine is a personalized vaccine that was previously developed 1) for subjects that express the HLA alleles and 2) for the particular identified HIV subtype. For example, the antigen-based vaccine can include 1, 2, 3, 4, 5, 6, 7, 8, 9, or 10 epitopes that are known to be expressed by the identified HIV subtype, each of the epitopes known or predicted to likely be presented by the proteins corresponding to expressed HLA alleles of the subjects. As another example, the antigen-based vaccine can include antigen-encoding nucleic acid sequences that encode for antigens that include the epitope sequences. Such epitope sequences are known or predicted to likely be presented by the proteins corresponding to expressed HLA alleles of the subjects.

At step 3450, the selected antigen-based vaccine is administered to the subject.

In some aspects, the steps in the flow process 3400 can be differently ordered than as shown in FIG. 34. For example, the HIV subtype may be identified (step 3420) prior to determining the subject's expression of one or more HLA alleles (step 3410).

VII.D. Alternate Embodiment for Selecting a Subject for Administering an Antigen-Based Vaccine

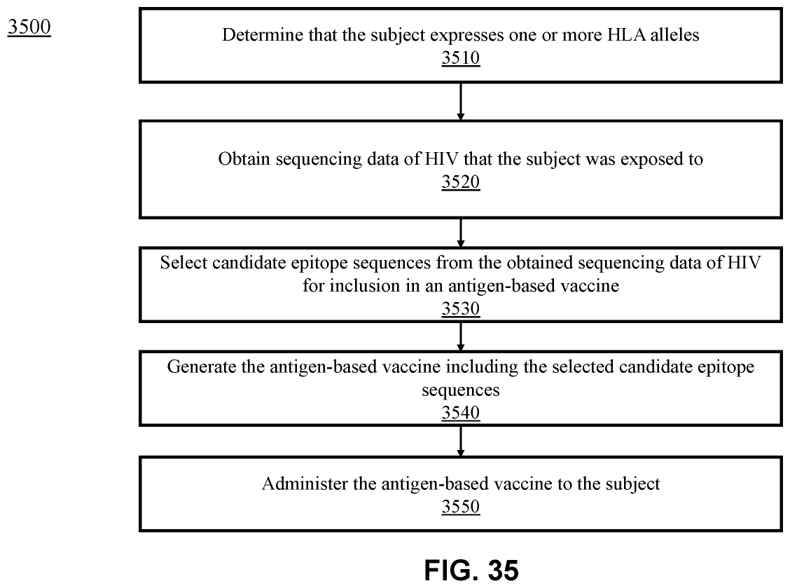

Reference is made to FIG. 35 depicts a flow process 3500 for providing an antigen-based vaccine to the subject, in accordance with a second embodiment. Given the high mutation rate of HIV, in some scenarios, particular epitope sequences of proteins derived from HIV may be mutated. Such mutations may have arisen in the HIV after the HIV infected the subject. Additionally, these mutated epitope sequences may be presented by HLA alleles of a subject. Thus, FIG. 35 depicts a flow process for providing a personalized antigen-based vaccine to a subject, where the antigen-based vaccine includes antigens with mutated epitope sequences corresponding to HIV that the subject was previously exposed to.

At step 3510, whether the subject expresses one or more HLA alleles is determined. Similar to step 3410 shown in FIG. 34, the determination of whether the subject expresses one or more HLA alleles involves performing ancestral population-based analysis or involves identifying the haplotype of the patient.

At step 3520, sequencing data of HIV that the subject was exposed to is obtained. In one aspect, a sample containing HIV can be obtained from the subject and the HIV is then sequenced. As an example, the sample can be obtained from the subject's lymph nodes and the HIV can be sequenced according to the methods described above in the section entitled “Identifying HIV epitope sequences.”

At step 3530, candidate epitope sequences are selected for inclusion in an antigen-based vaccine. To identify candidate epitope sequences, a presentation model can be applied to the sequencing data of HIV. The presentation model is described in further detail below. In some aspects, the candidate epitope sequences include mutated epitope sequences identified from the obtained sequencing data of HIV. Such mutated epitope sequences may not appear in Tables 35-45. In some aspects, the candidate epitope sequences include any of the epitope sequences shown in Tables 35-45 (e.g., any of SEQ ID Nos: 325-22349). In some aspects, the candidate epitope sequences include validated HIV epitope sequences. In some aspects, the candidate epitope sequences include any combination of mutated epitope sequences, epitope sequences shown in Tables 35-45 (any of SEQ ID Nos: 325-22349), and validated HIV epitope sequences.

At step 3540, the antigen-based vaccine is generated, the vaccine including the selected candidate epitope sequences. Thus, the antigen-based vaccine is a personalized vaccine for the subject as it includes the mutated epitope sequences that are specific for the mutated epitope sequences expressed by HIV that has infected the subject.

At step 3550, the antigen-based vaccine is administered to the subject.

### VIII. Vaccine Manufacturing

Also disclosed is a method of manufacturing an antigen-based vaccine, comprising performing the steps of a method disclosed herein; and producing an antigen-based vaccine comprising a plurality of antigens or a subset of the plurality of antigens.

Antigens disclosed herein can be manufactured using methods known in the art. For example, a method of producing an antigen or a vector (e.g., a vector including at least one sequence encoding one or more antigens) disclosed herein can include culturing a host cell under conditions suitable for expressing the antigen or vector wherein the host cell comprises at least one polynucleotide encoding the antigen or vector, and purifying the antigen or vector. Standard purification methods include chromatographic techniques, electrophoretic, immunological, precipitation, dialysis, filtration, concentration, and chromatofocusing techniques.

Host cells can include a Chinese Hamster Ovary (CHO) cell, NS0 cell, yeast, or a HEK293 cell. Host cells can be transformed with one or more polynucleotides comprising at least one nucleic acid sequence that encodes an antigen or vector disclosed herein, optionally wherein the isolated polynucleotide further comprises a promoter sequence operably linked to the at least one nucleic acid sequence that encodes the antigen or vector. In certain embodiments the isolated polynucleotide can be cDNA.

### IX. Vaccination Protocol

A vaccination protocol can be used to dose a subject with one or more antigens. A priming vaccine and a boosting vaccine can be used to dose the subject. In various embodiments, the priming vaccine can be based on C68 (e.g., the sequences shown in SEQ ID NO:1 or 2) or srRNA (e.g., the sequences shown in SEQ ID NO:3 or 4) and the boosting vaccine can be based on C68 (e.g., the sequences shown in SEQ ID NO:1 or 2) or srRNA (e.g., the sequences shown in SEQ ID NO:3 or 4). In various embodiments, the priming vaccine can be based on alphavirus and the boosting vaccine can be based on alphavirus. In various embodiments, the priming vaccine can be based on C68 and the boosting vaccine can be based on alphavirus.

Each vector typically includes a cassette that includes antigens. Cassettes can include about 20 antigens, separated by spacers such as the natural sequence that normally surrounds each antigen or other non-natural spacer sequences such as AAY. Cassettes can also include MHCII antigens such a tetanus toxoid antigen and PADRE antigen, which can be considered universal class II antigens. Cassettes can also include a targeting sequence such as a ubiquitin targeting sequence. In addition, each vaccine dose can be administered to the subject in conjunction with (e.g., concurrently, before, or after) anti-retrovirals such as nucleoside reverse transcriptase inhibitors (NRTIs), nonnucleoside reverse transcriptase inhibitors (NNRTIs), protease inhibitors (PIs), fusion inhibitors, Entry inhibitors—CCr5 co-receptor antagonist, or HIV integrase strand transfer inhibitors.

A priming vaccine can be injected (e.g., intramuscularly) in a subject. Bilateral injections per dose can be used. For example, one or more injections of ChAdV68 (C68) can be used (e.g., total dose 1×10¹² viral particles); one or more injections of self-replicating RNA (srRNA) at low vaccine dose selected from the range 0.001 to 1 ug RNA, in particular 0.1 or 1 ug can be used; or one or more injections of srRNA at high vaccine dose selected from the range 1 to 100 ug RNA, in particular 10 or 100 ug can be used.

A vaccine boost (boosting vaccine) can be injected (e.g., intramuscularly) after prime vaccination. A boosting vaccine can be administered about every 1, 2, 3, 4, 5, 6, 7, 8, 9, or 10 weeks, e.g., every 4 weeks and/or 8 weeks after the prime. Bilateral injections per dose can be used. For example, one or more injections of ChAdV68 (C68) can be used (e.g., total dose 1×10¹² viral particles); one or more injections of self-replicating RNA (srRNA) at low vaccine dose selected from the range 0.001 to 1 ug RNA, in particular 0.1 or 1 ug can be used; or one or more injections of srRNA at high vaccine dose selected from the range 1 to 100 ug RNA, in particular 10 or 100 ug can be used.

Immune monitoring can be performed before, during, and/or after vaccine administration. Such monitoring can inform safety and efficacy, among other parameters.

To perform immune monitoring, PBMCs are commonly used. PBMCs can be isolated before prime vaccination, and after prime vaccination (e.g. 4 weeks and 8 weeks). PBMCs can be harvested just prior to boost vaccinations and after each boost vaccination (e.g. 4 weeks and 8 weeks).

T cell responses can be assessed as part of an immune monitoring protocol. T cell responses can be measured using one or more methods known in the art such as ELISpot, intracellular cytokine staining, cytokine secretion and cell surface capture, T cell proliferation, MHC multimer staining, or by cytotoxicity assay. T cell responses to epitopes encoded in vaccines can be monitored from PBMCs by measuring induction of cytokines, such as IFN-gamma, using an ELISpot assay. Specific CD4 or CD8 T cell responses to epitopes encoded in vaccines can be monitored from PBMCs by measuring induction of cytokines captured intracellularly or extracellularly, such as IFN-gamma, using flow cytometry. Specific CD4 or CD8 T cell responses to epitopes encoded in the vaccines can be monitored from PBMCs by measuring T cell populations expressing T cell receptors specific for epitope/MHC class I complexes using MHC multimer staining. Specific CD4 or CD8 T cell responses to epitopes encoded in the vaccines can be monitored from PBMCs by measuring the ex vivo expansion of T cell populations following 3H-thymidine, bromodeoxyuridine and carboxyfluoresceine-diacetate-succinimidylester (CFSE) incorporation. The antigen recognition capacity and lytic activity of PBMC-derived T cells that are specific for epitopes encoded in vaccines can be assessed functionally by chromium release assay or alternative colorimetric cytotoxicity assays.

### X. Identifying Candidate Antigens

Candidate antigens can be identified using computational prediction models that predict how likely different antigens are to be presented by HLA alleles. The training and deployment of such computational prediction models, also referred to as presentation models or machine learning models, is discussed in the following sections.

X.A. Presentation Model

Presentation models, also referred to as machine learning models, can be used to identify likelihoods of peptide presentation in patients. Various presentation models are known to those skilled in the art, for example the presentation models described in more detail in international patent application publications WO/2017/106638, WO/2018/195357, WO/2018/208856, WO2016187508, US patent application US20110293637, and PCT/US19/33830, each herein incorporated by reference, in their entirety, for all purposes.

X.B. Training Module

Training modules can be used to construct one or more presentation models based on training data sets that generate likelihoods of whether peptide sequences will be presented by MHC alleles associated with the peptide sequences. Various training modules are known to those skilled in the art, for example the presentation models described in more detail in international patent application publications WO/2017/106638, WO/2018/195357, WO/2018/208856, and PCT/US19/33830, each herein incorporated by reference, in their entirety, for all purposes. A training module can construct a presentation model to predict presentation likelihoods of peptides on a per-allele basis. A training module can also construct a presentation model to predict presentation likelihoods of antigens in a multiple-allele setting where two or more MHC alleles are present.

X.C. Prediction Module

A prediction module can be used to receive sequence data and select candidate epitope sequences in the sequence data using a presentation model. Specifically, the sequence data may be DNA sequences, RNA sequences, and/or protein sequences corresponding to the HIV genome. For example, sequence data may be a HIV epitope sequence (e.g., 8-11 amino acid residues in length) encoded by a gene in the HIV genome.

Generally, a presentation module can apply one or more presentation models to estimate presentation likelihoods of each peptide sequence. The prediction module selects one or more candidate epitope sequences that are likely to be presented on HLA molecules based on the estimated presentation likelihoods. In one embodiment, the presentation module applies presentation models to epitope sequences to estimate presentation likelihoods. In some embodiments, the presentation module applies presentation models to encoded representations of epitope sequences to estimate presentation likelihoods. Such encoded representations may be feature vectors of the peptide sequences. The presentation model outputs estimated presentation likelihoods of antigen presentation in patients.

In one implementation, the presentation module selects candidate epitope sequences that have estimated presentation likelihoods above a predetermined threshold. In another implementation, the presentation model selects the N candidate epitope sequences that have the highest estimated presentation likelihoods (where N is generally the maximum number of epitopes that can be delivered in a vaccine).

In some aspects, the presentation module may further prioritize the candidate epitope sequences by analyzing the structure of antigens that include the candidate epitope sequences. For example, the presentation module may analyze the structure of HIV antigens that include the candidate epitope sequences in order to identify particular amino acid residues or mutations of particular amino acid residues that are highly influential in HIV activity (e.g., viral replication/infection and ability to escape the immune system). Epitope sequences with these identified particular amino acid residues can be ranked more highly. Example analysis, also referred to as structure-based network analysis, is described in further detail in “Structural topology defines protective CD8+ T cell epitopes in the HIV proteome,” which is hereby incorporated by reference in its entirety.¹⁰⁶

### XI. Cassette Design Module

A cassette design module can generate a vaccine cassette sequence based on selected candidate peptides. For example, the cassette design module can select, for inclusion in the vaccine cassette sequence, antigen-encoding nucleic acid sequences that encode for the selected candidate peptides. Various cassette design modules are known to those skilled in the art, for example the cassette design modules described in more detail in international patent application publications WO/2017/106638, WO/2018/195357, and WO/2018/208856, each herein incorporated by reference, in their entirety, for all purposes.

A set of therapeutic epitopes may be generated based on the selected peptides determined by a prediction module associated with presentation likelihoods above a predetermined threshold, where the presentation likelihoods are determined by the presentation models. However it is appreciated that in other embodiments, the set of therapeutic epitopes may be generated based on any one or more of a number of methods (alone or in combination), for example, based on binding affinity or predicted binding affinity to HLA class I or class II alleles of the patient, binding stability or predicted binding stability to HLA class I or class II alleles of the patient, random sampling, and the like.

Therapeutic epitopes may correspond to selected peptides themselves. Therapeutic epitopes may also include C- and/or N-terminal flanking sequences in addition to the selected peptides. N- and C-terminal flanking sequences can be the native N- and C-terminal flanking sequences of the therapeutic vaccine epitope in the context of its source protein. Therapeutic epitopes can represent a fixed-length epitope. Therapeutic epitopes can represent a variable-length epitope, in which the length of the epitope can be varied depending on, for example, the length of the C- or N-flanking sequence. For example, the C-terminal flanking sequence and the N-terminal flanking sequence can each have varying lengths of 2-5 residues, resulting in 16 possible choices for the epitope.

A cassette design module can also generate cassette sequences by taking into account presentation of junction epitopes that span the junction between a pair of therapeutic epitopes in the cassette. Junction epitopes are novel non-self but irrelevant epitope sequences that arise in the cassette due to the process of concatenating therapeutic epitopes and linker sequences in the cassette. The novel sequences of junction epitopes are different from the therapeutic epitopes of the cassette themselves.

A cassette design module can generate a cassette sequence that reduces the likelihood that junction epitopes are presented in the patient. Specifically, when the cassette is injected into the patient, junction epitopes have the potential to be presented by HLA class I or HLA class II alleles of the patient, and stimulate a CD8 or CD4 T-cell response, respectively. Such reactions are often times undesirable because T-cells reactive to the junction epitopes have no therapeutic benefit, and may diminish the immune response to the selected therapeutic epitopes in the cassette by antigenic competition.⁷⁶

A cassette design module can iterate through one or more candidate cassettes, and determine a cassette sequence for which a presentation score of junction epitopes associated with that cassette sequence is below a numerical threshold. The junction epitope presentation score is a quantity associated with presentation likelihoods of the junction epitopes in the cassette, and a higher value of the junction epitope presentation score indicates a higher likelihood that junction epitopes of the cassette will be presented by HLA class I proteins or HLA class II proteins or both.

In one embodiment, a cassette design module may determine a cassette sequence associated with the lowest junction epitope presentation score among the candidate cassette sequences.

A cassette design module may iterate through one or more candidate cassette sequences, determine the junction epitope presentation score for the candidate cassettes, and identify an optimal cassette sequence associated with a junction epitope presentation score below the threshold.

A cassette design module may further check the one or more candidate cassette sequences to identify if any of the junction epitopes in the candidate cassette sequences are self-epitopes for a given patient for whom the vaccine is being designed. To accomplish this, the cassette design module checks the junction epitopes against a known database such as BLAST. In one embodiment, the cassette design module may be configured to design cassettes that avoid junction self-epitopes.

A cassette design module can perform a brute force approach and iterate through all or most possible candidate cassette sequences to select the sequence with the smallest junction epitope presentation score. However, the number of such candidate cassettes can be prohibitively large as the capacity of the vaccine increases. For example, for a vaccine capacity of 20 epitopes, the cassette design module has to iterate through ˜10¹⁸ possible candidate cassettes to determine the cassette with the lowest junction epitope presentation score. This determination may be computationally burdensome (in terms of computational processing resources required), and sometimes intractable, for the cassette design module to complete within a reasonable amount of time to generate the vaccine for the patient. Moreover, accounting for the possible junction epitopes for each candidate cassette can be even more burdensome. Thus, a cassette design module may select a cassette sequence based on ways of iterating through a number of candidate cassette sequences that are significantly smaller than the number of candidate cassette sequences for the brute force approach.

A cassette design module can generate a subset of randomly or at least pseudo-randomly generated candidate cassettes, and selects the candidate cassette associated with a junction epitope presentation score below a predetermined threshold as the cassette sequence. Additionally, the cassette design module may select the candidate cassette from the subset with the lowest junction epitope presentation score as the cassette sequence. For example, the cassette design module may generate a subset of ˜1 million candidate cassettes for a set of 20 selected epitopes, and select the candidate cassette with the smallest junction epitope presentation score. Although generating a subset of random cassette sequences and selecting a cassette sequence with a low junction epitope presentation score out of the subset may be sub-optimal relative to the brute force approach, it requires significantly less computational resources thereby making its implementation technically feasible. Further, performing the brute force method as opposed to this more efficient technique may only result in a minor or even negligible improvement in junction epitope presentation score, thus making it not worthwhile from a resource allocation perspective. A cassette design module can determine an improved cassette configuration by formulating the epitope sequence for the cassette as an asymmetric traveling salesman problem (TSP). Given a list of nodes and distances between each pair of nodes, the TSP determines a sequence of nodes associated with the shortest total distance to visit each node exactly once and return to the original node. For example, given cities A, B, and C with known distances between each other, the solution of the TSP generates a closed sequence of cities, for which the total distance traveled to visit each city exactly once is the smallest among possible routes. The asymmetric version of the TSP determines the optimal sequence of nodes when the distance between a pair of nodes are asymmetric. For example, the “distance” for traveling from node A to node B may be different from the “distance” for traveling from node B to node A. By solving for an improved optimal cassette using an asymmetric TSP, the cassette design module can find a cassette sequence that results in a reduced presentation score across the junctions between epitopes of the cassette. The solution of the asymmetric TSP indicates a sequence of therapeutic epitopes that correspond to the order in which the epitopes should be concatenated in a cassette to minimize the junction epitope presentation score across the junctions of the cassette. A cassette sequence determined through this approach can result in a sequence with significantly less presentation of junction epitopes while potentially requiring significantly less computational resources than the random sampling approach, especially when the number of generated candidate cassette sequences is large. Illustrative examples of different computational approaches and comparisons for optimizing cassette design are described in more detail in international patent application publications WO/2017/106638, WO/2018/195357, and WO/2018/208856, each herein incorporated by reference, in their entirety, for all purposes.

### XII. Example Computer

A computer can be used for any of the computational methods described herein. One skilled in the art will recognize a computer can have different architectures. Examples of computers are known to those skilled in the art, for example the computers described in more detail in international patent application publications WO/2017/106638, WO/2018/195357, and WO/2018/208856, each herein incorporated by reference, in their entirety, for all purposes.

### EXAMPLES

### XIII. Example 1: Antigen Delivery Vector Example

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

XIII.A. Antigen Cassette Design

Through vaccination, multiple class I MHC restricted HIV-specific antigens that induce an immune response can be delivered. In one example, a vaccine cassette was engineered to encode multiple epitope sequences as a single gene product where the epitopes were either embedded within their natural, surrounding peptide sequence or spaced by non-natural linker sequences. Several design parameters were identified that could potentially impact antigen processing and presentation and therefore the magnitude and breadth of the TSNA specific CD8 T cell responses. In the present example, several model cassettes were designed and constructed to evaluate: (1) whether robust T cell responses could be generated to multiple epitopes incorporated in a single expression cassette; (2) what makes an optimal linker placed between the TSNAs within the expression cassette—that leads to optimal processing and presentation of all epitopes; (3) if the relative position of the epitopes within the cassette impact T cell responses; (4) whether the number of epitopes within a cassette influences the magnitude or quality of the T cell responses to individual epitopes; (5) if the addition of cellular targeting sequences improves T cell responses.

Two readouts were developed to evaluate antigen presentation and T cell responses specific for marker epitopes within the model cassettes: (1) an in vitro cell-based screen which allowed assessment of antigen presentation as gauged by the activation of specially engineered reporter T cells (Aarnoudse et al., 2002; Nagai et al., 2012); and (2) an in vivo assay that used HLA-A2 transgenic mice (Vitiello et al., 1991) to assess post-vaccination immunogenicity of cassette-derived epitopes of human origin by their corresponding epitope-specific T cell responses (Comet et al., 2006; Depla et al., 2008; Ishioka et al., 1999).

XIII.B. Antigen Cassette Design Evaluation

XIII.B.1. Methods and Materials

### TCR and Cassette Design and Cloning

The selected TCRs recognize peptides NLVPMVATV (SEQ ID NO: 95) (PDB #5D2N), CLGGLLTMV (SEQ ID NO: 96) (PDB #3REV), GILGFVFTL (SEQ ID NO: 97) (PDB #1OGA) LLFGYPVYV (SEQ ID NO: 98) (PDB #1AO7) when presented by proteins of A*0201 allele. Transfer vectors were constructed that contain 2A peptide-linked TCR subunits (beta followed by alpha), the EMCV IRES, and 2A-linked CD8 subunits (beta followed by alpha and by the puromycin resistance gene). Open reading frame sequences were codon-optimized and synthesized by GeneArt.

### Cell Line Generation for In Vitro Epitope Processing and Presentation Studies

Peptides were purchased from ProImmune or Genscript diluted to 10 mg/mL with 10 mM tris(2-carboxyethyl)phosphine (TCEP) in water/DMSO (2:8, v/v). Cell culture medium and supplements, unless otherwise noted, were from Gibco. Heat inactivated fetal bovine serum (FBShi) was from Seradigm. QUANTI-Luc Substrate, Zeocin, and Puromycin were from InvivoGen. Jurkat-Lucia NFAT Cells (InvivoGen) were maintained in RPMI 1640 supplemented with 10% FBShi, Sodium Pyruvate, and 100 μg/mL Zeocin. Once transduced, these cells additionally received 0.3 μg/mL Puromycin. T2 cells (ATCC CRL-1992) were cultured in Iscove's Medium (IMDM) plus 20% FBShi. U-87 MG (ATCC HTB-14) cells were maintained in MEM Eagles Medium supplemented with 10% FBShi.

Jurkat-Lucia NFAT cells contain an NFAT-inducible Lucia reporter construct. The Lucia gene, when activated by the engagement of the T cell receptor (TCR), causes secretion of a coelenterazine-utilizing luciferase into the culture medium. This luciferase can be measured using the QUANTI-Luc luciferase detection reagent. Jurkat-Lucia cells were transduced with lentivirus to express antigen-specific TCRs. The HIV-derived lentivirus transfer vector was obtained from GeneCopoeia, and lentivirus support plasmids expressing VSV-G (pCMV-VsvG), Rev (pRSV-Rev) and Gag-pol (pCgpV) were obtained from Cell Design Labs.

Lentivirus was prepared by transfection of 50-80% confluent T75 flasks of HEK293 cells with Lipofectamine 2000 (Thermo Fisher), using 40 μl of lipofectamine and 20 μg of the DNA mixture (4:2:1:1 by weight of the transfer plasmid:pCgpV:pRSV-Rev:pCMV-VsvG). 8-10 mL of the virus-containing media were concentrated using the Lenti-X system (Clontech), and the virus resuspended in 100-200 μl of fresh medium. This volume was used to overlay an equal volume of Jurkat-Lucia cells (5×10E4-1×10E6 cells were used in different experiments). Following culture in 0.3 μg/ml puromycin-containing medium, cells were sorted to obtain clonality. These Jurkat-Lucia TCR clones were tested for activity and selectivity using peptide loaded T2 cells.

### In Vitro Epitope Processing and Presentation Assay

T2 cells are routinely used to examine antigen recognition by TCRs. T2 cells lack a peptide transporter for antigen processing (TAP deficient) and cannot load endogenous peptides in the endoplasmic reticulum for presentation on the MHC. However, the T2 cells can easily be loaded with exogenous peptides. The five marker peptides (NLVPMVATV (SEQ ID NO: 99), CLGGLLTMV (SEQ ID NO: 100), GLCTLVAML (SEQ ID NO: 101), LLFGYPVYV (SEQ ID NO: 102), GILGFVFTL (SEQ ID NO: 103)) and two irrelevant peptides (WLSLLVPFV (SEQ ID NO: 104), FLLTRICT (SEQ ID NO: 105)) were loaded onto T2 cells. Briefly, T2 cells were counted and diluted to 1×106 cells/mL with IMDM plus 1% FBShi. Peptides were added to result in 10 μg peptide/1×106 cells. Cells were then incubated at 37° C. for 90 minutes. Cells were washed twice with IMDM plus 20% FBShi, diluted to 5×10E5 cells/mL and 100 μL plated into a 96-well Costar tissue culture plate. Jurkat-Lucia TCR clones were counted and diluted to 5×10E5 cells/mL in RPMI 1640 plus 10% FBShi and 100 μL added to the T2 cells. Plates were incubated overnight at 37° C., 5% CO2. Plates were then centrifuged at 400 g for 3 minutes and 20 μL supernatant removed to a white flat bottom Greiner plate. QUANTI-Luc substrate was prepared according to instructions and 50 μL/well added. Luciferase expression was read on a Molecular Devices SpectraMax iE3x.

To test marker epitope presentation by the adenoviral cassettes, U-87 MG cells were used as surrogate antigen presenting cells (APCs) and were transduced with the adenoviral vectors. U-87 MG cells were harvested and plated in culture media as 5×10E5 cells/100 μl in a 96-well Costar tissue culture plate. Plates were incubated for approximately 2 hours at 37° C. Adenoviral cassettes were diluted with MEM plus 10% FBShi to an MOI of 100, 50, 10, 5, 1 and 0 and added to the U-87 MG cells as 5 μl/well. Plates were again incubated for approximately 2 hours at 37° C. Jurkat-Lucia TCR clones were counted and diluted to 5×10E5 cells/mL in RPMI plus 10% FBShi and added to the U-87 MG cells as 100 μL/well. Plates were then incubated for approximately 24 hours at 37° C., 5% CO2. Plates were centrifuged at 400 g for 3 minutes and 20 μL supernatant removed to a white flat bottom Greiner plate. QUANTI-Luc substrate was prepared according to instructions and 50 μL/well added. Luciferase expression was read on a Molecular Devices SpectraMax iE3x.

### Mouse Strains for Immunogenicity Studies

Transgenic HLA-A2.1 (HLA-A2 Tg) mice were obtained from Taconic Labs, Inc. These mice carry a transgene consisting of a chimeric class I molecule comprised of the human HLA-A2.1 leader, α1, and α2 domains and the murine H2-Kb α3, transmembrane, and cytoplasmic domains (Vitiello et al., 1991). Mice used for these studies were the first generation offspring (F1) of wild type BALB/cAnNTac females and homozygous HLA-A2.1 Tg males on the C57Bl/6 background.

### Adenovirus Vector (Ad5v) Immunizations

HLA-A2 Tg mice were immunized with 1×10¹⁰ to 1×10⁶ viral particles of adenoviral vectors via bilateral intramuscular injection into the tibialis anterior. Immune responses were measured at 12 days post-immunization.

### Lymphocyte Isolation

Lymphocytes were isolated from freshly harvested spleens and lymph nodes of immunized mice. Tissues were dissociated in RPMI containing 10% fetal bovine serum with penicillin and streptomycin (complete RPMI) using the GentleMACS tissue dissociator according to the manufacturer's instructions.

### Ex Vivo Enzyme-Linked Immunospot (ELISPOT) Analysis

ELISPOT analysis was performed according to ELISPOT harmonization guidelines (Janetzki et al., 2015) with the mouse IFNg ELISpotPLUS kit (MABTECH). 1×10⁵ splenocytes were incubated with 10 uM of the indicated peptides for 16 hours in 96-well IFNg antibody coated plates. Spots were developed using alkaline phosphatase. The reaction was timed for 10 minutes and was quenched by running the plate under tap water. Spots were counted using an AID vSpot Reader Spectrum. For ELISPOT analysis, wells with saturation >50% were recorded as “too numerous to count”. Samples with deviation of replicate wells >10% were excluded from analysis. Spot counts were then corrected for well confluency using the formula: spot count+2×(spot count×% confluence/[100%−% confluence]). Negative background was corrected by subtraction of spot counts in the negative peptide stimulation wells from the antigen stimulated wells. Finally, wells labeled too numerous to count were set to the highest observed corrected value, rounded up to the nearest hundred.

### Ex Vivo Intracellular Cytokine Staining (ICS) and Flow Cytometry Analysis

Freshly isolated lymphocytes at a density of 2-5×10⁶ cells/mL were incubated with 10 uM of the indicated peptides for 2 hours. After two hours, brefeldin A was added to a concentration of 5 ug/ml and cells were incubated with stimulant for an additional 4 hours. Following stimulation, viable cells were labeled with fixable viability dye eFluor780 according to manufacturer's protocol and stained with anti-CD8 APC (clone 53-6.7, BioLegend) at 1:400 dilution. Anti-IFNg PE (clone XMG1.2, BioLegend) was used at 1:100 for intracellular staining. Samples were collected on an Attune NxT Flow Cytometer (Thermo Scientific). Flow cytometry data was plotted and analyzed using FlowJo. To assess degree of antigen-specific response, both the percent IFNg+ of CD8+ cells and the total IFNg+ cell number/1×10⁶ live cells were calculated in response to each peptide stimulant.

XIII.B.2. In Vitro Evaluation of Antigen Cassette Designs

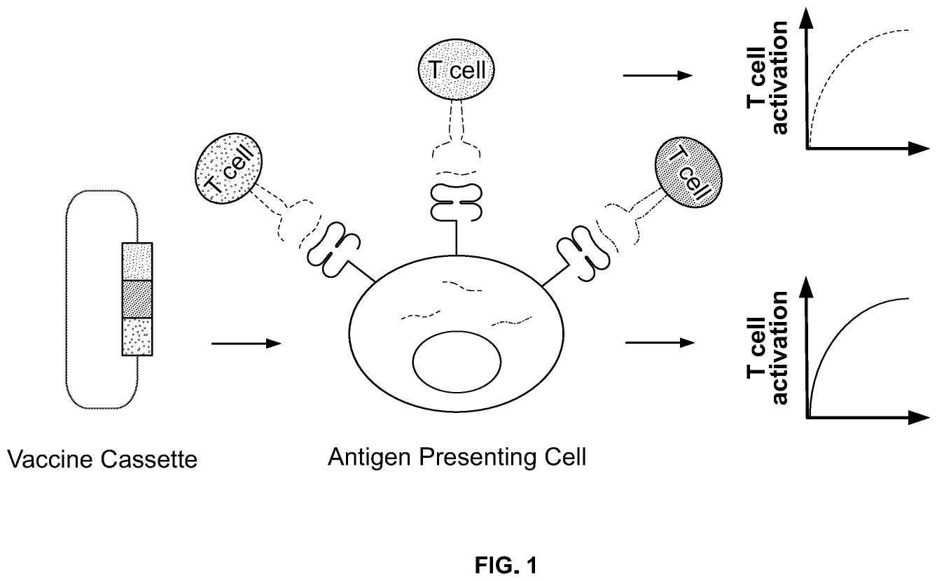

As an example of antigen cassette design evaluation, an in vitro cell-based assay was developed to assess whether selected human epitopes within model vaccine cassettes were being expressed, processed, and presented by antigen-presenting cells (FIG. 1). Upon recognition, Jurkat-Lucia reporter T cells that were engineered to express one of five TCRs specific for well-characterized peptide-HLA combinations become activated and translocate the nuclear factor of activated T cells (NFAT) into the nucleus which leads to transcriptional activation of a luciferase reporter gene. Antigenic stimulation of the individual reporter CD8 T cell lines was quantified by bioluminescence.

Individual Jurkat-Lucia reporter lines were modified by lentiviral transduction with an expression construct that includes an antigen-specific TCR beta and TCR alpha chain separated by a P2A ribosomal skip sequence to ensure equimolar amounts of translated product (Banu et al., 2014). The addition of a second CD8 beta-P2A-CD8 alpha element to the lentiviral construct provided expression of the CD8 co-receptor, which the parent reporter cell line lacks, as CD8 on the cell surface is crucial for the binding affinity to target pMHC molecules and enhances signaling through engagement of its cytoplasmic tail (Lyons et al., 2006; Yachi et al., 2006).

After lentiviral transduction, the Jurkat-Lucia reporters were expanded under puromycin selection, subjected to single cell fluorescence assisted cell sorting (FACS), and the monoclonal populations tested for luciferase expression. This yielded stably transduced reporter cell lines for specific peptide antigens 1, 2, 4, and 5 with functional cell responses. (Table 3A).

TABLE 3A
Development of an in vitro T cell activation assay.
Peptide-specific T cell recognition as measured by
induction of luciferase indicates effective processing
and presentation of the vaccine cassette antigens.

| | Short Cassette Design |
|---|---|
| Epitope | AAY |
| 1 | 24.5 ± 0.5 |
| 2 | 11.3 ± 0.4 |
| 3* | n/a |
| 4 | 26.1 ± 3.1 |
| 5 | 46.3 ± 1.9 |

*Reporter T cell for epitope 3 not yet generated

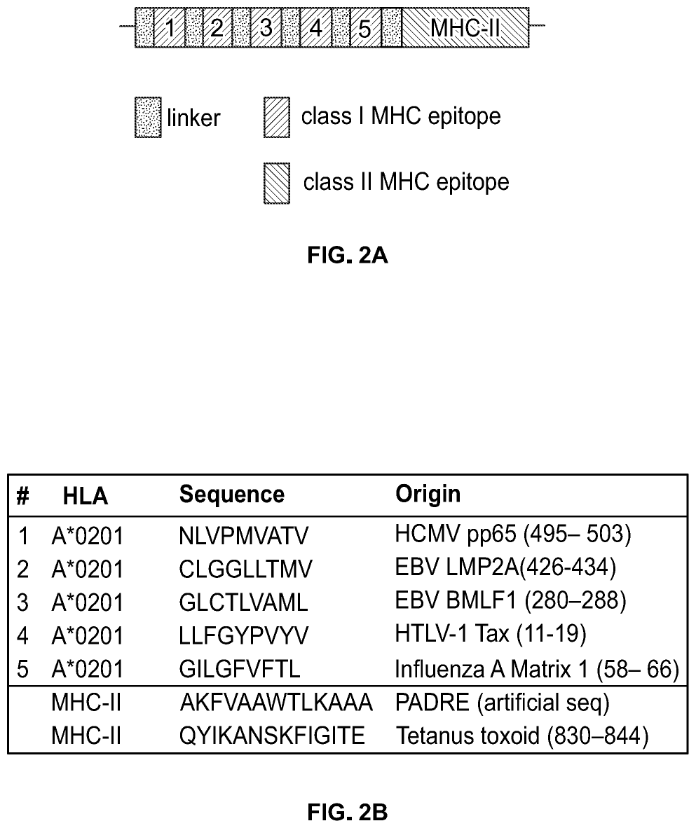

In another example, a series of short cassettes, all marker epitopes were incorporated in the same position (FIG. 2A) and only the linkers separating the HLA-A*0201 restricted epitopes (FIG. 2B) were varied. Reporter T cells were individually mixed with U-87 antigen-presenting cells (APCs) that were infected with adenoviral constructs expressing these short cassettes, and luciferase expression was measured relative to uninfected controls. All four antigens in the model cassettes were recognized by matching reporter T cells, demonstrating efficient processing and presentation of multiple antigens. The magnitude of T cell responses follow largely similar trends for the natural and AAY-linkers. The antigens released from the RR-linker based cassette show lower luciferase inductions (Table 3B). The DPP-linker, designed to disrupt antigen processing, produced a vaccine cassette that led to low epitope presentation (Table 3B).

TABLE 3B
Evaluation of linker sequences in short cassettes. Luciferase induction in the in
vitro T cell activation assay indicated that, apart from the DPP-based cassette, all linkers
facilitated efficient release of the cassette antigens. T cell epitope only (no linker) = 9AA,
natural linker one side = 17AA, natural linker both sides = 25AA, non-natural linkers =
AAY, RR, DPP

| | Short Cassette Designs | | | | | |
|---|---|---|---|---|---|---|
| Epitope | 9AA | 17AA | 25AA | AAY | RR | DPP |
| 1 | 33.6 ± 0.9 | 42.8 ± 2.1 | 42.3 ± 2.3 | 24.5 ± 0.5 | 21.7 ± 0.9 | 0.9 ± 0.1 |
| 2 | 12.0 ± 0.9 | 10.3 ± 0.6 | 14.6 ± 04 | 11.3 ± 0.4 | 8.5 ± 0.3 | 1.1 ± 0.2 |
| 3* | n/a | n/a | n/a | n/a | n/a | n/a |
| 4 | 26.6 ± 2.5 | 16.1 ± 0.6 | 16.6 ± 0.8 | 26.1 ± 3.1 | 12.5 ± 0.8 | 1.3 ± 0.2 |
| 5 | 29.7 ± 0.6 | 21.2 ± 0.7 | 24.3 ± 1.4 | 46.3 ± 1.9 | 19.7 ± 0.4 | 1.3 ± 0.1 |

*Reporter T cell for epitope 3 not yet generated

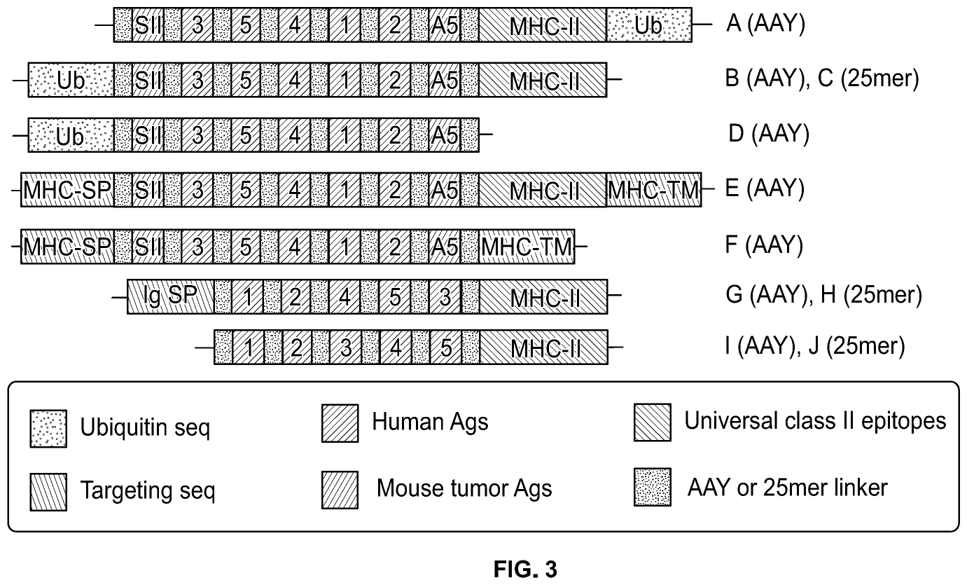

In another example, an additional series of short cassettes were constructed that, besides human and mouse epitopes, contained targeting sequences such as ubiquitin (Ub), MHC and Ig-kappa signal peptides (SP), and/or MHC transmembrane (TM) motifs positioned on either the N- or C-terminus of the cassette. (FIG. 3). When delivered to U-87 APCs by adenoviral vector, the reporter T cells again demonstrated efficient processing and presentation of multiple cassette-derived antigens. However, the magnitude of T cell responses were not substantially impacted by the various targeting features (Table 4).

TABLE 4
Evaluation of cellular targeting sequences added to model vaccine cassettes.
Employing the in vitro T cell activation assay demonstrated that the four HLA-A*0201
restricted marker epitopes are liberated efficiently from the model cassettes and targeting
sequences did not substantially improve T cell recognition and activation.

| | Short Cassette Designs | | | | | | | | | |
|---|---|---|---|---|---|---|---|---|---|---|
| Epitope | A | B | C | D | E | F | G | H | I | J |
| 1 | 32.5 ± | 31.8 ± | 29.1 ± | 29.1 ± | 28.4 ± | 20.4 ± | 35.0 ± | 30.3 ± | 22.5 ± | 38.1 ± |
| | 1.5 | 0.8 | 1.2 | 1.1 | 0.7 | 0.5 | 1.3 | 2.0 | 0.9 | 1.6 |
| 2 | 6.1 ± | 6.3 ± | 7.6 ± | 7.0 ± | 5.9 ± | 3.7 ± | 7.6 ± | 5.4 ± | 6.2 ± | 6.4 ± |
| | 0.2 | 0.2 | 0.4 | 0.5 | 0.2 | 0.2 | 0.4 | 0.3 | 0.4 | 0.3 |
| 3* | n/a | n/a | n/a | n/a | n/a | n/a | n/a | n/a | n/a | n/a |
| 4 | 12.3 ± | 14.1 ± | 12.2 ± | 13.7 ± | 11.7 ± | 10.6 ± | 11.0 ± | 7.6 ± | 16.1 ± | 8.7 ± |
| | 1.1 | 0.7 | 0.8 | 1.0 | 0.8 | 0.4 | 0.6 | 0.6 | 0.5 | 0.5 |
| 5 | 44.4 ± | 53.6 ± | 49.9 ± | 50.5 ± | 41.7 ± | 36.1 ± | 46.5 ± | 31.4 ± | 75.4 ± | 35.7 ± |
| | 2.8 | 1.6 | 3.3 | 2.8 | 2.8 | 1.1 | 2.1 | 0.6 | 1.6 | 2.2 |

*Reporter T cell for epitope 3 not yet generated

XIII.B.3. In Vivo Evaluation of Antigen Cassette Designs

As another example of antigen cassette design evaluation, vaccine cassettes were designed to contain 5 well-characterized human class I MHC epitopes known to stimulate CD8 T cells in an HLA-A*02:01 restricted fashion (FIG. 2A, 3, 5A). For the evaluation of their in vivo immunogenicity, vaccine cassettes containing these marker epitopes were incorporated in adenoviral vectors and used to infect HLA-A2 transgenic mice. This mouse model carries a transgene consisting partly of human HLA-A*0201 and mouse H2-Kb thus encoding a chimeric class I MHC molecule consisting of the human HLA-A2.1 leader, α1 and α2 domains ligated to the murine α3, transmembrane and cytoplasmic H2-Kb domain (Vitiello et al., 1991). The chimeric molecule allows HLA-A*02:01-restricted antigen presentation whilst maintaining the species-matched interaction of the CD8 co-receptor with the α3 domain on the MHC.

For the short cassettes, all marker epitopes generated a T cell response, as determined by IFN-gamma ELISPOT, that was approximately 10-50× stronger of what has been commonly reported (Comet et al., 2006; Depla et al., 2008; Ishioka et al., 1999). Of all the linkers evaluated, the concatamer of 25mer sequences, each containing a minimal epitope flanked by their natural amino acids sequences, generated the largest and broadest T cell response (Table 5). Intracellular cytokine staining (ICS) and flow cytometry analysis revealed that the antigen-specific T cell responses are derived from CD8 T cells.

TABLE 5
In vivo evaluation of linker sequences in short cassettes. ELISPOT data indicated
that HLA-A2 transgenic mice, 17 days post-infection with 1e11 adenovirus viral particles,
generated a T cell response to all class I MHC restricted epitopes in the cassette.

| | Short Cassette Designs | | | | | |
|---|---|---|---|---|---|---|
| Epitope | 9AA | 17AA | 25AA | AAY | RR | DPP |
| 1 | 2020 +/− 583 | 2505 +/− 1281 | 6844 +/− 956 | 1489 +/− 762 | 1675 +/− 690 | 1781 +/− 774 |
| 2 | 4472 +/− 755 | 3792 +/− 1319 | 7629 +/− 996 | 3851 +/− 1748 | 4726 +/− 1715 | 5868 +/− 1427 |
| 3 | 5830 +/− 315 | 3629 +/− 862 | 7253 +/− 491 | 4813 +/− 1761 | 6779 +/− 1033 | 7328 +/− 1700 |
| 4 | 5536 +/− 375 | 2446 +/− 955 | 2961 +/− 1487 | 4230 +/− 1759 | 6518 +/− 909 | 7222 +/− 1824 |
| 5 | 8800 +/− 0 | 7943 +/− 821 | 8423 +/− 442 | 8312 +/− 696 | 8800 +/− 0 | 1836 +/− 328 |

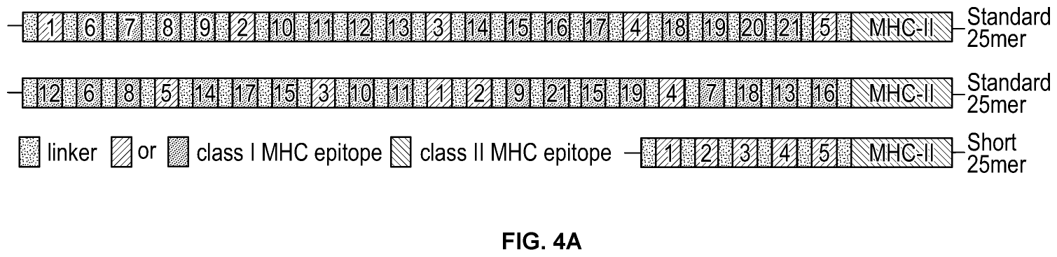

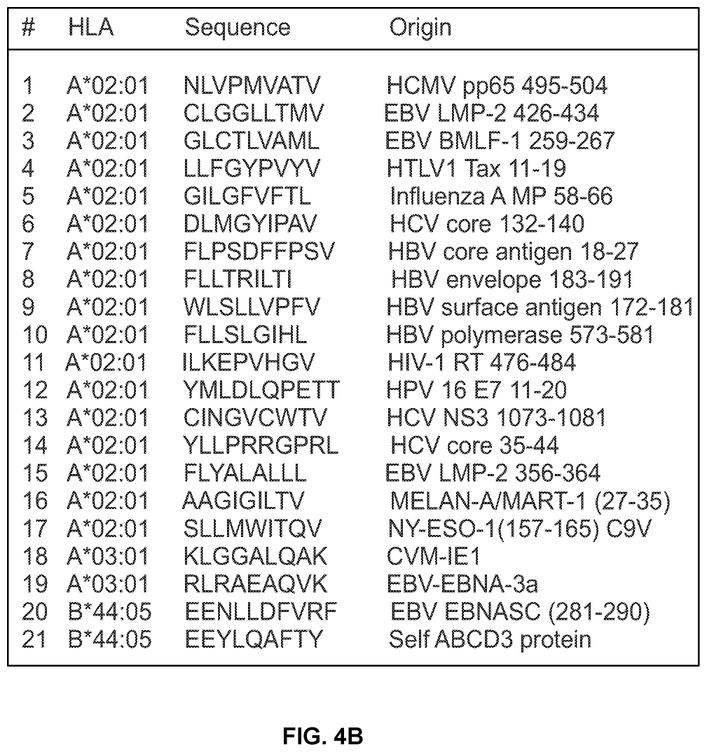

In another example, a series of long vaccine cassettes was constructed and incorporated in adenoviral vectors that, next to the original 5 marker epitopes, contained an additional 16 HLA-A*02:01, A*03:01 and B*44:05 epitopes with known CD8 T cell reactivity (FIG. 4A, B). The size of these long cassettes closely mimicked the final clinical cassette design, and only the position of the epitopes relative to each other was varied. The CD8 T cell responses were comparable in magnitude and breadth for both long and short vaccine cassettes, demonstrating that (a) the addition of more epitopes did not substantially impact the magnitude of immune response to the original set of epitopes, and (b) the position of an epitope in a cassette did not substantially influence the ensuing T cell response to it (Table 6).

TABLE 6
In vivo evaluation of the impact of epitope position in long
cassettes. ELISPOT data indicated that HLA-A2 transgenic
mice, 17 days post-infection with 5e10 adenovirus viral
particles, generated a T cell response comparable in
magnitude for both long and short vaccine cassettes.

| | Long Cassette Designs | | |
|---|---|---|---|
| Epitope | Standard | Scrambled | Short |
| 1 | 863 +/− 1080 | 804 +/− 1113 | 1871 +/− 2859 |
| 2 | 6425 +/− 1594 | 28 +/− 62 | 5390 +/− 1357 |
| 3* | 23 +/− 30 | 36 +/− 18 | 0 +/− 48 |
| 4 | 2224 +/− 1074 | 2727 +/− 644 | 2637 +/− 1673 |
| 5 | 7952 +/− 297 | 8100 +/− 0 | 8100 +/− 0 |

*Suspected technical error caused an absence of a T cell response.

XIII.B.4. Antigen Cassette Design for Immunogenicity and Toxicology Studies

In summary, the findings of the model cassette evaluations (FIG. 2-5, Tables 2-6) demonstrated that, for model vaccine cassettes, robust immunogenicity was achieved when a “string of beads” approach was employed that encodes around 20 epitopes in the context of an adenovirus-based vector. The epitopes were assembled by concatenating 25mer sequences, each embedding a minimal CD8 T cell epitope (e.g. 9 amino acid residues) that were flanked on both sides by its natural, surrounding peptide sequence (e.g. 8 amino acid residues on each side). As used herein, a “natural” or “native” flanking sequence refers to the N- and/or C-terminal flanking sequence of a given epitope in the naturally occurring context of that epitope within its source protein. For example, the HCMV pp65 MHC I epitope NLVPMVATV (SEQ ID NO: 106) is flanked on its 5′ end by the native 5′ sequence WQAGILAR (SEQ ID NO: 107) and on its 3′ end by the native 3′ sequence QGQNLKYQ (SEQ ID NO: 108), thus generating the WQAGILARNLVPMVATVQGQNLKYQ (SEQ ID NO: 109) 25mer peptide found within the HCMV pp65 source protein. The natural or native sequence can also refer to a nucleotide sequence that encodes an epitope flanked by native flanking sequence(s). Each 25mer sequence is directly connected to the following 25mer sequence. In instances where the minimal CD8 T cell epitope is greater than or less than 9 amino acids, the flanking peptide length can be adjusted such that the total length is still a 25mer peptide sequence. For example, a 10 amino acid CD8 T cell epitope can be flanked by an 8 amino acid sequence and a 7 amino acid. The concatamer was followed by two universal class II MHC epitopes that were included to stimulate CD4 T helper cells and improve overall in vivo immunogenicity of the vaccine cassette antigens. (Alexander et al., 1994; Panina-Bordignon et al., 1989) The class II epitopes were linked to the final class I epitope by a GPGPG amino acid linker (SEQ ID NO:56). The two class II epitopes were also linked to each other by a GPGPG (SEQ ID NO: 110) amino acid linker, as a well as flanked on the C-terminus by a GPGPG (SEQ ID NO: 111) amino acid linker. Neither the position nor the number of epitopes appeared to substantially impact T cell recognition or response. Targeting sequences also did not appear to substantially impact the immunogenicity of cassette-derived antigens.

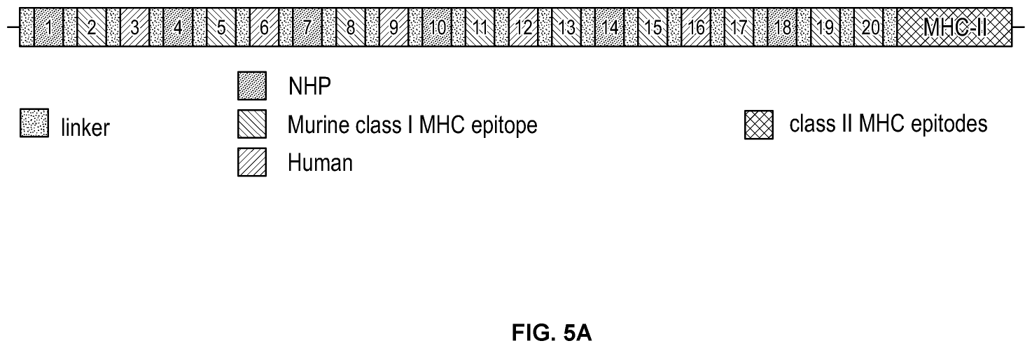

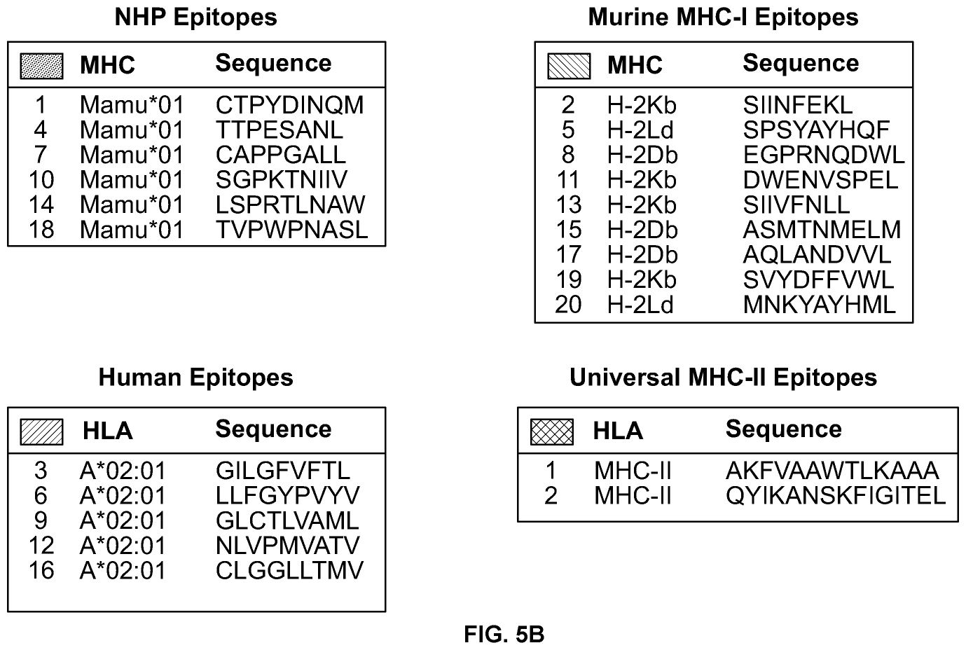

As a further example, based on the in vitro and in vivo data obtained with model cassettes (FIG. 2-5, Tables 2-6), a cassette design was generated that alternates well-characterized T cell epitopes known to be immunogenic in nonhuman primates (NHPs), mice and humans. The 20 epitopes, all embedded in their natural 25mer sequences, are followed by the two universal class II MHC epitopes that were present in all model cassettes evaluated (FIG. 5A, 5B). This cassette design was used to study immunogenicity as well as pharmacology and toxicology studies in multiple species.

XIII.B.5. Antigen Cassette Design and Evaluation for 30, 40, and 50 Antigens

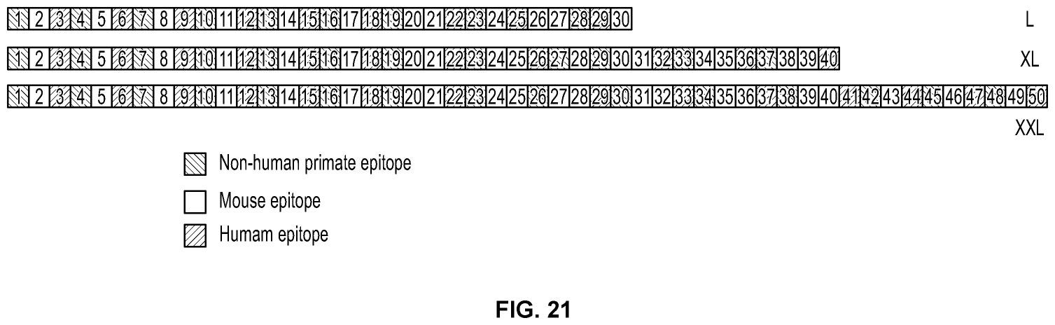

Large antigen cassettes were designed that had either 30 (L), 40 (XL) or 50 (XXL) epitopes, each 25 amino acids in length. The epitopes were a mix of human, NHP and mouse epitopes to model infectious disease antigens. FIG. 21 illustrates the general organization of the epitopes from the various species. The model antigens used are described in Tables 7, 8 and 9 for human, primate, and mouse model epitopes, respectively. Each of Tables 7, 8 and 9 described the epitope position, name, minimal epitope description, and MHC class.

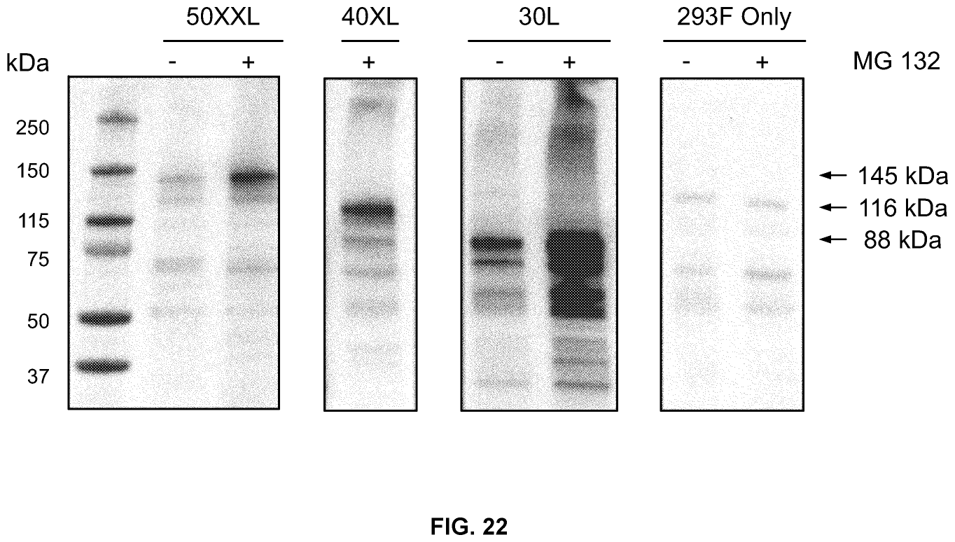

These cassettes were cloned into the chAd68 and alphavirus vaccine vectors as described to evaluate the efficacy of longer multiple-epitope cassettes. FIG. 22 shows that each of the large antigen cassettes were expressed from a ChAdV vector as indicated by at least one major band of the expected size by Western blot.

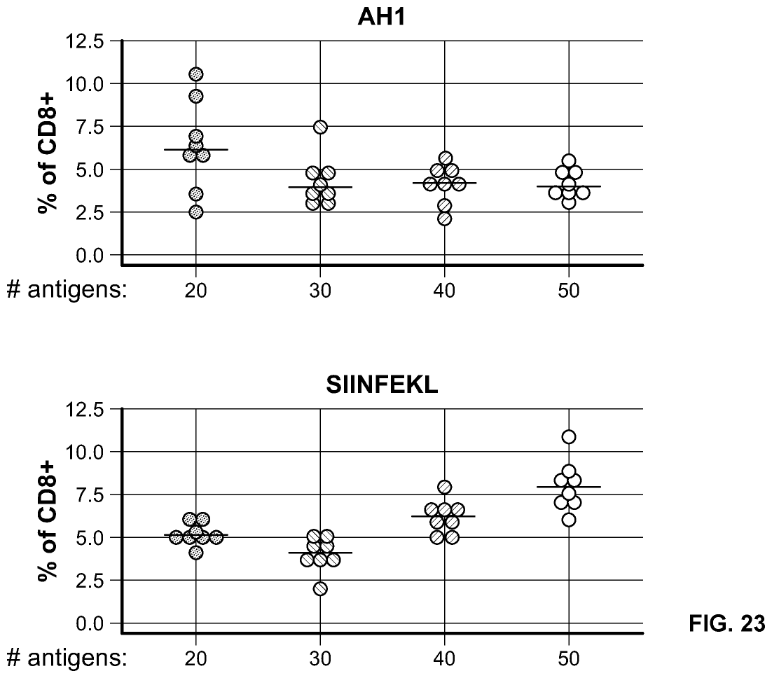

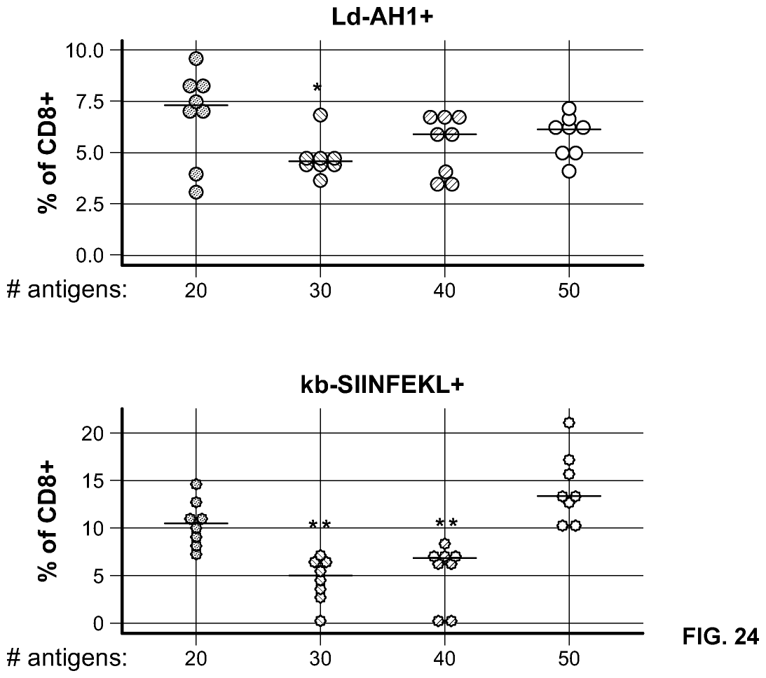

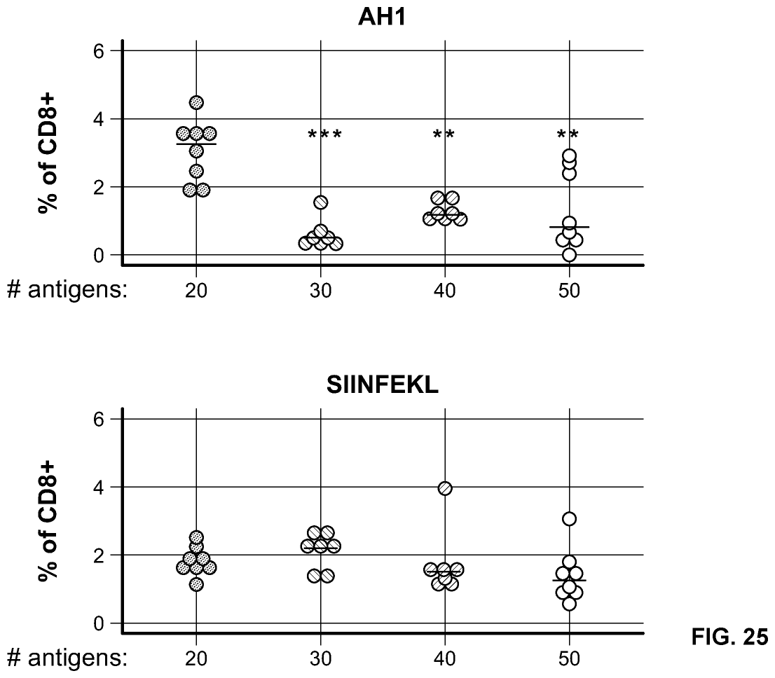

Mice were immunized as described to evaluate the efficacy of the large cassettes. T cell responses were analyzed by ICS and tetramer staining following immunization with a chAd68 vector (FIG. 23/Table 10 and FIG. 24/Table 11, respectively) and by ICS following immunization with a srRNA vector (FIG. 25/Table 12) for epitopes AH1 (top panels) and SINNFEKL (SEQ ID NO: 112) (bottom panels). Immunizations using chAd68 and srRNA vaccine vectors expressing either 30 (L), 40 (XL) or 50 (XXL) epitopes induced CD8+ immune responses to model disease epitopes.

TABLE 7
Human epitopes in large cassettes (SEQ ID NOS 174-203, respectively, in order of columns)

| Epitope | | | | | | | | | |
|---|---|---|---|---|---|---|---|---|---|
| position | | | | | | | | | |
| in each | | | | | | | | | |
| cassette | | | | Minimal | | | Restric- | | |
| L | XL | XXL | Name | epitope | 25mer | MHC | tion | Strain | Species |
| 3 | 3 | 3 | 5.influenza | GILGFVFTL | PILSPLTKGILGFVFTITVPSERGL | Class I | A*02:01 | Human | Human |
| | | | M | | | | | | |
| 6 | 6 | 6 | 4.HTLV-1 | LLFGYPVYV | HFPGFGQSLLFGYPVYVFGDCVQGD | Class I | A*02:01 | Human | Human |
| | | | Tax | | | | | | |
| 9 | 9 | 9 | 3.EBV | GLCTLVAML | RMQAIQNAGLCTLVAMLEETIFWLQ | Class I | A*02:01 | Human | Human |
| | | | BMLF1 | | | | | | |
| 12 | 12 | 12 | 1.HCMV | NLVPMVATV | WQAGILARNLVPMVATVQGQNLKYQ | Class I | A*02:01 | Human | Human |
| | | | pp65 | | | | | | |
| 15 | 15 | 15 | 2.EBV | CLGGLLTMV | RTYGPVFMCLGGLLTMVAGAVWLTV | Class I | A*02:01 | Human | Human |
| | | | LMP2A | | | | | | |
| 18 | 18 | 18 | CT83 | NTDNNLAVY | SSSGLINSNTDNNLAVYDLSRDILN | Class I | A*01:01 | Human | Human |
| | 21 | 21 | MAGEA6 | EVDPIGHVY | LVFGIELMEVDPIGHVYIFATCLGL | Class I | B*35.01 | Human | Human |
| 21 | 25 | 25 | CT83 | LLASSILCA | MNFYLLLASSILCALIVFWKYRRFQ | Class I | A*02:01 | Human | Human |
| 24 | 31 | 28 | FOXE1 | AIFPGAVPAA | AAAAAAAAIFPGAVPAARPPYPGAV | Class I | A*02:01 | Human | Human |
| 27 | 35 | 32 | CT83 | VYDISRDIL | SNTDNNLAVYDLSRDILNNFPHSIA | Class I | A*24:02 | Human | Human |
| | 38 | 36 | MAGE3/6 | ASSLPTTMNY | DPPQSPQGASSLPTTMNYPLWSQSY | Class I | A*01:01 | Human | Human |
| 30 | 40 | 40 | Influenza | PKYVKQNTLK | ITYGACPKYVKQNTLKLATGMRNVP | Class II | DRB1*0101 [symbol] | Human | Human |
| | | | HA | LAT | | | | | |
| | | 44 | CMV pp65 | LPLKMLNIPS | SIYVYALPLKMLNIPSINVHHYPSA | Class II | DRB1*0101 | Human | Human |
| | | | | INVH | | | | | |
| | | 47 | EBV EBNA3A | PEQWMFQGAP | EGPWVPEQWMFQGAPPSQGTDVVQH | Class II | DRB1*0102 | Human | Human |
| | | | | PSQGT | | | | | |
| | | 50 | CMV pp65 | EHPTFTSQYR | RGPQYSEHPTFTSQYRIQGKLEYRH | Class II | DRB1*1101 | Human | Human |
| | | | | IQGKL | | | | | |

[symbol]

indicates data missing or illegible when filed

TABLE 8
NHP epitopes in large cassettes (SEQ ID NOS 204-233, respectively, in order of columns)

| Epitope | | | | | | | | | |
|---|---|---|---|---|---|---|---|---|---|
| position | | | | | | | | | |
| in each | | | | | | | | | |
| cassette | | | | Minimal | | | Restric- | | |
| L | XL | XXL | Name | epitope | 25mer | MHC | tion | Strain | Species |
| 1 | 1 | 1 | Gag CM9 | CTPYDINQM | MFQALSEGCTPYDINQMLNVLGDHQ | Class I | Mamu-A*01 | Rhesus | NHP |
| 4 | 4 | 4 | Tat TL8 | TTPESANL | SCISEADATTPESANLGEEILSQLY | Class I | Mamu-A*01 | Rhesus | NHP |
| 7 | 7 | 7 | Env CL9 | CAPPGYALL | WDAIRFRYCAPPGYALLRCNDTNYS | Class I | Mamu-A*01 | Rhesus | NHP |
| 10 | 10 | 10 | Pol SV9 | SGPKTNIIV | AFLMALTDSGPKTNIIVDSQYVMGI | Class I | Mamu-A*01 | Rhesus | NHP |
| 13 | 13 | 13 | Gag LW9 | LSPRTLNAW | GNVWVHTPLSPRTLNAWVKAVEEKK | Class I | Mamu-A*01 | Rhesus | NHP |
| | | 16 | Env_TL9 | TVPWPNASL | AFRQVCHTTVPWPNASLTPKWNNET | Class I | Mamu-A*01 | Rhesus | NHP |
| 16 | 16 | 19 | Ag85B | PNGTHSWEYWGAQLN | VFNEPPNGTHSWEYWGAQLNAMKGD | Class II | Mamu-DR*W [symbol] | Rhesus | NHP |
| 19 | 19 | 23 | HIV-1 | YKYKVVKIEPLGV | NWRSELYKYKVVKIEPLGVAPTKAK | Class II | Mamu-DR*W [symbol] | Rhesus | NHP |
| | | | Env | | | | | | |
| | | 26 | Gag | TEEAKQIVQRHLVVE | EKVKHTEEAKQIVQRHLVVETGTTE | Class II | Mamu-DRB* [symbol] | Rhesus | NHP |
| | | | TE15 | | | | | | |
| | 23 | 30 | CFP-10 | AGSLQGQWRGAAG | DQVESTAGSLQGQWRGAAGTAAQAA | Class II | Mafa-DRB1* [symbol] | Cyno | NHP |
| | | | 36-48 | | | | | | |
| | 27 | 34 | CFP-10 | EISTNIRQAGVQYSRA | QELDEISTNIRQAGVQYSRADEEQQ | Class II | Mafa-DRB1* [symbol] | Cyno | NHP |
| | | | 71-86 | | | | | | |
| 22 | 29 | 38 | Env | RPKQAWCWF | FHSQPINERPKQAWCWEGGSWKEAI | Class I | Mafa-A1*06 [symbol] | Cyno | NHP |
| | | | 338-346 | | | | | | |
| 25 | 33 | 42 | Nef | RPKVPLRTM | DDIDEEDDDLVGVSVRPKVPLRTMS | Class I | Mafa-A1*06 [symbol] | Cyno | NHP |
| | | | 103-111 | | | | | | |
| 28 | 37 | 45 | Gag | GPRKPIKCW | PFAAAQQRGPRKPIKCWNCGKEGHS | Class I | Mafa-A1*06 [symbol] | Cyno | NHP |
| | | | 386-394 | | | | | | |
| | | 48 | Nef LT9 | LNMADKKET | RRLTARGLLNMADKKETRTPKKAKA | Class I | Mafa-B*104 [symbol] | Cyno | NHP |

[symbol]

indicates data missing or illegible when filed

TABLE 9
Mouse epitopes in large cassettes (SEQ ID NOS 234-273, respectively, in order of columns)

| Epitope | | | | | | | | | |
|---|---|---|---|---|---|---|---|---|---|
| position | | | | | | | | | |
| in each | | | | | | | | | |
| cassette | | | | Minimal | | | Restric- | | |
| L | XL | XXL | Name | epitope | 25mer | MHC | tion | Strain | Species |
| 2 | 2 | 2 | OVA257 | SIINFEKL | VSGLEQLESIINFEKLTEWTSSNVM | Class I | H2-Kb | B6 | Mouse |
| | | 5 | B16-EGP | EGPRNQDWL | ALLAVGALEGPRNQDWLGVPRQLVT | Class I | H2-Db | B6 | Mouse |
| | | 8 | B16-TRP1 | TAPDNLGYM | VTNTEMFVTAPDNIGYMYEVQWPGQ | Class I | H2-Db | B6 | Mouse |
| | | | 455-463 | | | | | | |
| | | 11 | Trp2180-188 | SVYDFFVWL | TQPQIANCSVYDFFVWLHYYSVRDT | Class I | H2-Kb | B6 | Mouse |
| 5 | 5 | 14 | CT26 AH1-A5 | SPSYAYMQF | LWPRVTYHSPSYAYHQFERRAKYKR | Class I | H2-Ld | Balb/C | Mouse |
| | 8 | 17 | CT26 AH1-39 | MNKYAYHML | LWPRVTYHMNKYAYHMLERRAKYKR | Class I | H2-Ld | Balb/C | Mouse |
| | 11 | 20 | MC38 Dpagt1 | SIIVFNLL | GQSLVISASIIVFNLLELEGDYRDD | Class I | H2-Kb | B6 | Mouse |
| | 14 | 22 | MC38 Adpgk | ASMTNMELM | GIPVHLELASMTNMELMSSIVHQQV | Class I | H2-Db | B6 | Mouse |
| | 17 | 24 | MC38 Reps1 | AQLANDVVL | RVLELFRAAQLANDVVLQIMELCGA | Class I | H2-Db | B6 | Mouse |
| 8 | 20 | 27 | P815 P1A 35-44 | LPYLGWLVF | HRYSLEEILPYLGWLVFAVVTTSFL | Class I | H2-Ld | DBA/2 | Mouse |
| 11 | 22 | 29 | P815 P1E | GYCGLRGTGV | YLSKNPDGYCGLRGTGVSCPMAIKK | Class I | H2-Kd | DBA/2 | Mouse |
| 14 | 24 | 31 | Panc02 | LSIFKHKL | NEIPFTYEQLSIFKHKLDKTYPQGY | Class I | H2-Kb | B6 | Mouse |
| | | | Mesotheli [symbol] | | | | | | |
| 17 | 26 | 33 | Panc02 | LIWIPALL | SRASLLGPGFVLIWIPALLPALRLS | Class I | H2-Kb | B6 | Mouse |
| | | | Mesotheli [symbol] | | | | | | |
| 20 | 28 | 35 | ID8 FRa | SSGHNECPV | NWHKGWNWSSGHNECPVGASCHPFT | Class I | H2-Kb | B6 | Mouse |
| | | | 161-169 | | | | | | |
| 23 | 30 | 37 | ID8 Mesothelin | GQKMMAQAI | KTLLKVSKGQKMNAQAIALVACYLR | Class I | H2-Db | B6 | Mouse |
| | | | 40 [symbol] | | | | | | |
| 26 | 32 | 39 | OVA-II | ISQAVHAAH | ESLKISQAVHAAHAEINEAGREVVG | Class II | I-Ab, | B6 | Mouse |
| | | | | AEINEAGR | | | I-Ad | | |
| 29 | 34 | 41 | ESAT-6 | MTEQQWNFAG | MTEQQWNFAGIEAAASAIQGNVTSI | Class II | I-Ab | B6 | Mouse |
| | | | | IEAAASAIQ | | | | | |
| | 36 | 43 | TT p30 | FNNFTVSFWLR | DMFNNFTVSFWLRVPKVSASHLEQY | Class II | I-Ad | Balb/C | Mouse |
| | | | | VPKVSASH [symbol] | | | | | |
| | 39 | 46 | HEL | DGSTDYGILQI | TNRNTDGSTDYGILQINSRWWCNDG | Class II | I-Ak | CBA | Mouse |
| | | | | NSRW | | | | | |
| | | 49 | MOG | MEVGWYRSPFS | TGMEVGWYRSPFSRVVHLYRNGKDQ | Class II | I-Ab | B6 | Mouse |
| | | | | RVVHLYRN | | | | | |

[symbol]

indicates data missing or illegible when filed

TABLE 10
Average IFNg+ cells in response to AH1 and
SIINFEKL (SEQ ID NO: 113) peptides in ChAd
large cassette treated mice. Data is presented
as % of total CD8 cells. Shown is average and
standard deviation per group and p-value by
ANOVA with Tukey's test. All p-values compared
to MAG 20-antigen cassette.

| # | | | Standard | | |
|---|---|---|---|---|---|
| antigens | Antigen | Average | deviation | p-value | N |
| 20 | SIINFEKL | 5.308 | 0.660 | n/a | 8 |
| | (SEQ ID | | | | |
| | NO: 114) | | | | |
| 30 | SIINFEKL | 4.119 | 1.019 | 0.978 | 8 |
| | (SEQ ID | | | | |
| | NO: 115) | | | | |
| 40 | SIINFEKL | 6.324 | 0.954 | 0.986 | 8 |
| | (SEQ ID | | | | |
| | NO: 116) | | | | |
| 50 | SIINFEKL | 8.169 | 1.469 | 0.751 | 8 |
| | (SEQ ID | | | | |
| | NO: 117) | | | | |
| 20 | AH1 | 6.405 | 2.664 | n/a | 8 |
| 30 | AH1 | 4.373 | 1.442 | 0.093 | 8 |
| 40 | AH1 | 4.126 | 1.135 | 0.050 | 8 |
| 50 | AH1 | 4.216 | 0.808 | 0.063 | 8 |

TABLE 11
Average tetramer+ cells for AH1 and SIINFEKL
(SEQ ID NO: 118) antigens in ChAd large
cassette treated mice. Data is presented as
% of total CD8 cells. Shown is average and
standard deviation per group and p-value by
ANOVA with Tukey's test. All p-values
compared to MAG 20-antigen cassette.

| # | | | Standard | | |
|---|---|---|---|---|---|
| antigens | Antigen | Average | deviation | p-value | N |
| 20 | SIINFEKL | 10.314 | 2.384 | n/a | 8 |
| | (SEQ ID | | | | |
| | NO: 119) | | | | |
| 30 | SIINFEKL | 4.551 | 2.370 | 0.003 | 8 |
| | (SEQ ID | | | | |
| | NO: 120) | | | | |
| 40 | SIINFEKL | 5.186 | 3.254 | 0.009 | 8 |
| | (SEQ ID | | | | |
| | NO: 121) | | | | |
| 50 | SIINFEKL | 14.113 | 3.660 | 0.072 | 8 |
| | (SEQ ID | | | | |
| | NO: 122) | | | | |
| 20 | AH1 | 6.864 | 2.207 | n/a | 8 |
| 30 | AH1 | 4.713 | 0.922 | 0.036 | 8 |
| 40 | AH1 | 5.393 | 1.452 | 0.223 | 8 |
| 50 | AH1 | 5.860 | 1.041 | 0.543 | 8 |

TABLE 12
Average IFNg+ cells in response to AH1 and
SIINFEKL (SEQ ID NO: 123) peptides in SAM
large cassette treated mice. Data is presented
as % of total CD8 cells. Shown is average and
standard deviation per group and p-value by
ANOVA with Tukey's test. All p-values compared
to MAG 20-antigen cassette.

| # | | | Standard | | |
|---|---|---|---|---|---|
| antigens | Antigen | Average | deviation | p-value | N |
| 20 | SIINFEKL | 1.843 | 0.422 | n/a | 8 |
| | (SEQ ID | | | | |
| | NO: 124) | | | | |
| 30 | SIINFEKL | 2.112 | 0.522 | 0.879 | 7 |
| | (SEQ ID | | | | |
| | NO: 125) | | | | |
| 40 | SIINFEKL | 1.754 | 0.978 | 0.995 | 7 |
| | (SEQ ID | | | | |
| | NO: 126) | | | | |
| 50 | SIINFEKL | 1.409 | 0.766 | 0.606 | 8 |
| | (SEQ ID | | | | |
| | NO: 127) | | | | |
| 20 | AH1 | 3.050 | 0.909 | n/a | 8 |
| 30 | AH1 | 0.618 | 0.427 | 1.91E−05 | 7 |
| 40 | AH1 | 1.286 | 0.284 | 0.001 | 7 |
| 50 | AH1 | 1.309 | 1.149 | 0.001 | 8 |

### XIV. Example 2: ChAd Antigen Cassette Delivery Vector

XIV.A. ChAd Antigen Cassette Delivery Vector Construction

In one example, Chimpanzee adenovirus (ChAd) was engineered to be a delivery vector for antigen cassettes. In a further example, a full-length ChAdV68 vector was synthesized based on AC_000011.1 (sequence 2 from U.S. Pat. No. 6,083,716) with E1 (nt 457 to 3014) and E3 (nt 27,816-31,332) sequences deleted. Reporter genes under the control of the CMV promoter/enhancer were inserted in place of the deleted E1 sequences. Transfection of this clone into HEK293 cells did not yield infectious virus. To confirm the sequence of the wild-type C68 virus, isolate VR-594 was obtained from the ATCC, passaged, and then independently sequenced (SEQ ID NO:10). When comparing the AC_000011.1 sequence to the ATCC VR-594 sequence (SEQ ID NO:10) of wild-type ChAdV68 virus, 6 nucleotide differences were identified. In one example, a modified ChAdV68 vector was generated based on AC_000011.1, with the corresponding ATCC VR-594 nucleotides substituted at five positions (ChAdV68.5WTnt SEQ ID NO:1).

In another example, a modified ChAdV68 vector was generated based on AC_000011.1 with E1 (nt 577 to 3403) and E3 (nt 27,816-31,332) sequences deleted and the corresponding ATCC VR-594 nucleotides substituted at four positions. A GFP reporter (ChAdV68.4WTnt.GFP; SEQ ID NO:11) or model antigen cassette (ChAdV68.4WTnt.MAG25mer; SEQ ID NO:12) under the control of the CMV promoter/enhancer was inserted in place of deleted E1 sequences.

In another example, a modified ChAdV68 vector was generated based on AC_000011.1 with E1 (nt 577 to 3403) and E3 (nt 27,125-31,825) sequences deleted and the corresponding ATCC VR-594 nucleotides substituted at five positions. A GFP reporter (ChAdV68.5WTnt.GFP; SEQ ID NO:13) or model antigen cassette (ChAdV68.5WTnt.MAG25mer; SEQ ID NO:2) under the control of the CMV promoter/enhancer was inserted in place of deleted E1 sequences

Relevant vectors are described below:
Full-Length ChAdVC68 sequence “ChAdV68.5WTnt” (SEQ ID NO:1); AC_000011.1 sequence with corresponding ATCC VR-594 nucleotides substituted at five positions.
ATCC VR-594 C68 (SEQ ID NO: 10); Independently sequenced; Full-Length C68
ChAdv68.4WTnt.GFP (SEQ ID NO:11 AC_000011.1 with E1 (nt 577 to 3403) and E3 (nt 27,816-31332) sequences deleted; corresponding ATCC VR-594 nucleotides substituted at four positions; GFP reporter under the control of the CMV promoter/enhancer inserted in place of deleted E1
ChAdV68.4WTnt.MAG25mer (SEQ ID NO:12); AC_000011.1 with E1 (nt577 to 3403) and E3 (nt 27,816-31,332) sequences deleted: corresponding ATCC VR-594 nucleotides substituted at four positions; model antigen cassette under the control of the CMV promoter/enhancer inserted in place of deleted E1
ChAdV68.5WTnt.GFP (SEQ ID NO: 13); AC_000011.1 with E1 (nt 577 to 3403) and E3 (nt 27,125-31,825) sequences deleted; corresponding ATCC VR-594 nucleotides substituted at five positions; GFP reporter under the control of the CMV promoter/enhancer inserted in place of deleted E1

XIV.B. ChAd Antigen Cassette Delivery Vector Testing

XIV.B.1. ChAd Vector Evaluation Methods and Materials

### Transfection of HEK293A Cells Using Lipofectamine

DNA for the ChAdV68 constructs (ChAdV68.4WTnt.GFP, ChAdV68.5WTnt.GFP, ChAdV68.4WTnt.MAG25mer and ChAdV68.5WTnt.MAG25mer) was prepared and transfected into HEK293A cells using the following protocol.

10 ug of plasmid DNA was digested with PacI to liberate the viral genome. DNA was then purified using GeneJet DNA cleanup Micro columns (Thermo Fisher) according to manufacturer's instructions for long DNA fragments, and eluted in 20 ul of pre-heated water; columns were left at 37 degrees for 0.5-1 hours before the elution step.

HEK293A cells were introduced into 6-well plates at a cell density of 10⁶ cells/well 14-18 hours prior to transfection. Cells were overlaid with 1 ml of fresh medium (DMEM-10% hiFBS with pen/strep and glutamate) per well. 1-2 ug of purified DNA was used per well in a transfection with twice the ul volume (2-4 ul) of Lipofectamine2000, according to the manufacturer's protocol. 0.5 ml of OPTI-MEM medium containing the transfection mix was added to the 1 ml of normal growth medium in each well, and left on cells overnight.

Transfected cell cultures were incubated at 37° C. for at least 5-7 days. If viral plaques were not visible by day 7 post-transfection, cells were split 1:4 or 1:6, and incubated at 37° C. to monitor for plaque development. Alternatively, transfected cells were harvested and subjected to 3 cycles of freezing and thawing and the cell lysates were used to infect HEK293A cells and the cells were incubated until virus plaques were observed.

Transfection of ChAdV68 Vectors into HEK293A Cells Using Calcium Phosphate and Generation of the Tertiary Viral Stock

DNA for the ChAdV68 constructs (ChAdV68.4WTnt.GFP, ChAdV68.5WTnt.GFP, ChAdV68.4WTnt.MAG25mer, ChAdV68.5WTnt.MAG25mer) was prepared and transfected into HEK293A cells using the following protocol.

HEK293A cells were seeded one day prior to the transfection at 10⁶ cells/well of a 6 well plate in 5% BS/DMEM/1XP/S, 1XGlutamax. Two wells are needed per transfection. Two to four hours prior to transfection the media was changed to fresh media. The ChAdV68.4WTnt.GFP plasmid was linearized with PacI. The linearized DNA was then phenol chloroform extracted and precipitated using one tenth volume of 3M Sodium acetate pH 5.3 and two volumes of 100% ethanol. The precipitated DNA was pelleted by centrifugation at 12,000×g for 5 min before washing 1× with 70% ethanol. The pellet was air dried and re-suspended in 50 μL of sterile water. The DNA concentration was determined using a NanoDrop™ (ThermoFisher) and the volume adjusted to 5 μg of DNA/50 μL.

169 μL of sterile water was added to a microfuge tube. 5 μL of 2M CaCl₂) was then added to the water and mixed gently by pipetting. 50 μL of DNA was added dropwise to the CaCl₂) water solution. Twenty six L of 2M CaCl₂) was then added and mixed gently by pipetting twice with a micro-pipetor. This final solution should consist of 5 μg of DNA in 250 μL of 0.25M CaCl₂). A second tube was then prepared containing 250 μL of 2×HBS (Hepes buffered solution). Using a 2 mL sterile pipette attached to a Pipet-Aid air was slowly bubbled through the 2×HBS solution. At the same time the DNA solution in the 0.25M CaCl₂) solution was added in a dropwise fashion. Bubbling was continued for approximately 5 seconds after addition of the final DNA droplet. The solution was then incubated at room temperature for up to 20 minutes before adding to 293A cells. 250 μL of the DNA/Calcium phosphate solution was added dropwise to a monolayer of 293A cells that had been seeded one day prior at 10⁶ cells per well of a 6 well plate. The cells were returned to the incubator and incubated overnight. The media was changed 24 h later. After 72 h the cells were split 1:6 into a 6 well plate. The monolayers were monitored daily by light microscopy for evidence of cytopathic effect (CPE). 7-10 days post transfection viral plaques were observed and the monolayer harvested by pipetting the media in the wells to lift the cells. The harvested cells and media were transferred to a 50 mL centrifuge tube followed by three rounds of freeze thawing (at −80° C. and 37° C.). The subsequent lysate, called the primary virus stock was clarified by centrifugation at full speed on a bench top centrifuge (4300×g) and a proportion of the lysate 10-50%) used to infect 293A cells in a T25 flask. The infected cells were incubated for 48 h before harvesting cells and media at complete CPE. The cells were once again harvested, freeze thawed and clarified before using this secondary viral stock to infect a T150 flask seeded at 1.5×10⁷ cells per flask. Once complete CPE was achieved at 72 h the media and cells were harvested and treated as with earlier viral stocks to generate a tertiary stock.

### Production in 293F Cells

ChAdV68 virus production was performed in 293F cells grown in 293 FreeStyle™ (ThermoFisher) media in an incubator at 8% C0₂. On the day of infection cells were diluted to 10⁶ cells per mL, with 98% viability and 400 mL were used per production run in 1 L Shake flasks (Corning). 4 mL of the tertiary viral stock with a target MOI of >3.3 was used per infection. The cells were incubated for 48-72 h until the viability was <70% as measured by Trypan blue. The infected cells were then harvested by centrifugation, full speed bench top centrifuge and washed in 1×PBS, re-centrifuged and then re-suspended in 20 mL of 10 mM Tris pH7.4. The cell pellet was lysed by freeze thawing 3× and clarified by centrifugation at 4,300×g for 5 minutes.

### Purification by CsCl Centrifugation

Viral DNA was purified by CsCl centrifugation. Two discontinuous gradient runs were performed. The first to purify virus from cellular components and the second to further refine separation from cellular components and separate defective from infectious particles.

10 mL of 1.2 (26.8 g CsCl dissolved in 92 mL of 10 mM Tris pH 8.0) CsCl was added to polyallomer tubes. Then 8 mL of 1.4 CsCl (53 g CsCl dissolved in 87 mL of 10 mM Tris pH 8.0) was carefully added using a pipette delivering to the bottom of the tube. The clarified virus was carefully layered on top of the 1.2 layer. If needed more 10 mM Tris was added to balance the tubes. The tubes were then placed in a SW-32Ti rotor and centrifuged for 2 h 30 min at 10° C. The tube was then removed to a laminar flow cabinet and the virus band pulled using an 18 gauge needle and a 10 mL syringe. Care was taken not to remove contaminating host cell DNA and protein. The band was then diluted at least 2× with 10 mM Tris pH 8.0 and layered as before on a discontinuous gradient as described above. The run was performed as described before except that this time the run was performed overnight. The next day the band was pulled with care to avoid pulling any of the defective particle band. The virus was then dialyzed using a Slide-a-Lyzer™ Cassette (Pierce) against ARM buffer (20 mM Tris pH 8.0, 25 mM NaCl, 2.5% Glycerol). This was performed 3×, 1 h per buffer exchange. The virus was then aliquoted for storage at −80° C.

### Viral Assays

VP concentration was performed by using an OD 260 assay based on the extinction coefficient of 1.1×10¹² viral particles (VP) is equivalent to an Absorbance value of 1 at OD260 nm. Two dilutions (1:5 and 1:10) of adenovirus were made in a viral lysis buffer (0.1% SDS, 10 mM Tris pH 7.4, 1 mM EDTA). OD was measured in duplicate at both dilutions and the VP concentration/mL was measured by multiplying the OD260 value X dilution factor X 1.1×10¹²VP.

An infectious unit (IU) titer was calculated by a limiting dilution assay of the viral stock. The virus was initially diluted 100× in DMEM/5% NS/1×PS and then subsequently diluted using 10-fold dilutions down to 1×10⁻⁷. 100 μL of these dilutions were then added to 293A cells that were seeded at least an hour before at 3e5 cells/well of a 24 well plate. This was performed in duplicate. Plates were incubated for 48 h in a CO2 (5%) incubator at 37° C. The cells were then washed with 1×PBS and were then fixed with 100% cold methanol (−20° C.). The plates were then incubated at −20° C. for a minimum of 20 minutes. The wells were washed with 1×PBS then blocked in 1×PBS/0.1% BSA for 1 h at room temperature. A rabbit anti-Ad antibody (Abcam, Cambridge, Mass.) was added at 1:8,000 dilution in blocking buffer (0.25 ml per well) and incubated for 1 h at room temperature. The wells were washed 4× with 0.5 mL PBS per well. A HRP conjugated Goat anti-Rabbit antibody (Bethyl Labs, Montgomery Tex.) diluted 1000× was added per well and incubated for 1 h prior to a final round of washing. 5 PBS washes were performed and the plates were developed using DAB (Diaminobenzidine tetrahydrochloride) substrate in Tris buffered saline (0.67 mg/mL DAB in 50 mM Tris pH 7.5, 150 mM NaCl) with 0.01% H₂O₂. Wells were developed for 5 min prior to counting. Cells were counted under a 10× objective using a dilution that gave between 4-40 stained cells per field of view. The field of view that was used was a 0.32 mm² grid of which there are equivalent to 625 per field of view on a 24 well plate. The number of infectious viruses/mL can be determined by the number of stained cells per grid multiplied by the number of grids per field of view multiplied by a dilution factor 10. Similarly, when working with GFP expressing cells florescent can be used rather than capsid staining to determine the number of GFP expressing virions per mL.

### Immunizations

C57BL/6J female mice and Balb/c female mice were injected with 1×10⁸ viral particles (VP) of ChAdV68.5WTnt.MAG25mer in 100 uL volume, bilateral intramuscular injection (50 uL per leg).

### Splenocyte Dissociation

Spleen and lymph nodes for each mouse were pooled in 3 mL of complete RPMI (RPMI, 10% FBS, penicillin/streptomycin). Mechanical dissociation was performed using the gentleMACS Dissociator (Miltenyi Biotec), following manufacturer's protocol. Dissociated cells were filtered through a 40 micron filter and red blood cells were lysed with ACK lysis buffer (150 mM NH₄Cl, 10 mM KHCO₃, 0.1 mM Na₂EDTA). Cells were filtered again through a 30 micron filter and then resuspended in complete RPMI. Cells were counted on the Attune NxT flow cytometer (Thermo Fisher) using propidium iodide staining to exclude dead and apoptotic cells. Cell were then adjusted to the appropriate concentration of live cells for subsequent analysis.

### Ex Vivo Enzyme-Linked Immunospot (ELISPOT) Analysis

ELISPOT analysis was performed according to ELISPOT harmonization guidelines {DOI: 10.1038/nprot.2015.068} with the mouse IFNg ELISpotPLUS kit (MABTECH). 5×10⁴ splenocytes were incubated with 10 uM of the indicated peptides for 16 hours in 96-well IFNg antibody coated plates. Spots were developed using alkaline phosphatase. The reaction was timed for 10 minutes and was terminated by running plate under tap water. Spots were counted using an AID vSpot Reader Spectrum. For ELISPOT analysis, wells with saturation >50% were recorded as “too numerous to count”. Samples with deviation of replicate wells >10% were excluded from analysis. Spot counts were then corrected for well confluency using the formula: spot count+2×(spot count×% confluence/[100%−% confluence]). Negative background was corrected by subtraction of spot counts in the negative peptide stimulation wells from the antigen stimulated wells. Finally, wells labeled too numerous to count were set to the highest observed corrected value, rounded up to the nearest hundred.

XIV.B.2. Production of ChAdV68 Viral Delivery Particles after DNA Transfection

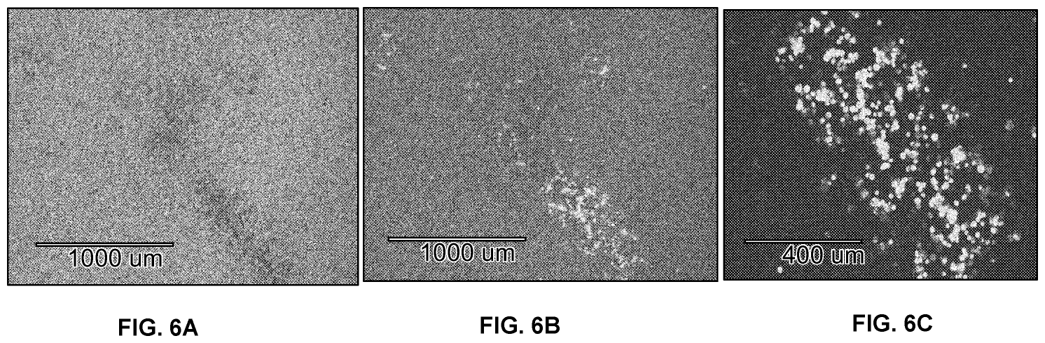

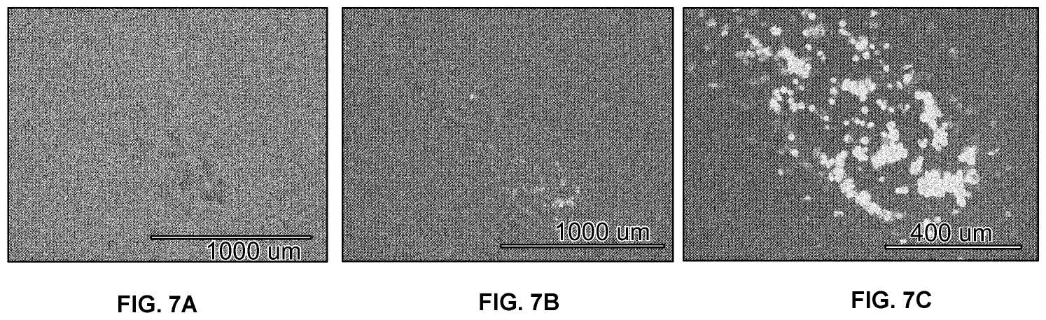

In one example, ChAdV68.4WTnt.GFP (FIG. 6) and ChAdV68.5WTnt.GFP (FIG. 7) DNA was transfected into HEK293A cells and virus replication (viral plaques) was observed 7-10 days after transfection. ChAdV68 viral plaques were visualized using light (FIGS. 6A and 7A) and fluorescent microscopy (FIG. 6B-C and FIG. 7B-C). GFP denotes productive ChAdV68 viral delivery particle production.

XIV.B.3. ChAdV68 Viral Delivery Particles Expansion

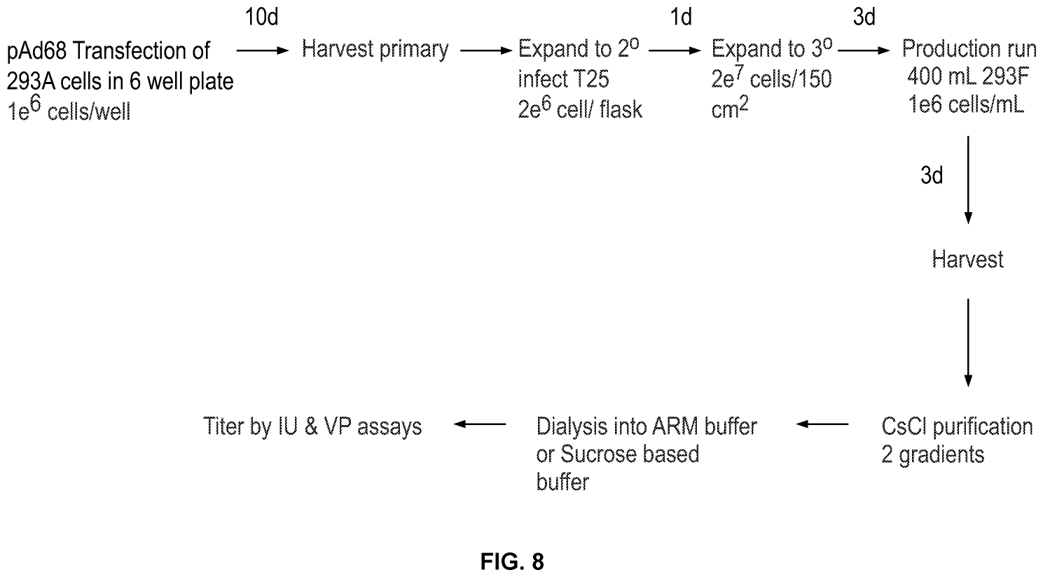

In one example, ChAdV68.4WTnt.GFP, ChAdV68.5WTnt.GFP, and ChAdV68.5WTnt.MAG25mer viruses were expanded in HEK293F cells and a purified virus stock produced 18 days after transfection (FIG. 8). Viral particles were quantified in the purified ChAdV68 virus stocks and compared to adenovirus type 5 (Ad5) and ChAdVY25 (a closely related ChAdV; Dicks, 2012, PloS ONE 7, e40385) viral stocks produced using the same protocol. ChAdV68 viral titers were comparable to Ad5 and ChAdVY25 (Table 13).

TABLE 13
Adenoviral vector production in 293F suspension cells

| Construct | Average VP/cell +/− SD |
|---|---|
| Ad5-Vectors (Multiple vectors) | 2.96e4 +/− 2.26e4 |
| Ad5-GFP | 3.89e4 |
| chAdY25-GFP | 1.75e3 +/− 6.03e1 |
| ChAdV68.4WTnt.GFP | 1.2e4 +/− 6.5e3 |
| ChAdV68.5WTnt.GFP | 1.8e3 |
| ChAdV68.5WTnt.MAG25mer | 1.39e3 +/− 1.1e3 |

*SD is only reported where multiple Production runs have been performed

XIV.B.4. Evaluation of Immunogenicity

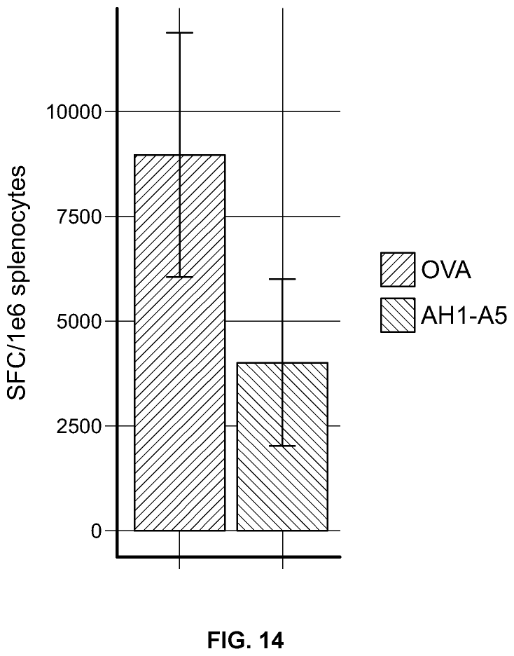

C68 vector expressing mouse tumor antigens were evaluated in mouse immunogenicity studies to demonstrate the C68 vector elicits T-cell responses. T-cell responses to the MHC class I epitope SIINFEKL (SEQ ID NO: 128) were measured in C57BL/6J female mice and the MHC class I epitope AH1-A5 (Slansky et al., 2000, Immunity 13:529-538) measured in Balb/c mice. As shown in FIG. 14, strong T-cell responses relative to control were measured after immunization of mice with ChAdV68.5WTnt.MAG25mer. Mean cellular immune responses of 8957 or 4019 spot forming cells (SFCs) per 10⁶ splenocytes were observed in ELISpot assays when C57BL/6J or Balb/c mice were immunized with ChAdV68.5WTnt.MAG25mer, respectively, 10 days after immunization.

Tumor infiltrating lymphocytes were also evaluated in CT26 tumor model evaluating ChAdV and co-administration of a an anti-CTLA4 antibody. Mice were implanted with CT26 tumors cells and 7 days after implantation, were immunized with ChAdV vaccine and treated with anti-CTLA4 antibody (clone 9D9) or IgG as a control. Tumor infiltrating lymphocytes were analyzed 12 days after immunization. Tumors from each mouse were dissociated using the gentleMACS Dissociator (Miltenyi Biotec) and mouse tumor dissociation kit (Miltenyi Biotec). Dissociated cells were filtered through a 30 micron filter and resuspended in complete RPMI. Cells were counted on the Attune NxT flow cytometer (Thermo Fisher) using propidium iodide staining to exclude dead and apoptotic cells. Cell were then adjusted to the appropriate concentration of live cells for subsequent analysis. Antigen specific cells were identified by MHC-tetramer complexes and co-stained with anti-CD8 and a viability marker. Tumors were harvested 12 days after prime immunization.

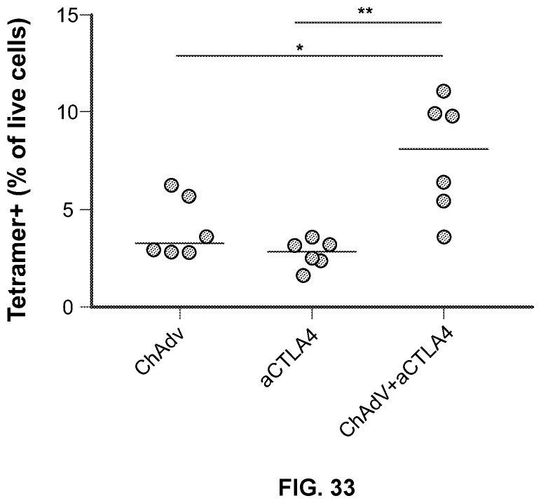

Antigen-specific CD8+ T cells within the tumor comprised a median of 3.3%, 2.2%, or 8.1% of the total live cell population in ChAdV, anti-CTLA4, and ChAdV+ anti-CTLA4 treated groups, respectively (FIG. 33 and Table 14). Treatment with anti-CTLA in combination with active ChAdV immunization resulted in a statistically significant increase in the antigen-specific CD8+ T cell frequency over both ChAdV alone and anti-CTLA4 alone demonstrating anti-CTLA4, when co-administered with the chAd68 vaccine, increased the number of infiltrating T cells within a tumor.

TABLE 14
Tetramer+ infiltrating CD8 T cell frequencies in CT26 tumors

| Treatment | Median % tetramer+ |
|---|---|
| ChAdV68.5WTnt.MAG25mer | 3.3 |
| (ChAdV) | |
| Anti-CTLA4 | 2.2 |
| ChAdV68.5WTnt.MAG25mer | 8.1 |
| (ChAdV) + anti-CTLA4 | |

### XV. Example 3: Alphavirus Antigen Cassette Delivery Vector

XV.A. Alphavirus Delivery Vector Evaluation Materials and Methods

### In Vitro Transcription to Generate RNA

For in vitro testing: plasmid DNA was linearized by restriction digest with PmeI, column purified following manufacturer's protocol (GeneJet DNA cleanup kit, Thermo) and used as template. In vitro transcription was performed using the RiboMAX Large Scale RNA production System (Promega) with the m⁷G cap analog (Promega) according to manufacturer's protocol. mRNA was purified using the RNeasy kit (Qiagen) according to manufacturer's protocol.

For in vivo studies: RNA was generated and purified by TriLInk Biotechnologies and capped with Enzymatic Cap1.

### Transfection of RNA

HEK293A cells were seeded at 6e4 cells/well for 96 wells and 2e5 cells/well for 24 wells, ˜16 hours prior to transfection. Cells were transfected with mRNA using MessengerMAX lipofectamine (Invitrogen) and following manufacturer's protocol. For 96-wells, 0.15 uL of lipofectamine and 10 ng of mRNA was used per well, and for 24-wells, 0.75 uL of lipofectamine and 150 ng of mRNA was used per well. A GFP expressing mRNA (TriLink Biotechnologies) was used as a transfection control.

### Luciferase Assay

Luciferase reporter assay was performed in white-walled 96-well plates with each condition in triplicate using the ONE-Glo luciferase assay (Promega) following manufacturer's protocol. Luminescence was measured using the SpectraMax.

qRT-PCR

Transfected cells were rinsed and replaced with fresh media 2 hours post transfection to remove any untransfected mRNA. Cells were then harvested at various timepoints in RLT plus lysis buffer (Qiagen), homogenized using a QiaShredder (Qiagen) and RNA was extracted using the RNeasy kit (Qiagen), all according to manufacturer's protocol. Total RNA was quantified using a Nanodrop (Thermo Scientific). qRT-PCR was performed using the Quantitect Probe One-Step RT-PCR kit (Qiagen) on the qTower³ (Analytik Jena) according to manufacturer's protocol, using 20 ng of total RNA per reaction. Each sample was run in triplicate for each probe. Actin or GusB were used as reference genes. Custom primer/probes were generated by IDT (Table 15).

TABLE 15
qPCR primers/probes

| Target | | |
|---|---|---|
| Luci | Primer1 | GTGGTGTGCAGCGAGAATAG (SEQ ID NO: |
| | | 129) |
| | Primer2 | CGCTCGTTGTAGATGTCGTTAG (SEQ ID NO: |
| | | 130) |
| | Probe | /56-FAM/TTGCAGTTC/ZEN/TTCATGCCCGTG |
| | | TTG/3IABkFQ/ (SEQ ID NO: 131) |
| GusB | Primer1 | GTTTTTGATCCAGACCCAGATG (SEQ ID NO: |
| | | 132) |
| | Primer2 | GCCCATTATTCAGAGCGAGTA (SEQ ID NO: |
| | | 133) |
| | Probe | /56-FAM/TGCAGGGTT/ZEN/TCACCAGGATCC |
| | | AC/3IABkFQ/ (SEQ ID NO: 134) |
| ActB | Primer1 | CCTTGCACATGCCGGAG (SEQ ID NO: 135) |
| | Primer2 | ACAGAGCCTCGCCTTTG (SEQ ID NO: 136) |
| | Probe | /56-FAM/TCATCCATG/ZEN/GTGAGCTGGCG |
| | | G/3IABkFQ/ (SEQ ID NO: 137) |
| MAG- | Primer1 | CTGAAAGCTCGGTTTGCTAATG (SEQ ID NO: |
| 25mer | | 138) |
| Set1 | Primer2 | CCATGCTGGAAGAGACAATCT (SEQ ID NO: |
| | | 139) |
| | Probe | /56-FAM/CGTTTCTGA/ZEN/TGGCGCTGACCG |
| | | ATA/3IABkFQ/ (SEQ ID NO: 140) |
| MAG- | Primer1 | TATGCCTATCCTGTCTCCTCTG (SEQ ID NO: |
| 25mer | | 141) |
| Set2 | Primer2 | GCTAATGCAGCTAAGTCCTCTC (SEQ ID NO: |
| | | 142) |
| | Probe | /56-FAM/TGTTTACCC/ZEN/TGACCGTGCCTT |
| | | CTG/3IABkFQ/ (SEQ ID NO: 143) |

### B16-OVA Tumor Model

C57BL/6J mice were injected in the lower left abdominal flank with 10⁵ B16-OVA cells/animal. Tumors were allowed to grow for 3 days prior to immunization.

### CT26 Tumor Model

Balb/c mice were injected in the lower left abdominal flank with 10⁶ CT26 cells/animal. Tumors were allowed to grow for 7 days prior to immunization.

### Immunizations

For srRNA vaccine, mice were injected with 10 ug of RNA in 100 uL volume, bilateral intramuscular injection (50 uL per leg). For Ad5 vaccine, mice were injected with 5×10¹⁰ viral particles (VP) in 100 uL volume, bilateral intramuscular injection (50 uL per leg). Animals were injected with anti-CTLA-4 (clone 9D9, BioXcell), anti-PD-1 (clone RMP1-14, BioXcell) or anti-IgG (clone MPC-11, BioXcell), 250 ug dose, 2 times per week, via intraperitoneal injection.

### In Vivo Bioluminescent Imaging

At each timepoint mice were injected with 150 mg/kg luciferin substrate via intraperitoneal injection and bioluminescence was measured using the IVIS In vivo imaging system (PerkinElmer) 10-15 minutes after injection.

### Splenocyte Dissociation

Spleen and lymph nodes for each mouse were pooled in 3 mL of complete RPMI (RPMI, 10% FBS, penicillin/streptomycin). Mechanical dissociation was performed using the gentleMACS Dissociator (Miltenyi Biotec), following manufacturer's protocol. Dissociated cells were filtered through a 40 micron filter and red blood cells were lysed with ACK lysis buffer (150 mM NH₄Cl, 10 mM KHCO₃, 0.1 mM Na₂EDTA). Cells were filtered again through a 30 micron filter and then resuspended in complete RPMI. Cells were counted on the Attune NxT flow cytometer (Thermo Fisher) using propidium iodide staining to exclude dead and apoptotic cells. Cell were then adjusted to the appropriate concentration of live cells for subsequent analysis.

### Ex Vivo Enzyme-Linked Immunospot (ELISPOT) Analysis

ELISPOT analysis was performed according to ELISPOT harmonization guidelines {DOI: 10.1038/nprot.2015.068} with the mouse IFNg ELISpotPLUS kit (MABTECH). 5×10⁴ splenocytes were incubated with 10 uM of the indicated peptides for 16 hours in 96-well IFNg antibody coated plates. Spots were developed using alkaline phosphatase. The reaction was timed for 10 minutes and was terminated by running plate under tap water. Spots were counted using an AID vSpot Reader Spectrum. For ELISPOT analysis, wells with saturation >50% were recorded as “too numerous to count”. Samples with deviation of replicate wells >10% were excluded from analysis. Spot counts were then corrected for well confluency using the formula: spot count+2×(spot count×% confluence/[100%−% confluence]). Negative background was corrected by subtraction of spot counts in the negative peptide stimulation wells from the antigen stimulated wells. Finally, wells labeled too numerous to count were set to the highest observed corrected value, rounded up to the nearest hundred.

XV.B. Alphavirus Vector

XV.B.1. Alphavirus Vector In Vitro Evaluation

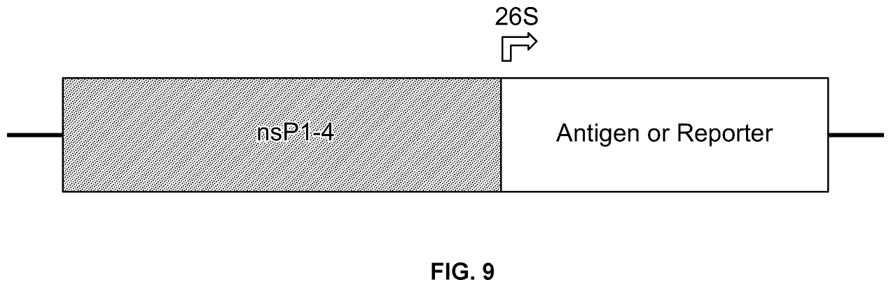

In one implementation of the present invention, a RNA alphavirus backbone for the antigen expression system was generated from a Venezuelan Equine Encephalitis (VEE) (Kinney, 1986, Virology 152: 400-413) based self-replicating RNA (srRNA) vector. In one example, the sequences encoding the structural proteins of VEE located 3′ of the 26S sub-genomic promoter were deleted (VEE sequences 7544 to 11,175 deleted; numbering based on Kinney et al 1986; SEQ ID NO:6) and replaced by antigen sequences (SEQ ID NO: 14 and SEQ ID NO:4) or a luciferase reporter (e.g., VEE-Luciferase, SEQ ID NO:15) (FIG. 9). RNA was transcribed from the srRNA DNA vector in vitro, transfected into HEK293A cells and luciferase reporter expression was measured. In addition, an (non-replicating) mRNA encoding luciferase was transfected for comparison. An ˜30,000-fold increase in srRNA reporter signal was observed for VEE-Luciferase srRNA when comparing the 23 hour measurement vs the 2 hour measurement (Table 16). In contrast, the mRNA reporter exhibited a less than 10-fold increase in signal over the same time period (Table 16).

TABLE 16
Expression of luciferase from VEE self-replicating vector
increases over time. HEK293A cells transfected with 10 ng
of VEE-Luciferase srRNA or 10 ng of non-replicating
luciferase mRNA (TriLink L-6307) per well in 96 wells.
Luminescence was measured at various times post
transfection. Luciferase expression is reported as relative
luminescence units (RLU). Each data point is the
mean +/− SD of 3 transfected wells.

| | Timepoint | | Standard Dev |
|---|---|---|---|
| Construct | (hr) | Mean RLU | (triplicate wells) |
| mRNA | 2 | 878.6666667 | 120.7904522 |
| mRNA | 5 | 1847.333333 | 978.515372 |
| mRNA | 9 | 4847 | 868.3271273 |
| mRNA | 23 | 8639.333333 | 751.6816702 |
| SRRNA | 2 | 27 | 15 |
| SRRNA | 5 | 4884.333333 | 2955.158935 |
| SRRNA | 9 | 182065.5 | 16030.81784 |
| SRRNA | 23 | 783658.3333 | 68985.05538 |

In another example, replication of the srRNA was confirmed directly by measuring RNA levels after transfection of either the luciferase encoding srRNA (VEE-Luciferase) or an srRNA encoding a multi-epitope cassette (VEE-MAG25mer) using quantitative reverse transcription polymerase chain reaction (qRT-PCR). An ˜150-fold increase in RNA was observed for the VEE-luciferase srRNA (Table 17), while a 30-50-fold increase in RNA was observed for the VEE-MAG25mer srRNA (Table 18). These data confirm that the VEE srRNA vectors replicate when transfected into cells.

TABLE 17
Direct measurement of RNA replication in VEE-Luciferase srRNA transfected
cells. HEK293A cells transfected with VEE-Luciferase srRNA (150 ng per well, 24-well) and
RNA levels quantified by qRT-PCR at various times after transfection. Each measurement
was normalized based on the Actin reference gene and fold-change relative to the 2 hour
timepoint is presented.

| Timepoint | | | | | | Relative Fold |
|---|---|---|---|---|---|---|
| (hr) | Luciferase Ct | Actin Ct | dCt | Ref dCt | ddCt | change |
| 2 | 20.51 | 18.14 | 2.38 | 2.38 | 0.00 | 1.00 |
| 4 | 20.09 | 18.39 | 1.70 | 2.38 | −0.67 | 1.59 |
| 6 | 15.50 | 18.19 | −2.69 | 2.38 | −5.07 | 33.51 |
| 8 | 13.51 | 18.36 | −4.85 | 2.38 | −7.22 | 149.43 |

TABLE 18
Direct measurement of RNA replication in VEE-MAG25mer srRNA transfected
cells. HEK293 cells transfected with VEE-MAG25mer srRNA (150 ng per well, 24-well) and
RNA levels quantified by qRT-PCR at various times after transfection. Each measurement
was normalized based on the GusB reference gene and fold-change relative to the 2 hour
timepoint is presented. Different lines on the graph represent 2 different qPCR primer/probe
sets, both of which detect the epitope cassette region of the srRNA.

| Primer/ | Timepoint | | GusB | | | | Relative |
|---|---|---|---|---|---|---|---|
| probe | (hr) | Ct | Ct | dCt | Ref dCt | ddCt | Fold-Change |
| Set1 | 2 | 18.96 | 22.41 | −3.45 | −3.45 | 0.00 | 1.00 |
| Set1 | 4 | 17.46 | 22.27 | −4.81 | −3.45 | −1.37 | 2.58 |
| Set1 | 6 | 14.87 | 22.04 | −7.17 | −3.45 | −3.72 | 13.21 |
| Set1 | 8 | 14.16 | 22.19 | −8.02 | −3.45 | −4.58 | 23.86 |
| Set1 | 24 | 13.16 | 22.01 | −8.86 | −3.45 | −5.41 | 42.52 |
| Set1 | 36 | 13.53 | 22.63 | −9.10 | −3.45 | −5.66 | 50.45 |
| Set2 | 2 | 17.75 | 22.41 | −4.66 | −4.66 | 0.00 | 1.00 |
| Set2 | 4 | 16.66 | 22.27 | −5.61 | −4.66 | −0.94 | 1.92 |
| Set2 | 6 | 14.22 | 22.04 | −7.82 | −4.66 | −3.15 | 8.90 |
| Set2 | 8 | 13.18 | 22.19 | −9.01 | −4.66 | −4.35 | 20.35 |
| Set2 | 24 | 12.22 | 22.01 | −9.80 | −4.66 | −5.13 | 35.10 |
| Set2 | 36 | 13.08 | 22.63 | −9.55 | −4.66 | −4.89 | 29.58 |

XV.B.2. Alphavirus Vector In Vivo Evaluation

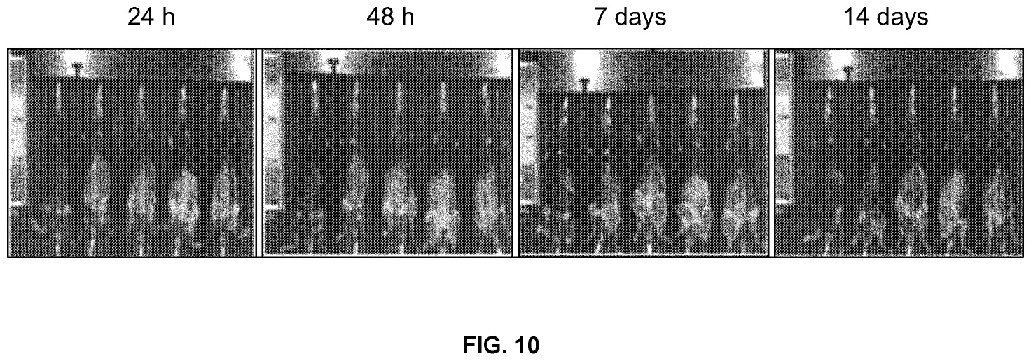

In another example, VEE-Luciferase reporter expression was evaluated in vivo. Mice were injected with 10 ug of VEE-Luciferase srRNA encapsulated in lipid nanoparticle (MC3) and imaged at 24 and 48 hours, and 7 and 14 days post injection to determine bioluminescent signal. Luciferase signal was detected at 24 hours post injection and increased over time and appeared to peak at 7 days after srRNA injection (FIG. 10).

XV.B.3. Alphavirus Vector Tumor Model Evaluation

In one implementation, to determine if the VEE srRNA vector directs antigen-specific immune responses in vivo, a VEE srRNA vector was generated (VEE-UbAAY, SEQ ID NO:14) that expresses 2 different MHC class I mouse tumor epitopes, SIINFEKL (SEQ ID NO: 144) and AH1-A5 (Slansky et al., 2000, Immunity 13:529-538). The SFL (SIINFEKL (SEQ ID NO: 145)) epitope is expressed by the B16-OVA melanoma cell line, and the AH1-A5 (SPSYAYHQF (SEQ ID NO: 146); Slansky et al., 2000, Immunity) epitope induces T cells targeting a related epitope (AH1/SPSYVYHQF (SEQ ID NO: 147); Huang et al., 1996, Proc Natl Acad Sci USA 93:9730-9735) that is expressed by the CT26 colon carcinoma cell line. In one example, for in vivo studies, VEE-UbAAY srRNA was generated by in vitro transcription using T7 polymerase (TriLink Biotechnologies) and encapsulated in a lipid nanoparticle (MC3).

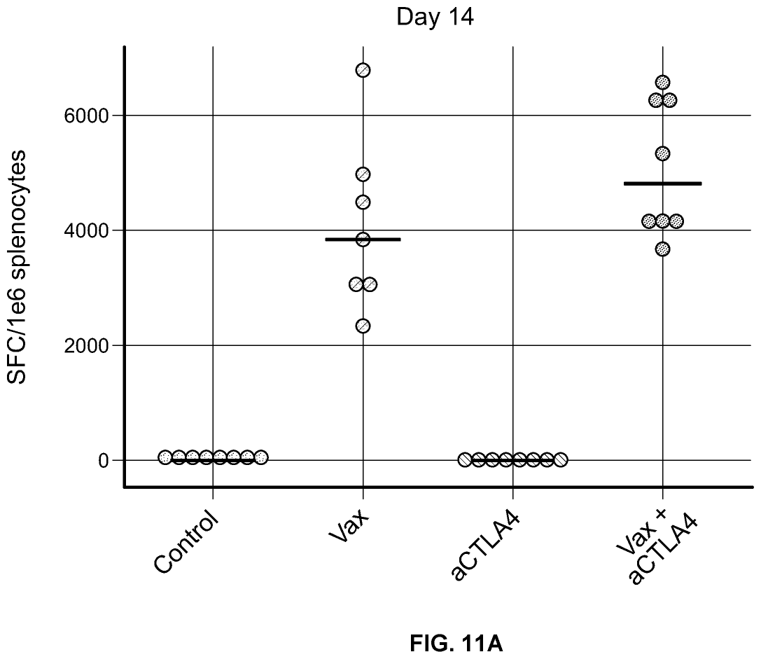

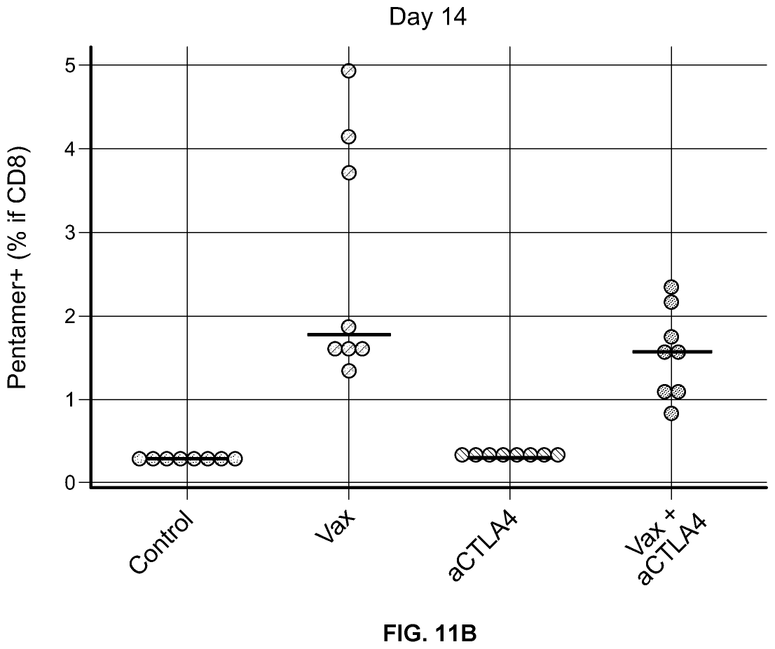

A strong antigen-specific T-cell response targeting SFL, relative to control, was observed two weeks after immunization of B16-OVA tumor bearing mice with MC3 formulated VEE-UbAAY srRNA. In one example, a median of 3835 spot forming cells (SFC) per 10⁶ splenocytes was measured after stimulation with the SFL peptide in ELISpot assays (FIG. 11A, Table 19) and 1.8% (median) of CD8 T-cells were SFL antigen-specific as measured by pentamer staining (FIG. 11B, Table 19). In another example, co-administration of an anti-CTLA-4 monoclonal antibody (mAb) with the VEE srRNA vaccine resulted in a moderate increase in overall T-cell responses with a median of 4794.5 SFCs per 10⁶ splenocytes measured in the ELISpot assay (FIG. 11A, Table 19).

TABLE 19
Results of ELISPOT and MHCI-pentamer staining assays 14 days post VEE
srRNA immunization in B16-OVA tumor bearing C57BL/6J mice.

| | | | Pentamer | | | | Pentamer |
|---|---|---|---|---|---|---|---|
| | | SFC/1e6 | positive (% | | | SFC/1e6 | positive (% |
| Group | Mouse | splenocytes | of CD8) | Group | Mouse | splenocytes | of CD8) |
| Control | 1 | 47 | 0.22 | Vax | 1 | 6774 | 4.92 |
| | 2 | 80 | 0.32 | | 2 | 2323 | 1.34 |
| | 3 | 0 | 0.27 | | 3 | 2997 | 1.52 |
| | 4 | 0 | 0.29 | | 4 | 4492 | 1.86 |
| | 5 | 0 | 0.27 | | 5 | 4970 | 3.7 |
| | 6 | 0 | 0.25 | | 6 | | 4.13 |
| | 7 | 0 | 0.23 | | 7 | 3835 | 1.66 |
| | 8 | 87 | 0.25 | | 8 | 3119 | 1.64 |
| aCTLA4 | 1 | 0 | 0.24 | Vax + | 1 | 6232 | 2.16 |
| | 2 | 0 | 0.26 | aCTLA4 | 2 | 4242 | 0.82 |
| | 3 | 0 | 0.39 | | 3 | 5347 | 1.57 |
| | 4 | 0 | 0.28 | | 4 | 6568 | 2.33 |
| | 5 | 0 | 0.28 | | 5 | 6269 | 1.55 |
| | 6 | 0 | 0.28 | | 6 | 4056 | 1.74 |
| | 7 | 0 | 0.31 | | 7 | 4163 | 1.14 |
| | 8 | 6 | 0.26 | | 8 | 3667 | 1.01 |

* Note
that results from mouse #6 in the Vax group were excluded from analysis due to high variability between triplicate wells.

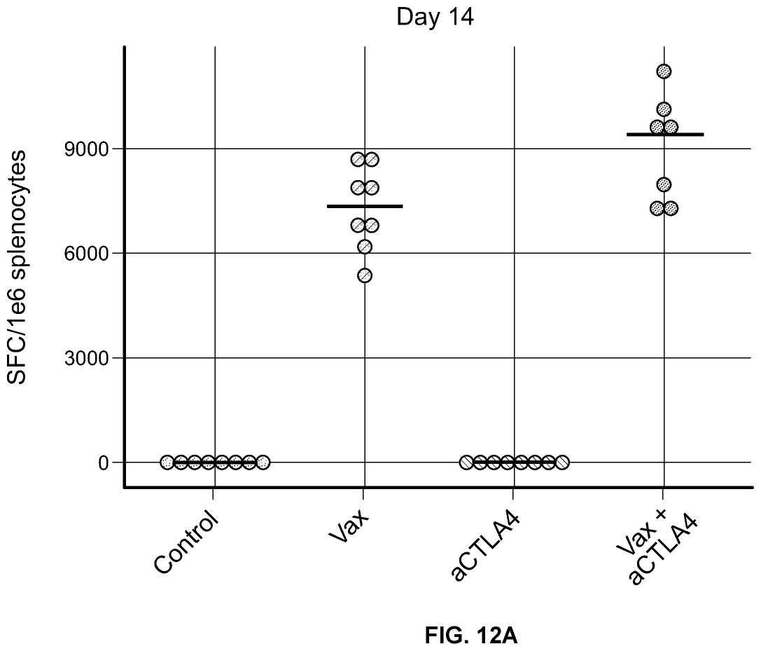

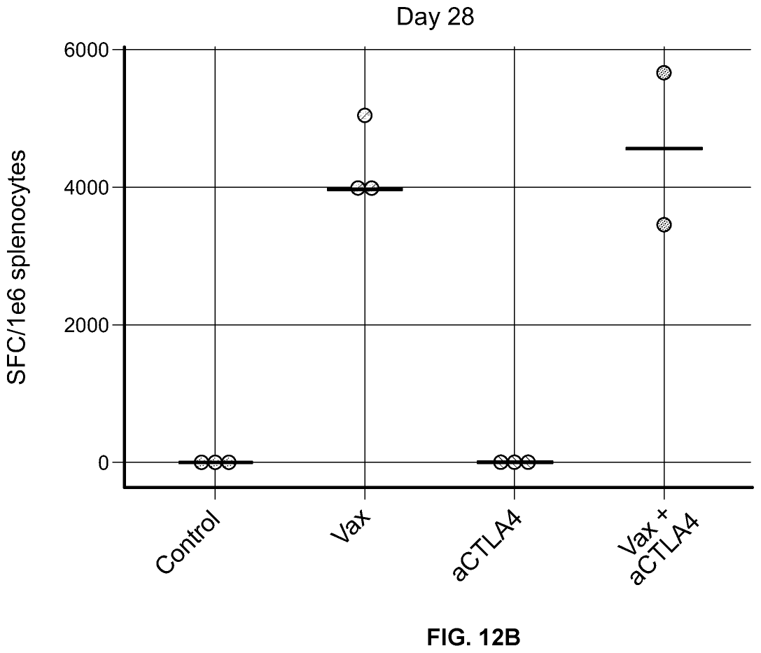

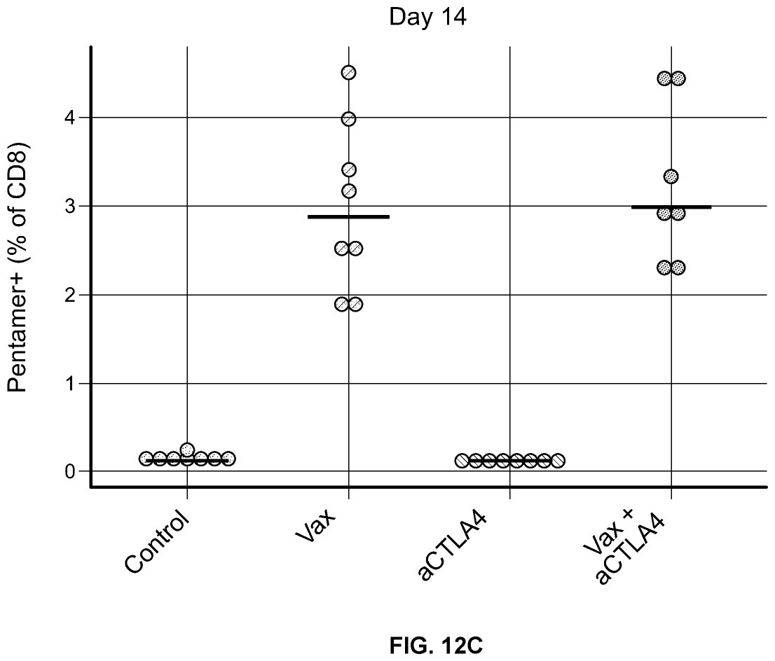

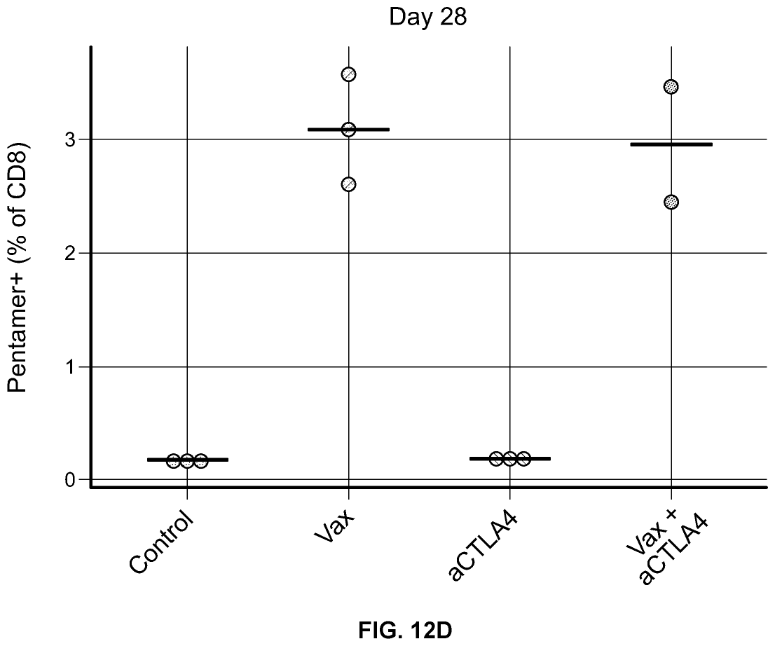

In another implementation, to mirror a clinical approach, a heterologous prime/boost in the B16-OVA and CT26 mouse tumor models was performed, where tumor bearing mice were immunized first with adenoviral vector expressing the same antigen cassette (Ad5-UbAAY), followed by a boost immunization with the VEE-UbAAY srRNA vaccine 14 days after the Ad5-UbAAY prime. In one example, an antigen-specific immune response was induced by the Ad5-UbAAY vaccine resulting in 7330 (median) SFCs per 10⁶ splenocytes measured in the ELISpot assay (FIG. 12A, Table 20) and 2.9% (median) of CD8 T-cells targeting the SFL antigen as measured by pentamer staining (FIG. 12C, Table 20). In another example, the T-cell response was maintained 2 weeks after the VEE-UbAAY srRNA boost in the B16-OVA model with 3960 (median) SFL-specific SFCs per 10⁶ splenocytes measured in the ELISpot assay (FIG. 121B, Table 20) and 3.1% (median) of CD8 T-cells targeting the SFL antigen as measured by pentamer staining (FIG. 12D, Table 20).

TABLE 20
Immune monitoring of B16-OVA mice following heterologous prime/boost with
Ad5 vaccine prime and srRNA boost.

| | | | | | | | |
|---|---|---|---|---|---|---|---|
| | | | Pentamer | | | | Pentamer |
| | | SFC/1e6 | positive | | | SFC/1e6 | positive |
| Group | Mouse | splenocytes | (% of CD8) | Group | Mouse | splenocytes | (% of CD8) |

Day 14

| | | | | | | | |
|---|---|---|---|---|---|---|---|
| Control | 1 | 0 | 0.10 | Vax | 1 | 8514 | 1.87 |
| | 2 | 0 | 0.09 | | 2 | 7779 | 1.91 |
| | 3 | 0 | 0.11 | | 3 | 6177 | 3.17 |
| | 4 | 46 | 0.18 | | 4 | 7945 | 3.41 |
| | 5 | 0 | 0.11 | | 5 | 8821 | 4.51 |
| | 6 | 16 | 0.11 | | 6 | 6881 | 2.48 |
| | 7 | 0 | 0.24 | | 7 | 5365 | 2.57 |
| | 8 | 37 | 0.10 | | 8 | 6705 | 3.98 |
| aCTLA4 | 1 | 0 | 0.08 | Vax + | 1 | 9416 | 2.35 |
| | 2 | 29 | 0.10 | aCTLA4 | 2 | 7918 | 3.33 |
| | 3 | 0 | 0.09 | | 3 | 10153 | 4.50 |
| | 4 | 29 | 0.09 | | 4 | 7212 | 2.98 |
| | 5 | 0 | 0.10 | | 5 | 11203 | 4.38 |
| | 6 | 49 | 0.10 | | 6 | 9784 | 2.27 |
| | 7 | 0 | 0.10 | | 8 | 7267 | 2.87 |
| | 8 | 31 | 0.14 | | | | |

Day 28

| | | | | | | | |
|---|---|---|---|---|---|---|---|
| Control | 2 | 0 | 0.17 | Vax | 1 | 5033 | 2.61 |
| | 4 | 0 | 0.15 | | 2 | 3958 | 3.08 |
| | 6 | 20 | 0.17 | | 4 | 3960 | 3.58 |
| aCTLA4 | 1 | 7 | 0.23 | Vax + | 4 | 3460 | 2.44 |
| | 2 | 0 | 0.18 | aCTLA4 | 5 | 5670 | 3.46 |
| | 3 | 0 | 0.14 | | | | |

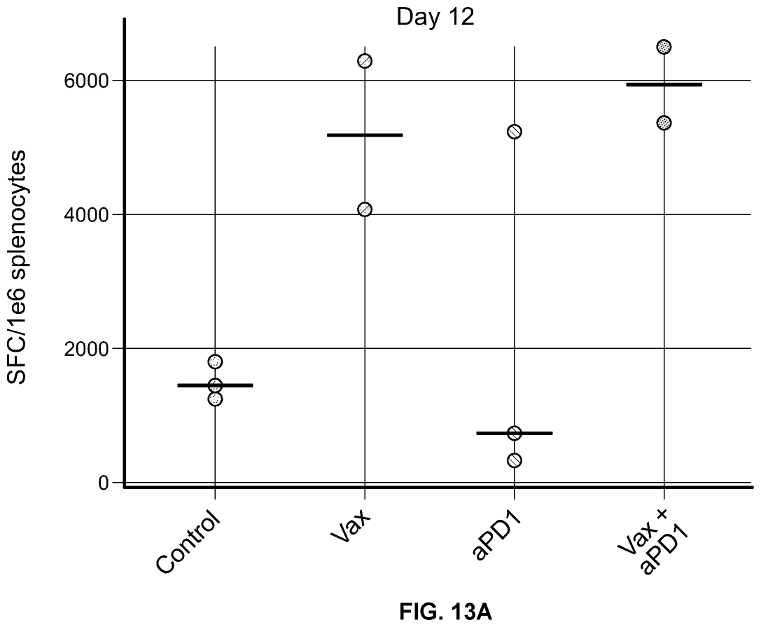

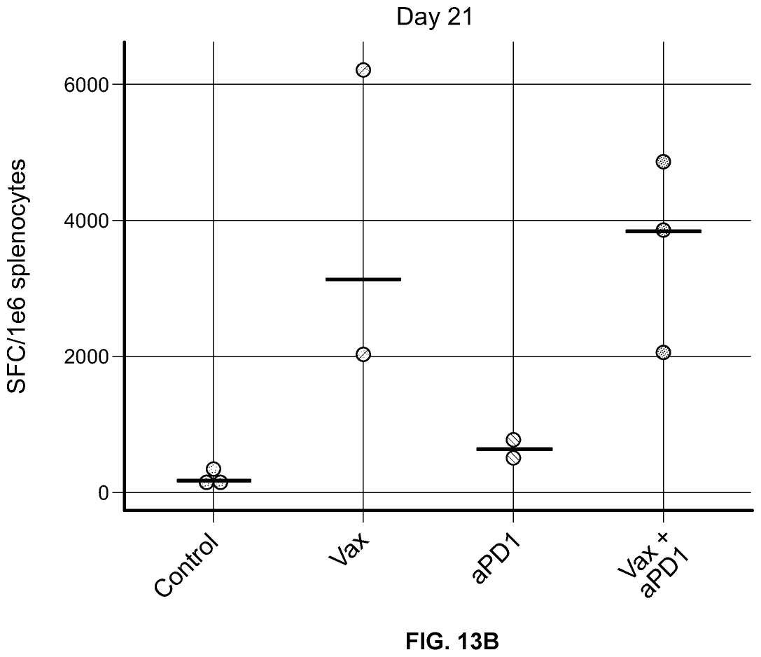

In another implementation, similar results were observed after an Ad5-UbAAY prime and VEE-UbAAY srRNA boost in the CT26 mouse model. In one example, an AH1 antigen-specific response was observed after the Ad5-UbAAY prime (day 14) with a mean of 5187 SFCs per 10⁶ splenocytes measured in the ELISpot assay (FIG. 13A, Table 21) and 3799 SFCs per 10⁶ splenocytes measured in the ELISpot assay after the VEE-UbAAY srRNA boost (day 28) (FIG. 131B, Table 21).

TABLE 21
Immune monitoring after heterologous prime/boost
in CT26 tumor mouse model.
Day 12

| | | SFC/1e6 |
|---|---|---|
| Group | Mouse | splenocytes |
| Control | 1 | 1799 |
| | 2 | 1442 |
| | 3 | 1235 |
| aPD1 | 1 | 737 |
| | 2 | 5230 |
| | 3 | 332 |
| Vax | 1 | 6287 |
| | 2 | 4086 |
| Vax + | 1 | 5363 |
| aPD1 | 2 | 6500 |

Day 21

| | | SFC/1e6 |
|---|---|---|
| Group | Mouse | splenocytes |
| Control | 9 | 167 |
| | 10 | 115 |
| | 11 | 347 |
| aPD1 | 8 | 511 |
| | 11 | 758 |
| Vax | 9 | 3133 |
| | 10 | 2036 |
| | 11 | 6227 |
| Vax + | 8 | 3844 |
| aPD1 | 9 | 2071 |
| | 11 | 4888 |

### XVI. Example 4: Non-Human Primate Studies

Various dosing protocols using ChAdV68 and self-replicating RNA (srRNA) were evaluated in non-human primates (NHP).

Materials and Methods

A priming vaccine was injected intramuscularly (IM) in each NHP to initiate the study (vaccine prime). One or more boosting vaccines (vaccine boost) were also injected intramuscularly in each NHP. Bilateral injections per dose were administered according to groups outlined in tables and summarized below.

### Immunizations

Mamu-A*01 Indian rhesus macaques were immunized bilaterally with 1×10¹² viral particles (5×10¹¹ viral particles per injection) of ChAdV68.5WTnt.MAG25mer, 30 ug of VEE-MAG25MER srRNA, 100 ug of VEE-MAG25mer srRNA or 300 ug of VEE-MAG25mer srRNA formulated in LNP-1 or LNP-2. Vaccine boosts of 30 ug, 100 ug or 300 ug VEE-MAG25mer srRNA were administered intramuscularly at the indicated time after prime vaccination.

### Immune Monitoring

PBMCs were isolated at indicated times after prime vaccination using Lymphocyte Separation Medium (LSM, MP Biomedicals) and LeucoSep separation tubes (Greiner Bio-One) and resuspended in RPMI containing 10% FBS and penicillin/streptomycin. Cells were counted on the Attune NxT flow cytometer (Thermo Fisher) using propidium iodide staining to exclude dead and apoptotic cells. Cell were then adjusted to the appropriate concentration of live cells for subsequent analysis. For each monkey in the studies, T cell responses were measured using ELISpot or flow cytometry methods. T cell responses to 6 different rhesus macaque Mamu-A*01 class I epitopes encoded in the vaccines were monitored from PBMCs by measuring induction of cytokines, such as IFN-gamma, using ex vivo enzyme-linked immunospot (ELISpot) analysis. ELISpot analysis was performed according to ELISPOT harmonization guidelines {DOI: 10.1038/nprot.2015.068} with the monkey IFNg ELISpotPLUS kit (MABTECH). 200,000 PBMCs were incubated with 10 uM of the indicated peptides for 16 hours in 96-well IFNg antibody coated plates. Spots were developed using alkaline phosphatase. The reaction was timed for 10 minutes and was terminated by running plate under tap water. Spots were counted using an AID vSpot Reader Spectrum. For ELISPOT analysis, wells with saturation >50% were recorded as “too numerous to count”. Samples with deviation of replicate wells >10% were excluded from analysis. Spot counts were then corrected for well confluency using the formula: spot count+2×(spot count×% confluence/[100%−% confluence]). Negative background was corrected by subtraction of spot counts in the negative peptide stimulation wells from the antigen stimulated wells. Finally, wells labeled too numerous to count were set to the highest observed corrected value, rounded up to the nearest hundred.

Specific CD4 and CD8 T cell responses to 6 different rhesus macaque Mamu-A*01 class I epitopes encoded in the vaccines were monitored from PBMCs by measuring induction of intracellular cytokines, such as IFN-gamma, using flow cytometry. The results from both methods indicate that cytokines were induced in an antigen-specific manner to epitopes.

Immunogenicity in Rhesus Macaques

This study was designed to (a) evaluate the immunogenicity and preliminary safety of VEE-MAG25mer srRNA 30 μg and 100 μg doses as a homologous prime/boost or heterologous prime/boost in combination with ChAdV68.5WTnt.MAG25mer; (b) compare the immune responses of VEE-MAG25mer srRNA in lipid nanoparticles using LNP1 versus LNP2; (c) evaluate the kinetics of T-cell responses to VEE-MAG25mer srRNA and ChAdV68.5WTnt.MAG25mer immunizations.

The study arm was conducted in Mamu-A*01 Indian rhesus macaques to demonstrate immunogenicity. Select antigens used in this study are only recognized in Rhesus macaques, specifically those with a Mamu-A*01 MHC class I haplotype. Mamu-A*01 Indian rhesus macaques were randomized to the different study arms (6 macaques per group) and administered an IM injection bilaterally with either ChAdV68.5WTnt.MAG25mer or VEE-MAG25mer srRNA vector encoding model antigens that includes multiple Mamu-A*01 restricted epitopes. The study arms were as described below.

TABLE 22
Non-GLP immunogenicity study in Indian Rhesus Macaques

| Group | Prime | Boost 1 | Boost 2 |
|---|---|---|---|
| 1 | VEE-MAG25mer | VEE-MAG25mer | VEE-MAG25mer |
| | srRNA-LNP1 | srRNA-LNP1 | srRNA-LNP1 |
| | (30 μg) | (30 μg) | (30 μg) |
| 2 | VEE-MAG25mer | VEE-MAG25mer | VEE-MAG25mer |
| | srRNA-LNP1 | srRNA-LNP1 | srRNA-LNP1 |
| | (100 μg) | (100 μg) | (100 μg) |
| 3 | VEE-MAG25mer | VEE-MAG25mer | VEE-MAG25mer |
| | srRNA-LNP2 | srRNA-LNP2 | srRNA-LNP2 |
| | (100 μg) | (100 μg) | (100 μg) |
| 4 | ChAdV68.5WTnt. | VEE-MAG25mer | VEE-MAG25mer |
| | MAG25mer | srRNA-LNP1 | srRNA-LNP1 |
| | | (100 μg) | (100 μg) |

PBMCs were collected prior to immunization and on weeks 1, 2, 3, 4, 5, 6, 8, 9, and 10 after the initial immunization for immune monitoring.

Results

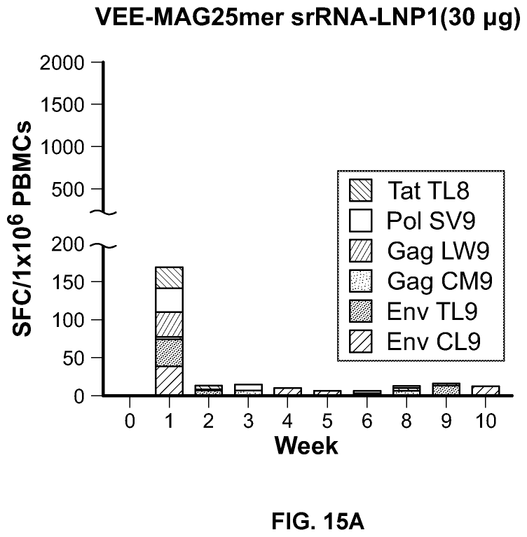

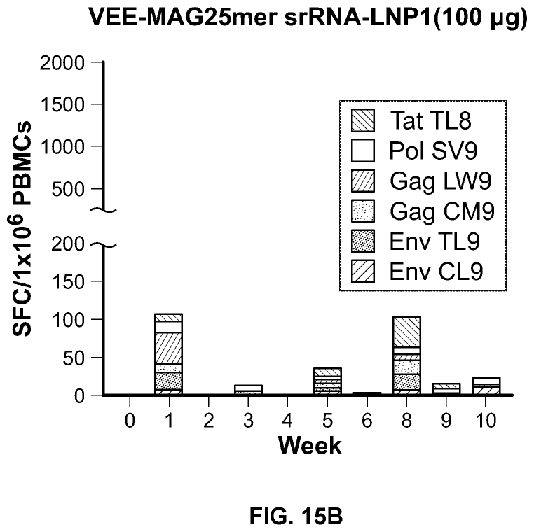

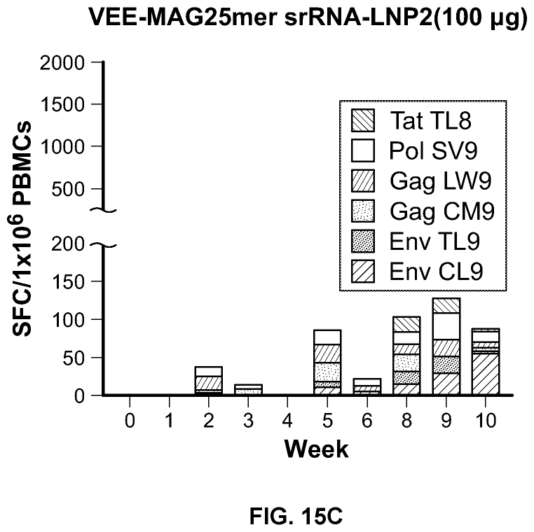

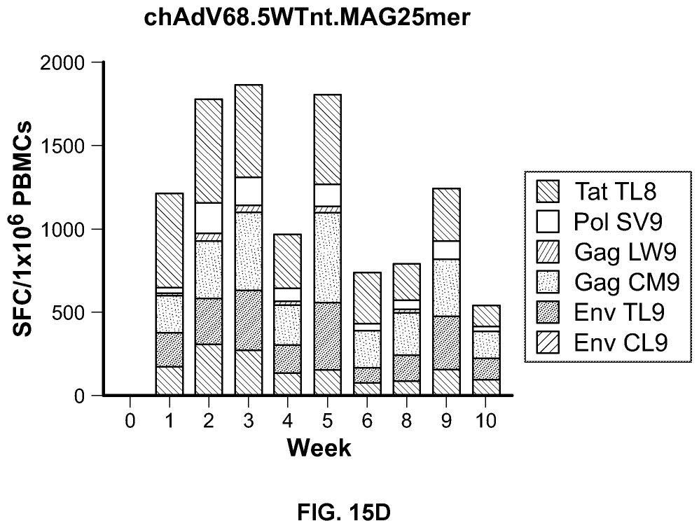

Antigen-specific cellular immune responses in peripheral blood mononuclear cells (PBMCs) were measured to six different Mamu-A*01 restricted epitopes prior to immunization and 1, 2, 3, 4, 5, 6, 8, 9, and 10 weeks after the initial immunization. Animals received a boost immunization with VEE-MAG25mer srRNA on weeks 4 and 8 with either 30 μg or 100 μg doses, and either formulated with LNP1 or LNP2, as described in Table 22. Combined immune responses to all six epitopes were plotted for each immune monitoring timepoint (FIG. 15A-D and Tables 23-26).

Combined antigen-specific immune responses were observed at all measurements with 170, 14, 15, 11, 7, 8, 14, 17, 12 SFCs per 10⁶ PBMCs (six epitopes combined) 1, 2, 3, 4, 5, 6, 8, 9, or 10 weeks after an initial VEE-MAG25mer srRNA-LNP1 (30 μg) prime immunization, respectively (FIG. 15A). Combined antigen-specific immune responses were observed at all measurements with 108, −3, 14, 1, 37, 4, 105, 17, 25 SFCs per 10⁶ PBMCs (six epitopes combined) 1, 2, 3, 4, 5, 6, 8, 9, or 10 weeks after an initial VEE-MAG25mer srRNA-LNP1 (100 μg) prime immunization, respectively (FIG. 15B). Combined antigen-specific immune responses were observed at all measurements with −17, 38, 14, −2, 87, 21, 104, 129, 89 SFCs per 10⁶ PBMCs (six epitopes combined) 1, 2, 3, 4, 5, 6, 8, 9, or 10 weeks after an initial VEE-MAG25mer srRNA-LNP2 (100 μg) prime immunization, respectively (FIG. 15C). Negative values are a result of normalization to pre-bleed values for each epitope/animal.

Combined antigen-specific immune responses were observed at all measurements with 1218, 1784, 1866, 973, 1813, 747, 797, 1249, and 547 SFCs per 10⁶ PBMCs (six epitopes combined) 1, 2, 3, 4, 5, 6, 8, 9, or 10 weeks after an initial ChAdV68.5WTnt.MAG25mer prime immunization, respectively (FIG. 15D). The immune response showed the expected profile with peak immune responses measured ˜2-3 weeks after the prime immunization followed by a contraction in the immune response after 4 weeks. Combined antigen-specific cellular immune responses of 1813 SFCs per 10⁶ PBMCs (six epitopes combined) were measured 5 weeks after the initial immunization with ChAdV68.5WTnt.MAG25mer (i.e., 1 week after the first boost with VEE-MAG25mer srRNA). The immune response measured 1 week after the first boost with VEE-MAG25mer srRNA (week 5) was comparable to the peak immune response measured for the ChAdV68.5WTnt.MAG25mer prime immunization (week 3) (FIG. 15D). Combined antigen-specific cellular immune responses of 1249 SFCs per 10⁶ PBMCs (six epitopes combined) was measured 9 weeks after the initial immunization with ChAdV68.5WTnt.MAG25mer, respectively (i.e., 1 week after the second boost with VEE-MAG25mer srRNA). The immune responses measured 1 week after the second boost with VEE-MAG25mer srRNA (week 9) was ˜2-fold higher than that measured just before the boost immunization (FIG. 15D).

TABLE 23
Mean spot forming cells (SFC) per 10⁶ PBMCs for each epitope ± SEM
for VEE-MAG25mer srRNA-LNP1 (30 μg) (Group 1)

| | Antigen | | | | | |
|---|---|---|---|---|---|---|
| Wk | Env CL9 | Env TL9 | Gag CM9 | Gag LW9 | Pol SV9 | Tat TL8 |
| 1 | 0 ± 0 | 0 ± 0 | 0 ± 0 | 0 ± 0 | 0 ± 0 | 0 ± 0 |
| 2 | 39.7 ± 22.7 | 35.4 ± 25.1 | 3.2 ± 3.6 | 33 ± 28.1 | 30.9 ± 20.3 | 28.3 ± 17.5 |
| 3 | 2 ± 2.4 | 0.2 ± 1.8 | 1.8 ± 2.4 | 3.7 ± 1.9 | 1.7 ± 2.8 | 4.9 ± 2.3 |
| 4 | 1 ± 1.8 | 0.3 ± 1.2 | 5.5 ± 3.6 | 2.3 ± 2.2 | 5.7 ± 2.7 | 0.8 ± 0.8 |
| 5 | 0.5 ± 0.9 | 1.4 ± 3.8 | 3.1 ± 1.6 | 2.3 ± 2.7 | 1.9 ± 2 | 1.4 ± 1.2 |
| 6 | 1.9 ± 1.8 | −0.3 ± 3 | 1.7 ± 1.2 | 1.4 ± 1.4 | 0.8 ± 1.1 | 1.1 ± 1 |
| 8 | −0.4 ± 0.8 | −0.9 ± 2.9 | 0.5 ± 1.3 | 3 ± 1.1 | 2.2 ± 2.1 | 3.7 ± 2 |
| 9 | 1 ± 1.7 | 1.2 ± 4.2 | 7.2 ± 3.9 | 0.5 ± 0.7 | 1.6 ± 3 | 3 ± 1 |
| 10 | 3.8 ± 1.8 | 11 ± 5 | −1.1 ± 1.1 | 1.9 ± 0.9 | 1.3 ± 1.6 | 0.2 ± 0.5 |

TABLE 24
Mean spot forming cells (SFC) per 10⁶ PBMCs for each epitope ± SEM
for VEE-MAG25mer srRNA-LNP1 (100 μg) (Group 2)

| | Antigen | | | | | |
|---|---|---|---|---|---|---|
| Wk | Env CL9 | Env TL9 | Gag CM9 | Gag LW9 | Pol SV9 | Tat TL8 |
| 1 | 0 ± 0 | 0 ± 0 | 0 ± 0 | 0 ± 0 | 0 ± 0 | 0 ± 0 |
| 2 | 7.9 ± 17.2 | 23.2 ± 17.4 | 11.4 ± 4.9 | 41.7 ± 16.5 | 15 ± 13.5 | 8.9 ± 6.2 |
| 3 | −3.1 ± 4.6 | −7.2 ± 6.5 | 2.3 ± 2.3 | −0.3 ± 2.7 | 2.7 ± 5.1 | 2.2 ± 1.4 |
| 4 | 1.9 ± 3.8 | −6.2 ± 7.6 | 10.5 ± 4.1 | 1.2 ± 2.9 | 5.6 ± 4.9 | 1.1 ± 0.8 |
| 5 | −2.6 ± 7 | −8 ± 5.9 | 1.5 ± 1.7 | 6.4 ± 2.3 | 0.7 ± 4.3 | 3.3 ± 1.3 |
| 6 | 6.3 ± 6.3 | 4.4 ± 8.3 | 6.6 ± 4.4 | 5.2 ± 5.2 | 3.9 ± 5 | 10.8 ± 6.9 |
| 8 | −3.6 ± 7.2 | −6.8 ± 7.3 | −0.8 ± 1.2 | 3.4 ± 4.2 | 6.4 ± 7.5 | 5.7 ± 2.7 |
| 9 | 8.1 ± 2.4 | 20.6 ± 23.4 | 18.9 ± 5.7 | 8.1 ± 8.9 | 9 ± 11.2 | 40 ± 17.6 |
| 10 | 3.1 ± 8 | −3.9 ± 8.5 | 3.3 ± 1.8 | 0.6 ± 2.9 | 7.4 ± 6.4 | 6.1 ± 2.5 |

TABLE 25
Mean spot forming cells (SFC) per 10⁶ PBMCs for each epitope ± SEM
for VEE-MAG25mer srRNA-LNP2 (100 μg) (Group 3)

| | Antigen | | | | | |
|---|---|---|---|---|---|---|
| Wk | Env CL9 | Env TL9 | Gag CM9 | Gag LW9 | Pol SV9 | Tat TL8 |
| 1 | 0 ± 0 | 0 ± 0 | 0 ± 0 | 0 ± 0 | 0 ± 0 | 0 ± 0 |
| 2 | −5.9 ± 3.8 | −0.3 ± 0.5 | −0.5 ± 1.5 | −5.7 ± 6.1 | −1 ± 1.3 | −3.2 ± 5.5 |
| 3 | 0.7 ± 5.2 | 3.4 ± 2.4 | 4.2 ± 4.6 | 18.3 ± 15.5 | 11.9 ± 5.1 | −0.4 ± 8.2 |
| 4 | −3.8 ± 5.5 | 2.3 ± 1.8 | 11.3 ± 6.1 | −3.1 ± 5.6 | 8.5 ± 4 | −1.5 ± 6.1 |
| 5 | −3.7 ± 5.7 | −0.1 ± 0.7 | −0.2 ± 1.6 | 3.4 ± 8.5 | 3 ± 3.1 | −4.6 ± 5 |
| 6 | 12.3 ± 15 | 7.8 ± 4.9 | 24.7 ± 19.8 | 23.2 ± 22.5 | 18.7 ± 15.8 | 0.5 ± 6.2 |
| 8 | 5.9 ± 12.3 | −0.1 ± 0.7 | −0.5 ± 1.3 | 8.8 ± 14.4 | 8.7 ± 8 | −1.3 ± 4 |
| 9 | 16.1 ± 13.4 | 16.5 ± 4 | 22.9 ± 4.2 | 13 ± 13.2 | 16.4 ± 7.8 | 19.6 ± 9.2 |
| 10 | 29.9 ± 21.8 | 22 ± 19.5 | 0.5 ± 2.6 | 22.2 ± 22.6 | 35.3 ± 15.8 | 19.4 ± 17.3 |

TABLE 26
Mean spot forming cells (SFC) per 10⁶ PBMCs for each epitope ± SEM for
ChAdV68.5WTnt.MAG25mer prime

| | Antigen | | | | | |
|---|---|---|---|---|---|---|
| Wk | Env CL9 | Env TL9 | Gag CM9 | Gag LW9 | Pol SV9 | Tat TL8 |
| 1 | 178 ± 68.7 | 206.5 ± 94.8 | 221.2 ± 120 | 15.4 ± 16.7 | 33.3 ± 25.9 | 563.5 ± 174.4 |
| 2 | 311.2 ± 165.5 | 278.8 ± 100.9 | 344.6 ± 110.8 | 46.3 ± 13.5 | 181.6 ± 76.8 | 621.4 ± 220.9 |
| 3 | 277.3 ± 101.1 | 359.6 ± 90.5 | 468.2 ± 106.6 | 41.7 ± 11.1 | 169.8 ± 57.8 | 549.4 ± 115.7 |
| 4 | 140 ± 46.5 | 169.6 ± 46.8 | 239.4 ± 37 | 26.5 ± 11.4 | 75 ± 31.6 | 322.2 ± 50.7 |
| 5 | 155.6 ± 62.1 | 406.7 ± 96.4 | 542.7 ± 143.3 | 35.1 ± 16.6 | 134.2 ± 53.7 | 538.5 ± 91.9 |
| 6 | 78.9 ± 42.5 | 95.5 ± 29.4 | 220.9 ± 75.3 | −1.4 ± 5.3 | 43.4 ± 19.6 | 308.1 ± 42.6 |
| 8 | 88.4 ± 30.4 | 162.1 ± 30.3 | 253.4 ± 78.6 | 21.4 ± 11.2 | 53.7 ± 22.3 | 217.8 ± 45.2 |
| 9 | 158.5 ± 69 | 322.3 ± 87.2 | 338.2 ± 137.1 | 5.6 ± 12.4 | 109.2 ± 17.9 | 314.8 ± 43.4 |
| 10 | 97.3 ± 32.5 | 133.2 ± 27 | 154.9 ± 59.2 | 10 ± 6 | 26 ± 16.7 | 125.5 ± 27.7 |

Non-GLP RNA Dose Ranging Study (Higher Doses) in Indian Rhesus Macaques

This study was designed to (a) evaluate the immunogenicity of VEE-MAG25mer srRNAat a dose of 300 μg as a homologous prime/boost or heterologous prime/boost in combination with ChAdV68.5WTnt.MAG25mer; (b) compare the immune responses of VEE-MAG25mer srRNA in lipid nanoparticles using LNP1 versus LNP2 at the 300 μg dose; and (c) evaluate the kinetics of T-cell responses to VEE-MAG25mer srRNA and ChAdV68.5WTnt.MAG25mer immunizations.

The study arm was conducted in Mamu-A*01 Indian rhesus macaques to demonstrate immunogenicity. Vaccine immunogenicity in nonhuman primate species, such as Rhesus, is the best predictor of vaccine potency in humans. Furthermore, select antigens used in this study are only recognized in Rhesus macaques, specifically those with a Mamu-A*01 MHC class I haplotype. Mamu-A*01 Indian rhesus macaques were randomized to the different study arms (6 macaques per group) and administered an IM injection bilaterally with either ChAdV68.5-WTnt.MAG25mer or VEE-MAG25mer srRNA encoding model antigens that includes multiple Mamu-A*01 restricted antigens. The study arms were as described below.

PBMCs were collected prior to immunization and 4, 5, 6, 7, 8, 10, 11, 12, 13, 14, 15, 16, 17, 18, 19, 20, 21, 22, 23 or 24 weeks after the initial immunization for immune monitoring for group 1 (heterologous prime/boost). PBMCs were collected prior to immunization and 4, 5, 7, 8, 10, 11, 12, 13, 14, or 15 weeks after the initial immunization for immune monitoring for groups 2 and 3 (homologous prime/boost).

TABLE 27
Non-GLP immunogenicity study in Indian Rhesus Macaques

| Group | Prime | Boost 1 | Boost 2 | Boost 3 |
|---|---|---|---|---|
| 1 | ChAdV68.5WTnt. | VEE-MAG25mer | VEE-MAG25mer | VEE-MAG25mer |
| | MAG25mer | srRNA-LNP2 | srRNA-LNP2 | srRNA-LNP2 |
| | | (300 μg) | (300 μg) | (300 μg) |
| 2 | VEE-MAG25mer | VEE-MAG25mer | VEE-MAG25mer | |
| | srRNA-LNP2 | srRNA-LNP2 | srRNA-LNP2 | |
| | (300 μg) | (300 μg) | (300 μg) | |
| 3 | VEE-MAG25mer | VEE-MAG25mer | VEE-MAG25mer | |
| | srRNA-LNP1 | srRNA-LNP1 | srRNA-LNP1 | |
| | (300 μg) | (300 μg) | (300 μg) | |

Results

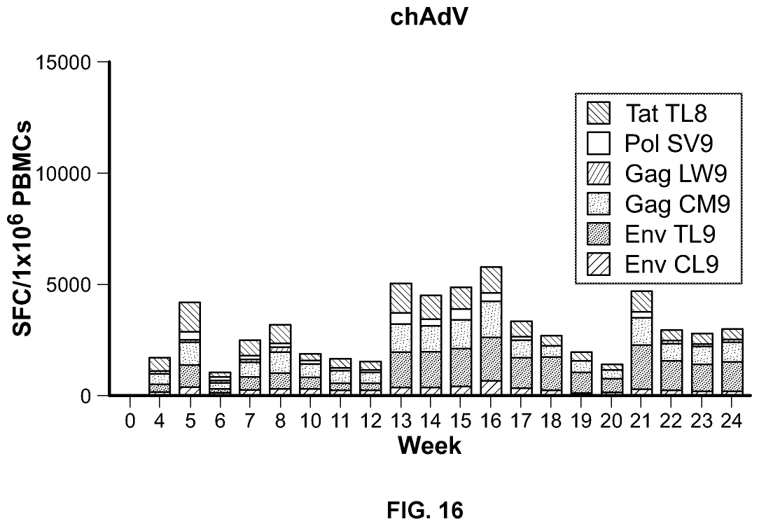

Mamu-A*01 Indian rhesus macaques were immunized with ChAdV68.5-WTnt.MAG25mer. Antigen-specific cellular immune responses in peripheral blood mononuclear cells (PBMCs) were measured to six different Mamu-A*01 restricted epitopes prior to immunization and 4, 5, 6, 7, 8, 10, 11, 12, 13, 14, 15, 16, 17, 18, 19, 20, 21, 22, 23 or 24 weeks after the initial immunization (FIG. 16 and Table 28). Animals received boost immunizations with VEE-MAG25mer srRNA using the LNP2 formulation on weeks 4, 12, and 20. Combined antigen-specific immune responses of 1750, 4225, 1100, 2529, 3218, 1915, 1708, 1561, 5077, 4543, 4920, 5820, 3395, 2728, 1996, 1465, 4730, 2984, 2828, or 3043 SFCs per 10⁶ PBMCs (six epitopes combined) were measured 4, 5, 6, 7, 8, 10, 11, 12, 13, 14, 15, 16, 17, 18, 19, 20, 21, 22, 23 or 24 weeks after the initial immunization with ChAdV68.5WTnt.MAG25mer (FIG. 16). Immune responses measured 1 week after the second boost immunization (week 13) with VEE-MAG25mer srRNA were ˜3-fold higher than that measured just before the boost immunization (week 12). Immune responses measured 1 week after the third boost immunization (week 21) with VEE-MAG25mer srRNA, were ˜3-fold higher than that measured just before the boost immunization (week 20), similar to the response observed for the second boost.

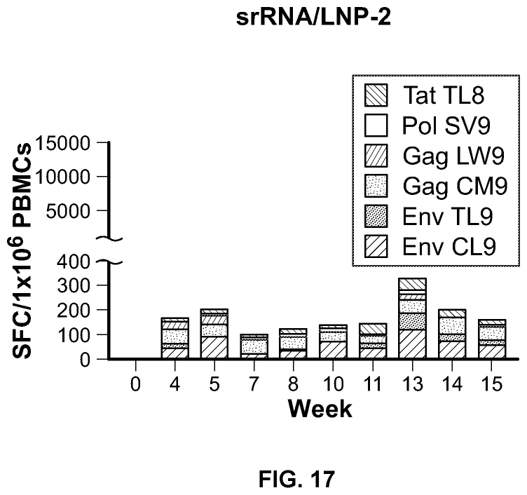

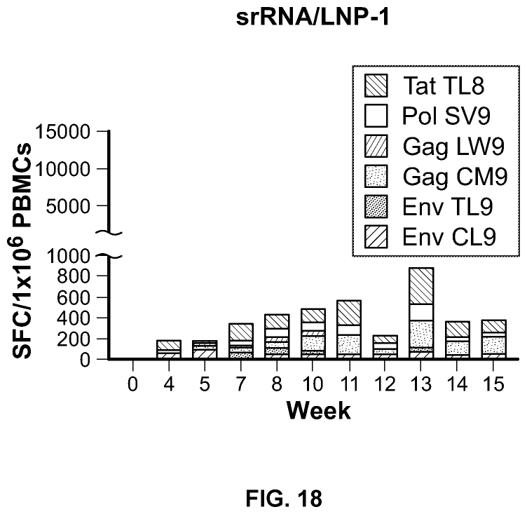

Mamu-A*01 Indian rhesus macaques were also immunized with VEE-MAG25mer srRNA using two different LNP formulations (LNP1 and LNP2). Antigen-specific cellular immune responses in peripheral blood mononuclear cells (PBMCs) were measured to six different Mamu-A*01 restricted epitopes prior to immunization and 4, 5, 6, 7, 8, 10, 11, 12, 13, 14, or 15 weeks after the initial immunization (FIGS. 22 and 23, Tables 29 and 30). Animals received boost immunizations with VEE-MAG25mer srRNA using the respective LNP1 or LNP2 formulation on weeks 4 and 12. Combined antigen-specific immune responses of 168, 204, 103, 126, 140, 145, 330, 203, and 162 SFCs per 106 PBMCs (six epitopes combined) were measured 4, 5, 7, 8, 10, 11, 13, 14, 15 weeks after the immunization with VEE-MAG25mer srRNA-LNP2 (FIG. 17). Combined antigen-specific immune responses of 189, 185, 349, 437, 492, 570, 233, 886, 369, and 381 SFCs per 10⁶ PBMCs (six epitopes combined) were measured 4, 5, 7, 8, 10, 11, 12, 13, 14, 15 weeks after the immunization with VEE-MAG25mer srRNA-LNP1 (FIG. 18).

TABLE 28
Mean spot forming cells (SFC) per 10⁶ PBMCs for each epitope ± SEM for
priming vaccination with ChAdV68.5WTnt.MAG25mer (Group 1)

| | Antigen | | | | | |
|---|---|---|---|---|---|---|
| Wk | Env CL9 | Env TL9 | Gag CM9 | Gag LW9 | Pol SV9 | Tat TL8 |
| 4 | 173 ± 41.6 | 373.5 ± 87.3 | 461.4 ± 74.2 | 38.4 ± 26.1 | 94.5 ± 26 | 609.2 ± 121.9 |
| 5 | 412.7 ± 138.4 | 987.8 ± 283.3 | 1064.4 ± 266.9 | 85.6 ± 31.2 | 367.2 ± 135.2 | 1306.8 ± 332.8 |
| 6 | 116.2 ± 41.2 | 231.1 ± 46.3 | 268.3 ± 90.7 | 86.1 ± 42 | 174.3 ± 61 | 223.9 ± 38.1 |
| 7 | 287.4 ± 148.7 | 588.9 ± 173.9 | 693.2 ± 224.8 | 92.1 ± 33.5 | 172.9 ± 55.6 | 694.6 ± 194.8 |
| 8 | 325.4 ± 126.6 | 735.8 ± 212 | 948.9 ± 274.5 | 211.3 ± 62.7 | 179.1 ± 50 | 817.3 ± 185.2 |
| 10 | 312 ± 129.7 | 543.2 ± 188.4 | 618.6 ± 221.7 | −5.7 ± 4.1 | 136.5 ± 51.3 | 309.9 ± 85.6 |
| 11 | 248.5 ± 81.1 | 348.7 ± 129.8 | 581.1 ± 205.5 | −3.1 ± 4.4 | 119 ± 51.2 | 413.7 ± 144.8 |
| 12 | 261.9 ± 68.2 | 329.9 ± 83 | 486.5 ± 118.6 | −1.2 ± 5.1 | 132.8 ± 31.8 | 350.9 ± 69.3 |
| 13 | 389.3 ± 167.7 | 1615.8 ± 418.3 | 1244.3 ± 403.6 | 1.3 ± 8.1 | 522.5 ± 155 | 1303.3 ± 385.6 |
| 14 | 406.3 ± 121.6 | 1616 ± 491.7 | 1142.3 ± 247.2 | 6.6 ± 11.1 | 322.7 ± 94.1 | 1048.6 ± 215.6 |
| 15 | 446.8 ± 138.7 | 1700.8 ± 469.1 | 1306.3 ± 294.4 | 43 ± 24.5 | 421.2 ± 87.9 | 1001.5 ± 236.4 |
| 16 | 686.8 ± 268.8 | 1979.5 ± 541.7 | 1616.8 ± 411.8 | 2.4 ± 7.8 | 381.9 ± 116.4 | 1152.8 ± 352.7 |
| 17 | 375.8 ± 109.3 | 1378.6 ± 561.2 | 773.1 ± 210.3 | −1.4 ± 4.3 | 177.6 ± 93.7 | 691.7 ± 245 |
| 18 | 255.9 ± 99.7 | 1538.4 ± 498.1 | 498.7 ± 152.3 | −5.3 ± 3.3 | 26.2 ± 13.4 | 413.9 ± 164.8 |
| 19 | 133 ± 62.6 | 955.9 ± 456.8 | 491.1 ± 121.8 | −5.7 ± 4.1 | 50.3 ± 25.4 | 371.2 ± 123.7 |
| 20 | 163.7 ± 55.8 | 641.7 ± 313.5 | 357.9 ± 91.1 | 2.6 ± 7.5 | 41.4 ± 24.2 | 257.8 ± 68.9 |
| 21 | 319.9 ± 160.5 | 2017.1 ± 419.9 | 1204.8 ± 335.2 | −3.7 ± 5.1 | 268.1 ± 109.6 | 924.1 ± 301 |
| 22 | 244.7 ± 105.6 | 1370.9 ± 563.5 | 780.3 ± 390 | −3.6 ± 5.1 | 118.2 ± 68.1 | 473.3 ± 249.3 |
| 23 | 176.7 ± 81.8 | 1263.7 ± 527.3 | 838.6 ± 367.9 | −5.7 ± 4.1 | 73.6 ± 49 | 480.9 ± 163.9 |
| 24 | 236.5 ± 92 | 1324.7 ± 589.3 | 879.7 ± 321 | −0.4 ± 5.7 | 104 ± 53.1 | 498 ± 135.8 |

TABLE 29
Mean spot forming cells (SFC) per 10⁶ PBMCs for each epitope ± SEM for
priming vaccination with VEE-MAG25mer srRNA-LNP2 (300 μg) (Group 2)

| | Antigen | | | | | |
|---|---|---|---|---|---|---|
| Wk | Env CL9 | Env TL9 | Gag CM9 | Gag LW9 | Pol SV9 | Tat TL8 |
| 4 | 46 ± 27.1 | 18.4 ± 6.8 | 58.3 ± 45.8 | 29.9 ± 20.8 | 4.9 ± 2.3 | 10.7 ± 4 |
| 5 | 85.4 ± 54 | 5.2 ± 5.8 | 52.4 ± 51.2 | 34.5 ± 35 | 11.8 ± 12.2 | 14.4 ± 7.9 |
| 7 | 18.6 ± 32.5 | 1.9 ± 1.7 | 59.4 ± 55.7 | 9.3 ± 10.7 | 3.3 ± 3 | 10.7 ± 6.1 |
| 8 | 36.6 ± 39.4 | 6.3 ± 3.9 | 48.7 ± 39.9 | 13.5 ± 8.8 | 3.8 ± 3.6 | 17.2 ± 9.7 |
| 10 | 69.1 ± 59.1 | 4.4 ± 1.9 | 39.3 ± 38 | 14.7 ± 10.8 | 4.4 ± 5.3 | 8.5 ± 5.3 |
| 11 | 43 ± 38.8 | 22.6 ± 21.1 | 30.2 ± 26.2 | 3.3 ± 2.2 | 5.8 ± 3.5 | 40.3 ± 25.5 |
| 13 | 120.4 ± 78.3 | 68.2 ± 43.9 | 54.2 ± 36.8 | 21.8 ± 7.4 | 17.7 ± 6.1 | 47.4 ± 27.3 |
| 14 | 76 ± 44.8 | 28 ± 19.5 | 65.9 ± 64.3 | −0.3 ± 1.3 | 2.5 ± 2 | 31.1 ± 26.5 |
| 15 | 58.9 ± 41.4 | 19.5 ± 15.1 | 55.4 ± 51 | 2.5 ± 2 | 5.5 ± 3.6 | 20.1 ± 15.7 |

TABLE 30
Mean spot forming cells (SFC) per 10⁶ PBMCs for each epitope ± SEM for
priming vaccination with VEE-MAG25mer srRNA-LNP1 (300 μg) (Group 3)

| | Antigen | | | | | |
|---|---|---|---|---|---|---|
| Wk | Env CL9 | Env TL9 | Gag CM9 | Gag LW9 | Pol SV9 | Tat TL8 |
| 4 | 19.5 ± 8.7 | 13.3 ± 13.1 | 16.5 ± 15.3 | 10.5 ± 7.3 | 35.9 ± 24.8 | 92.9 ± 91.6 |
| 5 | 87.9 ± 43.9 | 12.7 ± 11.7 | 37.2 ± 31.9 | 21.1 ± 23.8 | 13.2 ± 13.7 | 12.6 ± 13.7 |
| 7 | 21.1 ± 13.3 | 48.8 ± 48.4 | 51.7 ± 39.5 | 9.1 ± 10.5 | 58.6 ± 55.8 | 159.4 ± 159 |
| 8 | 47.7 ± 21.7 | 66.4 ± 52.2 | 59.8 ± 57.4 | 49.4 ± 28 | 79.4 ± 63 | 133.8 ± 132.1 |
| 10 | 49 ± 30.2 | 42.2 ± 41.1 | 139.3 ± 139.3 | 51.6 ± 51.2 | 78.2 ± 75.8 | 131.7 ± 131.6 |
| 11 | 42 ± 26.8 | 20.9 ± 21.4 | 177.1 ± 162 | −6.3 ± 4.3 | 104.3 ± 104.1 | 231.5 ± 230.1 |
| 12 | 40.2 ± 19 | 20.3 ± 11.9 | 42.2 ± 46.7 | 3.7 ± 6.7 | 57 ± 44.7 | 70 ± 69.2 |
| 13 | 81.2 ± 48.9 | 38.2 ± 37.6 | 259.4 ± 222.2 | −4 ± 4.1 | 164.1 ± 159.3 | 347.3 ± 343.5 |
| 14 | 34.5 ± 31.8 | 5.3 ± 11.6 | 138.6 ± 137.3 | −4.7 ± 5.2 | 52.3 ± 52.9 | 142.6 ± 142.6 |
| 15 | 49 ± 24 | 6.7 ± 9.8 | 167.1 ± 163.8 | −6.4 ± 4.2 | 47.8 ± 42.3 | 116.6 ± 114.5 |

srRNA Dose Ranging Study

In one implementation of the present invention, an srRNA dose ranging study can be conducted in mamu A01 Indian rhesus macaques to identify which srRNA dose to progress to NHP immunogenicity studies. In one example, Mamu A01 Indian rhesus macaques can be administered with an srRNA vector encoding model antigens that includes multiple mamu A01 restricted epitopes by IM injection. In another example, an anti-CTLA-4 monoclonal antibody can be administered SC proximal to the site of IM vaccine injection to target the vaccine draining lymph node in one group of animals. PBMCs can be collected every 2 weeks after the initial vaccination for immune monitoring. The study arms are described in below (Table 31).

TABLE 31
Non-GLP RNA dose ranging study in Indian Rhesus Macaques

| Group | Prime | Boost 1 | Boost 2 |
|---|---|---|---|
| 1 | srRNA-LNP | srRNA-LNP | srRNA-LNP |
| | (Low Dose) | (Low Dose) | (Low Dose) |
| 2 | srRNA-LNP | srRNA-LNP | srRNA-LNP |
| | (Mid Dose) | (Mid Dose) | (Mid Dose) |
| 3 | srRNA-LNP | srRNA-LNP | srRNA-LNP |
| | (High Dose) | (High Dose) | (High Dose) |
| 4 | srRNA-LNP | srRNA-LNP | srRNA-LNP |
| | (High Dose) + | (High Dose) + | (High Dose) + |
| | anti-CTLA-4 | anti-CTLA-4 | anti-CTLA-4 |

* Dose range of srRNA to be determined with the high dose ≤300 μg.

Immunogenicity Study in Indian Rhesus Macaques

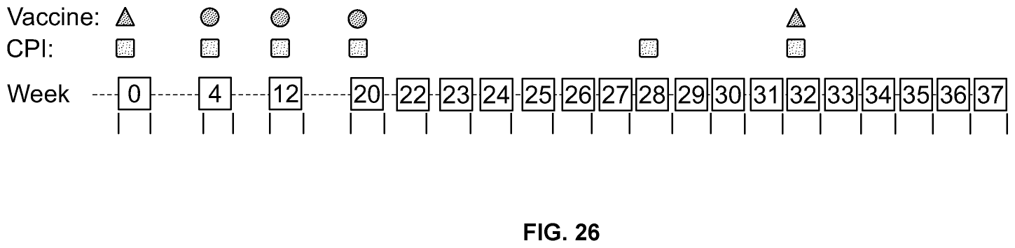

Vaccine studies were conducted in mamu A01 Indian rhesus macaques (NHPs) to demonstrate immunogenicity using the antigen vectors. FIG. 26 illustrates the vaccination strategy. Three groups of NHPs were immunized with ChAdV68.5-WTnt.MAG25mer and either with the checkpoint inhibitor anti-CTLA-4 antibody Ipilimumab (Groups 5 & 6) or without the checkpoint inhibitor (Group 4). The antibody was administered either intra-venously (group 5) or subcutaneously (group 6). Triangles indicate chAd68 vaccination (1e12 vp/animal) at weeks 0 & 32. Circles represent alphavirus vaccination at weeks 0, 4, 12, 20, 28 and 32.

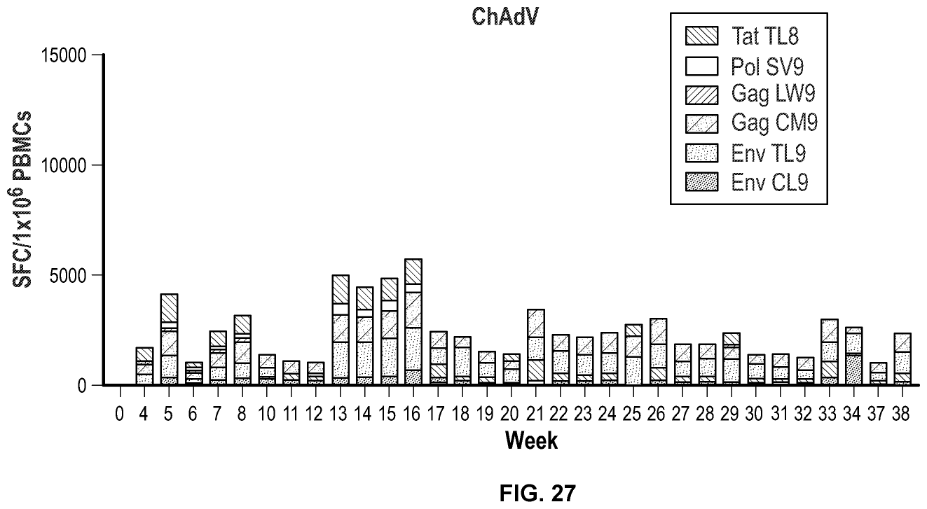

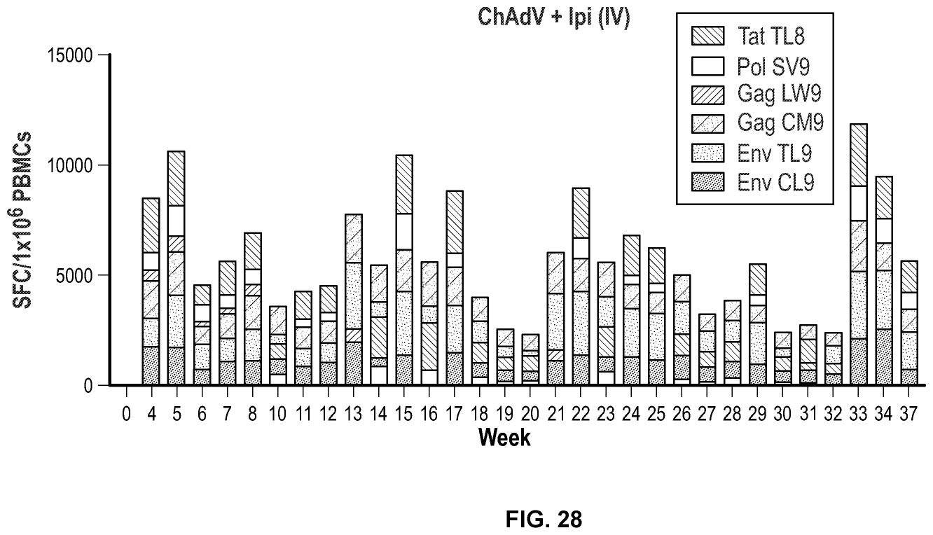

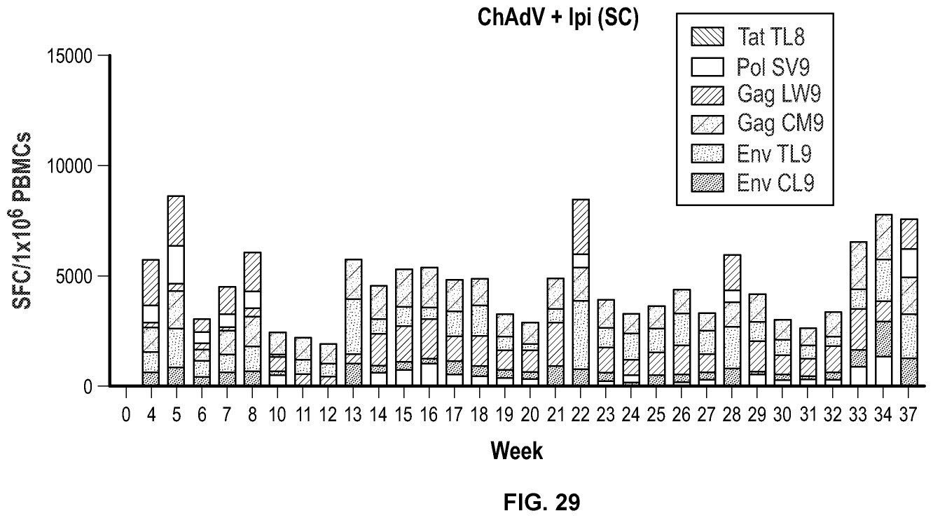

The time course of CD8+ anti-epitope responses in the immunized NHPs are presented for chAd-MAG immunization alone (FIG. 27 and Table 32A), chAd-MAG immunization with the checkpoint inhibitor delivered IV (FIG. 28 and Table 32B), and chAd-MAG immunization with the checkpoint inhibitor delivered SC (FIG. 29 and Table 32C). The results demonstrate chAd68 vectors efficiently primed CD8+ responses in primates, alphavirus vectors efficiently boosted the chAD68 vaccine priming response, checkpoint inhibitor whether delivered IV or SC amplified both priming and boosting responses, and chAd vectors readministered post vaccination to effectively boosted the immune responses.

TABLE 32A
CD8+ anti-epitope responses in Rhesus Macaques dosed with chAd-MAG
(Group 4). Mean SFC/1e6 splenocytes +/− the standard error is shown

| | Antigen | | | | | |
|---|---|---|---|---|---|---|
| Wk | Env CL9 | Env TL9 | Gag CM9 | Gag LW9 | Pol SV9 | Tat TL8 |
| 4 | 173 ± 41.6 | 373.5 ± 87.3 | 461.4 ± 74.2 | 38.4 ± 26.1 | 94.5 ± 26 | 609.2 ± 121.9 |
| 5 | 412.7 ± 138.4 | 987.8 ± 283.3 | 1064.4 ± 266.9 | 85.6 ± 31.2 | 367.2 ± 135.2 | 1306.8 ± 332.8 |
| 6 | 116.2 ± 41.2 | 231.1 ± 46.3 | 268.3 ± 90.7 | 86.1 ± 42 | 174.3 ± 61 | 223.9 ± 38.1 |
| 7 | 287.4 ± 148.7 | 588.9 ± 173.9 | 693.2 ± 224.8 | 92.1 ± 33.5 | 172.9 ± 55.6 | 694.6 ± 194.8 |
| 8 | 325.4 ± 126.6 | 735.8 ± 212 | 948.9 ± 274.5 | 211.3 ± 62.7 | 179.1 ± 50 | 817.3 ± 185.2 |
| 10 | 312 ± 129.7 | 543.2 ± 188.4 | 618.6 ± 221.7 | −5.7 ± 4.1 | 136.5 ± 51.3 | 309.9 ± 85.6 |
| 11 | 248.5 ± 81.1 | 348.7 ± 129.8 | 581.1 ± 205.5 | −3.1 ± 4.4 | 119 ± 51.2 | 413.7 ± 144.8 |
| 12 | 261.9 ± 68.2 | 329.9 ± 83 | 486.5 ± 118.6 | −1.2 ± 5.1 | 132.8 ± 31.8 | 350.9 ± 69.3 |
| 13 | 389.3 ± 167.7 | 1615.8 ± 418.3 | 1244.3 ± 403.6 | 1.3 ± 8.1 | 522.5 ± 155 | 1303.3 ± 385.6 |
| 14 | 406.3 ± 121.6 | 1616 ± 491.7 | 1142.3 ± 247.2 | 6.6 ± 11.1 | 322.7 ± 94.1 | 1048.6 ± 215.6 |
| 15 | 446.8 ± 138.7 | 1700.8 ± 469.1 | 1306.3 ± 294.4 | 43 ± 24.5 | 421.2 ± 87.9 | 1001.5 ± 236.4 |
| 16 | 686.8 ± 268.8 | 1979.5 ± 541.7 | 1616.8 ± 411.8 | 2.4 ± 7.8 | 381.9 ± 116.4 | 1152.8 ± 352.7 |
| 17 | 375.8 ± 109.3 | 1378.6 ± 561.2 | 773.1 ± 210.3 | −1.4 ± 4.3 | 177.6 ± 93.7 | 691.7 ± 245 |
| 18 | 255.9 ± 99.7 | 1538.4 ± 498.1 | 498.7 ± 152.3 | −5.3 ± 3.3 | 26.2 ± 13.4 | 413.9 ± 164.8 |
| 19 | 133 ± 62.6 | 955.9 ± 456.8 | 491.1 ± 121.8 | −5.7 ± 4.1 | 50.3 ± 25.4 | 371.2 ± 123.7 |
| 20 | 163.7 ± 55.8 | 641.7 ± 313.5 | 357.9 ± 91.1 | 2.6 ± 7.5 | 41.4 ± 24.2 | 257.8 ± 68.9 |
| 21 | 319.9 ± 160.5 | 2017.1 ± 419.9 | 1204.8 ± 335.2 | −3.7 ± 5.1 | 268.1 ± 109.6 | 924.1 ± 301 |
| 22 | 244.7 ± 105.6 | 1370.9 ± 563.5 | 780.3 ± 390 | −3.6 ± 5.1 | 118.2 ± 68.1 | 473.3 ± 249.3 |
| 23 | 176.7 ± 81.8 | 1263.7 ± 527.3 | 838.6 ± 367.9 | −5.7 ± 4.1 | 73.6 ± 49 | 480.9 ± 163.9 |
| 24 | 236.5 ± 92 | 1324.7 ± 589.3 | 879.7 ± 321 | −0.4 ± 5.7 | 104 ± 53.1 | 498 ± 135.8 |
| 25 | 136.4 ± 52.6 | 1207.1 ± 501.6 | 924 ± 358.5 | 6.2 ± 10.5 | 74.1 ± 34.4 | 484.6 ± 116.7 |
| 26 | 278.2 ± 114.4 | 1645 ± 661.7 | 1170.2 ± 469.9 | −2.9 ± 5.7 | 80.6 ± 55.8 | 784.4 ± 214.1 |
| 27 | 159 ± 56.8 | 961.7 ± 547.1 | 783.6 ± 366.4 | −5 ± 4.3 | 63.6 ± 27.5 | 402.9 ± 123.4 |
| 28 | 189.6 ± 75.7 | 1073.1 ± 508.8 | 668.3 ± 312.5 | −5.7 ± 4.1 | 80.3 ± 38.3 | 386.4 ± 122 |
| 29 | 155.3 ± 69.1 | 1102.9 ± 606.1 | 632.9 ± 235 | 34.5 ± 24.2 | 80 ± 35.5 | 422.5 ± 122.9 |
| 30 | 160.2 ± 59.9 | 859 ± 440.9 | 455 ± 209.1 | −3 ± 5.3 | 60.5 ± 28.4 | 302.7 ± 123.2 |
| 31 | 122.2 ± 49.7 | 771.1 ± 392.7 | 582.2 ± 233.5 | −5.7 ± 4.1 | 55.1 ± 27.3 | 295.2 ± 68.3 |
| 32 | 119.3 ± 28.3 | 619.4 ± 189.7 | 566 ± 222.1 | −3.7 ± 5.1 | 21.9 ± 4.5 | 320.5 ± 76.4 |
| 33 | 380.5 ± 122 | 1636.1 ± 391.4 | 1056.2 ± 205.7 | −5.7 ± 4.1 | 154.5 ± 38.5 | 988.4 ± 287.7 |
| 34 | 1410.8 ± 505.4 | 972.4 ± 301.5 | 319.6 ± 89.6 | −4.8 ± 4.2 | 141.1 ± 49.8 | 1375.5 ± 296.7 |
| 37 | 130.8 ± 29.2 | 500 ± 156.9 | 424.9 ± 148.9 | −3.5 ± 4.7 | 77.7 ± 24.6 | 207.1 ± 42.4 |
| 38 | 167.7 ± 54.8 | 1390.8 ± 504.7 | 830.4 ± 329.1 | −5.5 ± 4.1 | 111.8 ± 43.2 | 516 ± 121.7 |

TABLE 32B
CD8+ anti-epitope responses in Rhesus Macaques dosed with chAd-MAG plus
anti-CTLA4 antibody (Ipilimumab) delivered IV. (Group 5). Mean SFC/1e6 splenocytes +/−
the standard error is shown

| | Antigen | | | | | |
|---|---|---|---|---|---|---|
| Wk | Env CL9 | Env TL9 | Gag CM9 | Gag LW9 | Pol SV9 | Tat TL8 |
| 4 | 1848.1 ± 432.2 | 1295.7 ± 479.7 | 1709.8 ± 416.9 | 513.7 ± 219.8 | 838.5 ± 221.1 | 2514.6 ± 246.5 |
| 5 | 1844.1 ± 410.2 | 2367.5 ± 334.4 | 1983.1 ± 370.7 | 732.1 ± 249.4 | 1429.7 ± 275.3 | 2517.7 ± 286.5 |
| 6 | 822.4 ± 216.7 | 1131.2 ± 194.7 | 796.8 ± 185.8 | 226.8 ± 70 | 802.2 ± 101.4 | 913.5 ± 222.7 |
| 7 | 1147.2 ± 332.9 | 1066 ± 311.2 | 1149.8 ± 467.3 | 267.4 ± 162.6 | 621.5 ± 283.2 | 1552.2 ± 395.1 |
| 8 | 1192.7 ± 188.8 | 1461.5 ± 237.7 | 1566.9 ± 310.5 | 522.5 ± 118.6 | 662.3 ± 142.4 | 1706 ± 216.7 |
| 10 | 1249 ± 220.3 | 1170.6 ± 227.7 | 1297.3 ± 264.7 | −0.3 ± 4.4 | 551.8 ± 90.5 | 1425.3 ± 142.6 |
| 11 | 934.2 ± 221.7 | 808 ± 191.3 | 1003.1 ± 293.4 | 1.9 ± 4.3 | 364.2 ± 76.6 | 1270.8 ± 191.6 |
| 12 | 1106.2 ± 216.6 | 896.7 ± 190.7 | 1020.1 ± 243.3 | 1.3 ± 3.9 | 436.6 ± 90 | 1222 ± 155.4 |
| 13 | 2023.8 ± 556.3 | 3696.7 ± 1.7 | 2248.5 ± 436.4 | −4.5 ± 3.5 | 2614 ± 406.1 | 3700 ± 0 |
| 14 | 1278.7 ± 240 | 2639.5 ± 387 | 1654.6 ± 381.1 | −6 ± 2.1 | 988.8 ± 197.9 | 2288.3 ± 298.7 |
| 15 | 1458.9 ± 281.8 | 2932.5 ± 488.7 | 1893.4 ± 499 | 74.6 ± 15.6 | 1657.8 ± 508.9 | 2709.1 ± 428.7 |
| 16 | 1556.8 ± 243 | 2143.8 ± 295.2 | 2082.8 ± 234.2 | −5.8 ± 2.5 | 1014.6 ± 161.4 | 2063.7 ± 86.7 |
| 17 | 1527 ± 495.1 | 2213 ± 677.1 | 1767.7 ± 391.8 | 15.1 ± 5.9 | 633.8 ± 133.9 | 2890.8 ± 433.9 |
| 18 | 1068.2 ± 279.9 | 1940.9 ± 204.1 | 1114.1 ± 216.1 | −5.8 ± 2.5 | 396.6 ± 77.6 | 1659.4 ± 171.7 |
| 19 | 760.7 ± 362.2 | 1099.5 ± 438.4 | 802.7 ± 192.5 | −2.4 ± 3.3 | 262.2 ± 62.2 | 1118.6 ± 224.2 |
| 20 | 696.3 ± 138.2 | 954.9 ± 198 | 765.1 ± 248.4 | −1.4 ± 4.4 | 279.6 ± 89.3 | 1139 ± 204.5 |
| 21 | 1201.4 ± 327.9 | 3096 ± 1.9 | 1901 ± 412.1 | −5.8 ± 2.5 | 1676.3 ± 311.5 | 2809.3 ± 195.8 |
| 22 | 1442.5 ± 508.3 | 2944.7 ± 438.6 | 1528.4 ± 349.6 | 2.8 ± 5.1 | 940.7 ± 160.5 | 2306.3 ± 218.6 |
| 23 | 1400.4 ± 502.2 | 2757.1 ± 452.9 | 1604.2 ± 450.1 | −5.1 ± 2.3 | 708.1 ± 162.6 | 2100.4 ± 362.9 |
| 24 | 1351 ± 585.1 | 2264.5 ± 496 | 1080.6 ± 253.8 | 0.3 ± 6.5 | 444.2 ± 126.4 | 1823.7 ± 306.5 |
| 25 | 1211.5 ± 505.2 | 2160.4 ± 581.8 | 970.8 ± 235.9 | 2.5 ± 3.8 | 450.4 ± 126.9 | 1626.2 ± 261.3 |
| 26 | 1443 ± 492.5 | 2485 ± 588 | 1252.5 ± 326.4 | −0.2 ± 6 | 360.2 ± 92.3 | 2081.9 ± 331.1 |
| 27 | 896.2 ± 413.3 | 1686 ± 559.5 | 751 ± 192.1 | −3.7 ± 2.5 | 247.4 ± 82.8 | 1364.1 ± 232 |
| 28 | 1147.8 ± 456.9 | 1912.1 ± 417.1 | 930.3 ± 211.4 | −5.8 ± 2.5 | 423.9 ± 79.6 | 1649.3 ± 315 |
| 29 | 1038.5 ± 431.9 | 1915.2 ± 626.1 | 786.8 ± 205.9 | 23.5 ± 8.3 | 462.8 ± 64 | 1441.5 ± 249.7 |
| 30 | 730.5 ± 259.3 | 1078.6 ± 211.5 | 699.1 ± 156.2 | −4.4 ± 2.7 | 234.4 ± 43.9 | 1160.6 ± 112.6 |
| 31 | 750.4 ± 328.3 | 1431 ± 549.9 | 650.6 ± 141.1 | −5.2 ± 3 | 243.4 ± 56.4 | 868.9 ± 142.8 |
| 32 | 581.4 ± 227.7 | 1326.6 ± 505.2 | 573.3 ± 138 | −3.2 ± 4.2 | 160.8 ± 49.2 | 936.4 ± 110.4 |
| 33 | 2198.4 ± 403.8 | 3093.4 ± 123.3 | 2391.8 ± 378.4 | 7.1 ± 8.5 | 1598.1 ± 343.1 | 2827.5 ± 289.5 |
| 34 | 2654.3 ± 337 | 2709.9 ± 204.3 | 1297.5 ± 291.4 | 0.4 ± 4.2 | 1091.8 ± 242.9 | 1924 ± 245.7 |
| 37 | 846.8 ± 301.7 | 1706.9 ± 196 | 973.6 ± 149.3 | 50.5 ± 45.2 | 777.3 ± 140.2 | 1478.8 ± 94.3 |

TABLE 32C
CD8+ anti-epitope responses in Rhesus Macaques dosed with chAd-MAG plus
anti-CTLA4 antibody (Ipilimumab) delivered SC (Group 6). Mean SFC/1e6 splenocytes +/−
the standard error is shown

| | Antigen | | | | | |
|---|---|---|---|---|---|---|
| Wk | Env CL9 | Env TL9 | Gag CM9 | Gag LW9 | Pol SV9 | Tat TL8 |
| 4 | 598.3 ± 157.4 | 923.7 ± 306.8 | 1075.6 ± 171.8 | 180.5 ± 74.1 | 752.3 ± 245.8 | 1955.3 ± 444.4 |
| 5 | 842.2 ± 188.5 | 1703.7 ± 514.2 | 1595.8 ± 348.4 | 352.7 ± 92.3 | 1598.9 ± 416.8 | 2163.7 ± 522.1 |
| 6 | 396.4 ± 45.3 | 728.3 ± 232.7 | 503.8 ± 151.9 | 282 ± 69 | 463.1 ± 135.7 | 555.2 ± 191.5 |
| 7 | 584.2 ± 177 | 838.3 ± 254.9 | 1013.9 ± 349.4 | 173.6 ± 64.3 | 507.4 ± 165.7 | 1222.8 ± 368 |
| 8 | 642.9 ± 134 | 1128.6 ± 240.6 | 1259.1 ± 163.8 | 366.1 ± 72.8 | 726.7 ± 220.9 | 1695.6 ± 359.4 |
| 10 | 660.4 ± 211.4 | 746.9 ± 222.7 | 944.8 ± 210.2 | −1.2 ± 1.9 | 523.4 ± 230.7 | 787.3 ± 308.3 |
| 11 | 571.2 ± 162 | 609.4 ± 194.3 | 937.9 ± 186.5 | −8.9 ± 2.3 | 511.6 ± 229.6 | 1033.3 ± 315.7 |
| 12 | 485.3 ± 123.7 | 489.4 ± 142.7 | 919.3 ± 214.1 | −8.9 ± 2.3 | 341.6 ± 139.4 | 1394.7 ± 432.1 |
| 13 | 986.9 ± 154.5 | 2811.9 ± 411.3 | 1687.7 ± 344.3 | −4.1 ± 5.1 | 1368.5 ± 294.2 | 2751 ± 501.9 |
| 14 | 945.9 ± 251.4 | 2027.7 ± 492.8 | 1386.7 ± 326.7 | −5.7 ± 2.8 | 708.9 ± 277.1 | 1588.2 ± 440.1 |
| 15 | 1075.2 ± 322.4 | 2386 ± 580.7 | 1606.3 ± 368.1 | −5.4 ± 3.2 | 763.3 ± 248.8 | 1896.5 ± 507.8 |
| 16 | 1171.8 ± 341.6 | 2255.1 ± 439.6 | 1672.2 ± 342.3 | −7.8 ± 2.4 | 1031.6 ± 228.8 | 1896.4 ± 419.9 |
| 17 | 1118.2 ± 415.4 | 2156.3 ± 476 | 1345.3 ± 377.7 | −1.1 ± 6.7 | 573.7 ± 118.8 | 1614.4 ± 382.3 |
| 18 | 861.3 ± 313.8 | 2668.2 ± 366.8 | 1157.2 ± 259.6 | −8.9 ± 2.3 | 481.2 ± 164 | 1725.8 ± 511.4 |
| 19 | 719.2 ± 294.2 | 1447.2 ± 285 | 968 ± 294.5 | −2.2 ± 4.6 | 395.6 ± 106.1 | 1199.6 ± 289.2 |
| 20 | 651.6 ± 184 | 1189.8 ± 242.8 | 947.4 ± 249.8 | −8.9 ± 2.3 | 355 ± 106.3 | 1234.7 ± 361.7 |
| 21 | 810.3 ± 301.9 | 2576.2 ± 283.7 | 1334 ± 363.1 | −8.9 ± 2.3 | 892.2 ± 305 | 1904.4 ± 448.1 |
| 22 | 775 ± 196.4 | 2949 ± 409.7 | 1421.8 ± 309.7 | 38 ± 27.8 | 577 ± 144.2 | 2330.6 ± 572.3 |
| 23 | 584.9 ± 240.2 | 1977.9 ± 361.4 | 1209.8 ± 405.1 | −7.3 ± 3.2 | 273.7 ± 93.3 | 1430.6 ± 363.9 |
| 24 | 485.4 ± 194.4 | 1819.8 ± 325.5 | 837.2 ± 261.4 | −3.4 ± 4.1 | 234.4 ± 71.1 | 943.9 ± 243.3 |
| 25 | 452.3 ± 175 | 2072 ± 405.7 | 957.1 ± 293.1 | −8.9 ± 2.3 | 163 ± 43.2 | 1341.2 ± 394.7 |
| 26 | 517.9 ± 179.1 | 2616 ± 567.5 | 1126.6 ± 289 | −8.3 ± 2.3 | 199.9 ± 89.2 | 1615.7 ± 385.6 |
| 27 | 592.8 ± 171.7 | 1838.3 ± 372.4 | 749.3 ± 170.4 | −7.3 ± 2.5 | 325.5 ± 98.7 | 1110.7 ± 308.8 |
| 28 | 793 ± 228.5 | 1795.4 ± 332.3 | 1068.7 ± 210.3 | 2.5 ± 4.1 | 553.1 ± 144.3 | 1480.8 ± 357.1 |
| 29 | 661.8 ± 199.9 | 2140.6 ± 599.3 | 1202.7 ± 292.2 | −8.7 ± 2.8 | 558.9 ± 279.2 | 1424.2 ± 408.6 |
| 30 | 529.1 ± 163.3 | 1528.2 ± 249.8 | 840.5 ± 218.3 | −8.9 ± 2.3 | 357.7 ± 149.4 | 1029.3 ± 335 |
| 31 | 464.8 ± 152.9 | 1332.2 ± 322.7 | 726.3 ± 194.3 | −8.9 ± 2.3 | 354.3 ± 158.6 | 884.4 ± 282 |
| 32 | 612.9 ± 175.3 | 1584.2 ± 390.2 | 1058.3 ± 219.8 | −8.7 ± 2.8 | 364.6 ± 149.8 | 1388.8 ± 467.3 |
| 33 | 1600.2 ± 416.7 | 2597.4 ± 367.9 | 2086.4 ± 414.8 | −6.3 ± 3.3 | 893.8 ± 266 | 2490.6 ± 416.4 |
| 34 | 2814.6 ± 376.2 | 2713.6 ± 380.8 | 1888.8 ± 499.4 | −7.5 ± 3.1 | 1288.9 ± 438.9 | 2428.1 ± 458.9 |
| 37 | 1245.9 ± 471.7 | 1877.7 ± 291.2 | 1606.6 ± 441.9 | 14.2 ± 13 | 1227.5 ± 348.1 | 1260.7 ± 342 |

Memory Phenotyping in Indian Rhesus Macaques

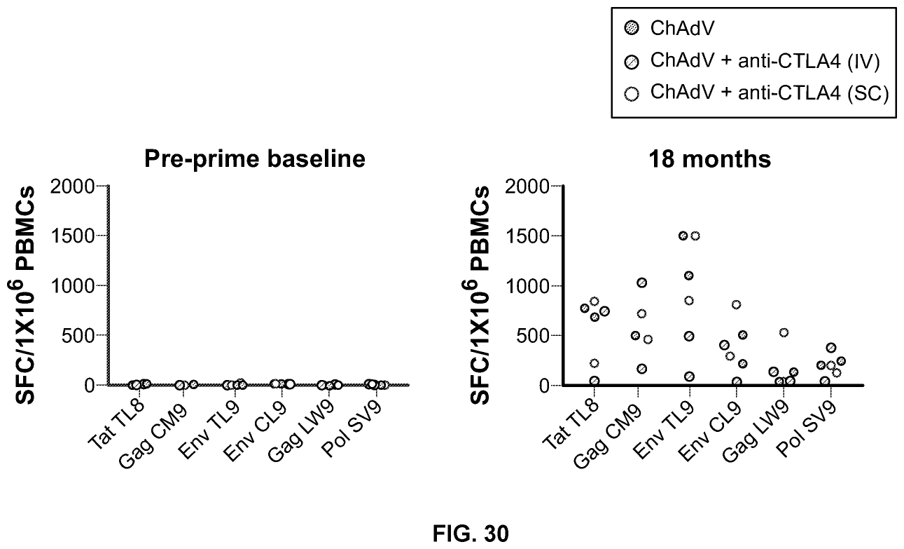

Rhesus macaque were immunized with ChAdV68.5WTnt.MAG25mer/VEE-MAG25mer srRNA heterologous prime/boost regimen with or without anti-CTLA4, and boosted again with ChAdV68.5WTnt.MAG25mer. Groups were assessed 11 months after the final ChAdV68 administration (study month 18). by ELISpot was performed as described. FIG. 30 and Table 33 shows cellular responses to six different Mamu-A*01 restricted epitopes as measured by ELISpot both pre-immunization (left panel) and after 18 months (right panel). The detection of responses to the restricted epitopes demonstrates antigen-specific memory responses were generated by ChAdV68/samRNA vaccine protocol.

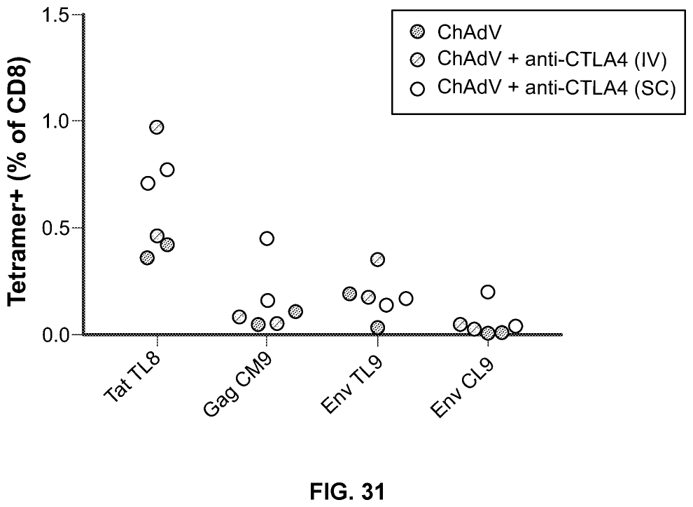

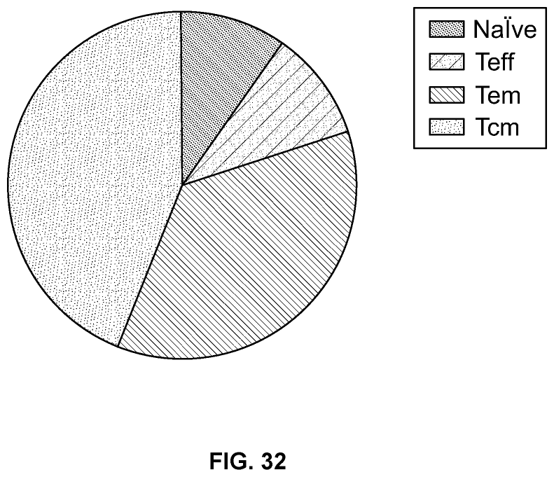

To assess memory, CD8+ T-cells recognizing 4 different rhesus macaque Mamu-A*01 class I epitopes encoded in the vaccines were monitored using dual-color Mamu-A*01 tetramer labeling, with each antigen being represented by a unique double positive combination, and allowed the identification of all 4 antigen-specific populations within a single sample. Memory cell phenotyping was performed by co-staining with the cell surface markers CD45RA and CCR7. FIG. 31 and Table 34 shows the results of the combinatorial tetramer staining and CD45RA/CCR7 co-staining for memory T-cells recognizing four different Mamu-A*01 restricted epitopes. The T cell phenotypes were also assessed by flow cytometry. FIG. 32 shows the distribution of memory cell types within the sum of the four Mamu-A*01 tetramer+CD8+ T-cell populations at study month 18. Memory cells were characterized as follows: CD45RA+CCR7+=naïve, CD45RA+CCR7-=effector (Teff), CD45RA-CCR7+=central memory (Tcm), CD45RA-CCR7-=effector memory (Tem). Collectively, the results demonstrate that memory responses were detected at least one year following the last boost indicating long lasting immunity, including effector, central memory, and effector memory populations.

TABLE 33
Mean spot forming cells (SFC) per 10⁶ PBMCs for each animal at both pre-prime
and memory assessment time points (18 months).

| | Pre-prime baseline | | | | | | 18 months | | | | | |
|---|---|---|---|---|---|---|---|---|---|---|---|---|
| | Tat | Gag | Env | Env | Gag | Pol | Tat | Gag | Env | Env | Gag | Pol |
| Animal | TL8 | CM9 | TL9 | CL9 | LW9 | SV9 | TL8 | CM9 | TL9 | CL9 | LW9 | SV9 |
| 1 | 1.7 | 0.0 | 0.0 | 5.0 | 0.0 | 13.7 | 683.0 | 499.2 | 1100.3 | 217.5 | 47.7 | 205.3 |
| 2 | 0.0 | 0.0 | 0.0 | 0.2 | 0.1 | 0.0 | 773.4 | ND | 1500.0 | 509.3 | 134.5 | 242.5 |
| 3 | 0.0 | 0.0 | 6.7 | 6.8 | 10.2 | 3.3 | 746.3 | 167.5 | 494.1 | 402.8 | 140.6 | 376.0 |
| 4 | 0.0 | 0.0 | 0.0 | 0.0 | 0.0 | 0.0 | 47.6 | 1023.9 | 85.1 | 44.2 | 44.2 | 47.6 |
| 5 | 15.3 | 6.7 | 18.6 | 15.6 | 5.2 | 12.1 | 842.4 | 467.7 | 1500.0 | 805.9 | 527.8 | 201.8 |
| 6 | 3.1 | 0.0 | 0.0 | 15.5 | 6.9 | 5.3 | 224.3 | 720.3 | 849.0 | 296.9 | 32.4 | 121.9 |

ND = not determined due to technical exclusion

TABLE 34
Percent Mamu-A*01 tetramer positive out of live CD8+ cells

| Animal | Tat TL8 | Gag CM9 | Env TL9 | Env CL9 |
|---|---|---|---|---|
| 1 | 0.42 | 0.11 | 0.19 | 0.013 |
| 2 | 0.36 | 0.048 | 0.033 | 0.00834 |
| 3 | 0.97 | 0.051 | 0.35 | 0.048 |
| 4 | 0.46 | 0.083 | 0.17 | 0.028 |
| 5 | 0.77 | 0.45 | 0.14 | 0.2 |
| 6 | 0.71 | 0.16 | 0.17 | 0.04 |

### XVII. Example 5: Identification of MHC/Peptide Target-Reactive T Cells and TCRs

Target reactive T cells and TCRs are identified for one or more of the HIV subtype/HLA allele/epitope sequence combinations shown in Tables 35-45.

T cells are isolated from blood, lymph nodes, or tissues of patients. T cells can be enriched for antigen-specific T cells, e.g., by sorting antigen-MHC tetramer binding cells or by sorting activated cells stimulated in an in vitro co-culture of T cells and antigen-pulsed antigen presenting cells. Various reagents are known in the art for antigen-specific T cell identification including antigen-loaded tetramers and other MHC-based reagents.

Antigen-relevant alpha-beta (or gamma-delta) TCR dimers can be identified by single cell sequencing of TCRs of antigen-specific T cells. Alternatively, bulk TCR sequencing of antigen-specific T cells can be performed and alpha-beta pairs with a high probability of matching can be determined using a TCR pairing method known in the art.

Alternatively or in addition, antigen-specific T cells can be obtained through in vitro priming of naïve T cells from healthy donors. T cells obtained from PBMCs, lymph nodes, or cord blood can be repeatedly stimulated by antigen-pulsed antigen presenting cells to prime differentiation of antigen-experienced T cells. TCRs can then be identified similarly as described above for antigen-specific T cells from patients.

### XVIII. Example 6: Identification of Candidate Antigens

Candidate HIV antigens were identified for inclusion in the antigen-based vaccine using a series of steps. For each HIV subtype (subtypes A1, A2, B, C, D, F1, F2, G, H, J, and K), sequences for each of nine HIV genes (env, gag, nef, pol, rev, tat, vif, vpr, and vpu) were obtained from Los Alamos National Lab's HIV database¹⁰⁴. Amino acid sequences (8-11 amino acids in length) are extracted from sequences of the nine HIV genes for each HIV subtype. Specifically, a sliding window is applied to the sequences of the nine HIV genes to obtain the amino acid sequences (8-11 amino acids in length). These amino acid sequences were applied to the EDGE prediction model (a deep learning model trained on HLA presented peptides sequenced by MS/MS, as described in international patent application publications WO/2017/106638, WO/2018/195357, WO/2018/208856, and PCT/US19/33830, each herein incorporated by reference, in their entirety, for all purposes) across all modeled HLA alleles. All epitope sequence/HLA allele pair that has an EDGE score>0.01 was recorded for each HIV subtype. A total of 7096 unique epitope sequences were identified and the corresponding HLA allele for each sequence are shown in Tables 35-45.

### XIX. Example 7: Validation of Candidate Antigen Presentation

Mass spectrometry (MS) validation of candidate antigens is performed using targeted mass spectrometry methods. HIV tissue samples are obtained, homogenized, and used for RNASeq transcriptome sequencing and immunoprecipitation of the HLA/peptide complexes. A peptide target list is generated for each sample by analysis of the transcriptome. The EDGE deep learning model of antigen presentation is applied to the mutation sequence and expression data to prioritize peptides for the targeting list. The peptides from the HLA molecules are eluted and collected using size exclusion to isolate the presented peptides prior to mass spectrometry. Synthetic heavy labeled peptide with the same amino acid sequence is co-loaded with each sample for targeted mass spectrometry. Both coelution of the heavy labeled peptide with the experimental peptide and analysis of the fragmentation pattern are used to validate a candidate epitope sequence. Mass spectrometry analysis methods are described in more detail in Gillete et al. (Nat Methods. 2013 January; 10(1):28-34), herein incorporated by reference in its entirety for all purposes.

MS data are further evaluated to assess the value of narrowly targeting patients with specific HLAs for treatment, e.g., requiring patients to have at least one validated or predicted HLA allele that presents an antigen contained in a vaccine cassette. For example, a candidate epitope sequence may be selected for inclusion because it was predicted to be presented by a particular HLA protein. However, if the MS data demonstrates the contrary and that the candidate epitope sequence was not presented by the HLA protein, then the epitope sequence/HLA protein pair can be excluded for purposes of selection criteria for the vaccine.

### XX. Example 8: Vaccine Cassette Antigen Selection

Antigens including epitope sequences for inclusion in an antigen-based vaccine were chosen. Although the subsequent description refers to selection of antigenic peptides and subsequent inclusion of sequences in an antigen cassette, the sequences encoding for such selected antigenic peptides, one skilled in the art may understand that the subsequent description can also be applied for the inclusion of the antigenic peptides themselves in the antigen-based vaccine.

First, for each HIV subtype (A1, A2, B, C, D, F1, F2, G, H, J, and K), a corresponding Table in Tables 35-45 was identified (e.g., A1—Table 35, A2—Table 36, B—Table 37, C—Table 38, D—Table 39, F1—Table 40, F2—Table 41, G—Table 42, H—Table 43, J—Table 44, and K—Table 45).

Next, for each HLA allele in the table, epitope sequences (or antigen-encoding sequences that encode for each of the epitope sequences) for inclusion in the antigen-based vaccine were selected by identifying rows in the table that list the particular HLA allele.

Specifically, for a HIV subtype and HLA allele A0101, the antigen-encoding sequence for inclusion in the vaccine was selected by reference to the table corresponding to the HIV subtype, where each relevant sequence considered is selected by identifying all rows in that table that list A0101 (e.g., any of SEQ ID NOs: 325-328, 2166-2178, 4107-4113, 6242-6248, 8390-8397, 10627-10633, 12811-12820, 15080-15086, 17175-17184, 19389-19396, or 21004-21009).

For a HIV subtype and HLA allele A0201, the antigen-encoding sequence for inclusion in the vaccine was selected by reference to the table corresponding to the HIV subtype, where each relevant sequence considered is selected by identifying all rows in that table that list A0201 (e.g., any of SEQ ID NOs: 329-353, 2179-2200, 4114-4134, 6249-6270, 8398-8415, 10634-10654, 12821-12850, 15087-15107, 17185-17213, 19397-19420, or 21010-21031).

For a HIV subtype and HLA allele A0203, the antigen-encoding sequence for inclusion in the vaccine was selected by reference to the table corresponding to the HIV subtype, where each relevant sequence considered is selected by identifying all rows in that table that list A0203 (e.g., any of SEQ ID NOs: 354-403, 2201-2248, 4135-4177, 6271-6315, 8416-8474, 10655-10700, 12851-12912, 15108-15155, 17214-17264, 19421-19463, or 21032-21064).

For a HIV subtype and HLA allele A0204, the antigen-encoding sequence for inclusion in the vaccine was selected by reference to the table corresponding to the HIV subtype, where each relevant sequence considered is selected by identifying all rows in that table that list A0204 (e.g., any of SEQ ID NOs: 404-469, 2249-2326, 4178-4261, 6316-6400, 8475-8558, 10701-10768, 12913-12994, 15156-15214, 17265-17349, 19464-19518, 21065-21117).

For a HIV subtype and HLA allele A0205, the antigen-encoding sequence for inclusion in the vaccine was selected by reference to the table corresponding to the HIV subtype, where each relevant sequence considered is selected by identifying all rows in that table that list A0205 (e.g., any of SEQ ID NOs: 470-526, 2327-2379, 6401-6450, 8559-8626, 10769-10822, 12995-13056, 15215-15263, 17350-17405, 19519-19570, and 21118-21161).

For a HIV subtype and HLA allele A0206, the antigen-encoding sequence for inclusion in the vaccine was selected by reference to the table corresponding to the HIV subtype, where each relevant sequence considered is selected by identifying all rows in that table that list A0206 (e.g., any of SEQ ID NOs: 527-565, 2380-2421, 6451-6492, 8627-8671, 10823-10867, 10357-13098, 15264-15292, 17406-17448, 19571-19604, and 21162-21192).

For a HIV subtype and HLA allele A0207, the antigen-encoding sequence for inclusion in the vaccine was selected by reference to the table corresponding to the HIV subtype, where each relevant sequence considered is selected by identifying all rows in that table that list A0207 (e.g., any of SEQ ID NOs: 566-587, 2422-2438, 6493-6509, 8672-8689, 10868-10887, 13099-13125, 15293-15307, 17449-17473, 19605-19618, and 21193-21205).

For a HIV subtype and HLA allele A0208, the antigen-encoding sequence for inclusion in the vaccine was selected by reference to the table corresponding to the HIV subtype, where each relevant sequence considered is selected by identifying all rows in that table that list A0208 (e.g., any of SEQ ID NOs: 588-630, 2439-2477, 6510-6548, 8690-8733, 10888-10931, 13126-13179, 15308-15336, 17474-17512, 19619-19649, and 21206-21233).

For a HIV subtype and HLA allele A0301, the antigen-encoding sequence for inclusion in the vaccine was selected by reference to the table corresponding to the HIV subtype, where each relevant sequence considered is selected by identifying all rows in that table that list A0301 (e.g., any of SEQ ID NOs: 631-650, 2478-2501, 6549-6573, 8734-8761, 10932-10969, 13180-13224, 15337-15354, 17513-17543, 19650-19665, and 21234-21247).

For a HIV subtype and HLA allele A0302, the antigen-encoding sequence for inclusion in the vaccine was selected by reference to the table corresponding to the HIV subtype, where each relevant sequence considered is selected by identifying all rows in that table that list A0302 (e.g., any of SEQ ID NOs: 651-682, 2502-2541, 6574-6618, 8762-8809, 10970-11026, 13225-13290, 15355-15396, 17544-17603, 19666-19697, and 21248-21274).

For a HIV subtype and HLA allele A1011, the antigen-encoding sequence for inclusion in the vaccine was selected by reference to the table corresponding to the HIV subtype, where each relevant sequence considered is selected by identifying all rows in that table that list A1011 (e.g., any of SEQ ID NOs: 683-726, 2542-2583, 6619-6668, 8810-8862, 11027-11087, 13291-13370, 15397-15451, 17604-17652, 19698-19726, and 21275-21309).

For a HIV subtype and HLA allele A2301, the antigen-encoding sequence for inclusion in the vaccine was selected by reference to the table corresponding to the HIV subtype, where each relevant sequence considered is selected by identifying all rows in that table that list A2301 (e.g., any of SEQ ID NOs: 727-741, 2584-2593, 6669-6685, 8863-8871, 11088-11103, 13371-13385, 15452-15465, 17653-17667, 19727-19738, and 21310-21317).

For a HIV subtype and HLA allele A2302, the antigen-encoding sequence for inclusion in the vaccine was selected by reference to the table corresponding to the HIV subtype, where each relevant sequence considered is selected by identifying all rows in that table that list A2402 (e.g., any of SEQ ID NOs: 742-755, 2594-2605, 6686-6698, 8872-8885, 11104-11116, 13386-13397, 15466-15479, 17668-17679, 19739-19750, and 21318-21323).

For a HIV subtype and HLA allele A2501, the antigen-encoding sequence for inclusion in the vaccine was selected by reference to the table corresponding to the HIV subtype, where each relevant sequence considered is selected by identifying all rows in that table that list A2501 (e.g., any of SEQ ID NOs: 756-769, 2606-2622, 6699-6711, 8886-8903, 11117-11132, 13398-13414, 15480-15505, 17680-17693, 19751-19760, and 21324-21333).

For a HIV subtype and HLA allele A2601, the antigen-encoding sequence for inclusion in the vaccine was selected by reference to the table corresponding to the HIV subtype, where each relevant sequence considered is selected by identifying all rows in that table that list A2601 (e.g., any of SEQ ID NOs: 770-783, 2623-2640, 6712-6728, 8904-8927, 11133-11155, 13415-13433, 15506-15533, 17694-17714, 19761-19773, and 21334-21346).

For a HIV subtype and HLA allele A2602, the antigen-encoding sequence for inclusion in the vaccine was selected by reference to the table corresponding to the HIV subtype, where each relevant sequence considered is selected by identifying all rows in that table that list A2602 (e.g., any of SEQ ID NOs: 784-790, 2641-2652, 6729-6739, 8928-8937, 11156-11168, 13434-13446, 1553-15550, 17715-17723, 19774-19782, and 21347-21353).

For a HIV subtype and HLA allele A2603, the antigen-encoding sequence for inclusion in the vaccine was selected by reference to the table corresponding to the HIV subtype, where each relevant sequence considered is selected by identifying all rows in that table that list A2603 (e.g., any of SEQ ID NOs: 791-802, 2653-2671, 6740-6759, 8938-8959, 11169-11189, 13447-13464, 15551-15569, 17724-17739, 19783-19797, and 21354-21360).

For a HIV subtype and HLA allele A2901, the antigen-encoding sequence for inclusion in the vaccine was selected by reference to the table corresponding to the HIV subtype, where each relevant sequence considered is selected by identifying all rows in that table that list A2901 (e.g., any of SEQ ID NOs: 803-814, 2672-2679, 6760-6768, 8960-8976, 11190-11195, 13465-13474, 15570-15588, 17740-17751, 19798-19808, and 21361-21366).

For a HIV subtype and HLA allele A2902, the antigen-encoding sequence for inclusion in the vaccine was selected by reference to the table corresponding to the HIV subtype, where each relevant sequence considered is selected by identifying all rows in that table that list A2902 (e.g., any of SEQ ID NOs: 815-828, 2680-2698, 6769-6784, 8977-9000, 11196-11210, 13475-13493, 15589-15612, 17752-17773, 19809-19821, and 21367-21376).

For a HIV subtype and HLA allele A3001, the antigen-encoding sequence for inclusion in the vaccine was selected by reference to the table corresponding to the HIV subtype, where each relevant sequence considered is selected by identifying all rows in that table that list A3001 (e.g., any of SEQ ID NOs: 829-842, 2699-2707, 6785-6793, 9001-9012, 11211-11216, 13494-13501, 15613-15617, 17774-17781, 19822-19828, and 21377-21383).

For a HIV subtype and HLA allele A3002, the antigen-encoding sequence for inclusion in the vaccine was selected by reference to the table corresponding to the HIV subtype, where each relevant sequence considered is selected by identifying all rows in that table that list A3002 (e.g., any of SEQ ID NOs: 843-857, 2708-2722, 6794-6807, 9013-9040, 11217-11235, 13502-13519, 15618-15636, 17782-17809, 19829-19843, and 21384-21390).

For a HIV subtype and HLA allele A3004, the antigen-encoding sequence for inclusion in the vaccine was selected by reference to the table corresponding to the HIV subtype, where each relevant sequence considered is selected by identifying all rows in that table that list A3004 (e.g., any of SEQ ID NOs: 858-864, 2723-2728, 6808-6817, 9041-9060, 11236-11246, 13520-13530, 15637-15649, 17810-17828, 19844-19850, and 21391-21393).

For a HIV subtype and HLA allele A3101, the antigen-encoding sequence for inclusion in the vaccine was selected by reference to the table corresponding to the HIV subtype, where each relevant sequence considered is selected by identifying all rows in that table that list A3101 (e.g., any of SEQ ID NOs: 865-895, 2729-2757, 6818-6846, 9061-9082, 11247-11272, 13531-13558, 15650-15683, 17829-17862, 19851-19869, and 21394-21407).

For a HIV subtype and HLA allele A3201, the antigen-encoding sequence for inclusion in the vaccine was selected by reference to the table corresponding to the HIV subtype, where each relevant sequence considered is selected by identifying all rows in that table that list A3201 (e.g., any of SEQ ID NOs: 896-899, 2758-2761, 6847-6850, 9083-9091, 11273-11275, 13559-13567, 15684-15688, 17863-17870, 19870-19874, and 21408-21409).

For a HIV subtype and HLA allele A3301, the antigen-encoding sequence for inclusion in the vaccine was selected by reference to the table corresponding to the HIV subtype, where each relevant sequence considered is selected by identifying all rows in that table that list A3301 (e.g., any of SEQ ID NOs: 900-920, 2762-2793, 6851-6880, 9092-9112, 11276-11300, 13568-13585, 15689-15707, 17871-17900, 19875-19898, and 21410-21425).

For a HIV subtype and HLA allele A3303, the antigen-encoding sequence for inclusion in the vaccine was selected by reference to the table corresponding to the HIV subtype, where each relevant sequence considered is selected by identifying all rows in that table that list A3303 (e.g., any of SEQ ID NOs: 921-955, 2794-2851, 6881-6935, 9113-9164, 11301-11346, 13586-13619, 15708-15742, 17901-17964, 19899-19933, and 21426-21459).

For a HIV subtype and HLA allele A6801, the antigen-encoding sequence for inclusion in the vaccine was selected by reference to the table corresponding to the HIV subtype, where each relevant sequence considered is selected by identifying all rows in that table that list A6801 (e.g., any of SEQ ID NOs: 956-997, 2852-2908, 6936-6986, 9165-9228, 11347-11410, 13620-13667, 15743-15785, 17965-18029, 19934-19986, and 21460-24192).

For a HIV subtype and HLA allele A6802, the antigen-encoding sequence for inclusion in the vaccine was selected by reference to the table corresponding to the HIV subtype, where each relevant sequence considered is selected by identifying all rows in that table that list A6802 (e.g., any of SEQ ID NOs: 998-1032, 2909-2946, 6897-7037, 9229-9292, 11411-11461, 13668-13715, 15786-15828, 18030-18068, 19987-20027, and 24193-21523).

For a HIV subtype and HLA allele B0702, the antigen-encoding sequence for inclusion in the vaccine was selected by reference to the table corresponding to the HIV subtype, where each relevant sequence considered is selected by identifying all rows in that table that list B0702 (e.g., any of SEQ ID NOs: 1033-1050, 2947-2969, 7038-7065, 9293-9312, 11462-11485, 13716-13738, 15829-15849, 18069-18091, 20028-20038, and 21524-21540).

For a HIV subtype and HLA allele B0801, the antigen-encoding sequence for inclusion in the vaccine was selected by reference to the table corresponding to the HIV subtype, where each relevant sequence considered is selected by identifying all rows in that table that list B0801 (e.g., any of SEQ ID NOs: 1051-1066, 2970-2984, 7066-7078, 9313-9325, 11486-11497, 13739-13752, 15850-15862, 18092-18112, 20039-20051, and 21541-21549).

For a HIV subtype and HLA allele B1301, the antigen-encoding sequence for inclusion in the vaccine was selected by reference to the table corresponding to the HIV subtype, where each relevant sequence considered is selected by identifying all rows in that table that list B1301 (e.g., any of SEQ ID NOs: 1067-1080, 2985-2999, 7079-7095, 9326-9347, 11498-11516, 13753-13767, 15863-15875, 18113-18128, 20052-20062, and 21550-21557).

For a HIV subtype and HLA allele B1302, the antigen-encoding sequence for inclusion in the vaccine was selected by reference to the table corresponding to the HIV subtype, where each relevant sequence considered is selected by identifying all rows in that table that list B1302 (e.g., any of SEQ ID NOs: 1081-1117, 3000-3052, 7096-7140, 9348-9406, 11517-11557, 13768-13821, 15876-15923, 18129-18178, 20063-20093, and 21558-21593).

For a HIV subtype and HLA allele B1401, the antigen-encoding sequence for inclusion in the vaccine was selected by reference to the table corresponding to the HIV subtype, where each relevant sequence considered is selected by identifying all rows in that table that list B1401 (e.g., any of SEQ ID NOs: 1118-1125, 3053-3058, 7141-7145, 9407-9411, 11558-11562, 13822-13827, 15924-15931, 18179-18185, 20094-20098, and 21594-21599).

For a HIV subtype and HLA allele B1402, the antigen-encoding sequence for inclusion in the vaccine was selected by reference to the table corresponding to the HIV subtype, where each relevant sequence considered is selected by identifying all rows in that table that list B1402 (e.g., any of SEQ ID NOs: 1126-1139, 3059-3070, 7146-7159, 9412-9418, 11563-11574, 13828-13837, 15932-15943, 18186-18197, 20099-20109, and 21600-21606).

For a HIV subtype and HLA allele B1501, the antigen-encoding sequence for inclusion in the vaccine was selected by reference to the table corresponding to the HIV subtype, where each relevant sequence considered is selected by identifying all rows in that table that list B1501 (e.g., any of SEQ ID NOs: 1140-1192, 3071-3111, 7160-7211, 9419-9481, 11575-11633, 13838-13895, 15944-16001, 18198-18259, 20110-20141, and 21607-21635).

For a HIV subtype and HLA allele B1502, the antigen-encoding sequence for inclusion in the vaccine was selected by reference to the table corresponding to the HIV subtype, where each relevant sequence considered is selected by identifying all rows in that table that list B1502 (e.g., any of SEQ ID NOs: 1193-1220, 3112-3135, 7212-7247, 9482-9501, 11634-11670, 13896-13937, 16002-16036, 18260-18300, 20142-20165, and 21636-21656).

For a HIV subtype and HLA allele B1503, the antigen-encoding sequence for inclusion in the vaccine was selected by reference to the table corresponding to the HIV subtype, where each relevant sequence considered is selected by identifying all rows in that table that list B1503 (e.g., any of SEQ ID NOs: 1221-1245, 3136-3152, 7248-7273, 9502-9526, 11671-11693, 13938-13968, 16037-16065, 18301-18324, 20166-20179, and 21657-21669).

For a HIV subtype and HLA allele B1510, the antigen-encoding sequence for inclusion in the vaccine was selected by reference to the table corresponding to the HIV subtype, where each relevant sequence considered is selected by identifying all rows in that table that list B1510 (e.g., any of SEQ ID NOs: 1246-1266, 3153-3178, 7274-7296, 9527-9548, 11694-11722, 13969-13995, 16066-16083, 18325-18352, 20180-20200, and 21670-21689).

For a HIV subtype and HLA allele B1513, the antigen-encoding sequence for inclusion in the vaccine was selected by reference to the table corresponding to the HIV subtype, where each relevant sequence considered is selected by identifying all rows in that table that list B1513 (e.g., any of SEQ ID NOs: 1267-1270, 3179-3183, 7297-7300, 9549-9551, 11723-11725, 13996-14005, 16084-16091, 18353-18358, 20201-20205, and 21690-21692).

For a HIV subtype and HLA allele B1801, the antigen-encoding sequence for inclusion in the vaccine was selected by reference to the table corresponding to the HIV subtype, where each relevant sequence considered is selected by identifying all rows in that table that list B1801 (e.g., any of SEQ ID NOs: 1271-1286, 3184-3203, 7301-7328, 9552-9565, 11726-11742, 14006-14024, 16092-16107, 18359-18375, 20206-20224, and 21693-21705).

For a HIV subtype and HLA allele B2702, the antigen-encoding sequence for inclusion in the vaccine was selected by reference to the table corresponding to the HIV subtype, where each relevant sequence considered is selected by identifying all rows in that table that list B2702 (e.g., any of SEQ ID NOs: 1287-1304, 3204-3225, 7329-7355, 9566-9594, 11743-11756, 14025-14048, 16108-16135, 18376-18408, 20225-20241, and 21706-21716).

For a HIV subtype and HLA allele B2705, the antigen-encoding sequence for inclusion in the vaccine was selected by reference to the table corresponding to the HIV subtype, where each relevant sequence considered is selected by identifying all rows in that table that list B2705 (e.g., any of SEQ ID NOs: 1305-1319, 3226-3234, 7356-7370, 9595-9610, 11757-11771, 14049-14063, 16136-16145, 18409-18422, 20242-20254, and 21717-21723)-.

For a HIV subtype and HLA allele B3501, the antigen-encoding sequence for inclusion in the vaccine was selected by reference to the table corresponding to the HIV subtype, where each relevant sequence considered is selected by identifying all rows in that table that list B3501 (e.g., any of SEQ ID NOs: 1320-1338, 3235-3260, 7371-7405, 9611-9641, 11772-11812, 14064-14095, 16146-16186, 18423-18463, 20255-20279, and 21724-21745).

For a HIV 4subtype and HLA allele B3502, the antigen-encoding sequence for inclusion in the vaccine was selected by reference to the table corresponding to the HIV subtype, where each relevant sequence considered is selected by identifying all rows in that table that list B3502 (e.g., any of SEQ ID NOs: 1339-1349, 3261-3272, 7406-7424, 9642-9661, 11813-11833, 14096-14112, 16187-16205, 18464-18482, 20280-20291, and 21746-21754).

For a HIV subtype and HLA allele B3503, the antigen-encoding sequence for inclusion in the vaccine was selected by reference to the table corresponding to the HIV subtype, where each relevant sequence considered is selected by identifying all rows in that table that list B3503 (e.g., any of SEQ ID NOs: 1350-1373, 3273-3298, 7425-7457, 9662-9697, 11834-11877, 14113-14148, 16206-16238, 18483-18513, 20292-20316, and 21755-21772).

For a HIV subtype and HLA allele B3508, the antigen-encoding sequence for inclusion in the vaccine was selected by reference to the table corresponding to the HIV subtype, where each relevant sequence considered is selected by identifying all rows in that table that list B3508 (e.g., any of SEQ ID NOs: 1374-1386, 3299-3309, 7458-7477, 9698-9719, 11878-11899, 14149-14166, 16239-16256, 18514-18538, 20317-20331, and 21773-21786).

For a HIV subtype and HLA allele B3512, the antigen-encoding sequence for inclusion in the vaccine was selected by reference to the table corresponding to the HIV subtype, where each relevant sequence considered is selected by identifying all rows in that table that list B3512 (e.g., any of SEQ ID NOs: 1387-1405, 3310-3326, 7478-7498, 9720-9744, 11900-11930, 14167-14185, 16257-16280, 18539-18560, 20332-20344, and 21787-21799).

For a HIV subtype and HLA allele B3701, the antigen-encoding sequence for inclusion in the vaccine was selected by reference to the table corresponding to the HIV subtype, where each relevant sequence considered is selected by identifying all rows in that table that list BB3701 (e.g., any of SEQ ID NOs: 1406-1425, 3327-3338, 7499-7512, 9745-9757, 11931-11944, 14186-14196, 16281-16291, 18561-18572, 20345-20359, and 21800-21808).

For a HIV subtype and HLA allele B3801, the antigen-encoding sequence for inclusion in the vaccine was selected by reference to the table corresponding to the HIV subtype, where each relevant sequence considered is selected by identifying all rows in that table that list B3801 (e.g., any of SEQ ID NOs: 1426-1451, 3339-3367, 7513-7533, 9758-9782, 11945-11970, 14197-14219, 16292-16310, 18573-18599, 20360-20381, and 21809-21828).

For a HIV subtype and HLA allele B3901, the antigen-encoding sequence for inclusion in the vaccine was selected by reference to the table corresponding to the HIV subtype, where each relevant sequence considered is selected by identifying all rows in that table that list B3901 (e.g., any of SEQ ID NOs: 1452-1476, 3368-3391, 7534-7551, 9783-9802, 11971-11992, 14220-14242, 16311-16323, 18600-18619, 20382-20395, and 21829-21844).

For a HIV subtype and HLA allele B3906, the antigen-encoding sequence for inclusion in the vaccine was selected by reference to the table corresponding to the HIV subtype, where each relevant sequence considered is selected by identifying all rows in that table that list B3906 (e.g., any of SEQ ID NOs: 1477-1499, 3392-3423, 7552-7571, 9803-9831, 11993-12020, 14243-14277, 16324-16349, 18620-18653, 20396-20411, and 21845-21861).

For a HIV subtype and HLA allele B4001, the antigen-encoding sequence for inclusion in the vaccine was selected by reference to the table corresponding to the HIV subtype, where each relevant sequence considered is selected by identifying all rows in that table that list B4001 (e.g., any of SEQ ID NOs: 1500-1527, 3424-3458, 7572-7614, 9832-9867, 12021-12057, 14278-14309, 16350-16384, 18654-18686, 20412-20431, and 21862-21888).

For a HIV subtype and HLA allele B4002, the antigen-encoding sequence for inclusion in the vaccine was selected by reference to the table corresponding to the HIV subtype, where each relevant sequence considered is selected by identifying all rows in that table that list B4002 (e.g., any of SEQ ID NOs: 1528-1576, 3459-3497, 7615-7665, 9868-9913, 12058-12110, 14310-14359, 16385-16431, 18687-18736, 20432-20460, and 21889-21924).

For a HIV subtype and HLA allele B4006, the antigen-encoding sequence for inclusion in the vaccine was selected by reference to the table corresponding to the HIV subtype, where each relevant sequence considered is selected by identifying all rows in that table that list B4006 (e.g., any of SEQ ID NOs: 1577-1593, 3498-3517, 7666-7689, 9914-9942, 12111-12136, 14360-14380, 16432-16463, 18737-18759, 20461-20479, and 21925-21940).

For a HIV subtype and HLA allele 4102, the antigen-encoding sequence for inclusion in the vaccine was selected by reference to the table corresponding to the HIV subtype, where each relevant sequence considered is selected by identifying all rows in that table that list B4102 (e.g., any of SEQ ID NOs: 1594-1642, 3518-3554, 7690-7742, 9943-9988, 12137-12175, 14381-14429, 16437-16510, 18760-18811, 20480-20512, and 21941-21975).

For a HIV subtype and HLA allele B4402, the antigen-encoding sequence for inclusion in the vaccine was selected by reference to the table corresponding to the HIV subtype, where each relevant sequence considered is selected by identifying all rows in that table that list B4402 (e.g., any of SEQ ID NOs: 1643-1663, 3555-3575, 7743-7772, 9989-10011, 12176-12202, 14430-14448, 16510-16527, 18812-18834, 20513-20530, and 21976-21992).

For a HIV subtype and HLA allele B4403, the antigen-encoding sequence for inclusion in the vaccine was selected by reference to the table corresponding to the HIV subtype, where each relevant sequence considered is selected by identifying all rows in that table that list B4403 (e.g., any of SEQ ID NOs: 1664-1697, 3576-3611, 7773-7826, 10012-10058, 12203-12254, 14449-14493, 16528-16562, 18835-18883, 20531-20564, and 21993-22024).

For a HIV subtype and HLA allele B4405, the antigen-encoding sequence for inclusion in the vaccine was selected by reference to the table corresponding to the HIV subtype, where each relevant sequence considered is selected by identifying all rows in that table that list B4405 (e.g., any of SEQ ID NOs: 1698-1745, 3612-3674, 7827-7903, 10059-10134, 12255-12327, 14494-14560, 16563-16633, 18884-18953, 20565-20613, and 22025-22067).

For a HIV subtype and HLA allele B4601, the antigen-encoding sequence for inclusion in the vaccine was selected by reference to the table corresponding to the HIV subtype, where each relevant sequence considered is selected by identifying all rows in that table that list B4601 (e.g., any of SEQ ID NOs: 1746-1752, 3675-3679, 7904-7910, 10135-10146, 12328-12339, 14561-14574, 16634-16645, 18954-18957, 20614-20619, and 22068-22069).

For a HIV subtype and HLA allele B4801, the antigen-encoding sequence for inclusion in the vaccine was selected by reference to the table corresponding to the HIV subtype, where each relevant sequence considered is selected by identifying all rows in that table that list B4801 (e.g., any of SEQ ID NOs: 1753-1785, 3680-3695, 7911-7926, 10147-10161, 12340-12359, 14575-14596, 16646-16664, 18958-18974, 20620-20636, and 22070-22081).

For a HIV subtype and HLA allele B4901, the antigen-encoding sequence for inclusion in the vaccine was selected by reference to the table corresponding to the HIV subtype, where each relevant sequence considered is selected by identifying all rows in that table that list B4901 (e.g., any of SEQ ID NOs: 1786-1824, 3696-3719, 7927-7967, 10162-10207, 12360-12395, 14597-14634, 16665-16709, 18975-19013, 20637-20656, and 22082-22109).

For a HIV subtype and HLA allele B5001, the antigen-encoding sequence for inclusion in the vaccine was selected by reference to the table corresponding to the HIV subtype, where each relevant sequence considered is selected by identifying all rows in that table that list B5001 (e.g., any of SEQ ID NOs: 1825-1855, 3720-3755, 7968-8008, 10208-10251, 12396-12438, 14635-14675, 16710-16748, 19014-19051, 20657-20682, and 22110-22129).

For a HIV subtype and HLA allele B5101, the antigen-encoding sequence for inclusion in the vaccine was selected by reference to the table corresponding to the HIV subtype, where each relevant sequence considered is selected by identifying all rows in that table that list B5101 (e.g., any of SEQ ID NOs: 1856-1872, 3756-3789, 8009-8037, 10252-10287, 12439-12467, 14676-14708, 16749-16783, 19052-19076, 20683-20711, and 22130-22158).

For a HIV subtype and HLA allele B5401, the antigen-encoding sequence for inclusion in the vaccine was selected by reference to the table corresponding to the HIV subtype, where each relevant sequence considered is selected by identifying all rows in that table that list B5401 (e.g., any of SEQ ID NOs: 1873-1900, 3790-3823, 8038-8075, 10288-10327, 12468-12507, 14709-14745, 16784-16826, 19077-19108, 207120-20748, and 22159-22178).

For a HIV subtype and HLA allele B5501, the antigen-encoding sequence for inclusion in the vaccine was selected by reference to the table corresponding to the HIV subtype, where each relevant sequence considered is selected by identifying all rows in that table that list B5501 (e.g., any of SEQ ID NOs: 1901-1907, 3824-3827, 8076-8088, 10328-10341, 12508-12520, 14746-14756, 16827-16841, 19109-19113, 20749-20759, and 22179-22184).

For a HIV subtype and HLA allele B5502, the antigen-encoding sequence for inclusion in the vaccine was selected by reference to the table corresponding to the HIV subtype, where each relevant sequence considered is selected by identifying all rows in that table that list B5502 (e.g., any of SEQ ID NOs: 1908-1924, 3828-3843, 8089-8109, 10342-10364, 12521-12543, 14757-14777, 16842-16867, 19114-19135, 20760-20785, and 22185-22194).

For a HIV subtype and HLA allele B5601, the antigen-encoding sequence for inclusion in the vaccine was selected by reference to the table corresponding to the HIV subtype, where each relevant sequence considered is selected by identifying all rows in that table that list B5601 (e.g., any of SEQ ID NOs: 1925-1945, 3844-3865, 8110-8136, 10365-10392, 12544-12565, 14778-14802, 16868-16897, 19136-19156, 20786-20810, and 22195-22209).

For a HIV subtype and HLA allele B5701, the antigen-encoding sequence for inclusion in the vaccine was selected by reference to the table corresponding to the HIV subtype, where each relevant sequence considered is selected by identifying all rows in that table that list B5701 (e.g., any of SEQ ID NOs: 1946-1985, 3866-3908, 8137-8188, 10393-10441, 12566-12606, 14803-14849, 16898-16956, 19157-19202, 20811-20848, and 22210-22234).

For a HIV subtype and HLA allele B5801, the antigen-encoding sequence for inclusion in the vaccine was selected by reference to the table corresponding to the HIV subtype, where each relevant sequence considered is selected by identifying all rows in that table that list B5801 (e.g., any of SEQ ID NOs: 1986-2019, 3909-3942, 8189-8218, 10442-10467, 12607-12632, 14850-14873, 16957-16992, 19203-19232, 20849-20875, and 22235-22252).

For a HIV subtype and HLA allele C0102, the antigen-encoding sequence for inclusion in the vaccine was selected by reference to the table corresponding to the HIV subtype, where each relevant sequence considered is selected by identifying all rows in that table that list C0102 (e.g., any of SEQ ID NOs: 2020-2026, 3943-3945, 8219-8224, 10468-10472, 12633-12644, 14874-14881, 16993-16996, 19233-19242, 20876-20880, and 22253-22255).

For a HIV subtype and HLA allele C0202, the antigen-encoding sequence for inclusion in the vaccine was selected by reference to the table corresponding to the HIV subtype, where each relevant sequence considered is selected by identifying all rows in that table that list C0202 (e.g., any of SEQ ID NOs: 2027-2028, 3946-3947, 8225-8227, 10473-10476, 12645-12647, 14882-14887, 16997-16999, 19243-19245, 20881-20883, and 22256-22262).

For a HIV subtype and HLA allele C0302, the antigen-encoding sequence for inclusion in the vaccine was selected by reference to the table corresponding to the HIV subtype, where each relevant sequence considered is selected by identifying all rows in that table that list C0302 (e.g., any of SEQ ID NOs: 2029-2034, 3948-3956, 8228-8233, 10477-10484, 12648-12657, 14888-14900, 17000-17007, 19246-19253, 20884-20888, and 22263-22266).

For a HIV subtype and HLA allele C0303, the antigen-encoding sequence for inclusion in the vaccine was selected by reference to the table corresponding to the HIV subtype, where each relevant sequence considered is selected by identifying all rows in that table that list C0303 (e.g., any of SEQ ID NOs: 2035-2039, 3957-3962, 8234-8239, 10485-10491, 12658-12663, 14901-14911, 17008-17016, 19254-19257, 20889-20893, and 22267-22272).

For a HIV subtype and HLA allele C0304, the antigen-encoding sequence for inclusion in the vaccine was selected by reference to the table corresponding to the HIV subtype, where each relevant sequence considered is selected by identifying all rows in that table that list C0304 (e.g., any of SEQ ID NOs: 2040-2047, 3963-3974, 8240-8250, 10492-10502, 12664-12676, 14912-14927, 17017-17029, 19258-19270, 20894-20901, and 22273-22274).

For a HIV subtype and HLA allele C0401, the antigen-encoding sequence for inclusion in the vaccine was selected by reference to the table corresponding to the HIV subtype, where each relevant sequence considered is selected by identifying all rows in that table that list C0401 (e.g., any of SEQ ID NOs: 2048-2052, 3975-3979, 8251-8257, 10503-10505, 12677-12680, 14928-14932, 17030-17033, 19271-19277, 20902-20903, and 22275-22281).

For a HIV subtype and HLA allele C0501, the antigen-encoding sequence for inclusion in the vaccine was selected by reference to the table corresponding to the HIV subtype, where each relevant sequence considered is selected by identifying all rows in that table that list C0501 (e.g., any of SEQ ID NOs: 2053-2057, 3980-3992, 8258-8262, 10506-10514, 12681-12692, 14933-14944, 17034-17041, 19278-19288, 20904-20911, and 22282-22283).

For a HIV subtype and HLA allele C0602, the antigen-encoding sequence for inclusion in the vaccine was selected by reference to the table corresponding to the HIV subtype, where each relevant sequence considered is selected by identifying all rows in that table that list C0602 (e.g., any of SEQ ID NOs: 2058-2059, 3993-3995, 8263, 10515-10518, 12693-12697, 14945-14948, 17042-17045, 19289-19290, 20912-20913, 22284-22295).

For a HIV subtype and HLA allele C0701, the antigen-encoding sequence for inclusion in the vaccine was selected by reference to the table corresponding to the HIV subtype, where each relevant sequence considered is selected by identifying all rows in that table that list C0701 (e.g., any of SEQ ID NOs: 8264 and 17046).

For a HIV subtype and HLA allele C0702, the antigen-encoding sequence for inclusion in the vaccine was selected by reference to the table corresponding to the HIV subtype, where each relevant sequence considered is selected by identifying all rows in that table that list C0702 (e.g., any of SEQ ID NOs: 2060, 3996-3997, 12698, and 14949).

For a HIV subtype and HLA allele C0704, the antigen-encoding sequence for inclusion in the vaccine was selected by reference to the table corresponding to the HIV subtype, where each relevant sequence considered is selected by identifying all rows in that table that list C0704 (e.g., any of SEQ ID NOs: 2061, 3998, 10519, and 17047).

For a HIV subtype and HLA allele C0801, the antigen-encoding sequence for inclusion in the vaccine was selected by reference to the table corresponding to the HIV subtype, where each relevant sequence considered is selected by identifying all rows in that table that list C0801 (e.g., any of SEQ ID NOs: 2062-2079, 3999-4013, 8265-8274, 10520-10533, 12699-12721, 14950-14974, 17048-17069, 19291-19304, 20914-20923, and 22284-22295).

For a HIV subtype and HLA allele C0802, the antigen-encoding sequence for inclusion in the vaccine was selected by reference to the table corresponding to the HIV subtype, where each relevant sequence considered is selected by identifying all rows in that table that list C0802 (e.g., any of SEQ ID NOs: 2080-2088, 4014-4031, 8275-8288, 10534-10545, 12722-12739, 14975-14987, 17070-1076, 19305-19321, 20924-20929, and 22296-22300).

For a HIV subtype and HLA allele C0803, the antigen-encoding sequence for inclusion in the vaccine was selected by reference to the table corresponding to the HIV subtype, where each relevant sequence considered is selected by identifying all rows in that table that list C0803 (e.g., any of SEQ ID NOs: 2089-2100, 4032-4035, 8289-8295, 10546-10548, 12740-12742, 14988-14997, 17077-17079, 19322-19324, 20930-20938, and 22301-22304).

For a HIV subtype and HLA allele C1203, the antigen-encoding sequence for inclusion in the vaccine was selected by reference to the table corresponding to the HIV subtype, where each relevant sequence considered is selected by identifying all rows in that table that list C1203 (e.g., any of SEQ ID NOs: 2101-2105, 4036-4043, 8296-8302, 10549-10555, 102743-12748, 14998-15007, 17080-17089, 19325-19332, 20939-20947, and 22305-22310).

For a HIV subtype and HLA allele C1402, the antigen-encoding sequence for inclusion in the vaccine was selected by reference to the table corresponding to the HIV subtype, where each relevant sequence considered is selected by identifying all rows in that table that list C1402 (e.g., any of SEQ ID NOs: 2106-2122, 4044-4058, 8303-8329, 10556-10574, 12749-12763, 15008-15025, 17090-17108, 19333-19348, 20948-20962, and 22311-22320).

For a HIV subtype and HLA allele C1403, the antigen-encoding sequence for inclusion in the vaccine was selected by reference to the table corresponding to the HIV subtype, where each relevant sequence considered is selected by identifying all rows in that table that list C1403 (e.g., any of SEQ ID NOs: 2123-2133, 4059-4069, 8330-8342, 10575-10587, 12764-12772 15026-15035, 17109-17124, 19349-19361, 20963-20970, and 22321-22327).

For a HIV subtype and HLA allele C1502, the antigen-encoding sequence for inclusion in the vaccine was selected by reference to the table corresponding to the HIV subtype, where each relevant sequence considered is selected by identifying all rows in that table that list C1502 (e.g., any of SEQ ID NOs: 2134-2138, 4070-4074, 8343-8354, 10588-10591, 12773-12778, 15036-15040, 17125-17135, 19362-19366, 20971-20978, and 22328-22332).

For a HIV subtype and HLA allele C1601, the antigen-encoding sequence for inclusion in the vaccine was selected by reference to the table corresponding to the HIV subtype, where each relevant sequence considered is selected by identifying all rows in that table that list C1601 (e.g., any of SEQ ID NOs: 2139-2143, 4075-4079, 8355-8358, 10592-10595, 12779-12782, 15041-15048, 17136-17144, 19367-19370, 20979-20983, and 22333-22334).

For a HIV subtype and HLA allele C1602, the antigen-encoding sequence for inclusion in the vaccine was selected by reference to the table corresponding to the HIV subtype, where each relevant sequence considered is selected by identifying all rows in that table that list C1602 (e.g., any of SEQ ID NOs: 2144-2151, 4080-4089, 8359-8367, 10596-10602, 12783-12792, 15049-15058, 17145-17157, 19371-19376, 20984-20992, and 22335-22340).

For a HIV subtype and HLA allele C1604, the antigen-encoding sequence for inclusion in the vaccine was selected by reference to the table corresponding to the HIV subtype, where each relevant sequence considered is selected by identifying all rows in that table that list C1604 (e.g., any of SEQ ID NOs: 2152-2160, 4090-4098, 8368-8381, 10603-10615, 12793-12803, 15059-15069, 17158-17165, 19377-19382, 20994-20998, and 22341-22345).

For a HIV subtype and HLA allele C1701, the antigen-encoding sequence for inclusion in the vaccine was selected by reference to the table corresponding to the HIV subtype, where each relevant sequence considered is selected by identifying all rows in that table that list C1701 (e.g., any of SEQ ID NOs: 2161-2165, 4099-4106, 8382-8389, 10616-10626, 12804-12810, 15070-15079, 17166-17174, 19383-19388, 20999-21003, and 22346-22349).

### XXI. Example 9: Evaluation of T Cell Recognition of Candidate Antigens

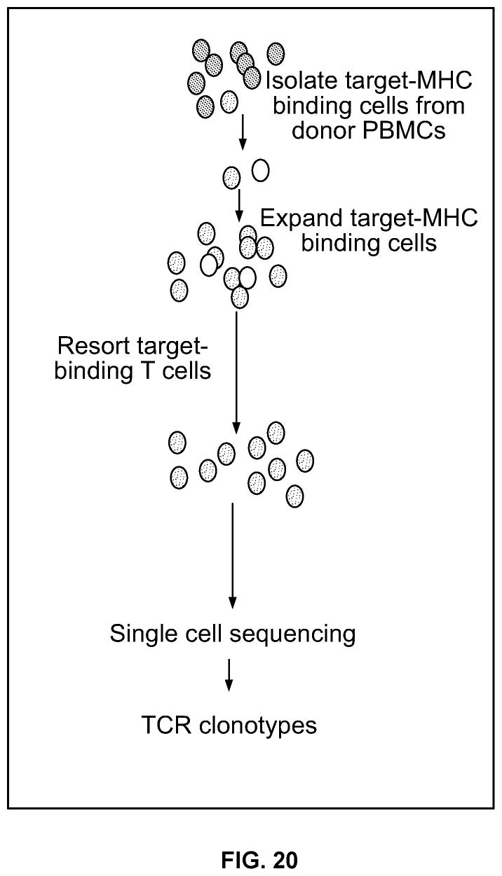

The candidate antigens are evaluated to determine whether they induce an immune response in patients. Specifically, peripheral blood mononuclear cells (PBMCs) from healthy donors are enriched for naïve CD8+ T cells. The healthy donors are confirmed to have the HLA allele B4102. The CD8+ T cells are stained with MHC multimers presenting several of the candidate antigens including epitope sequences present in the vaccine cassette: a first antigen including the epitope sequence “AEVVQKVTM (SEQ ID NO: 148)” and a second antigen including the epitope sequence “AEVVQKVVM (SEQ ID NO: 149).” HLA-peptide binding cells are sorted, expanded and their specificity for the antigens are confirmed. TCR sequencing of antigen-specific T cells is performed. FIG. 20 illustrates the general TCR sequencing strategy and workflow. TCR sequencing strategy reveal a polyclonal response. The naïve T cell repertoire analysis suggests the candidate antigens are expected to induce an immune response in select patients when administered by vaccination.

### XXI. Example 10: Performance of EDGE Machine Learning Model for Identifying HIV Epitopes

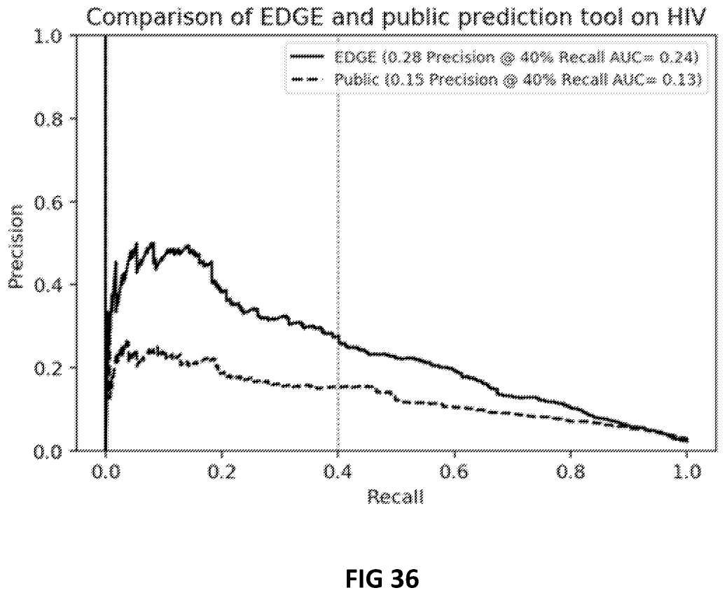

FIG. 36 depicts the predictive capacity of the EDGE machine learning model in comparison to a public prediction tool for predicting HIV epitopes that are presented by class I HLA alleles. Specifically, the EDGE machine learning model was trained on 507,502 peptides presented in mass spectrometry across 398 samples and covers 116 identified alleles. The EDGE machine learning model generates per-allele scores, each per-allele score representing a likelihood that a HIV epitope is presented by a particular class I HLA allele. Example alleles are shown in the column entitled “HLA” in any one of Tables 35-45. Additionally, these per-allele likelihoods can be aggregated to determine a likelihood that the HIV epitope is presented by at least one of the class I HLA alleles. In comparison, the public prediction tool for predicting HIV epitopes was the MHCflurry¹⁰⁷.

The EDGE model and MHCflurry were deployed on a test dataset of HIV CD8+ epitopes obtained from the Los Alamos HIV Database to predict HIV epitopes that are presented by at least one class I HLA allele. As shown in FIG. 36, the EDGE model outperforms MHCflurry. Specifically, at a 40% recall rate, the EDGE model exhibited a precision value of 0.28 whereas MHCflurry exhibited a precision value of 0.15. Additionally, EDGE outperforms MHCflurry with an area under the curve (AUC)=0.24 compared to 0.13.

Altogether, this Example demonstrates that the EDGE machine learning model is able to better predict HIV epitopes that are presented by one or more class I HLA alleles in comparison to a conventional, publicly available model. Therefore, in comparison to epitopes identified through conventional methods, epitopes identified through the use of the EDGE machine learning model, such as epitopes shown in the column entitled “Epitope sequence” in any one of Tables 35-45 (e.g., any one of SEQ ID Nos: 325-22349), are more likely to be presented by one or more class I HLA alleles.

### Certain Sequences

Vectors, cassettes, and antibodies referred to herein are described below and referred to by SEQ ID NO.

Tremelimumab VL (SEQ ID NO: 16)
Tremelimumab VH (SEQ ID NO: 17)
Tremelimumab VH CDR1 (SEQ ID NO: 18)
Tremelimumab VH CDR2 (SEQ ID NO: 19)
Tremelimumab VH CDR3 (SEQ ID NO: 20)
Tremelimumab VL CDR1 (SEQ ID NO: 21)
Tremelimumab VL CDR2 (SEQ ID NO: 22)
Tremelimumab VL CDR3 (SEQ ID NO: 23)
Durvalumab (MEDI4736) VL (SEQ ID NO: 24)
MEDI4736 VH (SEQ ID NO: 25)
MEDI4736 VH CDR1 (SEQ ID NO: 26)
MEDI4736 VH CDR2 (SEQ ID NO: 27)
MEDI4736 VH CDR3 (SEQ ID NO: 28)
MEDI4736 VL CDR1 (SEQ ID NO: 29)
MEDI4736 VL CDR2 (SEQ ID NO: 30)
MEDI4736 VL CDR3 (SEQ ID NO: 31)
UbA76-25merPDTT nucleotide (SEQ ID NO: 32)
UbA76-25merPDTT polypeptide (SEQ ID NO: 33)
MAG-25merPDTT nucleotide (SEQ ID NO: 34)
MAG-25merPDTT polypeptide (SEQ ID NO: 35)
Ub7625merPDTT_NoSFL nucleotide (SEQ ID NO: 36)
Ub7625merPDTT_NoSFL polypeptide (SEQ ID NO: 37)
ChAdV68.5WTnt.MAG25mer (SEQ ID NO: 2); AC_000011.1 with E1 (nt 577 to 3403)
and E3 (nt 27, 125-31, 825) sequences deleted; corresponding ATCC VR-594
nucleotides substituted at five positions; model antigen cassette under
the control of the CMV promoter/enhancer inserted in place of deleted E1;
SV40 polyA 3′ of cassette
Venezuelan equine encephalitis virus [VEE] (SEQ ID NO: 3) GenBank: L01442.2
VEE-MAG25mer (SEQ ID NO: 4); contains MAG-25merPDTT nucleotide (bases 30-1755)
Venezuelan equine encephalitis virus strain TC-83 [TC-83] (SEQ ID NO: 5)
GenBank: L01443.1
VEE Delivery Vector (SEQ ID NO: 6); VEE genome with nucleotides 7544-11175
deleted [alphavirus structural proteins removed]
TC-83 Delivery Vector (SEQ ID NO: 7); TC-83 genome with nucleotides 7544-
11175 deleted [alphavirus structural proteins removed]
VEE Production Vector (SEQ ID NO: 8); VEE genome with nucleotides 7544-
11175 deleted, plus 5′ T7-promoter, plus 3′ restriction sites
TC-83 Production Vector (SEQ ID NO: 9); TC-83 genome with nucleotides 7544-
11175 deleted, plus 5′ T7-promoter, plus 3′ restriction sites
VEE-UbAAY (SEQ ID NO: 14); VEE delivery vector with MHC class I mouse tumor
epitopes SIINFEKL (SEQ ID NO: 150) and AH1-A5 inserted
VEE-Luciferase (SEQ ID NO: 15); VEE delivery vector with luciferase gene
inserted at 7545
ubiquitin (SEQ ID NO: 38) > UbG76 0-228
Ubiquitin A76 (SEQ ID NO: 39) > UbA76 0-228
HLA-A2 (MHC class I) signal peptide (SEQ ID NO: 40) > MHC SignalPep 0-78
HLA-A2 (MHC class I) Trans Membrane domain (SEQ ID NO: 41) > HLA A2 TM Domain
0-201
IgK Leader Seq (SEQ ID NO: 42) > IgK Leader Seq 0-60
Human DC-Lamp (SEQ ID NO: 43) > HumanDCLAMP 0-3178
Mouse LAMP1 (SEQ ID NO: 44) > MouseLamp1 0-1858
Human Lamp1 cDNA (SEQ ID NO: 45) > Human Lamp1 0-2339
Tetanus toxoid nulceic acid sequence (SEQ ID NO: 46)
Tetanus toxoid amino acid sequence (SEQ ID NO: 47)
PADRE nulceotide sequence (SEQ ID NO: 48)
PADRE amino acid sequence (SEQ ID NO: 49)
WPRE (SEC ID NO: 50) > WPRE 0-593
IRES (SEQ ID NO: 51) > eGFP_IRES_SEAP_Insert 1746-2335
GFP (SEQ ID NO: 52)
SEAP (SEQ ID NO: 53)
Firefly Luciferase (SEQ ID NO: 54)
FMDV 2A (SEQ ID NO: 55)
Tremelimumab VL (SEQ ID NO: 16)
Tremelimumab VH (SEQ ID NO: 17)
Tremelimumab VH CDR1 (SEQ ID NO: 18)
Tremelimumab VH CDR2 (SEQ ID NO: 19)
Tremelimumab VH CDR3 (SEQ ID NO: 20)
Tremelimumab VL CDR1 (SEQ ID NO: 21)
Tremelimumab VL CDR2 (SEQ ID NO: 22)
Tremelimumab VL CDR3 (SEQ ID NO: 23)
Durvalumab (MEDI4736) VL (SEQ ID NO: 24)
MEDI4736 VH (SEQ ID NO: 25)
MEDI4736 VH CDR1 (SEQ ID NO: 26)
MEDI4736 VH CDR2 (SEQ ID NO: 27)
MEDI4736 VH CDR3 (SEQ ID NO: 28)
MEDI4736 VL CDR1 (SEQ ID NO: 29)
MEDI4736 VL CDR2 (SEQ ID NO: 30)
MEDI4736 VL CDR3 (SEQ ID NO: 31)
UbA76-25merPDTT nucleotide (SEQ ID NO: 32)
UbA76-25merPDTT polypeptide (SEQ ID NO: 33)
MAG-25merPDTT nucleotide (SEQ ID NO: 34)
MAG-25merPDTT polypeptide (SEQ ID NO: 35)
Ub7625merPDTT_NoSFL nucleotide (SEQ ID NO: 36)
Ub7625merPDTT_NoSFL polypeptide (SEQ ID NO: 37)
ChAdV68.5WTnt.MAG25mer (SEQ ID NO: 2); AC_000011.1 with E1 (nt 577 to 3403)
and E3 (nt 27, 125-31, 825) sequences deleted; corresponding ATCC VR-594
nucleotides substituted at five positions; model antigen cassette under
the control of the CMV promoter/enhancer inserted in place of deleted E1;
SV40 polyA 3′ of cassette
Venezuelan equine encephalitis virus [VEE] (SEQ ID NO: 3) GenBank: L01442.2
VEE-MAG25mer (SEQ ID NO: 4); contains MAG-25merPDTT nucleotide (bases 30-1755)
Venezuelan equine encephalitis virus strain TC-83 [TC-83] (SEQ ID NO: 5)
GenBank: L01443.1
VEE Delivery Vector (SEQ ID NO: 6); VEE genome with nucleotides 7544-11175
deleted [alphavirus structural proteins removed]
TC-83 Delivery Vector (SEQ ID NO: 7); TC-83 genome with nucleotides 7544-11175
deleted [alphavirus structural proteins removed]
VEE Production Vector (SEQ ID NO: 8); VEE genome with nucleotides 7544-11175
deleted, plus 5′ T7-promoter, plus 3′ restriction sites
TC-83 Production Vector (SEQ ID NO: 9); TC-83 genome with nucleotides 7544-11175
deleted, plus 5′ T7-promoter, plus 3′ restriction sites
VEE-UbAAY (SEQ ID NO: 14); VEE delivery vector with MHC class I mouse tumor
epitopes SIINFEKL and AH1-A5 inserted
VEE-Luciferase (SEQ ID NO: 15); VEE delivery vector with luciferase gene
inserted at 7545
ubiquitin (SEQ ID NO: 38) > UbG76 0-228
Ubiquitin A76 (SEQ ID NO: 39) > UbA76 0-228
HLA-A2 (MHC class I) signal peptide (SEQ ID NO: 40) > MHC SignalPep 0-78
HLA-A2 (MHC class I) Trans Membrane domain (SEQ ID NO: 41) > HLA A2 TM Domain
0-201
IgK Leader Seq (SEQ ID NO: 42) > IgK Leader Seq 0-60
Human DC-Lamp (SEQ ID NO: 43) > HumanDCLAMP 0-3178
Mouse LAMP1 (SEQ ID NO: 44) > MouseLamp1 0-1858
Human Lamp1 cDNA (SEQ ID NO: 45) > Human Lamp1 0-2339
Tetanus toxoid nulceic acid sequence (SEQ ID NO: 46)
Tetanus toxoid amino acid sequence (SEQ ID NO: 47)
PADRE nulceotide sequence (SEQ ID NO: 48)
PADRE amino acid sequence (SEQ ID NO: 49)
WPRE (SEQ ID NO: 50) > WPRE 0-593
IRES (SEQ ID NO: 51) > eGFP_IRES_SEAP_Insert 1746-2335
GFP (SEQ ID NO: 52)
SEAP (SEQ ID NO: 53)
Firefly Luciferase (SEQ ID NO: 54)
FMDV 2A (SEQ ID NO: 55)

### REFERENCES

1. Desrichard, A., Snyder, A. & Chan, T. A. Cancer Neoantigens and Applications for Immunotherapy. Clin. Cancer Res. Off. J. Am. Assoc. Cancer Res. (2015). doi: 10.1158/1078-0432.CCR-14-3175
2. Schumacher, T. N. & Schreiber, R. D. Neoantigens in cancer immunotherapy. Science 348, 69-74 (2015).
3. Gubin, M. M., Artyomov, M. N., Mardis, E. R. & Schreiber, R. D. Tumor neoantigens: building a framework for personalized cancer immunotherapy. J Clin. Invest. 125, 3413-3421 (2015).
4. Rizvi, N. A. et al. Cancer immunology. Mutational landscape determines sensitivity to PD-1 blockade in non-small cell lung cancer. Science 348, 124-128 (2015).
5. Snyder, A. et al. Genetic basis for clinical response to CTLA-4 blockade in melanoma. N. Engl. J Med. 371, 2189-2199 (2014).
6. Carreno, B. M. et al. Cancer immunotherapy. A dendritic cell vaccine increases the breadth and diversity of melanoma neoantigen-specific T cells. Science 348, 803-808 (2015).
7. Tran, E. et al. Cancer immunotherapy based on mutation-specific CD4+ T cells in a patient with epithelial cancer. Science 344, 641-645 (2014).
8. Hacohen, N. & Wu, C. J.-Y. United States Patent Application: 20110293637—COMPOSITIONS AND METHODS OF IDENTIFYING TUMOR SPECIFIC NEOANTIGENS. (A1). at <http://appft1.uspto.gov/netacgi/nph-Parser?Sect1=PTO1&Sect2=HITOFF&d=PG01&p=l&u=/netahtml/PTO/srchnum.html&r=l& f=G&l=50&s1=20110293637. PGNR.>
9. Lundegaard, C., Hoof, I., Lund, O. & Nielsen, M. State of the art and challenges in sequence based T-cell epitope prediction. Immunome Res. 6 Suppl 2, S3 (2010).
10. Yadav, M. et al. Predicting immunogenic tumour mutations by combining mass spectrometry and exome sequencing. Nature 515, 572-576 (2014).
11. Bassani-Stemberg, M., Pletscher-Frankild, S., Jensen, L. J. & Mann, M. Mass spectrometry of human leukocyte antigen class I peptidomes reveals strong effects of protein abundance and turnover on antigen presentation. Mol. Cell. Proteomics MCP 14, 658-673 (2015).
12. Van Allen, E. M. et al. Genomic correlates of response to CTLA-4 blockade in metastatic melanoma. Science 350, 207-211 (2015).
13. Yoshida, K. & Ogawa, S. Splicing factor mutations and cancer. Wiley Interdiscip. Rev. RNA 5, 445-459 (2014).
14. Cancer Genome Atlas Research Network. Comprehensive molecular profiling of lung adenocarcinoma. Nature 511, 543-550 (2014).
15. Rajasagi, M. et al. Systematic identification of personal tumor-specific neoantigens in chronic lymphocytic leukemia. Blood 124, 453-462 (2014).
16. Downing, S. R. et al. United States Patent Application: 0120208706—OPTIMIZATION OF MULTIGENE ANALYSIS OF TUMOR SAMPLES. (A1). at <http://appft1.uspto.gov/netacgi/nph-Parser?Sect1=PTO1&Sect2=HITOFF&d=PG01&p=l&u=/netahtml/PTO/srchnum.html&r=l& f=G&l=50&s1=20120208706. PGNR.>
17. Target Capture for NextGen Sequencing—IDT. at <http://www.idtdna.com/pages/products/nextgen/target-capture>
18. Shukla, S. A. et al. Comprehensive analysis of cancer-associated somatic mutations in class I HLA genes. Nat. Biotechnol. 33, 1152-1158 (2015).
19. Cieslik, M. et al. The use of exome capture RNA-seq for highly degraded RNA with application to clinical cancer sequencing. Genome Res. 25, 1372-1381 (2015).
20. Bodini, M. et al. The hidden genomic landscape of acute myeloid leukemia: subclonal structure revealed by undetected mutations. Blood 125, 600-605 (2015).
21. Saunders, C. T. et al. Strelka: accurate somatic small-variant calling from sequenced tumor-normal sample pairs. Bioinforma. Oxf. Engl. 28, 1811-1817 (2012).
22. Cibulskis, K. et al. Sensitive detection of somatic point mutations in impure and heterogeneous cancer samples. Nat. Biotechnol. 31, 213-219 (2013).
23. Wilkerson, M. D. et al. Integrated RNA and DNA sequencing improves mutation detection in low purity tumors. Nucleic Acids Res. 42, e107 (2014).
24. Mose, L. E., Wilkerson, M. D., Hayes, D. N., Perou, C. M. & Parker, J. S. ABRA: improved coding indel detection via assembly-based realignment. Bioinforma. Oxf Engl. 30, 2813-2815 (2014).
25. Ye, K., Schulz, M. H., Long, Q., Apweiler, R. & Ning, Z. Pindel: a pattern growth approach to detect break points of large deletions and medium sized insertions from paired-end short reads. Bioinforma. Oxf. Engl. 25, 2865-2871 (2009).
26. Lam, H. Y. K. et al. Nucleotide-resolution analysis of structural variants using BreakSeq and a breakpoint library. Nat. Biotechnol. 28, 47-55 (2010).
27. Frampton, G. M. et al. Development and validation of a clinical cancer genomic profiling test based on massively parallel DNA sequencing. Nat. Biotechnol. 31, 1023-1031 (2013).
28. Boegel, S. et al. HLA typing from RNA-Seq sequence reads. Genome Med. 4, 102 (2012).
29. Liu, C. et al. ATHLATES: accurate typing of human leukocyte antigen through exome sequencing. Nucleic Acids Res. 41, e142 (2013).
30. Mayor, N. P. et al. HLA Typing for the Next Generation. PloS One 10, e0127153 (2015).
31. Roy, C. K., Olson, S., Graveley, B. R., Zamore, P. D. & Moore, M. J. Assessing long-distance RNA sequence connectivity via RNA-templated DNA-DNA ligation. eLife 4, (2015).
32. Song, L. & Florea, L. CLASS: constrained transcript assembly of RNA-seq reads. BMC Bioinformatics 14 Suppl 5, S14 (2013).
33. Maretty, L., Sibbesen, J. A. & Krogh, A. Bayesian transcriptome assembly. Genome Biol. 15, 501 (2014).
34. Pertea, M. et al. StringTie enables improved reconstruction of a transcriptome from RNA-seq reads. Nat. Biotechnol. 33, 290-295 (2015).
35. Roberts, A., Pimentel, H., Trapnell, C. & Pachter, L. Identification of novel transcripts in annotated genomes using RNA-Seq. Bioinforma. Oxf. Engl. (2011). doi:10.1093/bioinformatics/btr355
36. Vitting-Seerup, K., Porse, B. T., Sandelin, A. & Waage, J. spliceR: an R package for classification of alternative splicing and prediction of coding potential from RNA-seq data. BMC Bioinformatics 15, 81 (2014).
37. Rivas, M. A. et al. Human genomics. Effect of predicted protein-truncating genetic variants on the human transcriptome. Science 348, 666-669 (2015).
38. Skelly, D. A., Johansson, M., Madeoy, J., Wakefield, J. & Akey, J. M. A powerful and flexible statistical framework for testing hypotheses of allele-specific gene expression from RNA-seq data. Genome Res. 21, 1728-1737 (2011).
39. Anders, S., Pyl, P. T. & Huber, W. HTSeq—a Python framework to work with high-throughput sequencing data. Bioinforma. Oxf. Engl. 31, 166-169 (2015).
40. Furney, S. J. et al. SF3B1 mutations are associated with alternative splicing in uveal melanoma. Cancer Discov. (2013). doi:10.1158/2159-8290.CD-13-0330
41. Zhou, Q. et al. A chemical genetics approach for the functional assessment of novel cancer genes. Cancer Res. (2015). doi:10.1158/0008-5472.CAN-14-2930
42. Maguire, S. L. et al. SF3B1 mutations constitute a novel therapeutic target in breast cancer. J. Pathol. 235, 571-580 (2015).
43. Carithers, L. J. et al. A Novel Approach to High-Quality Postmortem Tissue Procurement: The GTEx Project. Biopreservation Biobanking 13, 311-319 (2015).
44. Xu, G. et al. RNA CoMPASS: a dual approach for pathogen and host transcriptome analysis of RNA-seq datasets. PloS One 9, e89445 (2014).
45. Andreatta, M. & Nielsen, M. Gapped sequence alignment using artificial neural networks: application to the MHC class I system. Bioinforma. Oxf. Engl. (2015). doi:10.1093/bioinformatics/btv639
46. Jorgensen, K. W., Rasmussen, M., Buus, S. & Nielsen, M. NetMHCstab—predicting stability of peptide-MHC-I complexes; impacts for cytotoxic T lymphocyte epitope discovery. Immunology 141, 18-26 (2014).
47. Larsen, M. V. et al. An integrative approach to CTL epitope prediction: a combined algorithm integrating MHC class I binding, TAP transport efficiency, and proteasomal cleavage predictions. Eur. J. Immunol. 35, 2295-2303 (2005).
48. Nielsen, M., Lundegaard, C., Lund, O. & Keşmir, C. The role of the proteasome in generating cytotoxic T-cell epitopes: insights obtained from improved predictions of proteasomal cleavage. Immunogenetics 57, 33-41 (2005).
49. Boisvert, F.-M. et al. A Quantitative Spatial Proteomics Analysis of Proteome Turnover in Human Cells. Mol. Cell. Proteomics 11, M111.011429-M11.011429 (2012).
50. Duan, F. et al. Genomic and bioinformatic profiling of mutational neoepitopes reveals new rules to predict anticancer immunogenicity. J Exp. Med. 211, 2231-2248 (2014).
51. Janeway's Immunobiology: 9780815345312: Medicine & Health Science Books @ Amazon.com. at <http://www.amazon.com/Janeways-Immunobiology-Kenneth-Murphy/dp/0815345313>
52. Calis, J. J. A. et al. Properties of MHC Class I Presented Peptides That Enhance Immunogenicity. PLoS Comput. Biol. 9, e1003266 (2013).
53. Zhang, J. et al. Intratumor heterogeneity in localized lung adenocarcinomas delineated by multiregion sequencing. Science 346, 256-259 (2014)
54. Walter, M. J. et al. Clonal architecture of secondary acute myeloid leukemia. N. Engl. J. Med. 366, 1090-1098 (2012).
55. Hunt D F, Henderson R A, Shabanowitz J, Sakaguchi K, Michel H, Sevilir N, Cox A L, Appella E, Engelhard V H. Characterization of peptides bound to the class I MHC molecule HLA-A2.1 by mass spectrometry. Science 1992. 255: 1261-1263.
56. Zarling A L, Polefrone J M, Evans A M, Mikesh L M, Shabanowitz J, Lewis S T, Engelhard V H, Hunt D F. Identification of class I MHC-associated phosphopeptides as targets for cancer immunotherapy. Proc Natl Acad Sci USA. 2006 Oct. 3; 103(40):14889-94.
57. Bassani-Sternberg M, Pletscher-Frankild S, Jensen L J, Mann M. Mass spectrometry of human leukocyte antigen class I peptidomes reveals strong effects of protein abundance and turnover on antigen presentation. Mol Cell Proteomics. 2015 March; 14(3):658-73. doi: 10.1074/mcp.M114.042812.
58. Abelin J G, Trantham P D, Penny S A, Patterson A M, Ward S T, Hildebrand W H, Cobbold M, Bai D L, Shabanowitz J, Hunt D F. Complementary IMAC enrichment methods for HLA-associated phosphopeptide identification by mass spectrometry. Nat Protoc. 2015 September; 10(9):1308-18. doi: 10.1038/nprot.2015.086. Epub 2015 Aug. 6
59. Barnstable C J, Bodmer W F, Brown G, Galfre G, Milstein C, Williams A F, Ziegler A. Production of monoclonal antibodies to group A erythrocytes, HLA and other human cell surface antigens-new tools for genetic analysis. Cell. 1978 May; 14(1):9-20.
60. Goldman J M, Hibbin J, Kearney L, Orchard K, Th'ng K H. HLA-DR monoclonal antibodies inhibit the proliferation of normal and chronic granulocytic leukaemia myeloid progenitor cells. Br J Haematol. 1982 November; 52(3):411-20.
61. Eng J K, Jahan T A, Hoopmann M R. Comet: an open-source MS/MS sequence database search tool. Proteomics. 2013 January; 13(1):22-4. doi: 10.1002/pmic.201200439. Epub 2012 Dec. 4.
62. Eng J K, Hoopmann M R, Jahan T A, Egertson J D, Noble W S, MacCoss M J. A deeper look into Comet—implementation and features. J Am Soc Mass Spectrom. 2015 November; 26(11):1865-74. doi: 10.1007/s13361-015-1179-x. Epub 2015 Jun. 27.
63. Lukas Käll, Jesse Canterbury, Jason Weston, William Stafford Noble and Michael J. MacCoss. Semi-supervised learning for peptide identification from shotgun proteomics datasets. Nature Methods 4:923-925, November 2007
64. Lukas Käll, John D. Storey, Michael J. MacCoss and William Stafford Noble. Assigning confidence measures to peptides identified by tandem mass spectrometry. Journal of Proteome Research, 7(1):29-34, January 2008
65. Lukas Käll, John D. Storey and William Stafford Noble. Nonparametric estimation of posterior error probabilities associated with peptides identified by tandem mass spectrometry. Bioinformatics, 24(16):i42-i48, August 2008
66. Kinney R M, B J Johnson, V L Brown, D W Trent. Nucleotide Sequence of the 26 S mRNA of the Virulent Trinidad Donkey Strain of Venezuelan Equine Encephalitis Virus and Deduced Sequence of the Encoded Structural Proteins. Virology 152 (2), 400-413. 1986 Jul. 30.
67. Jill E Slansky, Frederique M Rattis, Lisa F Boyd, Tarek Fahmy, Elizabeth M Jaffee, Jonathan P Schneck, David H Margulies, Drew M Pardoll. Enhanced Antigen-Specific Antitumor Immunity with Altered Peptide Ligands that Stabilize the MHC-Peptide-TCR Complex. Immunity, Volume 13, Issue 4, 1 Oct. 2000, Pages 529-538.
68. A Y Huang, P H Gulden, A S Woods, M C Thomas, C D Tong, W Wang, V H Engelhard, G Pastemack, R Cotter, D Hunt, D M Pardoll, and E M Jaffee. The immunodominant major histocompatibility complex class I-restricted antigen of a murine colon tumor derives from an endogenous retroviral gene product. Proc Natl Acad Sci USA.; 93(18): 9730-9735, 1996 Sep. 3.
69. JOHNSON, BARBARA J. B., RICHARD M. KINNEY, CRYSTLE L. KOST AND DENNIS W. TRENT. Molecular Determinants of Alphavirus Neurovirulence: Nucleotide and Deduced Protein Sequence Changes during Attenuation of Venezuelan Equine Encephalitis Virus. J Gen Virol 67:1951-1960, 1986.
70. Aarnoudse, C. A., Kruse, M., Konopitzky, R., Brouwenstijn, N., and Schrier, P. I. (2002). TCR reconstitution in Jurkat reporter cells facilitates the identification of novel tumor antigens by cDNA expression cloning. Int J Cancer 99, 7-13.
71. Alexander, J., Sidney, J., Southwood, S., Ruppert, J., Oseroff, C., Maewal, A., Snoke, K., Serra, H. M., Kubo, R. T., and Sette, A. (1994). Development of high potency universal DR-restricted helper epitopes by modification of high affinity DR-blocking peptides. Immunity 1, 751-761.
72. Banu, N., Chia, A., Ho, Z. Z., Garcia, A. T., Paravasivam, K., Grotenbreg, G. M., Bertoletti, A., and Gehring, A. J. (2014). Building and optimizing a virus-specific T cell receptor library for targeted immunotherapy in viral infections. Scientific Reports 4, 4166.
73. Comet, S., Miconnet, I., Menez, J., Lemonnier, F., and Kosmatopoulos, K. (2006). Optimal organization of a polypeptide-based candidate cancer vaccine composed of cryptic tumor peptides with enhanced immunogenicity. Vaccine 24, 2102-2109.
74. Depla, E., van der Aa, A., Livingston, B. D., Crimi, C., Allosery, K., de Brabandere, V., Krakover, J., Murthy, S., Huang, M., Power, S., et al. (2008). Rational design of a multiepitope vaccine encoding T-lymphocyte epitopes for treatment of chronic hepatitis B virus infections. Journal of Virology 82, 435-450.
75. Ishioka, G. Y., Fikes, J., Hermanson, G., Livingston, B., Crimi, C., Qin, M., del Guercio, M. F., Oseroff, C., Dahlberg, C., Alexander, J., et al. (1999). Utilization of MHC class I transgenic mice for development of minigene DNA vaccines encoding multiple HLA-restricted CTL epitopes. J Immunol 162, 3915-3925.
76. Janetzki, S., Price, L., Schroeder, H., Britten, C. M., Welters, M. J. P., and Hoos, A. (2015). Guidelines for the automated evaluation of Elispot assays. Nat Protoc 10, 1098-1115.
77. Lyons, G. E., Moore, T., Brasic, N., Li, M., Roszkowski, J. J., and Nishimura, M. I. (2006). Influence of human CD8 on antigen recognition by T-cell receptor-transduced cells. Cancer Res 66, 11455-11461.
78. Nagai, K., Ochi, T., Fujiwara, H., An, J., Shirakata, T., Mineno, J., Kuzushima, K., Shiku, H., Melenhorst, J. J., Gostick, E., et al. (2012). Aurora kinase A-specific T-cell receptor gene transfer redirects T lymphocytes to display effective antileukemia reactivity. Blood 119, 368-376.
79. Panina-Bordignon, P., Tan, A., Termijtelen, A., Demotz, S., Corradin, G., and Lanzavecchia, A. (1989). Universally immunogenic T cell epitopes: promiscuous binding to human MHC class II and promiscuous recognition by T cells. Eur J Immunol 19, 2237-2242.
80. Vitiello, A., Marchesini, D., Furze, J., Sherman, L. A., and Chesnut, R. W. (1991). Analysis of the HLA-restricted influenza-specific cytotoxic T lymphocyte response in transgenic mice carrying a chimeric human-mouse class I major histocompatibility complex. J Exp Med 173, 1007-1015.
81. Yachi, P. P., Ampudia, J., Zal, T., and Gascoigne, N. R. J. (2006). Altered peptide ligands induce delayed CD8-T cell receptor interaction—a role for CD8 in distinguishing antigen quality. Immunity 25, 203-211.
82. Pushko P, Parker M, Ludwig G V, Davis N L, Johnston R E, Smith J F. Replicon-helper systems from attenuated Venezuelan equine encephalitis virus: expression of heterologous genes in vitro and immunization against heterologous pathogens in vivo. Virology. 1997 Dec. 22; 239(2):389-401.
83. Strauss, J H and E G Strauss. The alphaviruses: gene expression, replication, and evolution. Microbiol Rev. 1994 September; 58(3): 491-562.
84. Rhême C, Ehrengruber M U, Grandgirard D. Alphaviral cytotoxicity and its implication in vector development. Exp Physiol. 2005 January; 90(1):45-52. Epub 2004 Nov. 12.
85. Riley, Michael K. I I, and Wilfred Vermerris. Recent Advances in Nanomaterials for Gene Delivery-A Review. Nanomaterials 2017, 7(5), 94.
86. Frolov I, Hardy R, Rice C M. Cis-acting RNA elements at the 5′ end of Sindbis virus genome RNA regulate minus- and plus-strand RNA synthesis. RNA. 2001 November; 7(11):1638-51.
87. Jose J, Snyder J E, Kuhn R J. A structural and functional perspective of alphavirus replication and assembly. Future Microbiol. 2009 September; 4(7):837-56.
88. Bo Li and C. olin N. Dewey. RSEM: accurate transcript quantification from RNA-Seq data with or without a referenfe genome. BMC Bioinformatics, 12:323, August 2011
89. Hillary Pearson, Tariq Daouda, Diana Paola Granados, Chantal Durette, Eric Bonneil, Mathieu Courcelles, Anja Rodenbrock, Jean-Philippe Laverdure, Caroline Cöté, Sylvie Mader, Sebastien Lemieux, Pierre Thibault, and Claude Perreault. MHC class I-associated peptides derive from selective regions of the human genome. The Journal of Clinical Investigation, 2016,
90. Juliane Liepe, Fabio Marino, John Sidney, Anita Jeko, Daniel E. Bunting, Alessandro Sette, Peter M. Kloetzel, Michael P. H. Stumpf, Albert J. R. Heck, Michele Mishto. A large fraction of HLA class I ligands are proteasome-generated spliced peptides. Science, 21, October 2016.
91. Mommen G P., Marino, F., Meiring H D., Poelen, M C., van Gaans-van den Brink, J A., Mohammed S., Heck A J., and van Els C A. Sampling From the Proteome to the Human Leukocyte Antigen-DR (HLA-DR) Ligandome Proceeds Via High Specificity. Mol Cell Proteomics 15(4): 1412-1423, April 2016.
92. Sebastian Kreiter, Mathias Vormehr, Niels van de Roemer, Mustafa Diken, Martin Löwer, Jan Diekmann, Sebastian Boegel, Barbara Schrörs, Fulvia Vascotto, John C. Castle, Arbel D. Tadmor, Stephen P. Schoenberger, Christoph Huber, Özlem Türeci, and Ugur Sahin. Mutant MHC class II epitopes drive therapeutic immune responses to caner. Nature 520, 692-696, April 2015.
93. Tran E., Turcotte S., Gros A., Robbins P. F., Lu Y. C., Dudley M. E., Wunderlich J. R., Somerville R. P., Hogan K., Hinrichs C. S., Parkhurst M. R., Yang J. C., Rosenberg S. A. Cancer immunotherapy based on mutation-specific CD4+ T cells in a patient with epithelial cancer. Science 344(6184) 641-645, May 2014.
94. Andreatta M., Karosiene E., Rasmussen M., Stryhn A., Buus S., Nielsen M. Accurate pan-specific prediction of peptide-MHC class II binding affinity with improved binding core identification. Immunogenetics 67(11-12) 641-650, November 2015.
95. Nielsen, M., Lund, O. NN-align. An artificial neural network-based alignment algorithm for MHC class II peptide binding prediction. BMC Bioinformatics 10:296, September 2009.
96. Nielsen, M., Lundegaard, C., Lund, O. Prediction of MHC class II binding affinity using SMM-align, a novel stabilization matrix alignment method. BMC Bioinformatics 8:238, July 2007.
97. Zhang, J., et al. PEAKS D B: de novo sequencing assisted database search for sensitive and accurate peptide identification. Molecular & Cellular Proteomics. 11(4):1-8. Jan. 2, 2012.
98. Jensen, Kamilla Kjaergaard, et al. “Improved Methods for Prediting Peptide Binding Affinity to MHC Class II Molecules.” Immunology, 2018, doi:10.1111/imm.12889.
99. Carter, S. L., Cibulskis, K., Helman, E., McKenna, A., Shen, H., Zack, T., Laird, P. W., Onofrio, R. C., Winckler, W., Weir, B. A., et al. (2012). Absolute quantification of somatic DNA alterations in human cancer. Nat. Biotechnol. 30, 413-421
100. McGranahan, N., Rosenthal, R., Hiley, C. T., Rowan, A. J., Watkins, T. B. K., Wilson, G. A., Birkbak, N. J., Veeriah, S., Van Loo, P., Herrero, J., et al. (2017). Allele-Specific HLA Loss and Immune Escape in Lung Cancer Evolution. Cell 171, 1259-1271.e11.
101. Shukla, S. A., Rooney, M. S., Rajasagi, M., Tiao, G., Dixon, P. M., Lawrence, M. S., Stevens, J., Lane, W. J., Dellagatta, J. L., Steelman, S., et al. (2015). Comprehensive analysis of cancer-associated somatic mutations in class I HLA genes. Nat. Biotechnol. 33, 1152-1158.
102. Van Loo, P., Nordgard, S. H., Lingjorde, O. C., Russnes, H. G., Rye, I. H., Sun, W., Weigman, V. J., Marynen, P., Zetterberg, A., Naume, B., et al. (2010). Allele-specific copy number analysis of tumors. Proc. Natl. Acad. Sci. U.S.A 107, 16910-16915.
103. Van Loo, P., Nordgard, S. H., Lingjorde, O. C., Russnes, H. G., Rye, I. H., Sun, W., Weigman, V. J., Marynen, P., Zetterberg, A., Naume, B., et al. (2010). Allele-specific copy number analysis of tumors. Proc. Natl. Acad. Sci. U.S.A 107, 16910-16915.
104. HIV Sequence Compendium 2018 Foley B, Leitner T, Apetrei C, Hahn B, Mizrachi I, Mullins J, Rambaut A, Wolinsky S, and Korber B, Eds. Published by Theoretical Biology and Biophysics Group, Los Alamos National Laboratory, NM, LA-UR 18-25673.
105. Llano, A., Williams, A., Olvera, A., Silva-Arrieta, S., Brander, C., (2013). Best-Characterized HIV-1 CTL Epitopes: The 2013 Update. HIV Molecular Immunology, 3-25.
106. Gaiha, G., Rossin, E., Urbach, J., et al. (2019). Structural topology defines protective CD8+ T cell epitopes in the HIV proteome. Science 364, 480-484.
107. O'Donnell, T. J., Rubinsteyn, A., Bonsack, M., Riemer, A. B., Laserson, U., & Hammerbacher, J. (2018). MHCflurry: Open-Source Class I MHC Binding Affinity Prediction. Cell Systems, 7(1), 129-132.e4.
108. Los Almos National Security, LLC, “Best-defined CTL/CD8+ Epitope Summary”, 20 Nov. 2019, https://www.hiv.lanl.gov/content/immunology/tables/optimal_ctl_summary.html.
109. Llano A, Cedeño, S, Silva-Arrieta, S, Brander C (2019). The 2019 Optimal HIV CTL epitopes update: Growing diversity in epitope length and HLA restriction. in HIV Molecular Immunology 2019. Yusim, K, Korber B, Brander, C, Barouch, D, de Boer, R, Haynes, B F, Koup, R, Moore, J P, Walker, B D, Eds. Published by Theoretical Biology and Biophysics Group, Los Alamos National Laboratory, Los Alamos, N. Mex.

Lengthy table referenced here
US20230128001A1-20230427-T00001
Please refer to the end of the specification for access instructions.

Lengthy table referenced here
US20230128001A1-20230427-T00002
Please refer to the end of the specification for access instructions.

Lengthy table referenced here
US20230128001A1-20230427-T00003
Please refer to the end of the specification for access instructions.

Lengthy table referenced here
US20230128001A1-20230427-T00004
Please refer to the end of the specification for access instructions.

Lengthy table referenced here
US20230128001A1-20230427-T00005
Please refer to the end of the specification for access instructions.

Lengthy table referenced here
US20230128001A1-20230427-T00006
Please refer to the end of the specification for access instructions.

Lengthy table referenced here
US20230128001A1-20230427-T00007
Please refer to the end of the specification for access instructions.

Lengthy table referenced here
US20230128001A1-20230427-T00008
Please refer to the end of the specification for access instructions.

Lengthy table referenced here
US20230128001A1-20230427-T00009
Please refer to the end of the specification for access instructions.

Lengthy table referenced here
US20230128001A1-20230427-T00010
Please refer to the end of the specification for access instructions.

Lengthy table referenced here
US20230128001A1-20230427-T00011
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20230128001A1). An electronic copy of the table will also be available from the USPTO upon request and payment of the fee set forth in 37 CFR 1.19(b)(3).

1. A composition for delivery of an antigen expression system or for delivery of one or more antigens, wherein:
(A) the antigen expression system comprises a vector backbone comprising a chimpanzee adenovirus vector, optionally wherein the chimpanzee adenovirus vector is a ChAdV68 vector, or an alphavirus vector, optionally wherein the alphavirus vector is a Venezuelan equine encephalitis virus vector, the vector backbone comprising at least one HIV MHC class I antigen-encoding nucleic acid sequence comprising a MHC class I epitope encoding nucleic acid sequence, optionally wherein the MHC class I epitope encoding nucleic acid sequence encodes a MHC class I epitope comprising at least one HIV epitope selected from the group consisting of the sequences shown in SEQ ID NOs: 325-22349; or
(B) the composition for delivery of one or more antigens comprises one or more HIV MHC class I antigens or one or more nucleic acid sequences encoding one or more HIV MHC class I antigens, each HIV MHC class I antigen comprising a MHC class I epitope comprising at least one HIV epitope selected from the group consisting of the sequences shown in SEQ ID Nos: 325-22349.

2. The composition of claim 1, wherein the at least one HIV epitope is selected from the group consisting of the sequences shown in SEQ ID NOs: 4113, 4114, 4115, 4427, 4439, 4494, 4495, 4545, 4561, 4956, 4968, 4975, 4982, 5259, 5261, 5459, 5460, 5610, 5643, and 5661.

3. The composition of claim 1, wherein the antigen expression system comprises 2, 3, 4, 5, 6, 7, 8, 9, 10, 11, 12, 13, 14, 15, 16, 17, 18, 19, or 20 HIV MHC class I antigen-encoding nucleic acid sequences, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID Nos: 325-22349.

4. The composition of claim 3, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 4113, 4114, 4115, 4427, 4439, 4494, 4495, 4545, 4561, 4956, 4968, 4975, 4982, 5259, 5261, 5459, 5460, 5610, 5643, and 5661.

5. (canceled)

6. (canceled)

7. (canceled)

8. (canceled)

9. (canceled)

10. A composition for delivery of an antigen expression system comprising one or more vectors, the one or more vectors comprising:
(a) a vector backbone, wherein the backbone comprises:
(i) at least one promoter nucleotide sequence, and
(ii) at least one polyadenylation (poly(A)) sequence; and
(b) an antigen cassette, wherein the antigen cassette comprises:
(i) at least one antigen-encoding nucleic acid sequence, comprising:
(I) at least one HIV MHC class I antigen-encoding nucleic acid sequence, comprising:
(A) a MHC class I epitope encoding nucleic acid sequence, wherein the MHC class I epitope encoding nucleic acid sequence encodes a MHC class I epitope comprising at least one HIV epitope selected from the group consisting of the sequences shown in SEQ ID Nos: 325-22349,
(B) optionally, a 5′ linker sequence, and
(C) optionally, a 3′ linker sequence;
(ii) optionally, a second promoter nucleotide sequence operably linked to the antigen-encoding nucleic acid sequence; and
(iii) optionally, at least one MHC class II antigen-encoding nucleic acid sequence;
(iv) optionally, at least one nucleic acid sequence encoding a GPGPG (SEQ ID NO: 151) amino acid linker sequence; and
(v) optionally, at least one second poly(A) sequence, wherein the second poly(A) sequence is a native poly(A) sequence or an exogenous poly(A) sequence to the vector backbone.

11. The composition of claim 10, wherein the at least one antigen-encoding nucleic acid sequence comprises:
(I-a) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 325-2165;
or
(I-b) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 2166-4106;
or
(I-c) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 4107-6241:
or
(I-d) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 6242-8389: or
(I-e) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 8930-10626; or
(I-f) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 10627-12810; or
(I-g) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 12811-15079;
or
(I-h) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope selected from the group consisting of epitope sequences of any one of SEQ ID NOs: 15080-17174;
or
(I-i) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 17175-19388;
or
(I-j) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 19389-21003;
or
(I-k) at least 10, 11, 12, 13, 14, 15, 16, 17, 18, 19, or 20 HIV MHC class I antigen-encoding nucleic acid sequences linearly linked to each other, wherein each HIV MHC class I antigen-encoding nucleic acid sequence comprises a MHC class I epitope encoding nucleic acid sequence that encodes a MHC class I epitope comprising at least one HIV epitope selected from the group consisting of the sequences shown in SEQ ID NOs: 21004-22349, and
wherein each of the HIV MHC class I antigen-encoding nucleic acid sequences further comprises;
(A) optionally, a 5′ linker sequence, and
(B) optionally, a 3′ linker sequence.

12-21. (canceled)

22. The composition of claim 10, wherein:
(a) the vector backbone comprises:
(i) a chimpanzee adenovirus vector, optionally wherein the chimpanzee adenovirus vector is a ChAdV68 vector, or an alphavirus vector, optionally wherein the alphavirus vector is a Venezuelan equine encephalitis virus vector, and
(ii) a 26S promoter nucleotide sequence, and
(iii) a polyadenylation (poly(A)) sequence; and
(b) the antigen cassette is integrated between the 26S promoter nucleotide sequence and the poly(A) sequence, wherein the antigen cassette comprises:
(i) the at least one antigen-encoding nucleic acid sequence, comprising:
(I) the at least 10, 11, 12, 13, 14, 15, 16, 17, 18, 19, or 20 HIV MHC class I antigen-encoding nucleic acid sequences linearly linked to each other and each comprising:
(A) the optional 5′ linker sequence, wherein the 5′ linker sequence encodes a native N-terminal amino acid sequence of the MHC class I epitope, and wherein the 5′ linker sequence encodes a peptide that is at least 3 amino acids in length,
(B) the optional 3′ linker sequence, wherein the 3′ linker sequence encodes a native C-terminal acid sequence of the MHC class I epitope, and wherein the 3′ linker sequence encodes a peptide that is at least 3 amino acids in length, and
wherein the antigen cassette is operably linked to the 26S promoter nucleotide sequence, wherein each of the MHC class I antigen-encoding nucleic acid sequences encodes a polypeptide that is between 13 and 25 amino acids in length, and wherein each 3′ end of each MHC class I antigen-encoding nucleic acid sequence is linked to the 5′ end of the following MHC class I antigen-encoding nucleic acid sequence with the exception of the final MHC class I antigen-encoding nucleic acid sequence in the antigen cassette; and
(ii) at least two MHC class II antigen-encoding nucleic acid sequences comprising:
(I) a PADRE MHC class II sequence,
(II) a Tetanus toxoid MHC class II sequence,
(III) a first nucleic acid sequence encoding the at least one nucleic acid sequence encoding GPGPG (SEQ ID NO: 163) amino acid linker sequence linking the PADRE MHC class II sequence and the Tetanus toxoid MHC class II sequence,
(IV) a second nucleic acid sequence encoding the at least one nucleic acid sequence encoding the GPGPG (SEQ ID NO: 164) amino acid linker sequence linking the 5′ end of the at least two MHC class II antigen-encoding nucleic acid sequences to the HIV MHC class I antigen-encoding nucleic acid sequences,
(V) optionally, a third nucleic acid sequence encoding at least one nucleic acid sequence encoding the GPGPG (SEQ ID NO: 165) amino acid linker sequence at the 3′ end of the at least two MHC class II antigen-encoding nucleic acid sequences.

23. The composition of claim 10, wherein an ordered sequence of each element of the antigen cassette is described in the formula, from 5′ to 3′, comprising:
Pₐ-(L5_{b}-N_{c}-L3_{d})_{X}-(G5ₑ-U_{f})_{Y}-G3_{g}
wherein P comprises the second promoter nucleotide sequence, where a=0 or 1,
N comprises one of the MHC class I epitope encoding nucleic acid sequences, where c=1,
L5 comprises the 5′ linker sequence, where b=0 or 1,
L3 comprises the 3′ linker sequence, where d=0 or 1,
G5 comprises one of the at least one nucleic acid sequences encoding a GPGPG (SEQ ID NO: 166) amino acid linker, where e=0 or 1,
G3 comprises one of the at least one nucleic acid sequences encoding a GPGPG (SEQ ID NO: 167) amino acid linker, where g=0 or 1,
U comprises one of the at least one MHC class II antigen-encoding nucleic acid sequence, where f=1,
X=1 to 400, where for each X the corresponding N_{c} is a epitope encoding nucleic acid sequence, and
Y=0, 1, or 2, where for each Y the corresponding U_{f} is an antigen-encoding nucleic acid sequence.

24-38. (canceled)

39. The composition of claim 1, wherein the vector backbone the alphavirus vector comprising at least one nucleotide sequence of a Venezuelan equine encephalitis virus, wherein the Venezuelan equine encephalitis virus comprises the sequence of SEQ ID NO:3 or SEQ ID NO:5 further comprising a deletion between base pair 7544 and 11175, and wherein the cassette is inserted at position 7544 to replace the deletion between base pairs 7544 and 11175 as set forth in the sequence of SEQ ID NO:3 or SEQ ID NO:5.

40-49. (canceled)

50. The composition of claim 1, wherein the vector backbone comprises the chimpanzee adenovirus vector, wherein the chimpanzee adenovirus vector is a ChAdV68 vector, and wherein the ChAdV68 backbone comprises at least nucleotides 2 to 36,518 of the sequence set forth in SEQ ID NO:1, wherein the nucleotides 2 to 36,518 lack: (1) nucleotides 577 to 3403 of the sequence shown in SEQ ID NO:1 corresponding to an E1 deletion; (2) nucleotides 27,125 to 31,825 of the sequence shown in SEQ ID NO:1 corresponding to an E3 deletion; and (3) nucleotides 34,916 to 35,642 of the sequence shown in SEQ ID NO:1 corresponding to a partial E4 deletion: optionally wherein the antigen cassette is inserted within the E1 deletion.

51-98. (canceled)

99. The composition of claim 10, wherein the antigen cassette comprises junctional epitope sequences formed by adjacent sequences in the antigen cassette, wherein at least one or each junctional epitope sequence has an affinity of greater than 500 nM for MHC, optionally wherein each junctional epitope sequence is non-self.

100. (canceled)

101. (canceled)

102. (canceled)

103. (canceled)

104. (canceled)

105. A pharmaceutical composition comprising the composition claim 1 and a pharmaceutically acceptable carrier.

106. (canceled)

107. An isolated nucleotide sequence or set of isolated nucleotide sequences comprising the antigen cassette of claim 10 and one or more elements obtained from the sequence of SEQ ID NO:3 or SEQ ID NO:5, optionally wherein the one or more elements are selected from the group consisting of the sequences necessary for nonstructural protein-mediated amplification, the 26S promoter nucleotide sequence, the poly(A) sequence, and the nsP1-4 genes of the sequence set forth in SEQ ID NO:3 or SEQ ID NO:5, and optionally wherein the nucleotide sequence is cDNA, and optionally wherein the sequence or set of isolated nucleotide sequences comprises the antigen cassette of claim 10 inserted at position 7544 of the sequence set forth in SEQ ID NO:6 or SEQ ID NO:7.

108. (canceled)

109. (canceled)

110. (canceled)

111. (canceled)

112. An isolated cell comprising the nucleotide sequence or set of isolated nucleotide sequences of claim 107, optionally wherein the cell is a BHK-21, CHO, HEK293 or variants thereof, 911, HeLa, A549, LP-293, PER.C6, or AE1-2a cell.

113. A method for treating a subject with HIV, the method comprising administering to the subject the composition of claim 1.

114. A method for inducing an immune response in a subject, the method comprising administering to the subject the composition of claim 1.

115-130. (canceled)

131. A method of assessing a subject having HIV, comprising the steps of:
a) determining or having determined a HIV subtype of the HIV of the subject;
b) determining or having determined whether the subject expresses a HLA allele predicted or known to present a MHC class I epitope encoded by an antigen-encoding nucleic acid sequence in an antigen-based vaccine, and
c) determining or having determined that the subject is a candidate for therapy with the antigen-based vaccine when the subject expresses the HLA allele, and the HIV subtype expresses the MHC class I epitope encoded by the antigen-encoding nucleic acid sequence in the antigen-based vaccine,
wherein the MHC class I epitope comprises at least one MHC class I epitope sequence selected from the group consisting of epitope sequences from any one of SEQ ID Nos: 325-22349, and
d) optionally, administering or having administered the antigen-based vaccine to the subject.

132-189. (canceled)

190. The composition of claim 1, wherein each MHC class I epitope comprises at least one HIV epitope selected from the group consisting of the sequences shown in:
(a) SEQ ID NOs: 325-2165;
(b) SEQ ID NOs: 2166-4106;
(c) SEQ ID NOs: 4107-6241;
(d) SEQ ID NOs: 6242-8389;
(e) SEQ ID NOs: 8930-10626;
(f) SEQ ID NOs: 10627-12810;
(g) SEQ ID NOs: 12811-15079;
(h) SEQ ID NOs: 15080-17174;
(i) SEQ ID NOs: 17175-19388;
(j) SEQ ID NOs: 19389-21003; or selected from
(k) SEQ ID NOs: 21004-22349.

191-205. (canceled)

206. A method for treating a subject with HIV, the method comprising administering to the subject the composition of claim 10.

207. A method for inducing an immune response in a subject, the method comprising administering to the subject the composition of claim 10.

208. The method of claim 131, wherein:
if the HIV of the subject is HIV subtype A1 then the MHC class I epitope comprises at least one MHC class I epitope sequence comprising at least one HIV epitope selected from the group consisting of the sequences shown in SEQ ID NOs: 325-2165, and optionally the HLA allele expressed by the subject is selected from the group consisting of HLA alleles in Table 35; or
if the HIV of the subject is HIV subtype A2 then the MHC class I epitope comprises at least one MHC class I epitope sequence comprising at least one HIV epitope selected from the group consisting of the sequences shown in SEQ ID NOs: 2166-4106, and optionally the HLA allele expressed by the subject is selected from the group consisting of HLA alleles in Table 36; or
if the HIV of the subject is HIV subtype B then the MHC class I epitope comprises at least one MHC class I epitope sequence comprising at least one HIV epitope selected from the group consisting of the sequences shown in SEQ ID NOs: 4107-6241, and optionally wherein the HLA allele expressed by the subject is selected from the group consisting of HLA alleles in Table 37; or
if the HIV of the subject is HIV subtype C then the MHC class I epitope comprises at least one MHC class I epitope sequence comprising at least one HIV epitope selected from the group consisting of the sequences shown in SEQ ID NOs: 6242-8389, and optionally wherein the HLA allele expressed by the subject is selected from the group consisting of HLA alleles in Table 38; or
if the HIV of the subject is HIV subtype D then the MHC class I epitope comprises at least one MHC class I epitope sequence comprising at least one HIV epitope selected from the group consisting of the sequences shown in SEQ ID NOs: 8930-10626, and optionally the HLA allele expressed by the subject is selected from the group consisting of HLA alleles in Table 39; or
if the HIV of the subject is HIV subtype F1 then the MHC class I epitope comprises at least one HIV epitope selected from the group consisting of the sequences shown in SEQ ID NOs: 10627-12810, and optionally the HLA allele expressed by the subject is selected from the group consisting of HLA alleles in Table 40; or
if the HIV of the subject is HIV subtype F2 then the MHC class I epitope comprises at least one HIV epitope selected from the group consisting of the sequences shown in SEQ ID NOs: 12811-15079, and optionally the HLA allele expressed by the subject is selected from the group consisting of HLA alleles in Table 41; or
if the HIV of the subject is HIV subtype G then the MHC class I epitope comprises at least one HIV epitope selected from the group consisting of the sequences shown in SEQ ID NOs: 15080-17174, and optionally wherein the HLA allele expressed by the subject is selected from the group consisting of HLA alleles in Table 42; or
if the HIV of the subject is HIV subtype H, then the MHC class I epitope comprises at least one MHC class I epitope sequence comprising at least one HIV epitope selected from the group consisting of the sequences shown in SEQ ID NOs: 17175-19388, and optionally wherein the HLA allele expressed by the subject is selected from the group consisting of HLA alleles in Table 43; or
if the HIV of the subject is HIV subtype J then the MHC class I epitope comprises at least one MHC class I epitope sequence comprising at least one HIV epitope selected from the group consisting of the sequences shown in SEQ ID NOs: 19389-21003, and optionally wherein the HLA allele expressed by the subject is selected from the group consisting of HLA alleles in Table 44; or
if the HIV of the subject is HIV subtype K then the MHC class I epitope comprises at least one MHC class I epitope sequence comprising at least one HIV epitope selected from the group consisting of the sequences shown in SEQ ID NOs: 21004-22349, and optionally wherein the HLA allele expressed by the subject is selected from the group consisting of HLA alleles in Table 45.
